SEQUENCE LISTING (3649 sequences)

SEQ ID NO: 1-38 - skipped (38 entries)

SEQ ID NO: 39 - protein, 107 aa, Homo sapiens [sequence omitted]

SEQ ID NO: 40 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 41 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43-72 - skipped (30 entries)

SEQ ID NO: 73 - protein, 453 aa, synthetic construct
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLASSLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 74 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 75 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 76 - protein, 159 aa, Homo sapiens
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 77-2169 - skipped (2093 entries)

SEQ ID NO: 2170 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 2171-2179 - skipped (9 entries)

SEQ ID NO: 2180 - protein, 77 aa, synthetic construct
MAPRRARGCRTLGLPALLLLLLLRPPATRGITCPPPMSVEHADIWVKSYSLYSRERYICNSGFKRKAGTSSLTECVL

SEQ ID NO: 2181-2189 - skipped (9 entries)

SEQ ID NO: 2190 - protein, 20 aa, synthetic construct
SGGSGGGGSGGGSGGGGSLQ

SEQ ID NO: 2191 - protein, 133 aa, Homo sapiens [sequence omitted]

SEQ ID NO: 2192 - protein, 157 aa, Homo sapiens
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 2193 - protein, 133 aa, Homo sapiens [sequence omitted]

SEQ ID NO: 2194 - protein, 138 aa, Homo sapiens
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRG

SEQ ID NO: 2195-2269 - skipped (75 entries)

SEQ ID NO: 2270 - protein, 133 aa, Homo sapiens
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 2271-2279 - skipped (9 entries)

SEQ ID NO: 2280 - protein, 133 aa, Homo sapiens
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 2281-2289 - skipped (9 entries)

SEQ ID NO: 2290 - protein, 518 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 2291-2299 - skipped (9 entries)

SEQ ID NO: 2300 - protein, 340 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGGGSGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 2301-2309 - skipped (9 entries)

SEQ ID NO: 2310 - protein, 166 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 2311-2319 - skipped (9 entries)

SEQ ID NO: 2320 - protein, 114 aa, Homo sapiens
NWVNVISDLKKIEDLIQSMHIDATLYTESDVHPSCKVTAMKCFLLELQVISLASGDASIHDTVENLIILANNSLSSNGAVTESGCKECEELEEKNIKEFLQSFVHIVQMFINTS

SEQ ID NO: 2321-3290 - skipped (970 entries)

SEQ ID NO: 3291 - protein, 238 aa, Homo sapiens
DLKVEMMAGGTQITPLNDNVTIFCNIFYSQPLNITSMGITWFWKSLTFDKEVKVFEFFGDHQEAFRPGAIVSPWRLKSGDASLRLPGIQLEEAGEYRCEVVVTPLKAQGTVQLEVVASPASRLLLDQVGMKENEDKYMCESSGFYPEAINITWEKQTQKFPHPIEISEDVITGPTIKNMDGTFNVTSCLKLNSSQEDPGTVYQCVVRHASLHTPLRSNFTLTAARHSLSETEKTDNFS

SEQ ID NO: 3292 - protein, 284 aa, Homo sapiens
EPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSHW

SEQ ID NO: 3293 - protein, 287 aa, Homo sapiens
AEPHSLRYNLMVLSQDESVQSGFLAEGHLDGQPFLRYDRQKRRAKPQGQWAEDVLGAKTWDTETEDLTENGQDLRRTLTHIKDQKGGLHSLQEIRVCEIHEDSSTRGSRHFYYDGELFLSQNLETQESTVPQSSRAQTLAMNVTNFWKEDAMKTKTHYRAMQADCLQKLQRYLKSGVAIRRTVPPMVNVTCSEVSEGNITVTCRASSFYPRNITLTWRQDGVSLSHNTQQWGDVLPDGNGTYQTWVATRIRQGEEQRFTCYMEHSGNHGTHPVPSGKVLVLQSQRTD

SEQ ID NO: 3294 - protein, 191 aa, Homo sapiens
GWVDTHCLCYDFIITPKSRPEPQWCEVQGLVDERPFLHYDCVNHKAKAFASLGKKVNVTKTWEEQTETLRDVVDFLKGQLLDIQVENLIPIEPLTLQARMSCEHEAHGHGRGSWQFLFNGQKFLLFDSNNRKWTALHPGAKKMTEKWEKNRDVTMFFQKISLGDCKMWLEEFLMYWEQMLDPTKPPSLAPG

SEQ ID NO: 3295 - protein, 329 aa, Homo sapiens
QDVRVQVLPEVRGQLGGTVELPCHLLPPVPGLYISLVTWQRPDAPANHQNVAAFHPKMGPSFPSPKPGSERLSFVSAKQSTGQDTEAELQDATLALHGLTVEDEGNYTCEFATFPKGSVRGMTWLRVIAKPKNQAEAQKVTFSQDPTTVALCISKEGRPPARISWLSSLDWEAKETQVSGTLAGTVTVTSRFTLVPSGRADGVTVTCKVEHESFEEPALIPVTLSVRYPPEVSISGYDDNWYLGRTDATLSCDVRSNPEPTGYDWSTTSGTFPTSAVAQGSQLVIHAVDSLFNTTFVCTVTNAVGMGRAEQVIFVRETPNTAGAGATGG

SEQ ID NO: 3296 - protein, 323 aa, Homo sapiens
WPPPGTGDVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQVKEGPPSEHSGISRN

SEQ ID NO: 3297 - protein, 330 aa, Homo sapiens
QNLFTKDVTVIEGEVATISCQVNKSDDSVIQLLNPNRQTIYFRDFRPLKDSRFQLLNFSSSELKVSLTNVSISDEGRYFCQLYTDPPQESYTTITVLVPPRNLMIDIQKDTAVEGEEIEVNCTAMASKPATTIRWFKGNTELKGKSEVEEWSDMYTVTSQLMLKVHKEDDGVPVICQVEHPAVTGNLQTQRYLEVQYKPQVHIQMTYPLQGLTREGDALELTCEAIGKPQPVMVTWVRVDDEMPQHAVLSGPNLFINNLNKTDNGTYRCEASNIVGKAHSDYMLYVYDPPTTIPPPTTTTTTTTTTTTTILTIITDSRAGEEGSIRAVDH

SEQ ID NO: 3298 - protein, 155 aa, Homo sapiens
QRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 3299 - protein, 294 aa, Homo sapiens
WTYHYSEKPMNWQRARRFCRDNYTDLVAIQNKAEIEYLEKTLPFSRSYYWIGIRKIGGIWTWVGTNKSLTEEAENWGDGEPNNKKNKEDCVEIYIKRNKDAGKWNDDACHKLKAALCYTASCQPWSCSGHGECVEIINNYTCNCDVGYYGPQCQFVIQCEPLEAPELGTMDCTHPLGNFSFSSQCAFSCSEGTNLTGIEETTCGPFGNWSSPEPTCQVIQCEPLSAPDLGIMNCSHPLASFSFTSACTFICSEGTELIGKKKTICESSGIWSNPSPICQKLDKSFSMIKEGDYN

SEQ ID NO: 3300 - protein, 243 aa, Homo sapiens
RYLQVSQQLQQTNRVLEVTNSSLRQQLRLKITQLGQSAEDLQGSRRELAQSQEALQVEQRAHQAAEGQLQACQADRQKTKETLQSEEQQRRALEQKLSNMENRLKPFFTCGSADTCCPSGWIMHQKSCFYISLTSKNWQESQKQCETLSSKLATFSEIYPQSHSYYFLNSLLPNGGSGNSYWTGLSSNKDWKLTDDTQRTRTYAQSSKCNKVHKTWSWWTLESESCRSSLPYICEMTAFRFPD

SEQ ID NO: 3301 - protein, 124 aa, Homo sapiens
KLTRDSQSLCPYDWIGFQNKCYYFSKEEGDWNSSKYNCSTQHADLTIIDNIEEMNFLRRYKCSSDHWIGLKMAKNRTGQWVDGATFTKSFGMRGSEGCAYLSDDGAATARCYTERKWICRKRIH

SEQ ID NO: 3302 - protein, 194 aa, Homo sapiens
QGHLVHMTVVSGSNVTLNISESLPENYKQLTWFYTFDQKIVEWDSRKSKYFESKFKGRVRLDPQSGALYISKVQKEDNSTYIMRVLKKTGNEQEWKIKLQVLDPVPKPVIKIEKIEDMDDNCYLKLSCVIPGESVNYTWYGDKRPFPKELQNSVLETTLMPHNYSRCYTCQVSNSVSSKNGTVCLSPPCTLARS

SEQ ID NO: 3303 - protein, 133 aa, Homo sapiens
QVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 3304 - protein, 149 aa, Homo sapiens
MQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 3305 - protein, 205 aa, Homo sapiens
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 3306 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 3307 - protein, 5 aa, synthetic construct
GGGGS

SEQ ID NO: 3308 - protein, 10 aa, synthetic construct
GGGGSGGGGS

SEQ ID NO: 3309 - protein, 15 aa, synthetic construct
GGGGSGGGGSGGGGS

SEQ ID NO: 3310 - protein, 17 aa, synthetic construct
DVPSGPGGGGGSGGGGS

SEQ ID NO: 3311 - protein, 455 aa, synthetic construct; identical to SEQ ID NO: 3306

SEQ ID NO: 3312 - protein, 413 aa, synthetic construct
FRGPLLPNRPFTTVWNANTQWCLERHGVDVDVSVFDVVANPGQTFRGPDMTIFYSSQGTYPYYTPTGEPVFGGLPQNASLIAHLARTFQDILAAIPAPDFSGLAVIDWEAWRPRWAFNWDTKDIYRQRSRALVQAQHPDWPAPQVEAVAQDQFQGAARAWMAGTLQLGRALRPRGLWGFYGFPDCYNYDFLSPNYTGQCPSGIRAQNDQLGWLWGQSRALYPSIYMPAVLEGTGKSQMYVQHRVAEAFRVAVAAGDPNLPVLPYVQIFYDTTNHFLPLDELEHSLGESAAQGAAGVVLWVSWENTRTKESCQAIKEYMDTTLGPFILNVTSGALLCSQALCSGHGRCVRRTSHPKALLLLNPASFSIQLTPGGGPLSLRGALSLEDQAQMAVEFKCRCYPGWQAPWCERKSMW

SEQ ID NO: 3313 - protein, 551 aa, synthetic construct
YNFFPRKPKWDKNQITYRIIGYTPDLDPETVDDAFARAFQVWSDVTPLRFSRIHDGEADIMINFGRWEHGDGYPFDGKDGLLAHAFAPGTGVGGDSHFDDDELWTLGEGQVVRVKYGNADGEYCKFPFLFNGKEYNSCTDTGRSDGFLWCSTTYNFEKDGKYGFCPHEALFTMGGNAEGQPCKFPFRFQGTSYDSCTTEGRTDGYRWCGTTEDYDRDKKYGFCPETAMSTVGGNSEGAPCVFPFTFLGNKYESCTSAGRSDGKMWCATTANYDDDRKWGFCPDQGYSLFLVAAHEFGHAMGLEHSQDPGALMAPIYTYTKNFRLSQDDIKGIQELYGASPDIDLGTGPTPTLGPVTPEICKQDIVFDGIAQIRGEIFFFKDRFIWRTVTPRDKPMGPLLVATFWPELPEKIDAVYEAPQEEKAVFFAGNEYWIYSASTLERGYPKPLTSLGLPPDVQRVDAAFNWSKNKKTYIFAGDKFWRYNEVKKKMDPGFPKLIADAWNAIPDNLDAVVDLQGGGHSYFFKGAYYLKLENQSLKSVKFGSIKSDWLGC

SEQ ID NO: 3314 - protein, 14 aa, synthetic construct
AEAAAKEAAAKAAA

SEQ ID NO: 3315 - protein, 19 aa, synthetic construct
AEAAAKEAAAKEAAAKAAA

SEQ ID NO: 3316 - protein, 24 aa, synthetic construct
AEAAAKEAAAKEAAAKEAAAKAAA

SEQ ID NO: 3317 - protein, 29 aa, synthetic construct
AEAAAKEAAAKEAAAKEAAAKEAAAKAAA

SEQ ID NO: 3318-3436 - skipped (119 entries)

SEQ ID NO: 3437 - protein, 27 aa, synthetic construct
  VARIANT 2..26: note = This region may encompass 2-5 EAAAK units
AEAAAKEAAAKEAAAKEAAAKEAAAKA

SEQ ID NO: 3438 - skipped

SEQ ID NO: 3439 - protein, 116 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVLGWFDYWGQGTLVTVSS

SEQ ID NO: 3440 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGYPPDFTFGQGTKVEIK

SEQ ID NO: 3441 - protein, 119 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVAPYFAPFDYWGQGTLVTVSS

SEQ ID NO: 3442 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGNPPLYTFGQGTKVEIK

SEQ ID NO: 3443 - protein, 116 aa, synthetic construct
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARNGYLGDYWGQGTLVTVSS

SEQ ID NO: 3444 - protein, 111 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQAMQIPTFGQGTKVEIK

SEQ ID NO: 3445 - protein, 120 aa, synthetic construct
QVQLQQSGGGLVQPGGSLKLSCAASGIDFSRYWMSWVRRAPGKGLEWIGEINPDSSTINYAPSLKDKFIISRDNAKNTLYLQMSKVRSEDTALYYCASLYYDYGDAMDYWGQGTSVTVSS

SEQ ID NO: 3446 - protein, 108 aa, synthetic construct
DIVMTQSQRFMTTSVGDRVSVTCKASQSVDSNVAWYQQKPRQSPKALIFSASLRFSGVPARFTGSGSGTDFTLTISNLQSEDLAEYFCQQYNNYPLTFGAGTKLELKR

SEQ ID NO: 3447 - protein, 123 aa, synthetic construct
EVQLVESGGGLVKPGGSLRLSCAASGFTFGDYALSWFRQAPGKGLEWVGVSRSKAYGGTTDYAASVKGRFTISRDDSKSTAYLQMNSLKTEDTAVYYCASSGYSSGWTPFDYWGQGTLVTVSS

SEQ ID NO: 3448 - protein, 111 aa, synthetic construct
QSVLTQPPSASGTPGQRVTISCSGSSSNIGSNTVNWYQQLPGTAPKLLIFNYHQRPSGVPDRFSGSKSGSSASLAISGLQSEDEADYYCAAWDDSLNGWVFGGGTKLTVLG

SEQ ID NO: 3449-3642 - skipped (194 entries)

SEQ ID NO: 3643 - protein, 16 aa, synthetic construct
GGGGSGGGGSGGGGGS

SEQ ID NO: 3644 - protein, 107 aa, Homo sapiens [sequence omitted]

SEQ ID NO: 3645 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3646 - protein, 334 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKEPEA

SEQ ID NO: 3647 - protein, 330 aa, Homo sapiens; identical to SEQ ID NO: 41

SEQ ID NO: 3648 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3649 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

(19) United States
(12) Patent Application Publication
(10) Pub. No.: US 2024/0409636 A1
(43) Pub. Date: Dec. 12, 2024

(54) MULTIFUNCTIONAL MOLECULES BINDING TO TCR AND USES THEREOF

(71) Applicant: Marengo Therapeutics, Inc., Cambridge, MA (US)

(72) Inventors: Jonathan Hsu, Waltham, MA (US); Madan Katragadda, Acton, MA (US); Andrew Bayliffe, London (GB)

(21) Appl. No.: 18/749,969

(22) Filed: Jun. 21, 2024

Related U.S. Application Data
(63) Continuation of application No. PCT/US22/53705, filed on Dec. 21, 2022.
(60) Provisional application No. 63/292,859, filed on Dec. 22, 2021.

(51) Int. Cl. C07K 16/28; A61K 39/00; C07K 16/24

(52) U.S. Cl. CPC C07K 16/2809; C07K 16/246; A61K 2039/505; C07K 2317/31; C07K 2317/75

(57) ABSTRACT

Provides herein are multifunctional polypeptide molecules comprising T cell receptor variable alpha-binding moieties and cytokines and methods of treating conditions or diseases in a subject using the same.

### RELATED APPLICATIONS

This application is a continuation of International Application No. PCT/US22/53705 filed on Dec. 21, 2022, which claims the benefit of U.S. Provisional Patent Application No. 63/292,859 filed on Dec. 22, 2021, the entire contents of which are hereby incorporated by reference.

### SEQUENCE LISTING

The instant application contains a Sequence listing which has been submitted electronically in XML format and is hereby incorporated by reference in its entirety. Said XML copy, created on Jun. 21, 2024, is named 53676-749_601_SL.xml and is 786,345 bytes in size.

### BACKGROUND

Currently available molecules designed to redirect T cells to promote tumor cell lysis for cancer immunotherapy typically target the CD3 epsilon (CD3e) subunit of the T cell receptor (TCR). However, there are limitations to this approach. Previous studies have shown that, e.g., low doses of anti-CD3e monoclonal antibody (mAb) can cause T cell dysfunction and exert immunosuppressive effects. In addition, anti-CD3e mAbs bind to all T cells and thus activate a large number of T cells. Such non-physiological massive activation of T cells by these anti-CD3e mAbs can result in the production of proinflammatory cytokines such as IFN-gamma, IL-1-beta, IL-6, IL-10 and TNF-alpha, causing a “cytokine storm” known as the cytokine release syndrome (CRS), which is also associated with neurotoxicity (NT). Thus, there is a need for improved T cell receptor-binding molecules that redirect T cells for cancer immunotherapy.

### SUMMARY

Provided herein is a composition comprising a multispecific molecule comprising a T cell receptor alpha variable region (TCRαV)-binding moiety linked to a targeting moiety, wherein the TCRαV-binding moiety binds to a TCRαV of a T cell receptor (TCR) expressed by a T cell of a T cell population, wherein the targeting moiety binds to a target molecule other than the TCRαV on a target cell, and wherein when contacted to the T cell population, the multispecific molecule redirects the T cell to the target cell, activates the T cell, expands the T cell, or a combination thereof.

Also provided herein is a composition comprising a recombinant T cell receptor or a chimeric antigen receptor (CAR) comprising a domain that binds a TCRαV.

Also provided herein is a composition comprising a T cell genetically modified to express the CAR described herein.

Also provided herein is a pharmaceutical composition comprising a composition described herein and a pharmaceutically acceptable carrier, excipient, or stabilizer.

Also provided herein is a method of treating cancer in a subject in need thereof comprising administering a therapeutically effective amount of a pharmaceutical composition described herein to the subject.

Also provided herein is a method of expanding T cells that expresses a T cell receptor beta variable region (TCRαV) in a T cell population, the method comprising: contacting the T cell population with a composition comprising an anti-TCRαV molecule wherein the anti-TCRαV molecule contacts the TCRαV of a T cell receptor (TCR) expressed by the T cells in the T cell population, thereby expanding the T cells in the T cell population. In some embodiments, the T cell population is an in vivo T cell population.

Also provided herein is a method of expanding T cells that expresses a T cell receptor beta variable region (TCRαV) in a T cell population, the method comprising: contacting the T cell population with a composition comprising a multispecific molecule, wherein the multispecific molecule comprises a first domain that binds to a first target molecule and a second domain that binds to a second target molecule, wherein the first target molecule is a TCRαV and the second target molecule is a target molecule on a target cell that is different from the first target molecule, and wherein the first domain contacts the TCRαV of a T cell receptor (TCR) expressed by the T cells in the T cell population, thereby expanding the T cells in the T cell population. In some embodiments, the T cell population is an in vivo T cell population.

Also provided herein is a multifunctional polypeptide molecule comprising a first polypeptide, a second polypeptide, and at least one cytokine polypeptide or a variant thereof, wherein the first poly peptide and the second polypeptide are non-contiguous, wherein (i) the first polypeptide comprises a first portion of a dimerization module linked to (A) a first TCRαV-binding moiety comprising a first heavy chain variable domain (VH) and a first light chain variable domain (VL), or a single domain antibody, or (B) a first portion of a first TCRαV-binding moiety comprising a VH of the first TCRαV-binding moiety, wherein when the first polypeptide comprises the first portion of the first TCRαV-binding moiety, the multifunctional polypeptide molecule further comprises a third poly peptide comprising a second portion of the first TCRαV-binding moiety comprising a VL of the first TCRαV-binding moiety, wherein the third polypeptide is non-contiguous with the first polypeptide and the second polypeptide; and (ii) the second polypeptide comprises a second portion of the dimerization module; wherein (a) the multifunctional polypeptide molecule comprises a single TCRαV-binding moiety and the at least one cytokine polypeptide or the variant thereof is covalently linked to the second polypeptide, or (b) the multifunctional polypeptide molecule further comprises a second TCRαV-binding moiety and the at least one cytokine polypeptide or the variant thereof is covalently linked to the first polypeptide, the second polypeptide, the third polypeptide when the multifunctional polypeptide molecule further comprises the third polypeptide, or a combination thereof.

In some embodiments, the multifunctional polypeptide molecule comprises the second TCRαV-binding moiety, and the second portion of the dimerization module is linked to: (A) a second TCRαV-binding moiety comprising a second VH and a second VL, or a single domain antibody, or (B) a first portion of a second TCRαV-binding moiety comprising a VH of the second TCRαV-binding moiety, wherein when the second polypeptide comprises the first portion of the second TCRαV-binding moiety, the multifunctional polypeptide molecule further comprises a fourth polypeptide comprising a second portion of the second TCRαV-binding moiety comprising a VL of the second TCRαV-binding moiety, wherein the fourth polypeptide is non-contiguous with the first polypeptide, the second polypeptide, and the third polypeptide; wherein the at least one cytokine polypeptide or the variant thereof is covalently linked to the first polypeptide, the second polypeptide, the third polypeptide, the fourth polypeptide when the multifunctional poly peptide molecule further comprises the fourth poly peptide, or a combination thereof.

In some embodiments, the first portion of the dimerization module and the second portion of the dimerization module are dimerized.

In some embodiments, the first polypeptide comprises: (A) the first TCRαV-binding moiety comprising the first VH and the first VL, wherein the first TCRαV-binding moiety further comprises a first heavy chain constant domain 1 (CH1) linked to the first VH; or (B) the first portion of the first TCRαV-binding moiety comprising the VH of the first TCRαV-binding moiety, wherein the first portion of the first TCRαV-binding moiety further comprises a first CH1 linked to the VH of the first TCRαV-binding moiety.

In some embodiments, the first CH1 is linked to the C-terminus of the first VH or the C-terminus of the VH of the first TCRαV-binding moiety.

In some embodiments, the second polypeptide comprises: (A) the second TCRαV-binding moiety comprising the second VH and the second VL, wherein the second TCRαV-binding moiety further comprises a second CH1 linked to the second VH; or (B) the first portion of the second TCRαV-binding moiety comprising the VH of the second TCRαV-binding moiety, wherein the first portion of the second TCRαV-binding moiety further comprises a second CH1 linked to the VH of the second TCRαV-binding moiety.

In some embodiments, the second CH1 is linked to the C-terminus of the second VH or the C-terminus of the VH of the second TCRαV-binding moiety.

In some embodiments, the multifunctional polypeptide molecule comprises: (1) the first polypeptide comprising the first TCRαV-binding moiety that comprises the first VH and the first VL, wherein the first TCRαV-binding moiety further comprises a first light chain constant domain (CL) linked to the first VL; or (2) the first poly peptide comprising the first portion of the first TCRαV-binding moiety and the third polypeptide comprising the second portion of the first TCRαV-binding moiety, wherein the second portion of the first TCRαV-binding moiety further comprises a first CL linked to the VL of the first TCRαV-binding moiety.

In some embodiments, the first CL is linked to the C-terminus of the first VL or the C-terminus of the VL of the first TCRαV-binding moiety.

In some embodiments, the multifunctional polypeptide molecule comprises: (1) the second polypeptide comprising the second TCRαV-binding moiety that comprises the second VH and the second VL, wherein the second TCRαV-binding moiety further comprises a second CL linked to the second VL; or (2) the second polypeptide comprising the first portion of the second TCRαV-binding moiety and the fourth polypeptide comprising the second portion of the second TCRαV-binding moiety, wherein the second portion of the second TCRαV-binding moiety further comprises a second CL linked to the VL of the second TCRαV-binding moiety.

In some embodiments, the second CL is linked to the C-terminus of the second VL or the C-terminus of the VL of the second TCRαV-binding moiety.

In some embodiments, the first portion of the dimerization module is linked to the C-terminus of (A) the first TCRαV-binding moiety comprising the first VH and the first VL or the single domain antibody, or the C-terminus of (B) the first portion of the first TCRαV-binding moiety comprising the VH of the first TCRαV-binding moiety.

In some embodiments, the multifunctional polypeptide molecule comprises the second TCRαV-binding moiety, and the second portion of the dimerization module is linked to the C-terminus of (A) the second TCRαV-binding moiety comprising the second VH and the second VL or the single domain antibody, or the C-terminus of (B) the first portion of the second TCRαV-binding moiety comprising the VH of the second TCRαV-binding moiety.

In some embodiments, the multifunctional polypeptide molecule comprises a single TCRαV-binding moiety, and the at least one cytokine polypeptide or the variant thereof is covalently linked to the N-terminus of the second polypeptide, the C-terminus of the second polypeptide, or a combination thereof.

In some embodiments, the at least one cytokine polypeptide or the variant thereof is within a single contiguous polypeptide chain of the second polypeptide.

In some embodiments, (a) the N-terminus of the first poly peptide is linked to a cytokine polypeptide or a variant thereof; the C-terminus of the first polypeptide is linked to a cytokine polypeptide or a variant thereof; or a combination thereof; (b) the N-terminus of the second polypeptide is linked to a cytokine polypeptide or a variant thereof; the C-terminus of the second polypeptide is linked to a cytokine polypeptide or a variant thereof; or a combination thereof; (c) the N-terminus of the third polypeptide is linked to a cytokine poly peptide or a variant thereof; the C-terminus of the third polypeptide is linked to a cytokine polypeptide or a variant thereof; or a combination thereof; (d) the N-terminus of the fourth polypeptide is linked to a cytokine polypeptide or a variant thereof; the C-terminus of the fourth polypeptide is linked to an cytokine polypeptide or a variant thereof; or a combination thereof; or (e) a combination thereof.

In some embodiments, (a-1) the N-terminus of the first polypeptide is linked to a cytokine polypeptide or a variant thereof; the C-terminus of the first polypeptide is linked to a cytokine polypeptide or a variant thereof; or a combination thereof; and (a-2) the N-terminus of the second polypeptide is linked to a cytokine polypeptide or a variant thereof; the C-terminus of the second polypeptide is linked to a cytokine polypeptide or a variant thereof; or a combination thereof; (b-1) the N-terminus of the first polypeptide is linked to a cytokine polypeptide or a variant thereof; the C-terminus of the first polypeptide is linked to a cytokine polypeptide or a variant thereof; or a combination thereof; and (b-2) the N-terminus of the third polypeptide is linked to a cytokine polypeptide or a variant thereof; the C-terminus of the third polypeptide is linked to a cytokine polypeptide or a variant thereof; or a combination thereof; (c-1) the N-terminus of the first polypeptide is linked a cytokine polypeptide or a variant thereof; the C-terminus of the first polypeptide is linked to a cytokine polypeptide or a variant thereof; or a combination thereof; and (c-2) the N-terminus of the fourth poly peptide is linked to a cytokine polypeptide or a variant thereof; the C-terminus of the fourth polypeptide is linked to a cytokine polypeptide or a variant thereof; or a combination thereof; (d-1) the N-terminus of the second polypeptide is linked to a cytokine polypeptide or a variant thereof; the C-terminus of the second polypeptide is linked to a cytokine polypeptide or a variant thereof; or a combination thereof; and (d-2) the N-terminus of the third polypeptide is linked to a cytokine polypeptide or a variant thereof; the C-terminus of the third polypeptide is linked to a cytokine polypeptide or a variant thereof; or a combination thereof; (e-1) the N-terminus of the second polypeptide is linked to a cytokine polypeptide or a variant thereof; the C-terminus of the second polypeptide is linked to a cytokine polypeptide or a variant thereof; or a combination thereof; and (e-2) the N-terminus of the fourth polypeptide is linked to a cytokine polypeptide or a variant thereof; the C-terminus of the fourth polypeptide is linked to a cytokine polypeptide or a variant thereof; or a combination thereof; or (f-1) the N-terminus of the third polypeptide is linked to a cytokine polypeptide or a variant thereof; the C-terminus of the third polypeptide is linked to a cytokine polypeptide or a variant thereof; or a combination thereof; and (f-2) the N-terminus of the fourth polypeptide is linked to a cytokine polypeptide or a variant thereof; the C-terminus of the fourth polypeptide is linked to a cytokine polypeptide or a variant thereof.

In some embodiments, (a-1) the N-terminus of the first polypeptide is linked to a cytokine polypeptide or a variant thereof; the C-terminus of the first polypeptide is linked to a cytokine polypeptide or a variant thereof; or a combination thereof; (a-2) the N-terminus of the second poly peptide is linked to a cytokine polypeptide or a variant thereof; the C-terminus of the second polypeptide is linked to a cytokine polypeptide or a variant thereof; or a combination thereof; and (a-3) the N-terminus of the third polypeptide is linked to a cytokine polypeptide or a variant thereof; the C-terminus of the third polypeptide is linked to a cytokine polypeptide or a variant thereof; or a combination thereof; (b-1) the N-terminus of the first polypeptide is linked a cytokine polypeptide or a variant thereof; the C-terminus of the first polypeptide is linked to a cytokine polypeptide or a variant thereof; or a combination thereof; (b-2) the N-terminus of the second poly peptide is linked to a cytokine polypeptide or a variant thereof; the C-terminus of the second polypeptide is linked to a cytokine polypeptide or a variant thereof; or a combination thereof; and (b-3) the N-terminus of the fourth polypeptide is linked to a cytokine poly peptide or a variant thereof; the C-terminus of the fourth polypeptide is linked to a cytokine polypeptide or a variant thereof; or a combination thereof; or (c-1) the N-terminus of the second polypeptide is linked to a cytokine polypeptide or a variant thereof; the C-terminus of the second polypeptide is linked to a cytokine polypeptide or a variant thereof; or a combination thereof; (c-2) the N-terminus of the third polypeptide is linked to a cytokine polypeptide or a variant thereof; the C-terminus of the third polypeptide is linked to a cytokine polypeptide or a variant thereof; or a combination thereof; and (c-3) the N-terminus of the fourth polypeptide is linked to a cytokine polypeptide or a variant thereof; the C-terminus of the fourth polypeptide is linked to a cytokine polypeptide or a variant thereof; or a combination thereof.

In some embodiments, the N-terminus of the first polypeptide is linked to a cytokine polypeptide or a variant thereof; the C-terminus of the first polypeptide is linked to a cytokine polypeptide or a variant thereof; or a combination thereof; the N-terminus of the second polypeptide is linked to a cytokine polypeptide or a variant thereof; the C-terminus of the second poly peptide is linked to a cytokine polypeptide or a variant thereof; or a combination thereof; the N-terminus of the third polypeptide is linked to a cytokine polypeptide or a variant thereof; the C-terminus of the third poly peptide is linked to a cytokine polypeptide or a variant thereof; or a combination thereof; and the N-terminus of the fourth polypeptide is linked to a cytokine polypeptide or a variant thereof; the C-terminus of the fourth polypeptide is linked to a cytokine polypeptide or a variant thereof; or a combination thereof.

In some embodiments, the cytokine polypeptide or the variant thereof is within a single contiguous polypeptide chain of the first polypeptide, the second polypeptide, the third cytokine polypeptide, or the fourth cytokine polypeptide to which the cytokine polypeptide or the variant thereof is linked.

In some embodiments, the multifunctional polypeptide molecule further comprises: (i) a linker between the first portion of the dimerization module and the first TCRαV-binding moiety comprising the first VH and the first VL or the single domain antibody, or the first portion of the first TCRαV-binding moiety comprising the VH of the first TCRαV-binding moiety; (ii) a linker between the second portion of the dimerization module and the second TCRαV-binding moiety comprising the second VH and the second V L or the single domain antibody, or the first portion of the second TCRαV-binding moiety comprising the VH of the second TCRαV-binding moiety; (iii) a linker between the first VH and the first VL; (iv) a linker between the second VH and the second VL; (v) a linker between the first CH1 and the first VH, or the VH of the first TCRαV-binding moiety; (vi) a linker between the second CH1 and the second VH, or the VH of the second TCRαV-binding moiety; (vii) a linker between the first CL and the first VL, or the VL of the first TCRαV-binding moiety; (vii) a linker between the second CL and the second VL, or the VL of the second TCRαV-binding moiety; (viii) a linker between the at least one cytokine polypeptide or the variant thereof and the first polypeptide, a linker between the at least one cytokine polypeptide or the variant thereof and the second polypeptide, a linker between the at least one cytokine poly peptide or the variant thereof and the third polypeptide, a linker between the at least one cytokine polypeptide or the variant thereof and the fourth polypeptide, or a combination thereof; or (ix) a combination thereof.

In some embodiments, the linker is selected from the group consisting of a cleavable linker, a non-cleavable linker, a peptide linker, a flexible linker, a rigid linker, a helical linker, and a non-helical linker.

In some embodiments, the linker is the peptide linker and the linker comprises the sequence of SEQ ID NO: 3308 or SEQ ID NO: 3643.

In some embodiments, the multifunctional polypeptide molecule is an isolated multifunctional polypeptide molecule.

In some embodiments, the multifunctional polypeptide molecule comprises: (i) the first polypeptide comprising the first portion of the dimerization module linked to the C-terminus of the first portion of the first TCRαV-binding moiety; (ii) the second polypeptide comprising the second portion of the dimerization module; (iii) the third polypeptide comprising the second portion of the first TCRαV-binding moiety; and (iv) a cytokine polypeptide or a variant thereof covalently linked to the N-terminus of the second polypeptide, wherein the multifunctional polypeptide molecule comprises a single TCRαV-binding moiety.

In some embodiments, the multifunctional polypeptide molecule comprises: (i) the first polypeptide comprising the first portion of the dimerization module linked to the C-terminus of the first portion of the first TCRαV-binding moiety; (ii) the second polypeptide comprising the second portion of the dimerization module linked to the C-terminus of the first portion of the second TCRαV-binding moiety; (iii) the third polypeptide comprising the second portion of the first TCRαV-binding moiety; (iv) the fourth polypeptide comprising the second portion of the second TCRαV-binding moiety; (v) a cytokine polypeptide or a variant thereof covalently linked to the C-terminus of the third polypeptide, and (vi) a cytokine polypeptide or a variant thereof covalently linked to the C-terminus of the fourth polypeptide.

In some embodiments, the multifunctional polypeptide molecule comprises: (i) the first polypeptide comprising the first portion of the dimerization module linked to the C-terminus of the first portion of the first TCRαV-binding moiety; (ii) the second polypeptide comprising the second portion of the dimerization module linked to the C-terminus of the first portion of the second TCRαV-binding moiety; (iii) the third polypeptide comprising the second portion of the first TCRαV-binding moiety; (iv) the fourth polypeptide comprising the second portion of the second TCRαV-binding moiety; and (v) a cytokine polypeptide or a variant thereof covalently linked to the C-terminus of the third polypeptide or the C-terminus of the fourth poly peptide, but not to both.

In some embodiments, the multifunctional polypeptide molecule comprises: (i) the first poly peptide comprising the first portion of the dimerization module linked to the C-terminus of the first portion of the first TCRαV-binding moiety; (ii) the second polypeptide comprising the second portion of the dimerization module linked to the C-terminus of the first portion of the second TCRαV-binding moiety; (iii) the third polypeptide comprising the second portion of the first TCRαV-binding moiety; (iv) the fourth polypeptide comprising the second portion of the second TCRαV-binding moiety; and (v) a cytokine polypeptide or a variant thereof covalently linked to the C-terminus of the first polypeptide or the C-terminus of the second polypeptide, but not to both.

In some embodiments, the first TCRαV-binding moiety, the second TCRαV-binding moiety, or a combination thereof comprises any one selected from the group consisting of a Fab, a F(ab′)2, an Fv, a single chain Fv (scFv), a single domain antibody, a diabody (dAb), a camelid antibody, and a combination thereof.

In some embodiments, the first TCRαV-binding moiety, the second TCRαV-binding moiety, or a combination thereof comprises the Fab or the scFv.

In some embodiments, the TCRαV-binding moiety is the sole antigen-binding moiety of the multifunctional polypeptide molecule.

In some embodiments, the multifunctional polypeptide molecule comprises two or more of the at least one cytokine polypeptides.

In some embodiments, the at least one cytokine polypeptide comprises interleukin-2 (IL-2) or a fragment thereof.

In some embodiments, the at least one cytokine polypeptide comprises a sequence having at least 75% sequence identity to the sequence of SEQ ID NO: 2191.

In some embodiments, the variant is an IL-2 variant comprising a substitution mutation.

In some embodiments, the variant is an IL-2 variant comprising C125A mutation.

In some embodiments, the variant comprises a sequence having at least 75% sequence identity to the sequence of SEQ ID NO: 2270.

In some embodiments, the first portion of the dimerization module comprises a first immunoglobulin constant regions (Fc regions) and the second portion of the dimerization module comprises a second Fc region.

In some embodiments, the first Fc region, the second Fc region, or a combination thereof is selected from the group consisting of an IgG1 Fc region or a fragment thereof, an IgG2 Fc region or a fragment thereof, an IgG3 Fc region or a fragment thereof, an IgGA1 Fc region or a fragment thereof, an IgGA2 Fc region or a fragment thereof, an IgG4 Fc region or a fragment thereof, an IgJ Fc region or a fragment thereof, an IgM Fc region or a fragment thereof, an IgD Fc region or a fragment thereof, and an IgE Fc region or a fragment thereof.

In some embodiments, the first Fc region, the second Fc region, or a combination thereof is selected from the group consisting of a human IgG1 Fc region or a fragment thereof, a human IgG2 Fc region or a fragment thereof, and a human IgG4 Fc region or a fragment thereof.

In some embodiments, the first Fc region, the second Fc region, or a combination thereof comprises an Fc interface with one or more of: a paired cavity-protuberance, an electrostatic interaction, or a strand-exchange, wherein the dimerization of the first Fc region and the second Fc region is enhanced as indicated by a greater ratio of heteromultimer:homomultimer forms relative to a dimerization of Fc regions with a non-engineered interface.

In some embodiments, the first Fc region, the second Fc region, or a combination thereof comprises an amino acid substitution listed in Table 6.

In some embodiments, the first Fc region, the second Fc region, or a combination thereof comprises an Asn297Ala (N297A) mutation or a Leu234Ala/Leu235Ala (LALA) mutation.

In some embodiments, the first Fc region, the second Fc region, or a combination thereof comprises a sequence having at least 75% sequence identity to the sequence of SEQ ID NO: 40, SEQ ID NO: 42, SEQ ID NO: 3645, SEQ ID NO: 3646, SEQ ID NO: 3647, SEQ ID NO:3648, or SEQ ID NO: 3649.

In some embodiments, the first TCRαV-binding moiety, the second TCRαV-binding moiety, or a combination thereof binds to one or more of a TCRαV subfamily selected from the group consisting of: a TCRα V1 subfamily, a TCRα V2 subfamily, a TCRα V3 subfamily, a TCRα V4, a TCRα V5 subfamily, a TCRα V6 subfamily, a TCRα V7 subfamily, a TCRα V8 subfamily, a TCRα V9 subfamily, a TCRα V10 subfamily, a TCRα V12 subfamily, a TCRα V13 subfamily, a TCRα V14 subfamily, a TCRα V16 subfamily, a TCRα V17 subfamily, a TCRα V18 subfamily, a TCRα V19 subfamily, a TCRα V20 subfamily, a TCRα V21 subfamily, a TCRα V22 subfamily, a TCRα V23 subfamily, a TCRα V24 subfamily, TCRα V25 subfamily, a TCRα V26 subfamily, a TCRα V27 subfamily, a TCRα V29 subfamily, a TCRα V30 subfamily, a TCRα V34 subfamily, a TCRα V35 subfamily, a TCRα V36 subfamily, a TCRα V38 subfamily, a TCRα V39 subfamily, a TCRα V40 subfamily, a TCRα V41 subfamily, family members of said subfamilies, and variants thereof (e.g., a structural or functional variant thereof).

In some embodiments, the first TCRαV-binding moiety, the second TCRαV-binding moiety, or a combination thereof binds to one or more of a TCRαV subfamily selected from the group consisting of TCRαV1-1, TCRαV1-2, and variants thereof.

In some embodiments, the first TCRαV-binding moiety, the second TCRαV-binding moiety, or a combination thereof binds to one or more of a TCRαV subfamily selected from the group consisting of TCRαV8-1, TCRαV8-2, TCRαV8-3, TCRαV8-4, TCRαV8-6, and variants thereof.

In some embodiments, the first TCRαV-binding moiety, the second TCRαV-binding moiety, or a combination thereof binds to one or more of a TCRαV subfamily selected from the group consisting of a TCRαV9-1, TCRαV9-2, and variants thereof.

In some embodiments, the first TCRαV-binding moiety, the second TCRαV-binding moiety, or a combination thereof binds to one or more of a TCRαV subfamily selected from the group consisting of TCRαV12-1, TCRαV12-2, TCRαV12-3, and variants thereof.

In some embodiments, the first TCRαV-binding moiety, the second TCRαV-binding moiety, or a combination thereof binds to one or more of a TCRαV subfamily selected from the group consisting of TCRαV13-1, TCRαV13-2, and variants thereof.

In some embodiments, the first TCRαV-binding moiety, the second TCRαV-binding moiety, or a combination thereof binds to one or more of a TCRαV subfamily selected from the group consisting of TCRαV14/DV4, and variants thereof.

In some embodiments, the first TCRαV-binding moiety, the second TCRαV-binding moiety, or a combination thereof binds to one or more of a TCRαV subfamily selected from the group consisting of TCRαV23/DV6, and variants thereof.

In some embodiments, the first TCRαV-binding moiety, the second TCRαV-binding moiety, or a combination thereof binds to one or more of a TCRαV subfamily selected from the group consisting of TCRαV26-1, TCRαV26-2, and variants thereof.

In some embodiments, the first TCRαV-binding moiety, the second TCRαV-binding moiety, or a combination thereof binds to one or more of a TCRαV subfamily selected from the group consisting of TCRαV29/DV5, and variants thereof.

In some embodiments, the first TCRαV-binding moiety, the second TCRαV-binding moiety, or a combination thereof binds to one or more of a TCRαV subfamily selected from the group consisting of TCRαV236/DV7, and variants thereof.

In some embodiments, the first TCRαV-binding moiety, the second TCRαV-binding moiety, or a combination thereof binds to one or more of a TCRαV subfamily selected from the group consisting of TCRαV38-1, TCRαV38-2/DV8, and variants thereof.

In some embodiments, the multifunctional polypeptide molecule comprises the first TCRαV-binding moiety and the second TCRαV-binding moiety, and the first TCRαV-binding moiety and the second TCRαV-binding moiety are same.

In some embodiments, the multifunctional polypeptide molecule comprises the first TCRαV-binding moiety and the second TCRαV-binding moiety, and the first TCRαV-binding moiety and the second TCRαV-binding moiety are different.

In some embodiments, the first TCRαV-binding moiety and the second TCRαV-binding moiety binds: (i) one or more of a TCRα V12 subfamily member and one or more of a different TCRα subfamily member, respectively.

In some embodiments, the first TCRαV-binding moiety, the second TCRαV-binding moiety, or a combination thereof comprises: (i) a VH comprising a framework region (FR) comprising a framework 1 (FR1), a framework region 2 (FR2), a framework region 3 (FR3), and a framework region 4 (FR4) that have at least 75% sequence identity to a non-murine germline FR1, a non-murine germline FR2, a non-murine germline FR3, and a non-murine germline FR4; (ii) a VL comprising a FR comprising a FR1, a FR2, a FR3, and a FR4 that have at least 75% sequence identity to a non-murine germline FR1, a non-murine germline FR2, a non-murine germline FR3, and a non-murine germline FR4; or (iii) a combination thereof.

In some embodiments, the first polypeptide, the second polypeptide, or a combination thereof comprises a heavy chain constant region of an IgM or a fragment thereof.

In some embodiments, the heavy chain constant region of the IgM comprises a sequence having at least 75% sequence identity to the sequence of SEQ ID NO: 73.

In some embodiments, the first polypeptide, the second polypeptide, or a combination thereof comprises a heavy chain constant region of an IgJ or a fragment thereof.

In some embodiments, the heavy chain constant region of the IgJ comprises a sequence having at least 75% sequence identity to the sequence of SEQ ID NO: 76.

In some embodiments, the first polypeptide, the second polypeptide, a combination thereof comprises a heavy chain constant region of an IgGA1 or a fragment thereof.

In some embodiments, the heavy chain constant region of the IgGA1 comprises a sequence having at least 75% sequence identity to the sequence of SEQ ID NO: 74.

In some embodiments, the first polypeptide, the second polypeptide, or a combination thereof comprises a heavy chain constant region of an IgGA2 or a fragment thereof.

In some embodiments, the heavy chain constant region of the IgGA2 comprises a sequence having at least 75% sequence identity to the sequence of SEQ ID NO: 75.

In some embodiments, the first polypeptide, the second polypeptide, or a combination thereof comprises a heavy chain constant region of an IgG1 or a fragment thereof.

In some embodiments, the heavy chain constant region of the IgG1 comprises a sequence having at least 75% sequence identity to the sequence of SEQ ID NO: 41 or SEQ ID NO: 3645.

In some embodiments, the first polypeptide, the second polypeptide, the third polypeptide, the fourth polypeptide, or a combination thereof comprises a light chain constant region of a kappa chain or a fragment thereof.

In some embodiments, the light chain constant region of a kappa chain comprises a light chain constant region sequence listed in Table 1.

In some embodiments, the light chain constant region of the kappa chain comprises a sequence having at least 75% sequence identity to the sequence of SEQ ID NO: 39 or SEQ ID NO: 3644.

In some embodiments, the first TCRαV-binding moiety, the second TCRαV-binding moiety, or a combination thereof binds to an outward facing region on a TCRαV protein.

In some embodiments, the outward facing region on the TCRαV protein comprises a structurally conserved region of TCRαV having a similar structure across one or more TCRαV subfamilies.

In some embodiments, the third sequence is linked to the N-terminus of the first sequence.

In some embodiments, the third sequence is linked to the C-terminus of the second sequence.

In some embodiments, the third sequence is linked to the N-terminus of the first sequence.

In some embodiments, the third sequence is linked to the C-terminus of the second sequence.

In some embodiments, the third polypeptide, the fourth polypeptide, or a combination thereof further comprises the third sequence, wherein the third sequence is linked to the fourth sequence, the fifth sequence, or a combination thereof.

In some embodiments, the third sequence is linked to the N-terminus of the fourth sequence.

In some embodiments, the third sequence is linked to the C-terminus of the fifth sequence.

In some embodiments, the third sequence is linked to the N-terminus of the first sequence.

In some embodiments, the third sequence is linked to the C-terminus of the second sequence.

In some embodiments, the third polypeptide, the fourth polypeptide, or a combination thereof further comprises the third sequence, wherein the third sequence is linked to the fourth sequence, the fifth sequence, or a combination thereof.

In some embodiments, the third sequence is linked to the N-terminus of the fourth sequence.

In some embodiments, the third sequence is linked to the C-terminus of the fifth sequence.

Described herein, in certain embodiments, is a nucleic acid molecule comprising a nucleotide sequence encoding the multifunctional poly peptide molecule or the CAR as described herein.

In some embodiments, the nucleic acid molecule is an isolated nucleic acid molecule.

Described herein, in certain embodiments, is a vector comprising one or more of the nucleic acid molecules as described herein.

Described herein, in certain embodiments, is a cell comprising the nucleic acid molecules as described herein, or the vector as described herein.

Described herein, in certain embodiments, is a pharmaceutical composition comprising the multifunctional polypeptide molecule or the T cell genetically modified to express the CAR as described herein, the nucleic acid molecules as described herein, the vector as described herein, or the cell as described herein, and a pharmaceutically acceptable carrier, excipient, or diluent.

Described herein, in certain embodiments, is a method of treating a condition or disease in a subject in need therefor comprising administering to the subject a therapeutically effective amount of the multifunctional polypeptide molecule as described herein, the CAR as described herein, the nucleic acid molecules as described herein, the vector as described herein, the cell as described herein (e.g., the T cell genetically modified to express the CAR), the pharmaceutical composition as described herein, or a combination thereof, wherein the administering is effective to treat the condition or disease in the subject.

In some embodiments, the condition or disease is cancer.

In some embodiments, the cancer is a solid tumor, a hematological cancer, a metastatic cancer, a soft tissue tumor, or a combination thereof.

In some embodiments, the cancer is the solid tumor, and the solid tumor is selected from the group consisting of melanoma, pancreatic cancer, breast cancer, colorectal cancer, lung cancer, skin cancer, ovarian cancer, liver cancer, and a combination thereof.

In some embodiments, the cancer is the hematological cancer, and the hematological cancer is selected from the group consisting of Hodgkin's lymphoma, Non-Hodgkin's lymphoma, acute myeloid leukemia (AML), chronic myeloid leukemia, myelodysplastic syndrome, multiple myeloma, T-cell lymphoma, acute lymphocytic leukemia, and a combination thereof.

In some embodiments, the Non-Hodgkin's lymphoma is selected from the group consisting of B cell lymphoma, diffuse large B cell lymphoma (DLBCL), follicular lymphoma, chronic lymphocytic leukemia (B-CLL), mantle cell lymphoma, marginal zone B-cell lymphoma, Burkitt lymphoma, lymphoplasmacytic lymphoma, hairy cell leukemia, and a combination thereof.

In some embodiments, the T-cell lymphoma is peripheral T-cell lymphoma.

In some embodiments, the cancer is characterized by a cancer antigen present on the cancer.

In some embodiments, the cancer antigen is a tumor antigen, a stromal antigen, or a hematological antigen.

In some embodiments, the cancer antigen is selected from the group consisting of BCMA, CD19, CD20, CD22, FcRH5, PDL1, CD47, gangloside 2 (GD2), prostate stem cell antigen (PSCA), prostate specific membrane antigen (PMSA), prostate-specific antigen (PSA), carcinoembryonic antigen (CEA), Ron Kinase, c-Met, Immature laminin receptor, TAG-72, BING-4, Calcium-activated chloride channel 2. Cyclin-B1, 9D7, Ep-CAM, EphA3, Her2/neu, Telomerase, SAP-1, Survivin, NY-ESO-1/LAGE-1, PRAME, SSX-2, Melan-A/MART-1, Gp100/pmel17, Tyrosinase, TRP-1/-2, MC1R, β-catenin, BRCA1/2, CDK4, CML66, Fibronectin, p53, Ras, TGF-B receptor, AFP, ETA, MAGE, MUC-1, CA-125, BAGE, GAGE, NY-ESO-1, β-catenin, CDK4, CDC27, α actinin-4, TRP1/gp75, TRP2, gp100, Melan-A/MART1, gangliosides, WT1, EphA3, Epidermal growth factor receptor (EGFR), MART-2, MART-1, MUC1, MUC2, MUM1, MUM2, MUM3, NA88-1, NPM, OA1, OGT, RCC, RUI1, RUI2, SAGE, TRG, TRP1, TSTA, Folate receptor alpha, L1-CAM, CAIX, gpA33, GD3, GM2, VEGFR, Intergrins, carbohydrates, IGF1R, EPHA3, TRAILR1, TRAILR2, RANKL, FAP, TGF-beta, hyaluronic acid, collagen, tenascin C, and tenascin W.

In some embodiments, the method further comprises administering a second therapeutic agent or therapy to the subject.

In some embodiments, the second therapeutic agent or therapy comprises a chemotherapeutic agent, a biologic agent, a hormonal therapy, radiation, or surgery.

In some embodiments, the second therapeutic agent or therapy is administered in combination with the multifunctional polypeptide molecule as described herein, the CARs as described herein, the nucleic acid molecules as described herein, the vector as described herein, the cell as described herein (e.g., the T cell genetically modified to express the CAR), the pharmaceutical composition as described herein, sequentially, simultaneously, or concurrently.

Also provided herein is a composition comprising a recombinant T cell receptor or a chimeric antigen receptor (CAR) comprising a domain that binds a TCRαV.

Also provided herein is a composition comprising a T cell comprising a recombinant T cell receptor or a chimeric antigen receptor (CAR) comprising a domain that binds a TCRαV.

In some embodiments, the CAR comprises an extracellular domain comprising the domain that binds a TCRαV, a transmembrane domain; and an intracellular domain comprising an intracellular signaling domain.

In some embodiments, the extracellular domain comprises a CD8 or CD28 extracellular domain.

In some embodiments, the transmembrane domain comprises a CD8 or CD28 transmembrane domain.

In some embodiments, the intracellular domain comprises a CD3 zeta intracellular signaling domain.

In some embodiments, the domain that binds a TCRαV binds to one or more of a TCRαV subfamily selected from the group consisting of: a TCRα V1 subfamily, a TCRα V2 subfamily, a TCRα V3 subfamily, a TCRα V4, a TCRα V5 subfamily, a TCRα V6 subfamily, a TCRα V7 subfamily, a TCRα V8 subfamily, a TCRα V9 subfamily, a TCRα V10 subfamily, a TCRα V12 subfamily, a TCRα V13 subfamily, a TCRα V14 subfamily, a TCRα V16 subfamily, a TCRα V17 subfamily, a TCRα V18 subfamily, a TCRα V19 subfamily, a TCRα V20 subfamily, a TCRα V21 subfamily, a TCRα V22 subfamily, a TCRα V23 subfamily, a TCRα V24 subfamily, TCRα V25 subfamily, a TCRα V26 subfamily, a TCRα V27 subfamily, a TCRα V29 subfamily, a TCRα V30 subfamily, a TCRα V34 subfamily, a TCRα V35 subfamily, a TCRα V36 subfamily, a TCRα V38 subfamily, a TCRα V39 subfamily, a TCRα V40 subfamily, or a TCRα V41 subfamily, as well as family members of said subfamilies, and variants thereof.

Also provided herein is a pharmaceutical composition comprising a CAR or CAR-T cell composition described herein, and a pharmaceutically acceptable diluent, carrier, excipient, or stabilizer. Also provided herein is a method of treating a disease or condition in a subject in need thereof comprising administering a therapeutically effective amount of the pharmaceutical composition comprising a CAR or CAR-T cell composition described herein to the subject.

In some embodiments, the disease or condition is a cancer. In some embodiments, the cancer is breast cancer, and the domain that binds a TCRαV binds to a TCRα V1 subfamily; the cancer is ER+ breast cancer, and the domain that binds a TCRαV binds to a TCRα V1 subfamily; the cancer is ER+ breast cancer, and the domain that binds a TCRαV binds to a TCRα V3 subfamily; the cancer is diffuse large B-cell lymphoma, and the domain that binds a TCRαV binds to a TCRα V6 subfamily; the cancer is diffuse large B-cell lymphoma, and the domain that binds a TCRαV binds to a TCRα V8 subfamily; the cancer is breast cancer, and the domain that binds a TCRαV binds to a TCRα V9 subfamily; the cancer is diffuse large B-cell lymphoma, and the domain that binds a TCRαV binds to a TCRα V12 subfamily; the cancer is melanoma, and the domain that binds a TCRαV binds to a TCRα V12 subfamily; the cancer is multiple myeloma, and the domain that binds a TCRαV binds to a TCRα V19 subfamily; the cancer is diffuse large B-cell lymphoma, and the domain that binds a TCRαV binds to a TCRα V21 subfamily; the cancer is diffuse large B-cell lymphoma, and the domain that binds a TCRαV binds to a TCRα V22 subfamily; the cancer is diffuse large B-cell lymphoma, and the domain that binds a TCRαV binds to a TCRα V25 subfamily; the cancer is leukemia, and the domain that binds a TCRαV binds to a TCRα V29 subfamily; the cancer is melanoma, and the domain that binds a TCRαV binds to a TCRα V29 subfamily; the cancer is breast cancer, and the domain that binds a TCRαV binds to a TCRα V29 subfamily; the cancer is endometrial cancer, and the domain that binds a TCRαV binds to a TCRα V30 subfamily; the cancer is leukemia, and the domain that binds a TCRαV binds to a TCRα V38 subfamily and/or the cancer is esophageal squamous cell carcinoma, and the domain that binds a TCRαV binds to a TCRα V39 subfamily.

In some embodiments, the disease or condition is an autoimmune disease. In some embodiments, the autoimmune disease is multiple sclerosis, and the domain that binds a TCRαV binds to a TCRα V1 subfamily; the autoimmune disease is Crohn's disease, and the domain that binds a TCRαV binds to a TCRα V2 subfamily; the autoimmune disease is Sjögren's syndrome, and the domain that binds a TCRαV binds to a TCRα V13 subfamily; the autoimmune disease is celiac disease, and the domain that binds a TCRαV binds to a TCRα V20 subfamily; the autoimmune disease is Crohn's disease, and the domain that binds a TCRαV binds to a TCRα V22 subfamily; the autoimmune disease is celiac disease, and the domain that binds a TCRαV binds to a TCRα V26 subfamily; and/or the autoimmune disease is Crohn's disease, and/or the autoimmune disease Crohn's disease, and the domain that binds a TCRαV binds to a TCRα V40 subfamily.

In some embodiments, the disease or condition is an infection. In some embodiments, the infection is a S. parathyphi infection, and the domain that binds to a TCRαV binds to a TCRα V1 subfamily; the infection is a Bacteroidetes infection, and the domain that binds to a TCRαV binds to a TCRα V1 subfamily; the infection is a Proteobacteria infection, and the domain that binds to a TCRαV binds to a TCRα V1 subfamily; the infection is a M. tuberculosis infection, and the domain that binds to a TCRαV binds to a TCRα V1 subfamily; the infection is a respiratory virus infection, and the domain that binds to a TCRαV binds to a TCRα V6 subfamily; the infection is a SARS-CoV-2 infection, and the domain that binds to a TCRαV binds to a TCRα V7 subfamily; the infection is a SARS-CoV-2 infection, and the domain that binds to a TCRαV binds to a TCRα V9 subfamily; the infection is a SARS-CoV-2 infection, and the domain that binds to a TCRαV binds to a TCRα V11 subfamily; the infection is a S. pyogenes infection, and the domain that binds to a TCRαV binds to a TCRα V12 subfamily; the infection is a SARS-CoV-2 infection, and the domain that binds to a TCRαV binds to a TCRα V12 subfamily; the infection is yellow fever, and the domain that binds to a TCRαV binds to a TCRα V12 subfamily; the infection is influenza, and the domain that binds to a TCRαV binds to a TCRα V13 subfamily; the infection is a respiratory virus infection, and the domain that binds to a TCRαV binds to a TCRα V16 subfamily; the infection is a HIV infection, and the domain that binds to a TCRαV binds to a TCRα V17 subfamily; the infection is a Cytomegalovirus infection, and the domain that binds to a TCRαV binds to a TCRα V17 subfamily; the infection is a SARS-CoV-2 infection, and the domain that binds to a TCRαV binds to a TCRα V18 subfamily; the infection is a HIV infection, and the domain that binds to a TCRαV binds to a TCRα V24 subfamily; the infection influenza, and the domain that binds to a TCRαV binds to a TCRα V27 subfamily; the infection is an Epstein-Barr Virus infection, and the domain that binds to a TCRαV binds to a TCRα V38 subfamily.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 depicts exemplary graphs showing the average abundance of each depicted TRAV gene relative to total TRAV gene based on sequencing analysis of samples obtained from 5 healthy individuals. TCR repertoire sequencing was performed from total RNA isolated from healthy human purified T-cells. The MiXCR pipeline was used for alignment of reads to TCR clonotypes/germlines. Each TRAV gene is plotted against relative abundance (where 1 equals 100% of total TRAV gene). The average relative abundance for each TRAV is shown with each individual donor shown as (⋅). n=5.

FIG. 2A depicts exemplary flow cytometry results showing expansion of T-cells after stimulation with an anti-TRAV 12-1 antibody. Purified human T-cells isolated from a healthy individual were stimulated for 5 days with plate-bound anti-TCR V alpha 12.1 antibody (6D6.6). The FACS plots show expanded T-cells stained with an AF647-labeled anti-TCR V alpha 12.1 antibody. Unstimulated T-cells were stained for baseline expression. Data from 1 representative donor is shown.

FIG. 2B depicts exemplary flow cytometry results showing expansion of T-cells after stimulation with an anti-TRAV 12-1 antibody. Purified human T cells isolated from healthy individuals were stimulated for 10 days with 100 nM of plate-bound anti-TCR V alpha 12.1 antibody (6D6.6); followed by an additional 2-days in culture in the presence of 100 U/mL recombinant human IL-2. The FACS plots show expanded T cells stained with an AF647-labeled anti-TCR V alpha 12.1 antibody. Unstimulated T-cells were stained for baseline expression. Data from 2 representative donors are shown.

FIG. 3A depicts exemplary dot plots showing expansion of human CD4+ CD25+ and CD8+ CD25+ T-cells after stimulation with anti-TCRαV-19/IL-2 bispecific antibody. Human PBMCs were treated at with 0.001, 0.01, 0.1, 1, 10, or 100 nM of anti-TCRαV-19/IL-2 for 5 days at 37° C. Cells were stained with anti-CD4, anti-CD25 (IL2RA), and anti-CD8 antibodies and FACS was used to quantify percentage of CD4+ CD25+ and CD8+ CD25+ T-cell populations. Isotype controls were used for baseline comparison.

FIG. 3B depicts exemplary dot plots showing expansion of murine CD4+ CD25+ and CD8+ CD25+ T-cells after stimulation with anti-TCRαV-14/IL-2 bispecific antibody. Isolated murine T-cells were treated at with 0.001, 0.01, 0.1, 1, 10, or 100 nM of anti-TCRαV-14/IL-2 for 4 days at 37° C. Cells were stained with anti-CD4, anti-CD25 (IL2RA), and anti-CD8 antibodies and FACS was used to quantify percentage of CD4+ CD25+ and CD8+ CD25+ T-cell populations. Isotype controls were used for baseline comparison.

FIG. 3C depicts exemplary dot plots showing expansion of murine CD4+ CD25+ and CD8+ CD25+ T-cells after stimulation with anti-TCRαV-12/IL-2 bispecific antibody. Isolated murine T-cells were treated at with 0.001, 0.01, 0.1, 1, 10, or 100 nM of anti-TCRαV-12/IL-2 for 4 days at 37° C. Cells were stained with anti-CD4, anti-CD25 (IL2RA), and anti-CD8 antibodies and FACS was used to quantify percentage of CD4+ CD25+ and CD8+ CD25+ T-cell populations. Isotype controls were used for baseline comparison.

### DETAILED DESCRIPTION

### Definition

Certain specific details of this description are set forth in order to provide a thorough understanding of various embodiments. However, one skilled in the art will understand that the present disclosure may be practiced without these details. In other instances, well-known structures have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed disclosure.

As used in this specification and the appended claims, the singular forms “a,” “an.” and “the” include plural referents unless the content clearly dictates otherwise. The use of the words “a” or “an” when used in conjunction with the term “comprising” herein may mean “one,” but it is also consistent with the meaning of “one or more,” “at least one,” and “one or more than one.”

It should also be noted that the term “or” is generally employed in its sense including “and/or” unless the content clearly dictates otherwise.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below.

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods. As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values.

The term “acquire” or “acquiring” as the terms are used herein, refer to obtaining possession of a physical entity (e.g., a sample, a polypeptide, a nucleic acid, or a sequence), or a value, e.g., a numerical value, by “directly acquiring” or “indirectly acquiring” the physical entity or value. “Directly acquiring” means performing a process (e.g., performing a synthetic or analytical method) to obtain the physical entity or value. “Indirectly acquiring” refers to receiving the physical entity or value from another party or source (e.g., a third party laboratory that directly acquired the physical entity or value). Directly acquiring a physical entity includes performing a process that includes a physical change in a physical substance, e.g., a starting material. Directly acquiring a value includes performing a process that includes a physical change in a sample or another substance, e.g., performing an analytical process which includes a physical change in a substance, e.g., a sample.

“Antibody molecule” as used herein refers to a protein, e.g., an immunoglobulin chain or fragment thereof, comprising at least one immunoglobulin variable domain structure and/or sequence. An antibody molecule encompasses antibodies (e.g., full-length antibodies) and antibody fragments. In some embodiments, an antibody molecule comprises an antigen binding or functional fragment of a full length antibody, or a full length immunoglobulin chain. For example, a full-length antibody is an immunoglobulin (Ig) molecule (e.g., an IgG antibody) that is naturally occurring or formed by normal immunoglobulin gene fragment recombinatorial processes). In embodiments, an antibody molecule refers to an immunologically active, antigen-binding portion of an immunoglobulin molecule, such as an antibody fragment. An antibody fragment, e.g., functional fragment, is a portion of an antibody, e.g., Fab, Fab′, F(ab′)₂, F(ab)₂, variable fragment (Fv), domain antibody (dAb), or single chain variable fragment (scFv). A functional antibody fragment binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. Exemplary antibody molecules include full length antibodies and antibody fragments, e.g., dAb (domain antibody), single chain, Fab, Fab′, and F(ab′)₂ fragments, and single chain variable fragments (scFvs). In some embodiments, the antibody molecule is an antibody mimetic. In some embodiments, the antibody molecule is, or comprises, an antibody-like framework or scaffold, such as, fibronectins, ankyrin repeats (e.g., designed ankyrin repeat proteins (DARPins)), avimers, affibody affinity ligands, anticalins, or affilin molecules.

The term “human-like antibody molecule” as used herein refers to a humanized antibody molecule, human antibody molecule or an antibody molecule having at least 95% sequence identity with a non-murine germline framework region, e.g., FR1, FR2, FR3 and/or FR4. In some embodiments, the human-like antibody molecule comprises a framework region having at least 95% sequence identity to a human germline framework region, e.g., a FR1, FR2, FR3 and/or FR4 of a human germline framework region. In some embodiments, the human-like antibody molecule is a recombinant antibody. In some embodiments, the human-like antibody molecule is a humanized antibody molecule. In some embodiments, the human-like antibody molecule is human antibody molecule. In some embodiments, the human-like antibody molecule is a phage display or a yeast display antibody molecule. In some embodiments, the human-like antibody molecule is a chimeric antibody molecule. In some embodiments, the human-like antibody molecule is a CDR grafted antibody molecule.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

In embodiments, an antibody molecule is monospecific, e.g., it comprises binding specificity for a single epitope. In some embodiments, an antibody molecule is multispecific, e.g., it comprises a plurality of immunoglobulin variable domain sequences, where a first immunoglobulin variable domain sequence has binding specificity for a first epitope and a second immunoglobulin variable domain sequence has binding specificity for a second epitope. In some embodiments, an antibody molecule is a bispecific antibody molecule. “Bispecific antibody molecule” as used herein refers to an antibody molecule that has specificity for more than one (e.g., two, three, four, or more) epitope and/or antigen.

“Antigen” (Ag) as used herein refers to a molecule that can provoke an immune response, e.g., involving activation of certain immune cells and/or antibody generation. Any macromolecule, including almost all proteins or peptides, can be an antigen. Antigens can also be derived from genomic recombinant or DNA. For example, any DNA comprising a nucleotide sequence or a partial nucleotide sequence that encodes a protein capable of eliciting an immune response encodes an “antigen.” In embodiments, an antigen does not need to be encoded solely by a full length nucleotide sequence of a gene, nor does an antigen need to be encoded by a gene at all. In embodiments, an antigen can be synthesized or can be derived from a biological sample, e.g., a tissue sample, a tumor sample, a cell, or a fluid with other biological components. As used, herein a “tumor antigen” or interchangeably, a “cancer antigen” includes any molecule present on, or associated with, a cancer, e.g., a cancer cell or a tumor microenvironment that can provoke an immune response. As used, herein an “immune cell antigen” includes any molecule present on, or associated with, an immune cell that can provoke an immune response.

The “antigen-binding site,” or “binding portion” of an antibody molecule refers to the part of an antibody molecule, e.g., an immunoglobulin (Ig) molecule, that participates in antigen binding. In embodiments, the antigen binding site is formed by amino acid residues of the variable (V) regions of the heavy (H) and light (L) chains. Three highly divergent stretches within the variable regions of the heavy and light chains, referred to as hypervariable regions, ae disposed between more conserved flanking stretches called “framework regions,” (FRs). FRs are amino acid sequences that are naturally found between, and adjacent to, hypervariable regions in immunoglobulins. In embodiments, in an antibody molecule, the three hypervariable regions of a light chain and the three hypervariable regions of a heavy chain are disposed relative to each other in three dimensional space to form an antigen-binding surface, which is complementary to the three-dimensional surface of a bound antigen. The three hypervariable regions of each of the heavy and light chains are referred to as “complementarity-determining regions,” or “CDRs.” The framework region and CDRs have been defined and described, e.g., in Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917. Each variable chain (e.g., variable heavy chain and variable light chain) is typically made up of three CDRs and four FRs, arranged from amino-terminus to carboxy-terminus in the amino acid order: FR1, CDR1, FR2, CDR2, FR3, CDR3, and FR4.

As used herein, an “immune cell” refers to any of various cells that function in the immune system. e.g., to protect against agents of infection and foreign matter. In embodiments, this term includes leukocytes, e.g., neutrophils, eosinophils, basophils, lymphocytes, and monocytes. Innate leukocytes include phagocytes (e.g., macrophages, neutrophils, and dendritic cells), mast cells, eosinophils, basophils, and natural killer cells. Innate leukocytes identify and eliminate pathogens, either by attacking larger pathogens through contact or by engulfing and then killing microorganisms, and are mediators in the activation of an adaptive immune response. The cells of the adaptive immune system are special types of leukocytes, called lymphocytes. B cells and T cells are important types of lymphocytes and are derived from hematopoietic stem cells in the bone marrow. B cells are involved in the humoral immune response, whereas T cells are involved in cell-mediated immune response. The term “immune cell” includes immune effector cells.

“Immune effector cell,” as that term is used herein, refers to a cell that is involved in an immune response. e.g., in the promotion of an immune effector response. Examples of immune effector cells include, but are not limited to. T cells, e.g., alpha/beta T cells and gamma/delta T cells, B cells, natural killer (NK) cells, natural killer T (NK T) cells, and mast cells.

The term “effector function” or “effector response” refers to a specialized function of a cell. Effector function of a T cell, for example, may be cytolytic activity or helper activity including the secretion of cytokines.

The terms “polypeptide”, “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified: for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature. An isolated polynucleotide (ribonucleic acid (RNA), deoxyribonucleic acid (DNA)), or polypeptide is free of the genes/nucleic acids or sequences/amino acids that flank it in its naturally-occurring state.

The compositions and methods of the present disclosure encompass polypeptides and nucleic acids having the sequences specified, or sequences substantially identical or similar thereto, e.g., sequences at least 80%, 85%, 90%, 95% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 80%, 85%, 90%, 91%, 92%, 93%, 94%, 95%, 96%, 97%, 98% 99%, 99.5%, 99.9%, or 100% sequence identity to a reference sequence, e.g., a sequence provided herein. In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 80%, 85%, 90%, 91%, 92%, 93%, 94%, 95%, 96%, 97%, 98% 99%, 99.5%, 99.9%, or 100% sequence identity to a reference sequence, e.g., a sequence provided herein.

The term “variant” refers to a polypeptide that has a substantially identical amino acid sequence to a reference amino acid sequence, or is encoded by a substantially identical nucleotide sequence. In some embodiments, the variant is a functional variant. In some embodiments, a TCRαV variant can bind to TCRα and form a TCR α:β complex.

The term “functional variant” refers to a polypeptide that has a substantially identical amino acid sequence to a reference amino acid sequence, or is encoded by a substantially identical nucleotide sequence, and is capable of having one or more activities of the reference amino acid sequence.

Calculations of homology or sequence identity between sequences (the terms are used interchangeably herein) are performed as follows. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences. The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) C4BIOS, 4:11-17) which has been incorporated into the ALIGN program (version 2.0), using a PAM120 weight residue table, a gap length penalty of 12 and a gap penalty of 4. The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to a nucleic acid molecule of the disclosure. BLAST protein searches can be performed with the XBLAST program, score=50, wordlength=3 to obtain amino acid sequences homologous to protein molecules of the disclosure. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used.

It is understood that the molecules of the present disclosure may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

As used herein, the term “molecule” as used in, e.g., antibody molecule, cytokine molecule, receptor molecule, includes full-length, naturally-occurring molecules, as well as variants, e.g., functional variants (e.g., truncations, fragments, mutated (e.g., substantially similar sequences) or derivatized form thereof), so long as at least one function and/or activity of the unmodified (e.g., naturally-occurring) molecule remains.

As used herein, the term “mutation” refers to an alteration in the nucleotide sequence of the genome of an organism, virus, or extrachromosomal DNA. In some embodiments, the mutation may be a large-scale mutation, such as amplifications (or gene duplications) or repetitions of a chromosomal segment, deletions of large chromosomal regions, chromosomal rearrangements (e.g., chromosomal translocations, chromosomal inversions, non-homologous chromosomal crossover, and interstitial deletions), and loss of heterozygosity. In some embodiments, the mutation may be a small-scale mutation, such as insertions, deletions, and substitution mutations. As used herein, the term “substitution mutation” refers to the transition that exchange a single nucleotide for another.

“Interleukin-2” also known as IL2, IL-2, IL 2, TCGF, lymphokine, and interleukin 2, as referred to herein, includes any of the recombinant or naturally-occurring forms of IL-2 or variants or homologs thereof that have or maintain IL-2 activity (e.g., at least 40% 50%, 60%, 70%, 80%, 90%, 95%, 96%, 97%, 98%, 99% or 100% activity). In some aspects, the variants or homologs have at least 40%, 45%, 50%, 55%, 60%, 65%, 70%, 75%, 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring IL-2. In some embodiments, IL-2 is substantially identical to the protein identified by the UniProt reference number P60568 or a variant or homolog having substantial identity thereto.

### Anti-TCRαV Antibodies

### Hunan T Cell Receptor (TCR) Complex

TCR is a disulfide-linked membrane-anchored heterodimeric protein normally consisting of the highly variable alpha (α) and beta (β) chains expressed as part of a complex with the invariant CD3 chain molecules. TCR on αβ T cells is formed by a heterodimer of one alpha chain and one beta chain. Each alpha or beta chain consists of a constant domain and a highly variable domain classified as the Immunoglobulin superfamily (IgSF) fold. The TCRαV chains can be further classified into subfamilies (TRAV1-10, 12-14, 16-27, 29, 30, 34-36 an 38-41). Despite their high structural and functional homology, the amino acid sequence homology in the TRAV genes is very low. Nevertheless, TCRs formed between alpha and beta chains of highly diverse sequences show a remarkable structural and elicit a similar function, e.g., activation of T cells.

T cell receptors (TCR) can be found on the surface of T cells. TCRs recognize antigens, e.g., peptides, presented on, e.g., bound to, major histocompatibility complex (MHC) molecules on the surface of cells, e.g., antigen-presenting cells. TCRs are heterodimeric molecules and can comprise an alpha chain, a beta chain, a gamma chain or a delta chain. TCRs comprising an alpha chain and a beta chain are also referred to as TCRαβ. The TCR beta chain consists of the following regions (also known as segments), variable (V), diversity (D), joining (J) and constant (C) (see Mayer G. and Nyland J. (2010) Chapter 10: Major Histocompatibility Complex and T-cell Receptors-Role in Immune Responses. In: Microbiology and Immunology on-line, University of South Carolina School of Medicine). The TCR alpha chain consists of V, J and C regions. The rearrangement of the T-cell receptor (TCR) through somatic recombination of V (variable), D (diversity), J (joining), and C (constant) regions is a defining event in the development and maturation of a T cell. TCR gene rearrangement takes place in the thymus.

TCRs can comprise a receptor complex, known as the TCR complex, which comprises a TCR heterodimer comprising of an alpha chain and a beta chain, and dimeric signaling molecules, e.g., CD3 co-receptors, e.g., CD3δ/ε, and/or CD3γ/ε.

As used herein, the term “T cell receptor alpha variable chain” or “TCRαV,” or “TRAV,” refers to an extracellular region of the T cell receptor alpha chain which can comprise a portion of the antigen recognition domain of the T cell receptor. The term TCRαV includes isoforms, mammalian, e.g., human TCRαV, species homologs of human and analogs comprising at least one common epitope with TCRαV. Human TCRαV comprises a gene family comprising subfamilies including, but not limited to: a TCRα V1 subfamily, a TCRα V2 subfamily, a TCRα V3 subfamily, a TCRα V4, a TCRα V5 subfamily, a TCRα V6 subfamily, a TCRα V7 subfamily, a TCRα V8 subfamily, a TCRα V9 subfamily, a TCRα V10 subfamily, a TCRα V12 subfamily, a TCRα V13 subfamily, a TCRα V14 subfamily, a TCRα V16 subfamily, a TCRα V17 subfamily, a TCRα V18 subfamily, a TCRα V19 subfamily, a TCRα V20 subfamily, a TCRα V21 subfamily, a TCRα V22 subfamily, a TCRα V23 subfamily, a TCRα V24 subfamily, TCRα V25 subfamily, a TCRα V26 subfamily, a TCRα V27 subfamily, a TCRα V29 subfamily, a TCRα V30 subfamily, a TCRα V34 subfamily, a TCRα V35 subfamily, a TCRα V36 subfamily, a TCRα V38 subfamily, a TCRα V39 subfamily, a TCRα V40 subfamily, or a TCRα V41 subfamily, as well as family members of said subfamilies, and variants thereof (e.g., a structural or functional variant thereof).

In some embodiments, the TCRα V1 subfamily comprises: TCRαV1-1 or TCRαV1-2, or a variant thereof.

In some embodiments, the TCRα V8 subfamily comprises: TCRαV8-1, TCRαV8-2, TCRαV8-3, TCRαV8-4, or TCRαV8-6, or a variant thereof.

In some embodiments, the TCRα V9 subfamily comprises: TCRαV9-1 or TCRαV9-2, or a variant thereof.

In some embodiments, the TCRα V12 subfamily comprises: TCRαV12-1, TCRαV12-2, or TCRαV12-3, or a variant thereof.

In some embodiments, the TCRα V13 subfamily comprises: TCRαV13-1 or TCRαV13-2, or a variant thereof.

In some embodiments, the TCRα V14 subfamily comprises: TCRαV14/DV4, or a variant thereof.

In some embodiments, the TCRα V23 subfamily comprises: TCRαV23/DV6, or a variant thereof.

In some embodiments, the TCRα V26 subfamily comprises: TCRαV26-1 or TCRαV26-2, or a variant thereof.

In some embodiments, the TCRα V29 subfamily comprises: TCRαV29/DV5, or a variant thereof.

In some embodiments, the TCRα V36 subfamily comprises: TCRαV236/DV7, or a variant thereof.

In some embodiments, the TCRα V38 subfamily comprises: TCRαV38-1 or TCRαV38-2/DV8, or a variant thereof.

TCR alpha V (TCRαV)

Diversity in the immune system enables protection against a huge array of pathogens. Since the germline genome is limited in size, diversity is achieved not only by the process of V(D)J recombination but also by junctional (junctions between V-D and D-J segments) deletion of nucleotides and addition of pseudo-random, non-templated nucleotides. The TCR alpha gene undergoes gene arrangement to generate diversity.

The TCR V alpha repertoire varies between individuals and populations because of, e.g., 7 frequently occurring inactivating polymorphisms in functional gene segments and a large insertion/deletion-related polymorphism encompassing 2 V alpha gene segments.

Provided herein are, inter alia, antibody molecules and fragments thereof, that bind, e.g., specifically bind, to a human TCR alpha V chain (TCRαV), e.g., a TCRαV gene family (also referred to as a group), e.g., a TCRαV subfamily (also referred to as a subgroup), e.g., as described herein. TCR alpha V families and subfamilies are known in the art, e.g., as described in Yassai et al., (2009) Immunogenetics 61(7)pp:493-502: Wei S. and Concannon P. (1994) Human Immunology 41(3) pp: 201-206. The antibodies described herein can be recombinant antibodies, e.g., recombinant non-murine antibodies, e.g., recombinant human or humanized antibodies.

The terms TCRAV, TCRVA, TRAV, TCRαV, TCRVα or TRαV are used interchangeably herein and refer to a TCR alpha V chain, e.g., as described herein.

In some embodiments, provided herein is an anti-TCRαV antibody molecule that binds to human TCRαV, e.g., a TCRαV family, e.g., gene family or a variant thereof.

Exemplary amino acid sequences for TCRαV subfamily members can be found on the ImMunoGeneTics Information System website: http://www.imgt.org/, or in a similar resource.

### Anti-TCRαV Antibodies

Current bispecific constructs designed to redirect T cells to promote tumor cell lysis for cancer immunotherapy typically utilize antibody fragments (Fab, scFv, VH, single domain antibody, etc.) that are derived from monoclonal antibodies (mAb) directed against the CD3e subunit of the T cell receptor (TCR). However, there are limitations to this approach which may prevent the full realization of the therapeutic potential for such bispecific constructs. Previous studies have shown that even low “activating” doses of anti-CD3e mAb can cause long-term T cell dysfunction and exert immunosuppressive effects. In addition, anti-CD3e mAbs have been associated with side effects that result from massive T cell activation. The large number of activated T cells secrete substantial amounts of cytokines, the most important of which is Interferon gamma (IFNγ). This excess amount of IFNγ in turn activates macrophages which then overproduce proinflammatory cytokines such as IL-1beta, IL-6, IL-10 and TNF-alpha, causing a “cytokine storm” known as the cytokine release syndrome (CRS) (Shimabukuro-Vornhagen et al., J Immunother Cancer, 2018 Jun. 15; 6(1):56, herein incorporated by reference in its entirety). Thus, the need exists for developing antibodies that are capable of binding and activating only a subset of effector T cells, e.g., to re-duce the CRS and/or neurotoxicity (NT).

Described herein are molecules targeting the TCRαV chain of TCR and methods thereof. Without wishing to be bound by theory, such molecules are capable of binding, activating, and/or expanding only a subset of T cells, avoiding or reducing CRS and/or NT and minimizing potential immunosuppressive effects of anti-CD3 mAbs.

Described herein is a class of antibodies, i.e., anti-TCRαV antibody molecules as described herein, which despite having low sequence similarity (e.g., low sequence identity among the different antibody molecules that recognize different TCRαV subfamilies), recognize a structurally conserved, yet sequence-wise variable, region, e.g., domain, on the TCRαV protein and have a similar function (e.g., activation of T cells and a similar cytokine profile as described herein). Thus, the anti-TCRαV antibody molecules as described herein share a structure-function relationship.

In some embodiments, the anti-TCRαV antibody molecules as described herein do not recognize. e.g., bind to, an interface of a TCRβV:TCRalpha complex. In some embodiments, the anti-TCRαV antibody molecules as described herein do not recognize, e.g., bind to, a constant region of a TCRβV protein. In some embodiments, the anti-TCRαV antibody molecules as described herein do not recognize, e.g., bind to, one or more (e.g., all) of a complementarity determining region (e.g., CDR1, CDR2 and/or CDR3) of a TCRβV protein.

Provided herein are, inter aba, antibody molecules directed to the variable chain of the alpha subunit of TCR (TCRαV) which bind and, e.g., activate a subset of T cells. The anti-TCRαV antibody molecules as described herein result in lesser or no production of cytokines associated with CRS, e.g., IL-6, IL-1beta, IL-10 and TNF alpha; and enhanced and/or delayed production of IL-2 and IFNγ. In some embodiments, the anti-TCRαV antibodies as described herein have a cytokine profile, e.g., as described herein, which differs from a cytokine profile of a T cell engager that binds to a receptor or molecule other than a TCRαV region (“a non-TCRαV-binding T cell engager”). In some embodiments, the non-TCRαV-binding T cell engager comprises an antibody that binds to a CD3 molecule (e.g., CD3 epsilon (CD3e) molecule); or a TCR alpha (TCRα) molecule. In some embodiments, the non-TCRαV-binding T cell engager is an OKT3 antibody or an SP34-2 antibody.

In some embodiments, the anti-TCRαV antibodies as described herein result in expansion of TCRαV+ T cells, e.g., a subset of memory effector T cells known as TEMRA. Without wishing to be bound by theory, it is believed that in some embodiments, TEMRA cells can promote tumor cell lysis but not CRS. Accordingly, provided herein are methods of making said anti-TCRαV antibody molecules and uses thereof. Also described herein are multispecific molecules, e.g., bispecific molecules comprising said anti-TCRαV antibody molecules. In some embodiments, compositions comprising anti-TCRαV antibody molecules of the present disclosure, can be used, e.g., to: (1) activate and redirect T cells to promote tumor cell lysis for cancer immuno-therapy; and/or (2) expand TCRαV+ T cells. In some embodiments, compositions comprising anti-TCRαV antibody molecules as described herein limit the harmful side-effects of CRS and/or NT, e.g., CRS and/or NT associated with anti-CD3e targeting.

In some embodiments, the anti-TCRαV antibody molecule is a full antibody or fragment thereof (e.g., a Fab, F(ab′)₂, Fv, single domain antibody, or a single chain Fv fragment (scFv)). In embodiments, the anti-TCRαV antibody molecule is a monoclonal antibody or an antibody with single specificity. In some embodiments, the anti-TCRαV antibody molecule can also be a humanized, chimeric, camelid, shark, or an in vitro-generated antibody molecule. In some embodiments, the anti-TCRαV antibody molecule is a humanized antibody molecule. The heavy and light chains of the anti-TCRαV antibody molecule can be full-length (e.g., an antibody can include at least one, and preferably two, complete heavy chains, and at least one, and preferably two, complete light chains) or can include an antigen-binding fragment (e.g., a Fab, F(ab′)2, Fv, a single chain Fv fragment, a single domain antibody, a diabody (dAb), a bivalent antibody, or bispecific antibody or fragment thereof, a single domain variant thereof, or a camelid antibody).

In some embodiments, the anti-TCRαV antibody molecule is in the form of a multispecific molecule, e.g., a bispecific molecule, e.g., as described herein.

In some embodiments, the anti-TCRαV antibody molecule has a heavy chain constant region (Fc) chosen from, e.g., the heavy chain constant regions of IgG1, IgG2, IgG3, IgG4, IgM, IgA1, IgA2, IgD, and IgE. In some embodiments, the Fc region is chosen from the heavy chain constant regions of IgG1, IgG2, IgG3, and IgG4. In some embodiments, the Fc region is chosen from the heavy chain constant region of IgG1 or IgG2 (e.g., human IgG1, or IgG2). In some embodiments, the heavy chain constant region is human IgG1. In some embodiments, the Fc region comprises a Fc region variant, e.g., as described herein.

In some embodiments, the anti-TCRαV antibody molecule has a light chain constant region chosen from, e.g., the light chain constant regions of kappa or lambda, preferably kappa (e.g., human kappa). In some embodiments, the constant region is altered, e.g., mutated, to modify the properties of the anti-TCRαV antibody molecule (e.g., to increase or decrease one or more of: Fc receptor binding, antibody glycosylation, the number of cysteine residues, effector cell function, or complement function). For example, the constant region is mutated at positions 296 (M to Y), 298 (S to T), 300 (T to E), 477 (H to K) and 478 (N to F) to alter Fc receptor binding (e.g., the mutated positions correspond to positions 132(M to Y), 134 (S to T), 136 (T to E), 313 (H to K) and 314 (N to F) of SEQ ID NOs: 212 or 214; or positions 135 (M to Y), 137 (S to T), 139 (T to E), 316 (H to K) and 317 (N to F) of SEQ ID NOs: 215, 216, 217 or 218), e.g., relative to human IgG1.

The various TCRαV subfamilies and/or subfamily members can be expressed at different levels in individuals, e.g., healthy individuals, as disclosed in Kitaura K. et al (2016), BMC Immunology vol 17: 38, the entire contents of which are hereby incorporated by reference.

In some embodiments, the anti-TCRαV antibody molecule is a non-murine antibody molecule, e.g., a human or humanized antibody molecule. In some embodiments, the anti-TCRαV antibody molecule is a human antibody molecule. In some embodiments, the anti-TCRαV antibody molecule is a humanized antibody molecule.

In some embodiments, the anti-TCRαV antibody molecule is isolated or recombinant.

In some embodiments, the anti-TCRαV antibody molecule comprises a heavy chain constant region for an IgG4, e.g., a human IgG4. In still another embodiment, the anti-TCRαV antibody molecule includes a heavy chain constant region for an IgG1, e.g., a human IgG1. In some embodiments, the heavy chain constant region comprises an amino sequence set forth in Table 1, or a sequence substantially identical (e.g., at least 80%, 85%, 90%, 92%, 95%, 97%, 98%, 99% or higher identical) thereto.

In some embodiments, the anti-TCRαV antibody molecule includes a kappa light chain constant region, e.g., a human kappa light chain constant region. In some embodiments, the light chain constant region comprises an amino sequence set forth in Table 1, or a sequence substantially identical (e.g., at least 80%, 85%, 90%, 92%, 95%, 97%, 98%, 99% or higher identical) thereto.

In some embodiments, e.g., an embodiment comprising a variable region, a CDR (e.g., a combined CDR, Chothia CDR or Kabat CDR), or other sequence referred to herein, the antibody molecule is a monospecific antibody molecule, a bispecific antibody molecule, a bivalent antibody molecule, a biparatopic antibody molecule, or an antibody molecule that comprises an antigen binding fragment of an antibody, e.g., a half antibody or antigen binding fragment of a half antibody. In certain embodiments the antibody molecule comprises a multispecific molecule, e.g., a bispecific molecule, e.g., as described herein.

In some embodiments, the anti-TCRαV antibody molecule, is a non-murine antibody molecule, e.g., a human or humanized antibody molecule. In some embodiments, the anti-TCRαV antibody molecule is a human antibody molecule. In some embodiments, the anti-TCRαV antibody molecule is a humanized antibody molecule.

In some embodiments, the anti-TCRαV antibody molecule, is isolated or recombinant.

In some embodiments, the anti-TCRαV antibody molecule can contain any combination of CDRs or hypervariable loops according to the Kabat and Chothia definitions.

In some embodiments, e.g., an embodiment comprising a variable region, a CDR (e.g., a combined CDR, Chothia CDR or Kabat CDR), or other sequence referred to herein, the antibody molecule is a monospecific antibody molecule, a bispecific antibody molecule, a bivalent antibody molecule, a biparatopic antibody molecule, or an antibody molecule that comprises an antigen binding fragment of an antibody, e.g., a half antibody or antigen binding fragment of a half antibody. In certain embodiments the antibody molecule comprises a multispecific molecule, e.g., a bispecific molecule, e.g., as described herein.

In some embodiments, the anti-TCRαV antibody molecule comprises a light chain variable domain comprising: (a) a framework region 1 (FR1) comprising a change, e.g., a substitution (e.g., a conservative substitution) at one or more (e.g., all) positions as described herein according to Kabat numbering, and (b) a framework region 3 (FR3) comprising a change, e.g., a substitution (e.g., a conservative substitution) at one or more (e.g., all) position as described herein according to Kabat numbering. In some embodiments, the substitution is relative to a human germline light chain framework region sequence.

In some embodiments, the anti-TCRαV antibody molecule is a full antibody or fragment thereof (e.g., a Fab, F(ab′)₂, Fv, or a single chain Fv fragment (scFv)). In embodiments, the anti-TCRαV antibody molecule is a monoclonal antibody or an antibody with single specificity. In some embodiments, the anti-TCRαV antibody molecule can also be a humanized, chimeric, camelid, shark, or an in vitro-generated antibody molecule. In some embodiments, the anti-TCRαV antibody molecule is a humanized antibody molecule. The heavy and light chains of the anti-TCRαV antibody molecule can be full-length (e.g., an antibody can include at least one, and preferably two, complete heavy chains, and at least one, and preferably two, complete light chains) or can include an antigen-binding fragment (e.g., a Fab, F(ab′)2, Fv, a single chain Fv fragment, a single domain antibody, a diabody (dAb), a bivalent antibody, or bispecific antibody or fragment thereof, a single domain variant thereof, or a camelid antibody).

In some embodiments, the anti-TCRαV antibody molecule is in the form of a multispecific molecule, e.g., a bispecific molecule, e.g., as described herein.

In some embodiments, the anti-TCRαV antibody molecule has a heavy chain constant region (Fc) chosen from, e.g., the heavy chain constant regions of IgG1, IgG2, IgG3, IgG4, IgM, IgA1, IgA2, IgD, and IgE. In some embodiments, the Fc region is chosen from the heavy chain constant regions of IgG1, IgG2, IgG3, and IgG4. In some embodiments, the Fc region is chosen from the heavy chain constant region of IgG1 or IgG2 (e.g., human IgG1, or IgG2). In some embodiments, the heavy chain constant region is human IgG1.

In some embodiments, the anti-TCRαV antibody molecule has a light chain constant region chosen from, e.g., the light chain constant regions of kappa or lambda, preferably kappa (e.g., human kappa). In some embodiments, the constant region is altered, e.g., mutated, to modify the properties of the anti-TCRαV antibody molecule (e.g., to increase or decrease one or more of: Fc receptor binding, antibody glycosylation, the number of cysteine residues, effector cell function, or complement function). For example, the constant region is mutated at positions 296 (M to Y), 298 (S to T), 300 (T to E), 477 (H to K) and 478 (N to F) to alter Fc receptor binding (e.g., the mutated positions correspond to positions 132 (M to Y), 134 (S to T), 136 (T to E), 313 (H to K) and 314 (N to F) of SEQ ID NOs: 212 or 214; or positions 135 (M to Y), 137 (S to T), 139 (T to E), 316 (H to K) and 317 (N to F) of SEQ ID NOs: 215, 216, 217 or 218).

### Anti-TCRα V12 Antibodies

In one aspect, provided herein is an anti-TCRαV antibody molecule that binds to a human TCRα V12 subfamily member. In some embodiments, TCRα V12 subfamily is also known as TCRα V12. In some embodiments, the TCRα V12 subfamily comprises: TCRα V12-1, TCRα V12-2 or TCRα V12-3, or a variant thereof.

### Antibody-Like Frameworks or Scaffolds

A wide variety of antibody/immunoglobulin frameworks or scaffolds can be employed in the anti-TCRVA antibody molecules as described herein or multifunctional formats thereof so long as the resulting poly peptide includes at least one binding region which specifically binds to the target antigen, e.g., a TCRVA, a tumor antigen, among others. Such frameworks or scaffolds include the 5 main idiotypes of human immunoglobulins, or fragments thereof, and include immunoglobulins of other animal species, preferably having humanized aspects. Novel frameworks, scaffolds and fragments continue to be discovered and developed by those skilled in the art.

In some embodiments, the anti-TCRαV antibody molecules as described herein or multifunctional formats thereof include non-immunoglobulin based antibodies using non-immunoglobulin scaffolds onto which CDRs can be grafted. Any non-immunoglobulin frameworks and scaffolds may be employed, as long as they comprise a binding region specific for the target antigen (e.g., TCRαV or a tumor antigen). Exemplary non-immunoglobulin frameworks or scaffolds include, but are not limited to, fibronectin (Compound Therapeutics, Inc., Waltham, MA), ankyrin (Molecular Partners AG, Zurich. Switzerland), domain antibodies (Domantis. Ltd., Cambridge, MA, and Ablynx nv, Zwijnaarde, Belgium), lipocalin (Pieris Proteolab AG, Freising, Germany), small modular immuno-pharmaceuticals (Trubion Pharmaceuticals Inc., Seattle, WA), maxybodies (Avidia, Inc., Mountain View, CA), Protein A (Affibody AG, Sweden), and affilin (gamma-crystallin or ubiquitin)(Scil Proteins GmbH, Halle, Germany).

Fibronectin scaffolds are typically based on fibronectin type III domain (e.g., the tenth module of the fibronectin typeIII (10 Fn3 domain)). The fibronectin type III domain has 7 or 8 beta strands which are distributed between two beta sheets, which themselves pack against each other to form the core of the protein, and further containing loops (analogous to CDRs) which connect the beta strands to each other and are solvent exposed. There are at least three such loops at each edge of the beta sheet sandwich, where the edge is the boundary of the protein perpendicular to the direction of the beta strands (see U.S. Pat. No. 6,818,418). Because of this structure, the non-immunoglobulin antibody mimics antigen binding properties that are similar in nature and affinity to those of antibodies. These scaffolds can be used in a loop randomization and shuffling strategy in vitro that is similar to the process of affinity maturation of antibodies in vivo. These fibronectin-based molecules can be used as scaffolds where the loop regions of the molecule can be replaced with CDRs of the disclosure using standard cloning techniques.

The ankyrin technology is based on using proteins with ankyrin derived repeat modules as scaffolds for bearing variable regions which can be used for binding to different targets. The ankyrin repeat module typically is a about 33 amino acid polypeptide consisting of two anti-parallel α-helices and a β-turn. Binding of the variable regions can be optimized by using ribosome display.

Avimers are used by nature for protein-protein interactions and in human over 250 proteins are structurally based on A-domains. Avimers consist of a number of different “A-domain” monomers (2-10)linked via amino acid linkers. Avimers can be created that can bind to the target antigen using the methodology described in, for example, U.S. Patent Application Publication Nos. 20040175756; 20050053973, 20050048512; and 20060008844.

Affibody affinity ligands are small, simple proteins composed of a three-helix bundle based on the scaffold of one of the IgG-binding domains of Protein A. Protein A is a surface protein from the bacterium Staphylococcus aureus. This scaffold domain consists of 58 amino acids, 13 of which are randomized to generate affibody libraries with a large number of ligand variants (See e.g., U.S. Pat. No. 5,831,012). Affibody molecules mimic antibodies, they have a molecular weight of 6 kDa, compared to the molecular weight of antibodies, which is 150 kDa. In spite of its small size, the binding site of affibody molecules is similar to that of an antibody.

Anticalins are known commercially. e.g., Pieris ProteoLab AG. They are derived from lipocalins, a widespread group of small and robust proteins that are usually involved in the physiological transport or storage of chemically sensitive or insoluble compounds. Several natural lipocalins occur in human tissues or body liquids. The protein architecture is reminiscent of immunoglobulins, with hypervariable loops on top of a rigid framework. However, in contrast with antibodies or their recombinant fragments, lipocalins are composed of a single polypeptide chain with 160 to 180 amino acid residues, being just marginally bigger than a single immunoglobulin domain. The set of four loops, which makes up the binding pocket, shows pronounced structural plasticity and tolerates a variety of side chains. The binding site can thus be reshaped in a proprietary process in order to recognize prescribed target molecules of different shape with high affinity and specificity. One protein of lipocalin family, the bilin-binding protein (BBP) of Pieris Brassicae has been used to develop anticalins by mutagenizing the set of four loops. One example of a patent application describing anticalins is in PCT Publication No. WO 199916873.

Affilin molecules are small non-immunoglobulin proteins which are designed for specific affinities towards proteins and small molecules. New affilin molecules can be very quickly selected from two libraries, each of which is based on a different human derived scaffold protein. Affilin molecules do not show any structural homology to immunoglobulin proteins. Currently, two affilin scaffolds are employed, one of which is gamma crystalline, a human structural eye lens protein and the other is “ubiquitin” superfamily proteins. Both human scaffolds are very small, show high temperature stability and are almost resistant to pH changes and denaturing agents. This high stability is mainly due to the expanded beta sheet structure of the proteins. Examples of gamma crystalline derived proteins are described in WO200104144 and examples of “ubiquitin-like” proteins are described in WO2004106368.

Protein epitope mimetics (PEM) are medium-sized, cyclic, peptide-like molecules (MW 1-2 kDa) mimicking beta-hairpin secondary structures of proteins, the major secondary structure involved in protein-protein interactions.

Domain antibodies (dAbs) can be used in the anti-TCRVA antibody molecules as described herein or multifunctional formats thereof are small functional binding fragments of antibodies, corresponding to the variable regions of either the heavy or light chains of antibodies. Domain antibodies are well expressed in bacterial, yeast, and mammalian cell systems. Further details of domain antibodies and methods of production thereof are known in the art (see, for example, U.S. Pat. Nos. 6,291,158; 6,582,915; 6,593,081; 6,172,197; 6,696,245; European Patents 0368684 & 0616640: WO05/035572, WO04/101790, WO04/081026, WO04/058821, WO04/003019 and WO03/002609. Nanobodies are derived from the heavy chains of an antibody.

A nanobody typically comprises a single variable domain and two constant domains (CH2 and CH3) and retains antigen-binding capacity of the original antibody. Nanobodies can be prepared by methods known in the art (See e.g., U.S. Pat. Nos. 6,765,087, 6,838,254, WO 06/079372). Unibodies consist of one light chain and one heavy chain of an IgG4 antibody. Unibodies may be made by the removal of the hinge region of IgG4 antibodies. Further details of unibodies and methods of preparing them may be found in WO2007/059782.

### Anti-TCRVα Antibody Effector Function and Fc Variants

In some embodiments, an anti-TCRVα antibody as described herein comprises an Fc region, e.g., as described herein. In some embodiments, the Fc region is a wildtype Fc region, e.g., a wildtype human Fc region. In some embodiments, the Fc region comprises a variant, e.g., an Fc region comprising an addition, substitution, or deletion of at least one amino acid residue in the Fc region which results in, e.g., reduced or ablated affinity for at least one Fc receptor.

The Fc region of an antibody interacts with a number of receptors or ligands including Fc Receptors (e.g., FcγRI, FcγRIIA, FcγRIIIA), the complement protein CIq, and other molecules such as proteins A and G. These interactions are essential for a variety of effector functions and downstream signaling events including: antibody dependent cell-mediated cytotoxicity (ADCC), Antibody-dependent cellular phagocytosis (ADCP) and complement dependent cytotoxicity (CDC).

In some embodiments, an anti-TCRVα antibody comprising a variant Fc region has reduced, e.g., ablated, affinity for an Fc receptor, e.g., an Fc receptor described herein. In some embodiments, the reduced affinity is compared to an otherwise similar antibody with a wildtype Fc region.

In some embodiments, an anti-TCRVα antibody comprising a variant Fc region has one or more of the following properties: (1) reduced effector function (e.g., reduced ADCC, ADCP and/or CDC); (2) reduced binding to one or more Fc receptors; and/or (3) reduced binding to C1q complement. In some embodiments, the reduction in any one, or all of properties (1)-(3) is compared to an otherwise similar antibody with a wildtype Fc region.

In some embodiments, an anti-TCRVα antibody comprising a variant Fc region has reduced affinity to a human Fc receptor, e.g., FcγR I, FcγR II and/or FcγR III. In some embodiments, the anti-TCRVα antibody comprising a variant Fc region comprises a human IgG1 region or a human IgG4 region.

In some embodiments, an anti-TCRVα antibody comprising a variant Fc region activates and/or expands T cells, e.g., as described herein. In some embodiments, an anti-TCRVα antibody comprising a variant Fc region has a cytokine profile described herein, e.g., a cytokine profile that differs from a cytokine profile of a T cell engager that binds to a receptor or molecule other than a TCRαV region (“a non-TCRαV-binding T cell engager”). In some embodiments, the non-TCRαV-binding T cell engager comprises an antibody that binds to a CD3 molecule (e.g., CD3 epsilon (CD3e) molecule), or a TCR alpha (TCRα) molecule.

Exemplary Fc region variants are provided in Table 6 and also disclosed in Saunders O, (2019) Frontiers in Immunology: vol 10, article1296, the entire contents of which is hereby incorporated by reference.

In some embodiments, an anti-TCRVα antibody as described herein comprises any one or all, or any combination of Fc region variants disclosed in Table 6.

In some embodiments, an anti-TCRVα antibody as described herein comprises any one or all, or any combination of Fc region variants, e.g., mutations, disclosed in Table 6. In some embodiments, an anti-TCRVα antibody as described herein comprise an Asn297Ala (N297A) mutation. In some embodiments, an anti-TCRVα antibody as described herein comprise a Leu234Ala/Leu235Ala (LALA) mutation.

### Multifunctional Molecules

As used herein, a “multifunctional” or a “multispecific” molecule refers to molecule, e.g., a polypeptide, that has two or more functionalities, e.g., two or more binding specificities. In some embodiments, the functionalities can include one or more immune cell engagers, one or more tumor binding molecules, one or more cytokine molecules, one or more stromal modifiers, and other moieties described herein. In some embodiments, the multispecific molecule is a multispecific antibody molecule, e.g., a bispecific antibody molecule. In some embodiments, the multispecific molecule includes an anti-TCRVA antibody molecule as described herein.

Described herein, in certain embodiments, is a multifunctional polypeptide molecule comprising a first polypeptide, a second polypeptide, a third polypeptide, a fourth polypeptide, and at least one cytokine polypeptide or a variant thereof, wherein the first polypeptide, the second polypeptide, the third polypeptide, and the fourth polypeptide are non-contiguous, wherein: (i) the first polypeptide comprising a first portion of a first T cell receptor variable alpha (TCRαV)-binding moiety and a first dimerization module linked to the first portion of the first TCRαV-binding moiety; (ii) the second polypeptide comprising a second portion of the first TCRαV-binding moiety; (iii) the third polypeptide comprising a first portion of a second TCRαV-binding moiety and a second dimerization module linked to the first portion of the second TCRαV-binding moiety; and (iv) the fourth polypeptide comprising a second portion of the second TCRαV-binding moiety; and wherein the at least one cytokine polypeptide or the variant thereof is covalently linked to the first polypeptide, the second polypeptide, the third polypeptide, the fourth polypeptide, or a combination thereof.

Described herein, in certain embodiments, is a multifunctional polypeptide molecule comprising a first polypeptide, a second polypeptide, a third polypeptide, and at least one cytokine polypeptide or a variant thereof, wherein the first polypeptide, the second polypeptide, and the third polypeptide are non-contiguous, wherein: (i) the first polypeptide comprising a first portion of a first TCRαV-binding moiety and a first dimerization module linked to the first portion of the first TCRαV-binding moiety; (ii) the second polypeptide comprising a second portion of the first TCRαV-binding moiety; and (iii) the third polypeptide comprising a second dimerization module; and wherein the at least one cytokine polypeptide or the variant thereof is covalently linked to the first polypeptide, the second polypeptide, the third polypeptide, or a combination thereof.

Described herein, in certain embodiments, is a multifunctional polypeptide molecule comprising a first poly peptide, a second polypeptide, a third polypeptide, and at least one cytokine polypeptide or a variant thereof, wherein the first polypeptide, the second polypeptide, and the third polypeptide are non-contiguous, wherein: (i) the first polypeptide comprising a first portion of a first TCRαV-binding moiety and a first dimerization module linked to the first portion of the first TCRαV-binding moiety; (ii) the second polypeptide comprising a second portion of the first TCRαV-binding moiety; and (iii) the third polypeptide comprising a second dimerization module; wherein the at least one cytokine polypeptide or the variant thereof is covalently linked to the first polypeptide, the second polypeptide, the third polypeptide, or a combination thereof, and wherein the multifunctional polypeptide molecule does not comprise an additional TCRαV-binding moiety except the first TCRαV-binding moiety.

In some embodiments, the first portion of the first TCRαV-binding moiety comprises a first heavy chain variable domain (VH) and a first heavy chain constant domain 1 (CH1) linked to the first VH. In some embodiments, the first CH1 is linked to the C-terminus of the first VH. In some embodiments, the second portion of the first TCRαV-binding moiety comprises a first light chain variable domain (V L) and a first light chain constant domain (CL) linked to the first VL. In some embodiments, first CL is linked to the C-terminus of the first VL. In some embodiments, wherein the first dimerization module is linked to the first portion of the first TCRαV-binding moiety. In some embodiments, the first dimerization module is linked to the C-terminus of the first portion of the first TCRαV-binding moiety. In some embodiments, wherein the first portion of the second TCRαV-binding moiety comprises a second VH and a second CH1 linked to the second VH. In some embodiments, the second CH1 is linked to the C-terminus of the second VH. In some embodiments, the second portion of the second TCRαV-binding moiety comprises a second VL and a second CL linked to the second VL. In some embodiments, the second CL is linked to the C-terminus of the second VL. In some embodiments, the second dimerization module is linked to the first portion of the second TCRαV-binding moiety. In some embodiments, the second dimerization module is linked to the C-terminus of the first portion of the second TCRαV-binding moiety.

In some embodiments, (a) the N-terminus of the first polypeptide is linked to a first cytokine polypeptide or a variant thereof; the C-terminus of the first polypeptide is linked to a second cytokine polypeptide or a variant thereof; or a combination thereof; (b) the N-terminus of the second polypeptide is linked to a third cytokine polypeptide or a variant thereof; the C-terminus of the second polypeptide is linked to a fourth cytokine polypeptide or a variant thereof; or a combination thereof; (c) the N-terminus of the third polypeptide is linked to a fifth cytokine polypeptide or a variant thereof; the C-terminus of the third polypeptide is linked to a sixth cytokine polypeptide or a variant thereof; or a combination thereof; (d) the N-terminus of the fourth polypeptide is linked to a seventh cytokine polypeptide or a variant thereof; the C-terminus of the fourth polypeptide is linked to an eighth cytokine polypeptide or a variant thereof; or a combination thereof; or (e) a combination thereof.

In some embodiments, (a-1) the N-terminus of the first polypeptide is linked to the first cytokine polypeptide or the variant thereof; the C-terminus of the first polypeptide is linked to the second cytokine polypeptide or the variant thereof; or a combination thereof; and (a-2) the N-terminus of the second polypeptide is linked to the third cytokine polypeptide or the variant thereof; the C-terminus of the second polypeptide is linked to the fourth cytokine polypeptide or the variant thereof; or a combination thereof; (b-1) the N-terminus of the first polypeptide is linked to the first cytokine polypeptide or the variant thereof; the C-terminus of the first polypeptide is linked to the second cytokine polypeptide or the variant thereof; or a combination thereof; and (b-2) the N-terminus of the third polypeptide is linked to the fifth cytokine polypeptide or the variant thereof; the C-terminus of the third polypeptide is linked to the sixth cytokine polypeptide or the variant thereof; or a combination thereof; (c-1) the N-terminus of the first polypeptide is linked to the first cytokine polypeptide or the variant thereof; the C-terminus of the first polypeptide is linked to the second cytokine polypeptide or the variant thereof; or a combination thereof; and (c-2) the N-terminus of the fourth poly peptide is linked to the seventh cytokine polypeptide or the variant thereof; the C-terminus of the fourth polypeptide is linked to the eighth cytokine polypeptide or the variant thereof; or a combination thereof; (d-1) the N-terminus of the second polypeptide is linked to the third cytokine polypeptide or the variant thereof; the C-terminus of the second polypeptide is linked to the fourth cytokine polypeptide or the variant thereof; or a combination thereof; and (d-2) the N-terminus of the third polypeptide is linked to the fifth cytokine polypeptide or the variant thereof; the C-terminus of the third polypeptide is linked to the sixth cytokine polypeptide or the variant thereof; or a combination thereof; (e-1) the N-terminus of the second polypeptide is linked to the third cytokine polypeptide or the variant thereof; the C-terminus of the second polypeptide is linked to the fourth cytokine polypeptide or the variant thereof; or a combination thereof; and (e-2) the N-terminus of the fourth polypeptide is linked to the seventh cytokine polypeptide or the variant thereof; the C-terminus of the fourth polypeptide is linked to the eighth cytokine polypeptide or the variant thereof; or a combination thereof; or (f-1) the N-terminus of the third polypeptide is linked to the fifth cytokine polypeptide or the variant thereof; the C-terminus of the third polypeptide is linked to the sixth cytokine polypeptide or the variant thereof; or a combination thereof; and (f-2) the N-terminus of the fourth polypeptide is linked to the seventh cytokine polypeptide or the variant thereof; the C-terminus of the fourth polypeptide is linked to the eighth cytokine polypeptide or the variant thereof; or a combination thereof.

In some embodiments, (a-1) the N-terminus of the first polypeptide is linked to the first cytokine polypeptide or the variant thereof; the C-terminus of the first polypeptide is linked to the second cytokine polypeptide or the variant thereof; or a combination thereof; (a-2) the N-terminus of the second polypeptide is linked to the third cytokine polypeptide or the variant thereof; the C-terminus of the second polypeptide is linked to the fourth cytokine polypeptide or the variant thereof; or a combination thereof; and (a-3) the N-terminus of the third poly peptide is linked to the fifth cytokine polypeptide or the variant thereof; the C-terminus of the third polypeptide is linked to the sixth cytokine polypeptide or the variant thereof; or a combination thereof; (b-1) the N-terminus of the first polypeptide is linked to the first cytokine polypeptide or the variant thereof; the C-terminus of the first polypeptide is linked to the second cytokine polypeptide or the variant thereof; or a combination thereof; (b-2) the N-terminus of the second polypeptide is linked to the third cytokine polypeptide or the variant thereof; the C-terminus of the second polypeptide is linked to the fourth cytokine polypeptide or the variant thereof; or a combination thereof; and (b-3) the N-terminus of the fourth polypeptide is linked to the seventh cytokine polypeptide or the variant thereof; the C-terminus of the fourth polypeptide is linked to the eighth cytokine polypeptide or the variant thereof; or a combination thereof; or (c-1) the N-terminus of the second polypeptide is linked to the third cytokine polypeptide or the variant thereof; the C-terminus of the second polypeptide is linked to the fourth cytokine polypeptide or the variant thereof; or a combination thereof; (c-2) the N-terminus of the third polypeptide is linked to the fifth cytokine polypeptide or the variant thereof; the C-terminus of the third polypeptide is linked to the sixth cytokine polypeptide or the variant thereof; or a combination thereof; and (c-3) the N-terminus of the fourth polypeptide is linked to the seventh cytokine polypeptide or the variant thereof; the C-terminus of the fourth polypeptide is linked to the eighth cytokine polypeptide or the variant thereof; or a combination thereof.

In some embodiments, (1) the N-terminus of the first polypeptide is linked to the first cytokine polypeptide or the variant thereof; the C-terminus of the first polypeptide is linked to the second cytokine polypeptide or the variant thereof; or a combination thereof; (2) the N-terminus of the second polypeptide is linked to the third cytokine polypeptide or the variant thereof; the C-terminus of the second polypeptide is linked to the fourth cytokine polypeptide or the variant thereof; or a combination thereof; (3) the N-terminus of the third polypeptide is linked to the fifth cytokine polypeptide or the variant thereof; the C-terminus of the third polypeptide is linked to the sixth cytokine polypeptide or the variant thereof; or a combination thereof; and (4) the N-terminus of the fourth polypeptide is linked to the seventh cytokine polypeptide or the variant thereof; the C-terminus of the fourth polypeptide is linked to the eighth cytokine polypeptide or the variant thereof; or a combination thereof.

In some embodiments, the first cytokine polypeptide, the second cytokine polypeptide, or a combination thereof is within a single contiguous poly peptide chain of the first polypeptide, the third cytokine polypeptide, the fourth cytokine polypeptide, or a combination thereof is within a single contiguous polypeptide chain of the second polypeptide, the fifth cytokine polypeptide, the sixth cytokine polypeptide, or a combination thereof is within a single contiguous polypeptide chain of the third polypeptide, the seventh cytokine polypeptide, the eighth cytokine polypeptide, or a combination thereof is within a single contiguous polypeptide chain of the fourth polypeptide, or a combination thereof.

In some embodiments. (a) the N-terminus of the first polypeptide is linked to a first cytokine polypeptide or a variant thereof; the C-terminus of the first polypeptide is linked to a second cytokine polypeptide or a variant thereof; or a combination thereof; (b) the N-terminus of the second polypeptide is linked to a third cytokine polypeptide or a variant thereof; the C-terminus of the second polypeptide is linked to a fourth cytokine poly peptide or a variant thereof; or a combination thereof; (c) the N-terminus of the third polypeptide is linked to a fifth cytokine polypeptide or a variant thereof; the C-terminus of the third polypeptide is linked to a sixth cytokine polypeptide or a variant thereof; or a combination thereof; or (d) a combination thereof.

In some embodiments, (a-1) the N-terminus of the first polypeptide is linked to the first cytokine polypeptide or the variant thereof; the C-terminus of the first polypeptide is linked to the second cytokine polypeptide or the variant thereof; or a combination thereof; and (a-2) the N-terminus of the second polypeptide is linked to the third cytokine polypeptide or the variant thereof; the C-terminus of the second polypeptide is linked to the fourth cytokine polypeptide or the variant thereof; or a combination thereof; (b-1) the N-terminus of the first polypeptide is linked to the first cytokine polypeptide or the variant thereof; the C-terminus of the first polypeptide is linked to the second cytokine polypeptide or the variant thereof; or a combination thereof; and (b-2) the N-terminus of the third polypeptide is linked to the fifth cytokine polypeptide or the variant thereof; the C-terminus of the third polypeptide is linked to the sixth cytokine polypeptide or the variant thereof; or a combination thereof; or (c-1) the N-terminus of the second polypeptide is linked to the third cytokine polypeptide or the variant thereof; the C-terminus of the second polypeptide is linked to the fourth cytokine polypeptide or the variant thereof; or a combination thereof; and (c-2) the N-terminus of the third poly peptide is linked to the fifth cytokine polypeptide or the variant thereof; the C-terminus of the third polypeptide is linked to the sixth cytokine polypeptide or the variant thereof; or a combination thereof.

In some embodiments, (1) the N-terminus of the first polypeptide is linked to the first cytokine polypeptide or the variant thereof; the C-terminus of the first polypeptide is linked to the second cytokine polypeptide or the variant thereof; or a combination thereof; (2) the N-terminus of the second polypeptide is linked to the third cytokine polypeptide or the variant thereof; the C-terminus of the second polypeptide is linked to the fourth cytokine polypeptide or the variant thereof; or a combination thereof; and (3) the N-terminus of the third polypeptide is linked to the fifth cytokine polypeptide or the variant thereof; the C-terminus of the third polypeptide is linked to the sixth cytokine polypeptide or the variant thereof; or a combination thereof.

In some embodiments, the first cytokine polypeptide, the second cytokine polypeptide, or a combination thereof is within a single contiguous polypeptide chain of the first polypeptide, the third cytokine polypeptide, the fourth cytokine polypeptide, or a combination thereof is within a single contiguous polypeptide chain of the second polypeptide, the fifth cytokine polypeptide, the sixth cytokine polypeptide, or a combination thereof is within a single contiguous polypeptide chain of the third polypeptide, or a combination thereof.

In some embodiments, the multifunctional polypeptide molecule as described herein further comprises a linker between the first portion of the first TCRαV-binding moiety and the first dimerization module, a linker between the first portion of the second TCRαV-binding moiety and the second dimerization module, a linker between the first VH and the first CH1, a linker between the first VL and the first CL, a linker between the second VH and the second CH1, a linker between the second VL and the second CL, a linker between the at least one cytokine polypeptide or the variant thereof and the first polypeptide, a linker between the at least one cytokine polypeptide or the variant thereof and the second polypeptide, a linker between the at least one cytokine polypeptide or the variant thereof and the third polypeptide, a linker between the at least one cytokine polypeptide or the variant thereof and the fourth polypeptide, or a combination thereof.

In some embodiments, the multifunctional polypeptide molecule as described herein further comprises comprising a linker between the first portion of the first TCRαV-binding moiety and the first dimerization module, a linker between the first VH and the first CH1, a linker between the first VL and the first CL, a linker between the at least one cytokine polypeptide or the variant thereof and the first polypeptide, a linker between the at least one cytokine polypeptide or the variant thereof and the second polypeptide, a linker between the at least one cytokine polypeptide or the variant thereof and the third polypeptide, or a combination thereof. In some embodiments, linker is selected from the group consisting of a cleavable linker, a non-cleavable linker, a peptide linker, a flexible linker, a rigid linker, a helical linker, and a non-helical linker. In some embodiments, the linker is the peptide linker and wherein the linker is a GS linker. In some embodiments, the linker is the peptide linker and wherein the linker comprises the sequence of SEQ ID NO: 3308 or SEQ ID NO: 3643.

Described herein, in certain embodiments, is a multifunctional polypeptide molecule comprising a first polypeptide, a second polypeptide, a third polypeptide, a fourth polypeptide, a first cytokine polypeptide or a variant thereof, and a second cytokine polypeptide or a variant thereof, wherein the first polypeptide, the second polypeptide, the third polypeptide, and the fourth polypeptide are non-contiguous, wherein: (i) the first polypeptide comprising a first portion of a first TCRαV-binding moiety and a first dimerization module linked to the first portion of the first TCRαV-binding moiety; (ii) the second polypeptide comprising a second portion of the first TCRαV-binding moiety; (iii) the third polypeptide comprising a first portion of a second TCRαV-binding moiety and a second dimerization module linked to the first portion of the second TCRαV-binding moiety; and (iv) the fourth polypeptide comprising a second portion of the second TCRαV-binding moiety; and wherein the first cytokine polypeptide or the variant thereof is covalently linked to the C-terminus of the second polypeptide, and the second cytokine polypeptide or the variant thereof is covalently linked to the C-terminus of the fourth polypeptide.

Described herein, in certain embodiments, is a multifunctional polypeptide molecule comprising a first polypeptide, a second polypeptide, a third polypeptide, a fourth polypeptide, a cytokine polypeptide or a variant thereof, wherein the first polypeptide, the second polypeptide, the third polypeptide, and the fourth polypeptide are non-contiguous, wherein: (i) the first polypeptide comprising a first portion of a first TCRαV-binding moiety and a first dimerization module linked to the first portion of the first TCRαV-binding moiety; (ii) the second poly peptide comprising a second portion of the first TCRαV-binding moiety; (iii) the third polypeptide comprising a first portion of a second TCRαV-binding moiety and a second dimerization module linked to the first portion of the second TCRαV-binding moiety; and (iv) the fourth polypeptide comprising a second portion of the second TCRαV-binding moiety; and wherein the cytokine polypeptide or the variant thereof is covalently linked to the C-terminus of the second polypeptide or the C-terminus of the fourth polypeptide.

Described herein, in certain embodiments, is a multifunctional poly peptide molecule comprising a first polypeptide, a second polypeptide, a third polypeptide, a fourth polypeptide, a cytokine polypeptide or a variant thereof, wherein the first polypeptide, the second polypeptide, the third polypeptide, and the fourth polypeptide are non-contiguous, wherein: (i) the first polypeptide comprising a first portion of a first TCRαV-binding moiety and a first dimerization module linked to the first portion of the first TCRαV-binding moiety; (ii) the second poly peptide comprising a second portion of the first TCRαV-binding moiety; (iii) the third polypeptide comprising a first portion of a second TCRαV-binding moiety and a second dimerization module linked to the first portion of the second TCRαV-binding moiety; and (iv) the fourth polypeptide comprising a second portion of the second TCRαV-binding moiety; and wherein the cytokine polypeptide or the variant thereof is covalently linked to the C-terminus of the first polypeptide or the C-terminus of the third polypeptide.

Described herein, in certain embodiments, is a multifunctional polypeptide molecule comprising a first polypeptide, a second polypeptide, a third polypeptide, and a cytokine polypeptide or a variant thereof, wherein the first polypeptide, the second polypeptide, and the third polypeptide are non-contiguous, wherein: (i) the first polypeptide comprising a first portion of a first TCRαV-binding moiety and a first dimerization module linked to the first portion of the first TCRαV-binding moiety; (ii) the second polypeptide comprising a second portion of the first TCRαV-binding moiety; and (iii) the third polypeptide comprising a second dimerization module; wherein the at least one cytokine polypeptide or the variant thereof is covalently linked to the N terminus of the third polypeptide; and wherein the multifunctional polypeptide molecule does not comprise an additional TCRαV-binding moiety except the first TCRαV-binding moiety.

In some embodiments, the first portion of the first TCRαV-binding moiety comprises a first VH and a first CH1 linked to the first VH. In some embodiments, the first CH1 is linked to the C-terminus of the first VH.

In some embodiments, the second portion of the first TCRαV-binding moiety comprises a first VL and a first CL linked to the first VL. In some embodiments, first CL is linked to the C-terminus of the first VL.

In some embodiments, the first dimerization module is linked to the first portion of the first TCRαV-binding moiety. In some embodiments, the first dimerization module is linked to the C-terminus of the first portion of the first TCRαV-binding moiety. In some embodiments, the first portion of the second TCRαV-binding moiety comprises a second VH and a second CH1 linked to the second VH. In some embodiments, the second CH1 is linked to the C-terminus of the second VH. In some embodiments, the second portion of the second TCRαV-binding moiety comprises a second VL and a second CL linked to the second VL. In some embodiments, the second CL is linked to the C-terminus of the second VL. In some embodiments, the second dimerization module is linked to the first portion of the second TCRαV-binding moiety. In some embodiments, the second dimerization module is linked to the C-terminus of the first portion of the second TCRαV-binding moiety.

In some embodiments, the multifunctional polypeptide molecule as described herein further comprises a linker between the first portion of the first TCRαV-binding moiety and the first dimerization module, a linker between the first portion of the second TCRαV-binding moiety and the second dimerization module, a linker between the first VH and the first CH1, a linker between the first VL and the first CL, a linker between the second VH and the second CH1, a linker between the second VL and the second CL, a linker between the at least one cytokine polypeptide or the variant thereof and the first polypeptide, a linker between the at least one cytokine polypeptide or the variant thereof and the second poly peptide, a linker between the at least one cytokine polypeptide or the variant thereof and the third polypeptide, a linker between the at least one cytokine polypeptide or the variant thereof and the fourth polypeptide, or a combination thereof. In some embodiments, the multifunctional polypeptide molecule as described herein further comprises a linker between the first portion of the first TCRαV-binding moiety and the first dimerization module, a linker between the first VH and the first CH1, a linker between the first VL and the first CL, a linker between the at least one cytokine polypeptide or the variant thereof and the third polypeptide, or a combination thereof. In some embodiments, linker is selected from the group consisting of a cleavable linker, a non-cleavable linker, a peptide linker, a flexible linker, a rigid linker, a helical linker, and a non-helical linker. In some embodiments, the linker is the peptide linker and wherein the linker is a GS linker. In some embodiments, the linker is the peptide linker and wherein the linker comprises the sequence of SEQ ID NO: 3308 or SEQ ID NO: 3643.

In some embodiments, the first TCRαV-binding moiety, the second TCRαV-binding moiety, or a combination thereof comprises any one selected from the group consisting of a Fab, F(ab′)2, Fv, a single chain Fv (scFv), a single domain antibody, a diabody (dAb), a camelid antibody and a combination thereof. In some embodiments, the first TCRαV-binding moiety, the second TCRαV-binding moiety, or a combination thereof comprises a scFv or a Fab.

In some embodiments, the multifunctional polypeptide molecule does not comprise an additional antigen-binding moiety except the TCRαV-binding moiety. In some embodiments, the multifunctional poly peptide molecule further comprise an additional antigen-binding moiety that is not the TCRαV-binding moiety.

Described herein, in certain embodiments, is a multifunctional polypeptide molecule comprising a first polypeptide, a second polypeptide, and at least one cytokine polypeptide or a variant thereof, wherein the first polypeptide and the second polypeptide are non-contiguous, wherein: (i) the first polypeptide comprising a first TCRαV-binding moiety and a first dimerization module linked to the C-terminus of the first TCRαV-binding moiety, wherein the first TCRαV-binding moiety comprises a first VL and a first VH; and (ii) the second polypeptide comprising a second TCRαV-binding moiety and a second dimerization module linked to the C-terminus of the second TCRαV-binding moiety; wherein the at least one cytokine polypeptide or the variant thereof is covalently linked to the first polypeptide, the second polypeptide, or a combination thereof; wherein the first TCRαV-binding moiety, the second TCRαV-binding moiety, or a combination thereof comprises a scFv; and wherein the multifunctional polypeptide molecule does not comprise an additional antigen-binding moiety except the first TCRαV-binding moiety and the second TCRαV-binding moiety.

Described herein, in certain embodiments, is a multifunctional polypeptide molecule comprising a first polypeptide, a second polypeptide, and at least one cytokine polypeptide or a variant thereof, wherein the first polypeptide and the second polypeptide are non-contiguous, wherein: (i) the first polypeptide comprising a first TCRαV-binding moiety and a first dimerization module linked to the C-terminus of the first TCRαV-binding moiety, wherein the first TCRαV-binding moiety comprises a first VL and a first VH; and (ii) the second polypeptide comprising a second dimerization module; wherein the at least one cytokine polypeptide or the variant thereof is covalently linked to the first polypeptide, the second polypeptide, or a combination thereof; wherein the first TCRαV-binding moiety comprises a scFv; wherein the multifunctional polypeptide molecule does not comprise an additional antigen-binding moiety except the first TCRαV-binding moiety; and wherein the multifunctional polypeptide molecule does not comprise an additional TCRαV-binding moiety except the first TCRαV-binding moiety.

In some embodiments, (a) the N-terminus of the first polypeptide is linked to a first cytokine polypeptide or a variant thereof; the C-terminus of the first polypeptide is linked to a second cytokine polypeptide or a variant thereof; or a combination thereof; (b) the N-terminus of the second polypeptide is linked to a third cytokine polypeptide or a variant thereof; the C-terminus of the second poly peptide is linked to a fourth cytokine polypeptide or a variant thereof; or a combination thereof; or (e) a combination thereof.

In some embodiments, the first cytokine polypeptide, the second cytokine polypeptide, or a combination thereof is within a single contiguous poly peptide chain of the first poly peptide, the third cytokine polypeptide, the fourth cytokine polypeptide, or a combination thereof is within a single contiguous polypeptide chain of the second polypeptide, or a combination thereof.

In some embodiments, the multifunctional polypeptide molecule as described herein further comprises a linker between the first TCRαV-binding moiety and the first dimerization module, a linker between the second TCRαV-binding moiety and the second dimerization module, a linker between the at least one cytokine poly peptide or the variant thereof and the first polypeptide, a linker between the at least one cytokine polypeptide or the variant thereof and the second polypeptide, or a combination thereof.

In some embodiments, the multifunctional polypeptide molecule as described herein further comprises a linker between the first TCRαV-binding moiety and the first dimerization module, a linker between the at least one cytokine poly peptide or the variant thereof and the first polypeptide, a linker between the at least one cytokine polypeptide or the variant thereof and the second polypeptide, or a combination thereof. In some embodiments, the linker is selected from the group consisting of a cleavable linker, a non-cleavable linker, a peptide linker, a flexible linker, a rigid linker, a helical linker, and a non-helical linker. In some embodiments, the linker is the peptide linker and wherein the linker is a GS linker. In some embodiments, the linker is the peptide linker and wherein the linker comprises the sequence of SEQ ID NO: 3308 or SEQ ID NO: 3643.

In some embodiments, the multifunctional polypeptide molecule comprises at least two of the cytokine polypeptide. In some embodiments, the multifunctional polypeptide molecule comprises at least three of the cytokine polypeptide. In some embodiments, the multifunctional polypeptide molecule comprises at least four of the cytokine polypeptide. In some embodiments, the multifunctional poly peptide molecule comprises at least five of the cytokine polypeptide. In some embodiments, the multifunctional polypeptide molecule comprises at least six of the cytokine polypeptide. In some embodiments, the multifunctional polypeptide molecule comprises at least seven of the cytokine polypeptide. In some embodiments, the multifunctional polypeptide molecule comprises at least eight of the cytokine polypeptide. In some embodiments, the multifunctional polypeptide molecule comprises two of the cytokine polypeptide. In some embodiments, the multifunctional polypeptide molecule comprises three of the cytokine polypeptide. In some embodiments, the multifunctional polypeptide molecule comprises four of the cytokine poly peptide. In some embodiments, the multifunctional polypeptide molecule comprises five of the cytokine polypeptide. In some embodiments, the multifunctional polypeptide molecule comprises six of the cytokine polypeptide. In some embodiments, the multifunctional polypeptide molecule comprises seven of the cytokine polypeptide. In some embodiments, the multifunctional polypeptide molecule comprises eight of the cytokine polypeptide. In some embodiments, the multifunctional polypeptide molecule comprises two of the cytokine polypeptide, each of which is linked to the first polypeptide and the second polypeptide; the first polypeptide and the third polypeptide; the first polypeptide and the fourth polypeptide; the second and the third polypeptide; the second polypeptide and the fourth polypeptide; or the third polypeptide and the fourth polypeptide, respectively. In some embodiments, the multifunctional polypeptide molecule comprises three of the cytokine polypeptide, each of which is linked to the first polypeptide, the second polypeptide, and the third poly peptide; the first polypeptide, the second polypeptide, and the fourth polypeptide; the first polypeptide, the third polypeptide, and the fourth polypeptide; or the second polypeptide, the third polypeptide, and the fourth polypeptide, respectively. In some embodiments, the multifunctional polypeptide molecule comprises four of the cytokine polypeptide, each of which is linked to the first polypeptide, the second polypeptide, the third polypeptide, and the fourth polypeptide, respectively. In some embodiments, the cytokine polypeptide is not linked to the polypeptides that comprise the first TCRαV-binding moiety.

In some embodiments, the at least one cytokine polypeptide is selected from the group consisting of interleukin-2 (IL-2) or a fragment or a variant thereof, interleukin-7 (IL-7) or a fragment or a variant thereof, interleukin-12 (IL-12) or a fragment or a variant thereof, interleukin-15 (IL-15) or a fragment or a variant thereof, interleukin-18 (IL-18) or a fragment or a variant thereof, interleukin-21 (IL-21) or a fragment or a variant thereof, or interferon gamma or a fragment or a variant thereof, or a combination thereof.

In some embodiments, the at least one cytokine polypeptide comprises interleukin-2 (IL-2) or a fragment thereof. In some embodiments, the at least one cytokine polypeptide is interleukin-2 (IL-2) or a fragment thereof. In some embodiments, the at least one cytokine polypeptide comprises a sequence having at least 75%, 80%, 85%, 90%, 95%, 99%, 99.5%, 99.9%, or 100% sequence identity to the sequence of SEQ ID NO: 2191. In some embodiments, the at least one cytokine polypeptide comprises the sequence of SEQ ID NO: 2191. In some embodiments, the sequence of the at least one cytokine poly peptide is a sequence having at least 75%, 80%, 85%, 90%, 95%, 99%, 99.5%, 99.9%, or 100% sequence identity to the sequence of SEQ ID NO: 2191. In some embodiments, the sequence of the at least one cytokine polypeptide is the sequence of SEQ ID NO: 2191.

In some embodiments, the variant of the at least one cytokine poly peptide comprises an IL-2 variant comprising a mutation. In some embodiments, the mutation comprises an insertion mutation, a deletion mutation, or a substitution mutation. In some embodiments, the mutation comprises the substitution mutation. In some embodiments, the variant comprises an IL-2 variant comprising C125A mutation. In some embodiments, the variant of the at least one cytokine polypeptide is an IL-2 variant comprising a mutation. In some embodiments, the mutation is an insertion mutation, a deletion mutation, or a substitution mutation. In some embodiments, the mutation is the substitution mutation. In some embodiments, the variant is an IL-2 variant comprising C125A mutation. In some embodiments, the variant comprises a sequence having at least 75%, 80%, 85%, 90%, 95%, 99%, 99.5%, 99.9%, or 100% sequence identity to the sequence of SEQ ID NO: 2270. In some embodiments, the variant comprises the sequence of SEQ ID NO: 2270. In some embodiments, the sequence of the variant is a sequence having at least 75%, 80%, 85%, 90%, 95%, 99%, 99.5%, 99.9%, or 100% sequence identity to the sequence of SEQ ID NO: 2270. In some embodiments, the sequence of the variant is the sequence of SEQ ID NO: 2270.

In some embodiments, the first dimerization module comprises a first immunoglobulin constant regions (Fc regions) and the second dimerization module comprises a second Fc region. In some embodiments, the first dimerization module is a first immunoglobulin constant regions (Fc regions) and the second dimerization module is a second Fc region.

In some embodiments, the first Fc region, the second Fc region, or a combination thereof is selected from an IgG1 Fc region or a fragment thereof, an IgG2 Fc region or a fragment thereof, an IgG3 Fc region or a fragment thereof, an IgGA1 Fc region or a fragment thereof, an IgGA2 Fc region or a fragment thereof, an IgG4 Fc region or a fragment thereof, an IgJ Fc region or a fragment thereof, an IgM Fc region or a fragment thereof, an IgD Fc region or a fragment thereof, and an IgE Fc region or a fragment thereof.

In some embodiments, the first Fc region, the second Fc region, or a combination thereof is selected from a human IgG1 Fc region or a fragment thereof, a human IgG2 Fc region or a fragment thereof, and a human IgG4 Fc region or a fragment thereof.

In some embodiments, the first Fc region, the second Fc region, or a combination thereof comprises an Fc interface with one or more of: a paired cavity-protuberance, an electrostatic interaction, or a strand-exchange, wherein the dimerization of the first Fc region and the second Fc region is enhanced as indicated by a greater ratio of heteromultimer:homomultimer forms relative to a dimerization of Fc regions with a non-engineered interface. In some embodiments, the dimerization of the first Fc region and the second Fc region is enhanced at least by 1.1 fold, 1.2 fold, 1.3 fold, 1.4 fold, 1.5 fold, 1.6 fold, 1.7 fold, 1.8 fold, 1.9 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, 150 fold, 200 fold, 250 fold, 300 fold, 250 fold, 400 fold, 450 fold, 500 fold, 550 fold, 600 fold, 650 fold, 700 fold, 750 fold, 800 fold, 850 fold, 900 fold, 950 fold, 1000 fold, 2000 fold, 3000 fold, 4000 fold, 5000 fold, 6000 fold, 7000 fold, 8000 fold, 9000 fold, or 10000 fold relative to a dimerization of Fc regions with a non-engineered interface. In some embodiments, the dimerization of the first Fc region and the second Fc region is enhanced at most by 1.1 fold, 1.2 fold, 1.3 fold, 1.4 fold, 1.5 fold, 1.6 fold, 1.7 fold, 1.8 fold, 1.9 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, 150 fold, 200 fold, 250 fold, 300 fold, 250 fold, 400 fold, 450 fold, 500 fold, 550 fold, 600 fold, 650 fold, 700 fold, 750 fold, 800 fold, 850 fold, 900 fold, 950 fold, 1000 fold, 2000 fold, 3000 fold, 4000 fold, 5000 fold, 6000 fold, 7000 fold, 8000 fold, 9000 fold, or 10000 fold relative to a dimerization of Fc regions with a non-engineered interface. In some embodiments, the dimerization of the first Fc region and the second Fc region is enhanced by 1.1 fold, 1.2 fold, 1.3 fold, 1.4 fold, 1.5 fold, 1.6 fold, 1.7 fold, 1.8 fold, 1.9 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, 50 fold, 55 fold, 60 fold, 65 fold, 70 fold, 75 fold, 80 fold, 85 fold, 90 fold, 95 fold, 100 fold, 150 fold, 200 fold, 250 fold, 300 fold, 250 fold, 400 fold, 450 fold, 500 fold, 550 fold, 600 fold, 650 fold, 700 fold, 750 fold, 800 fold, 850 fold, 900 fold, 950 fold, 1000 fold, 2000 fold, 3000 fold, 4000 fold, 5000 fold, 6000 fold, 7000 fold, 8000 fold, 9000 fold, or 10000 fold relative to a dimerization of Fc regions with a non-engineered interface.

In some embodiments, the first Fc region, the second Fc region, or a combination thereof comprises an amino acid substitution listed in Table 6.

In some embodiments, the first Fc region, the second Fc region, or a combination thereof comprises an Asn297Ala (N297A) mutation or a Leu234Ala/Leu235Ala (LALA) mutation.

In some embodiments, the first Fc region, the second Fc region, or a combination thereof comprises a sequence having at least 75%, 80%, 85%, 90%, 95%, 99%, 99.5%, 99.9%, or 100% sequence identity to the sequence of SEQ ID NO: 40, SEQ ID NO: 42, SEQ ID NO: 3645, SEQ ID NO: 3646, SEQ ID NO: 3647, SEQ ID NO:3648, or SEQ ID NO: 3649. In some embodiments, the first Fc region, the second Fc region, or a combination thereof comprises the sequence of SEQ ID NO: 40, SEQ ID NO: 42, SEQ ID NO: 3645, SEQ ID NO: 3646, SEQ ID NO: 3647, SEQ ID NO:3648, or SEQ ID NO: 3649.

In some embodiments, the sequence of the first Fc region, the second Fc region, or a combination thereof is a sequence having at least 75%, 80%, 85%, 90%, 95%, 99%, 99.5%, 99.9%, or 100% sequence identity to the sequence of SEQ ID NO: 40, SEQ ID NO: 42, SEQ ID NO: 3645, SEQ ID NO: 3646, SEQ ID NO: 3647, SEQ ID NO:3648, or SEQ ID NO: 3649. In some embodiments, the sequence of the first Fc region, the second Fc region, or a combination thereof is the sequence of SEQ ID NO: 40, SEQ ID NO: 42, SEQ ID NO: 3645, SEQ ID NO: 3646, SEQ ID NO: 3647, SEQ ID NO:3648, or SEQ ID NO: 3649. [00277]1n some embodiments, the first TCRαV-binding moiety and the second TCRαV-binding moiety are same. In some embodiments, the first TCRαV-binding moiety and the second TCRαV-binding moiety are different.

In some embodiments, the first TCRαV-binding moiety, the second TCRαV-binding moiety, or a combination thereof comprises: (i) a VH comprising a framework region (FR) comprising a framework 1 (FR1), a framework region 2 (FR2), a framework region 3 (FR3), and a framework region 4 (FR4) that have at least 75%, 80%, 85%, 90%, 95%, 99%, 99.5%, 99.9%, or 100% sequence identity with a non-murine germline FR1, a non-murine germline FR2, a non-murine germline FR3, and a non-murine germline FR4; (ii) a VL comprising a FR comprising a FR1, a FR2, a FR3, and a FR4 that have at least 75%, 80%, 85%, 90%, 95%, 99%, 99.5%, 99.9%, or 100% sequence identity with a non-murine germline FR1, a non-murine germline FR2, a non-murine germline FR3, and a non-murine germline FR4; or (iii) a combination thereof. In some embodiments, the first TCRαV-binding moiety, the second TCRαV-binding moiety, or a combination thereof comprises: (i) a VH comprising a FR comprising a FR1, a FR2, a FR3, and a FR4 having the sequences of a non-murine germline FR1, a non-murine germline FR2, a non-murine germline FR3, and a non-murine germline FR4; (ii) a VL comprising a FR comprising a FR1, a FR2, a FR3, and a FR4 having the sequences of a non-murine germline FR1, a non-murine germline FR2, a non-murine germline FR3, and a non-murine germline FR4; or (iii) a combination thereof.

In some embodiments, the first TCRαV-binding moiety, the second TCRαV-binding moiety, or a combination thereof comprises: (i) a VH comprising a FR1, a FR2, a FR3, and a FR4 that have at least 75%, 80%, 85%, 90%, 95%, 99%, 99.5%, 99.9%, or 100% sequence identity with a non-murine germline FR1, a non-murine germline FR2, a non-murine germline FR3, and a non-murine germline FR4, respectively; (ii) a VL comprising a FR comprising a FR1, a FR2, a FR3, and a FR4 that have at least 75%, 80%, 85%, 90%, 95%, 99%, 99.5%, 99.9%, or 100% sequence identity with a non-murine germline FR1, a non-murine germline FR2, a non-murine germline FR3, and a non-murine germline FR4, respectively; or (iii) a combination thereof. In some embodiments, the first TCRαV-binding moiety, the second TCRαV-binding moiety, or a combination thereof comprises: (i) a VH comprising a FR comprising a FR1, a FR2, a FR3, and a FR4 having the sequences of a non-murine germline FR1, a non-murine germline FR2, a non-murine germline FR3, and a non-murine germline FR4, respectively; (ii) a VL comprising a FR comprising a FR1, a FR2, a FR3, and a FR4 having the sequences of a non-murine germline FR1, a non-murine germline FR2, a non-murine germline FR3, and a non-murine germline FR4, respectively; or (iii) a combination thereof.

In some embodiments, the first polypeptide, the second polypeptide, the third polypeptide, the fourth polypeptide, or a combination thereof comprises a heavy chain constant region having a sequence having at least 75%, 80%, 85%, 90%, 95%, 99%, 99.5%, 99.9%, or 100% sequence identity to any one of the sequences listed in Table 1 or a combination thereof. In some embodiments, the first polypeptide, the second polypeptide, the third polypeptide, the fourth polypeptide, or a combination thereof comprises a heavy chain constant region having any one of the sequences listed in Table 1 or a combination thereof. In some embodiments, the first polypeptide, the second polypeptide, the third polypeptide, the fourth polypeptide, or a combination thereof comprises a heavy chain constant region of which sequence is a sequence having at least 75%, 80%, 85%, 90%, 95%, 99%, 99.5%, 99.9%, or 100% sequence identity to any one of the sequences listed in Table 1 or a combination thereof. In some embodiments, the first polypeptide, the second polypeptide, the third polypeptide, the fourth polypeptide, or a combination thereof comprises a heavy chain constant region having any one of the heavy chain constant region sequences listed in Table 1 or a combination thereof. In some embodiments, the first polypeptide, the second polypeptide, the third polypeptide, the fourth polypeptide, or a combination thereof comprises a heavy chain constant region of an IgM or a fragment thereof. In some embodiments, the heavy chain constant region of the IgM comprises a sequence having at least 75%, 80%, 85%, 90%, 95%, 99%, 99.5%, 99.9%, or 100% sequence identity to the sequence of SEQ ID NO: 73. In some embodiments, the heavy chain constant region of the IgM comprises the sequence of SEQ ID NO: 73. In some embodiments, the sequence of the heavy chain constant region of the IgM is the sequence of SEQ ID NO: 73.

In some embodiments, the first polypeptide, the second polypeptide, the third polypeptide, the fourth polypeptide, or a combination thereof comprises a heavy chain constant region of an IgJ or a fragment thereof. In some embodiments, the heavy chain constant region of the IgJ comprises a sequence having at least 75%, 80%, 85%, 90%, 95%, 99%, 99.5%, 99.9%, or 100% sequence identity to the sequence of SEQ ID NO: 76. In some embodiments, the heavy chain constant region of the IgJ comprises the sequence of SEQ ID NO: 76. In some embodiments, the sequence of the heavy chain constant region of the IgJ is the sequence of SEQ ID NO: 76.

In some embodiments, the first polypeptide, the second polypeptide, the third polypeptide, the fourth polypeptide, or a combination thereof comprises a heavy chain constant region of an IgGA1 or a fragment thereof. In some embodiments, the heavy chain constant region of the IgGA1 comprises a sequence having at least 75%, 80%, 85%, 90%, 95%, 99%, 99.5%, 99.9%, or 100% sequence identity to the sequence of SEQ ID NO: 74. In some embodiments, the heavy chain constant region of the IgGA1 comprises the sequence of SEQ ID NO: 74. In some embodiments, the sequence of the heavy chain constant region of the IgGA1 is the sequence of SEQ ID NO: 74.

In some embodiments, the first polypeptide, the second polypeptide, the third poly peptide, the fourth polypeptide, or a combination thereof comprises a heavy chain constant region of an IgGA2 or a fragment thereof. In some embodiments, the heavy chain constant region of the IgGA2 comprises a sequence having at least 75%, 80%, 85%, 90%, 95%, 99%, 99.5%, 99.9%, or 100% sequence identity to the sequence of SEQ ID NO: 75. In some embodiments, the heavy chain constant region of the IgGA2 comprises the sequence of SEQ ID NO: 75. In some embodiments, the sequence of the heavy chain constant region of the IgGA2 is the sequence of SEQ ID NO: 75.

In some embodiments, the first polypeptide, the second polypeptide, the third polypeptide, the fourth polypeptide, or a combination thereof comprises a heavy chain constant region of an IgG1 or a fragment thereof. In some embodiments, the heavy chain constant region of the IgG1 comprises a sequence having at least 75%, 80%, 85%, 90%, 95%, 99%, 99.5%, 99.9%, or 100% sequence identity to the sequence of SEQ ID NO: 41. In some embodiments, the heavy chain constant region of the IgG1 comprises the sequence of SEQ ID NO: 41. In some embodiments, the sequence of the heavy chain constant region of the IgG1 is the sequence of SEQ ID NO: 41. In some embodiments, the heavy chain constant region of the IgG comprises a sequence having at least 75%, 80%, 85%, 90%, 95%, 99%, 99.5%, 99.9%, or 100% sequence identity to the sequence of SEQ ID NO: 3645. In some embodiments, the heavy chain constant region of the IgG1 comprises the sequence of SEQ ID NO: 3645. In some embodiments, the sequence of the heavy chain constant region of the IgG1 is the sequence of SEQ ID NO: 3645.

In some embodiments, the first polypeptide, the second polypeptide, the third polypeptide, the fourth polypeptide, or a combination thereof comprises a light chain constant region having a sequence having at least 75%, 80%, 85%, 90%, 95%, 99%, 99.5%, 99.9%, or 100% sequence identity to any one of the sequences listed in Table 1 or a combination thereof. In some embodiments, the first polypeptide, the second polypeptide, the third polypeptide, the fourth polypeptide, or a combination thereof comprises a light chain constant region having any one of the sequences listed in Table 1 or a combination thereof. In some embodiments, the first polypeptide, the second polypeptide, the third polypeptide, the fourth polypeptide, or a combination thereof comprises a light chain constant region having any one of the light chain constant region sequences listed in Table 1 or a combination thereof.

In some embodiments, the first polypeptide, the second polypeptide, the third polypeptide, the fourth polypeptide, or a combination thereof comprises a light chain constant region of a kappa chain or a fragment thereof. In some embodiments, the light chain constant region of a kappa chain comprises a light chain constant region sequence listed in Table 1.

In some embodiments, the light chain constant region of a kappa chain comprises a sequence having at least 75%, 80%, 85%, 90%, 95%, 99%, 99.5%, 99.9%, or 100% sequence identity to the sequence of SEQ ID NO: 39 or SEQ ID NO: 3644. In some embodiments, the light chain constant region of a kappa chain comprises the sequence of SEQ ID NO: 39 or SEQ ID NO: 3644. In some embodiments, the sequence of the light chain constant region of a kappa chain is a sequence having at least 75%, 80%, 85%, 90%, 95%, 99%, 99.5%, 99.9%, or 100% sequence identity to the sequence of SEQ ID NO: 39 or SEQ ID NO: 3644. In some embodiments, the sequence of the light chain constant region of a kappa chain is the sequence of SEQ ID NO: 39 or SEQ ID NO: 3644.

In some embodiments, the first TCRαV-binding moiety, the second TCRαV-binding moiety, or a combination thereof binds to an outward facing region on a TCRαV protein. In some embodiments, the outward facing region on the TCRαV protein comprises a structurally conserved region of TCRαV having a similar structure across one or more TCRαV subfamilies.

### Cytokine Molecules

In some embodiments, the multifunctional molecule includes a cytokine molecule. As used herein, a “cytokine molecule” refers to full length, a fragment or a variant of a cytokine; a cytokine further comprising a receptor domain, e.g., a cytokine receptor dimerizing domain; or an agonist of a cytokine receptor, e.g., an antibody molecule (e.g., an agonistic antibody) to a cytokine receptor, that elicits at least one activity of a naturally-occurring cytokine. In some embodiments the cytokine molecule is chosen from interleukin-2 (IL-2), interleukin-7 (IL-7), interleukin-12 (IL-12), interleukin-10 (IL-10), interleukin-15 (IL-15), interleukin-18 (IL-18), interleukin-21 (IL-21), or interferon gamma, or a fragment or variant thereof, or a combination of any of the aforesaid cytokines. The cytokine molecule can be a monomer or a dimer. In embodiments, the cytokine molecule can further include a cytokine receptor dimerizing domain. In other embodiments, the cytokine molecule is an agonist of a cytokine receptor, e.g., an antibody molecule (e.g., an agonistic antibody) to a cytokine receptor chosen from an IL-15Ra or IL-21R.

Cytokines are generally polypeptides that influence cellular activity, for example, through signal transduction pathways. Accordingly, a cytokine of the multispecific or multifunctional poly peptide is useful and can be associated with receptor-mediated signaling that transmits a signal from outside the cell membrane to modulate a response within the cell. Cytokines are proteinaceous signaling compounds that are mediators of the immune response. They control many different cellular functions including proliferation, differentiation and cell survival/apoptosis; cytokines are also involved in several pathophysiological processes including viral infections and autoimmune diseases. Cytokines are synthesized under various stimuli by a variety of cells of both the innate (monocytes, macrophages, dendritic cells) and adaptive (T- and B-cells) immune systems. Cytokines can be classified into two groups: pro- and anti-inflammatory. Pro-inflammatory cytokines, including IFNγ, IL-1, IL-6 and TNF-alpha, are predominantly derived from the innate immune cells and Th1 cells. Anti-inflammatory cytokines, including IL-10, IL-4, IL-13 and IL-5, are synthesized from Th2 immune cells.

Provided herein are, inter alia, multispecific (e.g., bi-, tri-, quad-specific) or multifunctional molecules, that include, e.g., are engineered to contain, one or more cytokine molecules, e.g., immunomodulatory (e.g., proinflammatory) cytokines and variants, e.g., functional variants, thereof. Accordingly, in some embodiments, the cytokine molecule is an interleukin or a variant, e.g., a functional variant thereof. In some embodiments the interleukin is a proinflammatory interleukin. In some embodiments the interleukin is chosen from interleukin-2 (IL-2), interleukin-12 (IL-12), interleukin-15 (IL-15), interleukin-18 (IL-18), interleukin-21 (IL-21), interleukin-7 (IL-7), or interferon gamma. In some embodiments, the cytokine molecule is a proinflammatory cytokine.

In certain embodiments, the cytokine is a single chain cytokine. In certain embodiments, the cytokine is a multichain cytokine (e.g., the cytokine comprises 2 or more (e.g., 2) polypeptide chains. An exemplary multichain cytokine is IL-12.

Examples of useful cytokines include, but are not limited to, GM-CSF, IL-1α, IL-1β, IL-2, IL-3, IL-4, IL-5, IL-6, IL-7, IL-8, IL-10, IL-12, IL-21, IFN-α, IFN-β, IFN-γ. MIP-1α, MIP-1β, TGF-β, TNF-α, and TNFβ. In some embodiments the cytokine of the multispecific or multifunctional polypeptide is a cytokine selected from the group of GM-CSF, IL-2, IL-7, IL-8, IL-10, IL-12, IL-15, IL-21, IFN-α, IFN-γ, MIP-1α, MIP-1β and TGF-β. In some embodiments the cytokine of the multispecific or multifunctional polypeptide is a cytokine selected from the group of IL-2, IL-7, IL-10, IL-12, IL-15, IFN-α, and IFN-γ. In certain embodiments the cytokine is mutated to remove N- and/or O-glycosylation sites. Elimination of glycosylation increases homogeneity of the product obtainable in recombinant production.

In some embodiments, the cytokine of the multispecific or multifunctional polypeptide is IL-2. In a specific embodiment, the IL-2 cytokine can elicit one or more of the cellular responses selected from the group consisting of: proliferation in an activated T lymphocyte cell, differentiation in an activated T lymphocyte cell, cytotoxic T cell (CTL) activity, proliferation in an activated B cell, differentiation in an activated B cell, proliferation in a natural killer (NK) cell, differentiation in a NK cell, cytokine secretion by an activated T cell or an NK cell, and NK/lymphocyte activated killer (LAK) antitumor cytotoxicity. In another particular embodiment the IL-2 cytokine is a mutant IL-2 cytokine having reduced binding affinity to the .alpha.-subunit of the IL-2 receptor. Together with the .beta.- and .gamma.-subunits (also known as CD122 and CD132, respectively), the .alpha.-subunit (also known as CD25) forms the heterotrimeric high-affinity IL-2 receptor, while the dimeric receptor consisting only of the β- and γ-subunits is termed the intermediate-affinity IL-2 receptor. As described in PCT patent application number PCT/EP2012/051991, which is incorporated herein by reference in its entirety, a mutant IL-2 polypeptide with reduced binding to the .alpha.-subunit of the IL-2 receptor has a reduced ability to induce IL-2 signaling in regulatory T cells, induces less activation-induced cell death (AICD) in T cells, and has a reduced toxicity profile in vivo, compared to a wild-type IL-2 polypeptide. The use of such an cytokine with reduced toxicity is particularly advantageous in a multispecific or multifunctional poly peptide according to the disclosure, having a long serum half-life due to the presence of an Fc domain. In some embodiments, the mutant IL-2 cytokine of the multispecific or multifunctional polypeptide according to the disclosure comprises at least one amino acid mutation that reduces or abolishes the affinity of the mutant IL-2 cytokine to the .alpha.-subunit of the IL-2 receptor (CD25) but preserves the affinity of the mutant IL-2 cytokine to the intermediate-affinity IL-2 receptor (consisting of the β and γ subunits of the IL-2 receptor), compared to the non-mutated IL-2 cytokine. In some embodiments the one or more amino acid mutations are amino acid substitutions. In a specific embodiment, the mutant IL-2 cytokine comprises one, two or three amino acid substitutions at one, two or three position(s) selected from the positions corresponding to residue 42, 45, and 72 of human IL-2. In a more specific embodiment, the mutant IL-2 cytokine comprises three amino acid substitutions at the positions corresponding to residue 42, 45 and 72 of human IL-2. In an even more specific embodiment, the mutant IL-2 cytokine is human IL-2 comprising the amino acid substitutions F42A, Y45A and L72G. In some embodiments the mutant IL-2 cytokine additionally comprises an amino acid mutation at a position corresponding to position 3 of human IL-2, which eliminates the O-glycosylation site of IL-2. Particularly, said additional amino acid mutation is an amino acid substitution replacing a threonine residue by an alanine residue. A particular mutant IL-2 cytokine useful in the disclosure comprises four amino acid substitutions at positions corresponding to residues 3, 42, 45 and 72 of human IL-2. Specific amino acid substitutions are T3A, F42A, Y45A and L72G. As demonstrated in PCT patent application number PCT/EP2012/051991 and in the appended Examples, said quadruple mutant IL-2 polypeptide (IL-2 qm) exhibits no detectable binding to CD25, reduced ability to induce apoptosis in T cells, reduced ability to induce IL-2 signaling in T.sub.reg cells, and a reduced toxicity profile in vivo. However, it retains ability to activate IL-2 signaling in effector cells, to induce proliferation of effector cells, and to generate IFN-7 as a secondary cytokine by NK cells.

The IL-2 or mutant IL-2 cytokine according to any of the above embodiments may comprise additional mutations that provide further advantages such as increased expression or stability. For example, the cysteine at position 125 may be replaced with a neutral amino acid such as alanine, to avoid the formation of disulfide-bridged IL-2 dimers. Thus, in certain embodiments the IL-2 or mutant IL-2 cytokine of the multispecific or multifunctional polypeptide according to the disclosure comprises an additional amino acid mutation at a position corresponding to residue 125 of human IL-2. In some embodiments said additional amino acid mutation is the amino acid substitution C125A.

In a specific embodiment the IL-2 cytokine of the multispecific or multifunctional polypeptide comprises the polypeptide sequence of SEQ ID NO: 2270 [APTSSSTKKTQLQLEHLLLDLQMILNGINNYKNPKLTRMLTFKFYMPKKATELKHLQC LEEELKPLEEVLNLAQSKNFHLRPRDLISNINVIVLELKGSETTFMCEYADETATIVEFLN RWITFAQSIISTLT].

In another specific embodiment the IL-2 cytokine of the multispecific or multifunctional polypeptide comprises the polypeptide sequence of SEQ ID NO: 2280 [APASSSTKKTQLQLEHLLLDLQMILNGINNYKNPKLTRMLTAKFAMPKKATELKHLQC LEEELKPLEEVLNGAQSKNFHLRPRDLISNINVIVLELKGSETTFMCEYADETATIVEFLN RWITFAQSIISTLT].

In another embodiment the cytokine of the multispecific or multifunctional polypeptide is IL-12. In a specific embodiment said IL-12 cytokine is a single chain IL-12 cytokine. In an even more specific embodiment the single chain IL-12 cytokine comprises the polypeptide sequence of SEQ ID NO: 2290 [IWELKKDVYVVELDWYPDAPGEMVVLTCDTPEEDGITWTLDQSSEVLGSGKTLTIQVK EFGDAGQYTCHKGGEVLSHSLLLLHKKEDGIWSTDILKDQKEPKNKTFLRCEAKNYSG RFTCWWLTTISTDLTFSVKSSRGSSDPQGVTCGAATLSAERVRGDNKEYEYSVECQEDS ACPAAEESLPIEVMVDAVHKLKYENYTSSFFIRDIIKPDPPKNLQLKPLKNSRQVEVSWE YPDTWSTPHSYFSLTFCVQVQGKSKREKKDRVFTDKTSATVICRKNASISVRAQDRYYS SSWSEWASVPCSGGGGSGGGGSGGGGSRNLPVATPDPGMFPCLHHSQNLLRAVSNML QKARQTLEFYPCTSEEIDHEDITKDKTSTVEACLPLELTKNESCLNSRETSFITNGSCLASR KTSFMMALCLSSIYEDLKMYQVEFKTMNAKLLMDPKRQIFLDQNMLAVIDELMQALNF NSETVPQKSSLEEPDFYKTKIKLCILLHAFRIRAVTIDRVMSYLNAS]. In some embodiments, the IL-12 cytokine can elicit one or more of the cellular responses selected from the group consisting of: proliferation in a NK cell, differentiation in a NK cell, proliferation in a T cell, and differentiation in a T cell.

In another embodiment the cytokine of the multispecific or multifunctional polypeptide is IL-10. In a specific embodiment said IL-10 cytokine is a single chain IL-10 cytokine. In an even more specific embodiment the single chain IL-10 cytokine comprises the polypeptide sequence of SEQ ID NO: 2300 [SPGQGTQSENSCTHFPGNLPNMLRDLRDAFSRVKTFFQMKDQLDNLLLKESLLEDFKG YLGCQALSEMIQFYLEEVMPQAENQDPDIKAHVNSLGENLKTLRLRLRRCHRFLPCENK SKAVEQVKNAFNKLQEKGIYKAMSEFDIFINYIEAYMTMKIRNGGGGSGGGGSGGGGS GGGGSSPGQGTQSENSCTHFPGNLPNMLRDLRDAFSRVKTFFQMKDQLDNLLLKESLLE DFKGYLGCQALSEMIQFYLEEVMPQAENQDPDIKAHVNSLGENLKTLRLRLRRCHRFLP CENKSKAVEQVKNAFNKLQEKGIYKAMSEFDIFINYIEAYMTMKIRN].

In another specific embodiment the IL-10 cytokine is a monomeric IL-10 cytokine. In a more specific embodiment the monomeric IL-10 cytokine comprises the polypeptide sequence of SEQ ID NO: 2310 [SPGQGTQSENSCTHFPGNLPNMLRDLRDAFSRVKTFFQMKDQLDNLLLKESLLEDFKG YLGCQALSEMIQFYLEEVMPQAENQDPDIKAHVNSLGENLKTLRLRLRRCHRFLPCENG GGSGGKSKAVEQVKNAFNKLQEKGIYKAMSEFDIFINYIEAYMTMKIRN]. In some embodiments, the IL-10 cytokine can elicit one or more of the cellular responses selected from the group consisting of: inhibition of cytokine secretion, inhibition of antigen presentation by antigen presenting cells, reduction of oxygen radical release, and inhibition of T cell proliferation. A multispecific or multifunctional polypeptide according to the disclosure wherein the cytokine is IL-10 is particularly useful for downregulation of inflammation, e.g. in the treatment of an inflammatory disorder.

In another embodiment, the cytokine of the multispecific or multifunctional polypeptide is IL-15. In a specific embodiment said IL-15 cytokine is a mutant IL-15 cytokine having reduced binding affinity to the α-subunit of the IL-15 receptor. Without wishing to be bound by theory, a mutant IL-15 polypeptide with reduced binding to the .alpha.-subunit of the IL-15 receptor has a reduced ability to bind to fibroblasts throughout the body, resulting in improved pharmacokinetics and toxicity profile, compared to a wild-type IL-15 polypeptide. The use of an cytokine with reduced toxicity, such as the described mutant IL-2 and mutant IL-15 effector moieties, is particularly advantageous in a multispecific or multifunctional polypeptide according to the disclosure, having a long serum half-life due to the presence of an Fc domain. In some embodiments the mutant IL-15 cytokine of the multispecific or multifunctional polypeptide according to the disclosure comprises at least one amino acid mutation that reduces or abolishes the affinity of the mutant IL-15 cytokine to the .alpha.-subunit of the IL-15 receptor but preserves the affinity of the mutant IL-15 cytokine to the intermediate-affinity IL-15/IL-2 receptor (consisting of the .beta.- and .gamma.-subunits of the IL-15/IL-2 receptor), compared to the non-mutated IL-15 cytokine. In some embodiments the amino acid mutation is an amino acid substitution. In a specific embodiment, the mutant IL-15 cytokine comprises an amino acid substitution at the position corresponding to residue 53 of human IL-15. In a more specific embodiment, the mutant IL-15 cytokine is human IL-15 comprising the amino acid substitution E53A. In some embodiments the mutant IL-15 cytokine additionally comprises an amino acid mutation at a position corresponding to position 79 of human IL-15, which eliminates the N-glycosylation site of IL-15. Particularly, said additional amino acid mutation is an amino acid substitution replacing an asparagine residue by an alanine residue. In an even more specific embodiment the IL-15 cytokine comprises the polypeptide sequence of SEQ ID NO: 2320 [NWVNVISDLKKIEDLIQSMHIDATLYTESDVHPSCKVTAMKCFLLELQVISLASGDASIH DTVENLIILANNSLSSNGAVTESGCKECEELEEKNIKEFLQSFVHIVQMFINTS]. In some embodiments, the IL-15 cytokine can elicit one or more of the cellular responses selected from the group consisting of: proliferation in an activated T lymphocyte cell, differentiation in an activated T lymphocyte cell, cytotoxic T cell (CTL) activity, proliferation in an activated B cell, differentiation in an activated B cell, proliferation in a natural killer (NK) cell, differentiation in a NK cell, cytokine secretion by an activated T cell or an NK cell, and NK/lymphocyte activated killer (LAK) antitumor cytotoxicity.

Mutant cytokine molecules useful as effector moieties in the multispecific or multifunctional polypeptide can be prepared by deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing. Substitution or insertion may involve natural as well as non-natural amino acid residues. Amino acid modification includes well known methods of chemical modification such as the addition or removal of glycosylation sites or carbohydrate attachments, and the like.

In some embodiments, the cytokine, particularly a single-chain cytokine, of the multispecific or multifunctional polypeptide is GM-CSF. In a specific embodiment, the GM-CSF cytokine can elicit proliferation and/or differentiation in a granulocyte, a monocyte or a dendritic cell. In some embodiments, the cytokine, particularly a single-chain cytokine, of the multispecific or multifunctional polypeptide is IFN-α. In a specific embodiment, the IFN-α cytokine can elicit one or more of the cellular responses selected from the group consisting of: inhibiting viral replication in a virus-infected cell, and upregulating the expression of major histocompatibility complex I (MHC I). In another specific embodiment, the IFN-α cytokine can inhibit proliferation in a tumor cell. In some embodiments the cytokine, particularly a single-chain cytokine, of the multispecific or multifunctional polypeptide is IFNγ. In a specific embodiment, the IFN-γ cytokine can elicit one or more of the cellular responses selected from the group of: increased macrophage activity, increased expression of MHC molecules, and increased NK cell activity. In some embodiments the cytokine, particularly a single-chain cytokine, of the multispecific or multifunctional polypeptide is IL-7. In a specific embodiment, the IL-7 cytokine can elicit proliferation of T and/or B lymphocytes. In some embodiments, the cytokine, particularly a single-chain cytokine, of the multispecific or multifunctional polypeptide is IL-8. In a specific embodiment, the IL-8 cytokine can elicit chemotaxis in neutrophils. In some embodiments, the cytokine, particularly a single-chain cytokine, of the multispecific or multifunctional polypeptide, is MIP-1α. In a specific embodiment, the MIP-1α cytokine can elicit chemotaxis in monocytes and T lymphocyte cells. In some embodiments, the cytokine, particularly a single-chain cytokine, of the multispecific or multifunctional polypeptide is MIP-1β. In a specific embodiment, the MIP-1β cytokine can elicit chemotaxis in monocytes and T lymphocyte cells. In some embodiments, the cytokine, particularly a single-chain cytokine, of the multispecific or multifunctional polypeptide is TGF-β. In a specific embodiment, the TGF-β cytokine can elicit one or more of the cellular responses selected from the group consisting of: chemotaxis in monocytes, chemotaxis in macrophages, upregulation of IL-1 expression in activated macrophages, and upregulation of IgA expression in activated B cells.

In some embodiments, the multispecific or multifunctional polypeptide of the disclosure binds to an cytokine receptor with a dissociation constant (K_{D}) that is at least about 1, 1.5, 2, 2.5, 3, 3.5, 4, 4.5, 5, 5.5, 6, 6.5, 7, 7.5, 8, 8.5, 9, 9.5 or 10 times greater than that for a control cytokine. In another embodiment, the multispecific or multifunctional polypeptide binds to an cytokine receptor with a K_{D} that is at least 2, 3, 4, 5, 6, 7, 8, 9, or 10 times greater than that for a corresponding multispecific or multifunctional polypeptide comprising two or more effector moieties. In another embodiment, the multispecific or multifunctional polypeptide binds to an cytokine receptor with a dissociation constant K_{D} that is about 10 times greater than that for a corresponding the multispecific or multifunctional polypeptide comprising two or more cytokines.

In some embodiments, the multispecific molecules as described herein include a cytokine molecule. In embodiments, the cytokine molecule includes a full length, a fragment or a variant of a cytokine; a cytokine receptor domain, e.g., a cytokine receptor dimerizing domain; or an agonist of a cytokine receptor, e.g., an antibody molecule (e.g., an agonistic antibody) to a cytokine receptor.

In some embodiments the cytokine molecule is chosen from IL-2, IL-12, IL-15, IL-18, IL-7, IL-21, or interferon gamma, or a fragment or variant thereof, or a combination of any of the aforesaid cytokines. The cytokine molecule can be a monomer or a dimer. In embodiments, the cytokine molecule can further include a cytokine receptor dimerizing domain.

In other embodiments, the cytokine molecule is an agonist of a cytokine receptor, e.g., an antibody molecule (e.g., an agonistic antibody) to a cytokine receptor chosen from an IL-15Ra or IL-21R.

In some embodiments, the cytokine molecule is IL-15, e.g., human IL-15 (e.g., comprising the amino acid sequence: NWVNVISDLKKIEDLIQSMHIDATLYTESDVHPSCKVTAMKCFLLELQVISLESGDASIH DTVENLIILANNSLSSNGNVTESGCKECEELEEKNIKEFLQSFVHIVQMFINTS (SEQ ID NO: 2170), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 2170.

In some embodiments, the cytokine molecule comprises a receptor dimerizing domain, e.g., an IL15Ralpha dimerizing domain. In some embodiments, the IL15Ralpha dimerizing domain comprises the amino acid sequence: MAPRRARGCRTLGLPALLLLLLLRPPATRGITCPPPMSVEHADIWVKSYSLYSRERYIC NSGFKRKAGTSSLTECVL (SEQ ID NO: 2180), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 2180. In some embodiments, the cytokine molecule (e.g., IL-15) and the receptor dimerizing domain (e.g., an IL15Ralpha dimerizing domain) of the multispecific molecule are covalently linked, e.g., via a linker (e.g., a Gly-Ser linker. e.g., a linker comprising the amino acid sequence SGGSGGGGSGGGSGGGGSLQ (SEQ ID NO: 2190). In other embodiments, the cytokine molecule (e.g., IL-15) and the receptor dimerizing domain (e.g., an IL15Ralpha dimerizing domain) of the multispecific molecule are not covalently linked, e.g., are non-covalently associated.

In other embodiments, the cytokine molecule is IL-2, e.g., human IL-2 (e.g., comprising the amino acid sequence: APTSSSTKKTQLQLEHLLLDLQMILNGINNYKNPKLTRMLTFKFYMPKKATELKHLQCL EEELKPLEEVLNLAQSKNFHLRPRDLISNINVIVLELKGSETFFMCEYADETATIVEFLNR WITFCQSIISTLT (SEQ ID NO: 2191), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO:2191).

In other embodiments, the cytokine molecule is IL-18, e.g., human IL-18 (e.g., comprising the amino acid sequence: YFGKLESKLSVIRNLNDQVLFIDQGNRPLFEDMTDSDCRDNAPRTIFIISMYKDSQPRGM AVTISVKCEKISTLSCENKIISFKEMNPPDNIKDTKSDIIFFQRSVPGHDNKMQFESSSYEG YFLACEKERDLFKLILKKEDELGDRSIMFTVQNED (SEQ ID NO: 2192), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 2192).

In other embodiments, the cytokine molecule is IL-21, e.g., human IL-21 (e.g., comprising the amino acid sequence: QGQDRHMIRMRQLIDIVDQLKNYVNDLVPEFLPAPEDVETNCEWSAFSCFQKAQLKSA NTGNNERIINVSIKKLKRKPPSTNAGRRQKHRLTCPSCDSYEKKPPKEFLERFKSLLQKMI HQHLSSRTHGSEDS (SEQ ID NO: 2193), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 2193).

In yet other embodiments, the cytokine molecule is interferon gamma, e.g., human interferon gamma (e.g., comprising the amino acid sequence: QDPYVKEAENLKKYFNAGHSDVADNGTLFLGILKNWKEESDRKIMQSQIVSFYFKLFK NFKDDQSIQKSVETIKEDMNVKFFNSNKKKRDDFEKLTNYSVTDLNVQRKAIHELIQVM AELSPAAKTGKRKRSQMLFRG (SEQ ID NO: 2194), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 2194).

### Immune Cell Engagers

In some embodiments, the multifunctional molecule further includes an immune cell engager. “An immune cell engager” refers to one or more binding specificities that bind and/or activate an immune cell, e.g., a cell involved in an immune response. In embodiments, the immune cell is chosen from a T cell, an NK cell, a B cell, a dendritic cell, and/or the macrophage cell. The immune cell engager can be an antibody molecule, a receptor molecule (e.g., a full length receptor, receptor fragment, or fusion thereof (e.g., a receptor-Fc fusion)), or a ligand molecule (e.g., a full length ligand, ligand fragment, or fusion thereof (e.g., a ligand-Fc fusion)) that binds to the immune cell antigen (e.g., the T cell, the NK cell antigen, the B cell antigen, the dendritic cell antigen, and/or the macrophage cell antigen). In embodiments, the immune cell engager specifically binds to the target immune cell, e.g., binds preferentially to the target immune cell. For example, when the immune cell engager is an antibody molecule, it binds to an immune cell antigen (e.g., a T cell antigen, an NK cell antigen, a B cell antigen, a dendritic cell antigen, and/or a macrophage cell antigen) with a dissociation constant of less than about 10 nM.

The immune cell engagers, e.g., first and/or second immune cell engager, of the multispecific or multifunctional molecules as described herein can mediate binding to, and/or activation of, an immune cell, e.g., an immune effector cell. In some embodiments, the immune cell is chosen from a T cell, an NK cell, a B cell, a dendritic cell, or a macrophage cell engager, or a combination thereof. In some embodiments, the immune cell engager is chosen from one, two, three, or all of a T cell engager, NK cell engager, a B cell engager, a dendritic cell engager, or a macrophage cell engager, or a combination thereof. The immune cell engager can be an agonist of the immune system. In some embodiments, the immune cell engager can be an antibody molecule, a ligand molecule (e.g., a ligand that further comprises an immunoglobulin constant region, e.g., an Fc region), a small molecule, a nucleotide molecule.

### Natural Killer Cell Engagers:

Natural Killer (NK) cells recognize and destroy tumors and virus-infected cells in an antibody-independent manner. The regulation of NK cells is mediated by activating and inhibiting receptors on the NK cell surface. One family of activating receptors is the natural cytotoxicity receptors (NCRs) which include NKp30, NKp44 and NKp46. The NCRs initiate tumor targeting by recognition of heparan sulfate on cancer cells. NKG2D is a receptor that provides both stimulatory and costimulatory innate immune responses on activated killer (NK) cells, leading to cytotoxic activity. DNAM1 is a receptor involved in intercellular adhesion, lymphocyte signaling, cytotoxicity and lymphokine secretion mediated by cytotoxic T-lymphocyte (CTL) and NK cell. DAP10 (also known as HCST) is a transmembrane adapter protein which associates with KLRK1 to form an activation receptor KLRK1-HCST in lymphoid and myeloid cells; this receptor plays a major role in triggering cytotoxicity against target cells expressing cell surface ligands such as MHC class I chain-related MICA and MICB, and U(optionally L1)6-binding proteins (ULBPs); it KLRK1-HCST receptor plays a role in immune surveillance against tumors and is required for cytolysis of tumors cells; indeed, melanoma cells that do not express KLRK1 ligands escape from immune surveillance mediated by NK cells. CD16 is a receptor for the Fc region of IgG, which binds complexed or aggregated IgG and also monomeric IgG and thereby mediates antibody-dependent cellular cytotoxicity (ADCC) and other antibody-dependent responses, such as phagocytosis.

In some embodiments, the NK cell engager is a viral hemagglutinin (HA), HA is a glycoprotein found on the surface of influenza viruses. It is responsible for binding the virus to cells with sialic acid on the membranes, such as cells in the upper respiratory tract or erythrocytes. HA has at least 18 different antigens. These subtypes are named H1 through H18. NCRs can recognize viral proteins. NKp46 has been shown to be able to interact with the HA of influenza and the HA-NA of Paramyxovirus, including Sendai virus and Newcastle disease virus. Besides NKp46, NKp44 can also functionally interact with HA of different influenza subtypes.

Provided herein are, inter alia, multispecific (e.g., bi-, tri-, quad-specific) or multifunctional molecules that are engineered to contain one or more NK cell engagers that mediate binding to and/or activation of an NK cell. Accordingly, in some embodiments, the NK cell engager is selected from an antigen binding domain or ligand that binds to (e.g., activates): NKp30, NKp40, NKp44, NKp46, NKG2D, DNAM1, DAP10, CD16 (e.g., CD16a, CD16b, or both), CRTAM, CD27, PSGL1, CD96, CD100 (SEMA4D), NKp80, CD244 (also known as SLAMF4 or 2B4), SLAMF6, SLAMF7, KIR2DS2, KIR2DS4, KIR3DS1, KIR2DS3, KIR2DS5, KIR2DS1, CD94, NKG2C, NKG2E, or CD160.

In some embodiments, the NK cell engager is a ligand of NKp30 is a B7-6, e.g., comprises the amino acid sequence of: DLKVEMMAGGTQITPLNDNVTIFCNIFYSQPLNITSMGITWFWKSLTFDKEVKVFEFFGD HQEAFRPGAIVSPWRLKSGDASLRLPGIQLEEAGEYRCEVVVTPLKAQGTVQLEVVASP ASRLLLDQVGMKENEDKYMCESSGFYPEAINITWEKQTQKFPHPIEISEDVITGPTIKNM DGTFNVTSCLKLNSSQEDPGTVYQCVVRHASLHTPLRSNFTLTAARHSLSETEKTDNFS (SEQ ID NO: 3291), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3291.

In other embodiments, the NK cell engager is a ligand of NKp44 or NKp46, which is a viral HA. Viral hemagglutinins (HA) are glyco proteins which are on the surface of viruses. HA proteins allow viruses to bind to the membrane of cells via sialic acid sugar moieties which contributes to the fusion of viral membranes with the cell membranes (see e.g., Eur J Immunol. 2001 September; 31(9):2680-9 “Recognition of viral hemagglutinins by NKp44 but not by NKp30”; and Nature. 2001 Feb. 22; 409(6823):1055-60 “Recognition of haemaglutinins on virus-infected cells by NKp46 activates lysis by human NK cells” the contents of each of which are incorporated by reference herein).

In other embodiments, the NK cell engager is a ligand of NKG2D chosen from MICA, MICB, or ULBP1, e.g., wherein: (i) MICA comprises the amino acid sequence: EPHSLRYNLTVLSWDGSVQSGFLTEVHLDGQPFLRCDRQKCRAKPQGQWAEDVLGNK TWDRETRDLTGNGKDLRMTLAHIKDQKEGLHSLQEIRVCEIHEDNSTRSSQHFYYDGEL FLSQNLETKEWTMPQSSRAQTLAMNVRNFLKEDAMKTKTHYHAMHADCLQELRRYL KSGVVLRRTVPPMVNVTRSEASEGNITVTCRASGFYPWNITLSWRQDGVSLSHDTQQW GDVLPDGNGTYQTWVATRICQGEEQRFTCYMEHSGNHSTHPVPSGKVLVLQSHW (SEQ ID NO: 3292), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3292; (ii) MICB comprises the amino acid sequence: AEPHSLRYNLMVLSQDESVQSGFLAEGHLDGQPFLRYDRQKRRAKPQGQWAEDVLGA KTWDTETEDLTENGQDLRRTLTHIKDQKGGLHSLQEIRVCEIHEDSSTRGSRHFYYDGE LFLSQNLETQESTVPQSSRAQTLAMNVTNFWKEDAMKTKTHYRAMQADCLQKLQRYL KSGVAIRRTVPPMVNVTCSEVSEGNITVTCRASSFYPRNITLTWRQDGVSLSHNTQQWG DVLPDGNGTYQTWVATRIRQGEEQRFTCYMEHSGNHGTHPVPSGKVLVLQSQRTD (SEQ ID NO: 3293), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3293; or (iii) ULBP1 comprises the amino acid sequence: GWVDTHCLCYDFIITPKSRPEPQWCEVQGLVDERPFLHYDCVNHKAKAFASLGKKVNV TKTWEEQTETLRDVVDFLKGQLLDIQVENLIPIEPLTLQARMSCEHEAHGHGRGSWQFL FNGQKFLLFDSNNRKWTALHPGAKKMTEKWEKNRDVTMFFQKISLGDCKMWLEEFL MYWEQMLDPTKPPSLAPG (SEQ ID NO: 3294), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3294.

In other embodiments, the NK cell engager is a ligand of DNAM1 chosen from NECTIN2 or NECL5, e.g., wherein: (i) NECTIN2 comprises the amino acid sequence: QDVRVQVLPEVRGQLGGTVELPCHLLPPVPGLYISLVTWQRPDAPANHQNVAAFHPKM GPSFPSPKPGSERLSFVSAKQSTGQDTEAELQDATLALHGLTVEDEGNYTCEFATFPKGS VRGMTWLRVIAKPKNQAEAQKVTFSQDPTTVALCISKEGRPPARISWLSSLDWEAKETQ VSGTLAGTVTVTSRFTLVPSGRADGVTVTCKVEHESFEEPALIPVTLSVRYPPEVSISGYD DNWYLGRTDATLSCDVRSNPEPTGYDWSTTSGTFPTSAVAQGSQLVIHAVDSLFNTTFV CTVTNAVGMGRAEQVIFVRETPNTAGAGATGG (SEQ ID NO: 3295), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3295; or (ii) NECL5 comprises the amino acid sequence: WPPPGTGDVVVQAPTQVPGFLGDSVTLPCYLQVPNMEVTHVSQLTWARHGESGSMAV FHQTQGPSYSESKRLEFVAARLGAELRNASLRMFGLRVEDEGNYTCLFVTFPQGSRSVD IWLRVLAKPQNTAEVQKVQLTGEPVPMARCVSTGGRPPAQITWHSDLGGMPNTSQVPG FLSGTVTVTSLWILVPSSQVDGKNVTCKVEHESFEKPQLLTVNLTVYYPPEVSISGYDNN WYLGQNEATLTCDARSNPEPTGYNWSTTMGPLPPFAVAQGAQLLIRPVDKPINTTLICN VTNALGARQAELTVQVKEGPPSEHSGISRN (SEQ ID NO: 3296), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3296.

In yet other embodiments, the NK cell engager is a ligand of DAP10, which is an adapter for NKG2D (see e.g., Proc Natl Acad Sci USA. 2005 May 24; 102(21): 7641-7646; and Blood, 15 Sep. 2011 Volume 118, Number 11, the full contents of each of which is incorporated by reference herein).

In other embodiments, the NK cell engager is a ligand of CD16, which is a CD16a/b ligand, e.g., a CD16a/b ligand further comprising an antibody Fc region (see e.g., Front Immunol. 2013; 4: 76 discusses how antibodies use the Fc to trigger NK cells through CD16, the full contents of which are incorporated herein).

In other embodiments, the NK cell engager is a ligand of CRTAM, which is NECL2, e.g., wherein NECL2 comprises the amino acid sequence: QNLFTKDVTVIEGEVATISCQVNKSDDSVIQLLNPNRQTIYFRDFRPLKDSRFQLLNFSSS ELKVSLTNVSISDEGRYFCQLYTDPPQESYTTITVLVPPRNLMIDIQKDTAVEGEEIEVNC TAMASKPATTIRWFKGNTELKGKSEVEEWSDMYTVTSQLMLKVHKEDDGVPVICQVE HPAVTGNLQTQRYLEVQYKPQVHIQMTYPLQGLTREGDALELTCEAIGKPQPVMVTWV RVDDEMPQHAVLSGPNLFINNLNKTDNGTYRCEASNIVGKAHSDYMLYVYDPPTTIPPP TTTTTTTTTTTTILTIIDSRAGEEGSIRAVDH (SEQ ID NO: 3297), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3297.

In other embodiments, the NK cell engager is a ligand of CD27, which is CD70, e.g., wherein CD70 comprises the amino acid sequence: QRFAQAQQQLPLESLGWDVAELQLNHTGPQQDPRLYWQGGPALGRSFLHGPELDKGQ LRIHRDGIYMVHIQVTLAICSSTTASRHHPTTLAVGICSPASRSISLLRLSFHQGCTIASQR LTPLARGDTLCTNLTGTLLPSRNTDETFFGVQWVRP (SEQ ID NO: 3298), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3298.

In other embodiments, the NK cell engager is a ligand of PSGL1, which is L-selectin (CD62L), e.g., wherein L-selectin comprises the amino acid sequence: WTYHYSEKPMNWQRARRFCRDNYTDLVAIQNKAEIEYLEKTLPFSRSYYWIGIRKIGGI WTWVGTNKSLTEEAENWGDGEPNNKKNKEDCVEIYIKRNKDAGKWNDDACHKLKAA LCYTASCQPWSCSGHGECVEIINNYTCNCDVGYYGPQCQFVIQCEPLEAPELGTMDCTH PLGNFSFSSQCAFSCSEGTNLTGIEETTCGPFGNWSSPEPTCQVIQCEPLSAPDLGIMNCSH PLASFSFTSACTFICSEGTELIGKKKTICESSGIWSNPSPICQKLDKSFSMIKEGDYN (SEQ ID NO: 3299), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3299.

In other embodiments, the NK cell engager is a ligand of CD96, which is NECL5, e.g., wherein NECL5 comprises the amino acid sequence: WPPPGTGDVVVQAPTQVPGFLGDSVTLPCYLQVPNMEVTHVSQLTWARHGESGSMAV FHQTQGPSYSESKRLEFVAARLGAELRNASLRMFGLRVEDEGNYTCLFVTFPQGSRSVD IWLRVLAKPQNTAEVQKVQLTGEPVPMARCVSTGGRPPAQITWHSDLGGMPNTSQVPG FLSGTVTVTSLWILVPSSQVDGKNVTCKVEHESFEKPQLLTVNLTVYYPPEVSISGYDNN WYLGQNEATLTCDARSNPEPTGYNWSTTMGPLPPFAVAQGAQLLIRPVDKPINTTLICN VTNALGARQAELTVQVKEGPPSEHSGISRN (SEQ ID NO: 3296), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3296.

In other embodiments, the NK cell engager is a ligand of CD100 (SEMA4D), which is CD72, e.g., wherein CD72 comprises the amino acid sequence: RYLQVSQQLQQTNRVLEVTNSSLRQQLRLKITQLGQSAEDLQGSRRELAQSQEALQVEQ RAHQAAEGQLQACQADRQKTKETLQSEEQQRRALEQKLSNMENRLKPFFTCGSADTCC PSGWIMHQKSCFYISLTSKNWQESQKQCETLSSKLATFSEIYPQSHSYYFLNSLLPNGGS GNSYWTGLSSNKDWKLTDDTQRTRTYAQSSKCNKVHKTWSWWTLESESCRSSLPYICE MTAFRFPD (SEQ ID NO: 3300), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3300.

In other embodiments, the NK cell engager is a ligand of NKp80, which is CLEC2B (AICL), e.g., wherein CLEC2B (AICL) comprises the amino acid sequence: KLTRDSQSLCPYDWIGFQNKCYYFSKEEGDWNSSKYNCSTQHADLTIIDNIEEMNFLRR YKCSSDHWIGLKMAKNRTGQWVDGATFTKSFGMRGSEGCAYLSDDGAATARCYTER KWICRKRIH (SEQ ID NO: 3301), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3301.

In other embodiments, the NK cell engager is a ligand of CD244, which is CD48, e.g., wherein CD48 comprises the amino acid sequence: QGHLVHMTVVSGSNVTLNISESLPENYKQLTWFYTFDQKIVEWDSRKSKYFESKFKGR VRLDPQSGALYISKVQKEDNSTYIMRVLKKTGNEQEWKIKLQVLDPVPKPVIKIEKIEDM DDNCYLKLSCVIPGESVNYTWYGDKRPFPKELQNSVLETTLMPHNYSRCYTCQVSNSVS SKNGTVCLSPPCTLARS (SEQ ID NO: 3302), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3302.

### T Cell Engagers

Provided herein are, inter alia, multispecific (e.g., bi-, tri-, quad-specific) or multifunctional molecules that are engineered to further contain one or more T cell engager that mediate binding to and/or activation of a T cell. In some embodiments, the T cell engager is an antigen binding domain that binds to, e.g., activates TCRα, e.g., a TCRαV region, as described herein. In some embodiments, the T cell engager is selected from an antigen binding domain or ligand that binds to (e.g., and in some embodiments activates) one or more of CD3, TCRα, TCRγ, TCRζ, ICOS, CD28, CD27, HVEM, LIGHT, CD40, 4-1BB, OX40, DR3, GITR, CD30, TIM1, SLAM, CD2, or CD226. In other embodiments, the T cell engager is selected from an antigen binding domain or ligand that binds to and does not activate one or more of CD3, TCRα, TCRγ, TCRζ, ICOS, CD28, CD27, HVEM, LIGHT, CD40, 4-1BB, OX40, DR3, GITR, CD30, TIM1, SLAM, CD2, or CD226.

### B Cell, Macrophage & Dendritic Cell Engagers

Broadly, B cells, also known as B lymphocytes, are a type of white blood cell of the lymphocyte subtype. They function in the humoral immunity component of the adaptive immune system by secreting antibodies. Additionally. B cells present antigen (they are also classified as professional antigen-presenting cells (APCs)) and secrete cytokines. Macrophages are a type of white blood cell that engulfs and digests cellular debris, foreign substances, microbes, cancer cells via phagocytosis. Besides phagocytosis, they play important roles in nonspecific defense (innate immunity) and also help initiate specific defense mechanisms (adaptive immunity) by recruiting other immune cells such as lymphocytes. For example, they are important as antigen presenters to T cells. Beyond increasing inflammation and stimulating the immune system, macrophages also play an important anti-inflammatory role and can decrease immune reactions through the release of cytokines. Dendritic cells (DCs) are antigen-presenting cells that function in processing antigen material and present it on the cell surface to the T cells of the immune system.

Provided herein are, inter alia, multispecific (e.g., bi-, tri-, quad-specific) or multifunctional molecules that further include, e.g., are engineered to contain, one or more B cell, macrophage, and/or dendritic cell engager that mediate binding to and/or activation of a B cell, macrophage, and/or dendritic cell.

Accordingly, in some embodiments, the immune cell engager comprises a B cell, macrophage, and/or dendritic cell engager chosen from one or more of CD40 ligand (CD40L) or a CD70 ligand; an antibody molecule that binds to CD40 or CD70; an antibody molecule to OX40; an OX40 ligand (OX40L); an agonist of a Toll-like receptor (e.g., as described herein, e.g., a TLR4, e.g., a constitutively active TLR4 (caTLR4), or a TLR9 agonists); a 41BB; a CD2; a CD47; or a STING agonist, or a combination thereof.

In some embodiments, the B cell engager is a CD40L, an OX40L, or a CD70 ligand, or an antibody molecule that binds to OX40, CD40 or CD70.

In some embodiments, the macrophage engager is a CD2 agonist. In some embodiments, the macrophage engager is an antigen binding domain that binds to: CD40L or antigen binding domain or ligand that binds CD40, a Toll like receptor (TLR) agonist (e.g., as described herein), e.g., a TLR9 or TLR4 (e.g., caTLR4 (constitutively active TLR4), CD47, or a STING agonist. In some embodiments, the STING agonist is a cyclic dinucleotide, e.g., cyclic di-GMP (cdGMP) or cyclic di-AMP (cdAMP). In some embodiments, the STING agonist is biotinylated.

In some embodiments, the dendritic cell engager is a CD2 agonist. In some embodiments, the dendritic cell engager is a ligand, a receptor agonist, or an antibody molecule that binds to one or more of: OX40L, 41BB, a TLR agonist (e.g., as described herein) (e.g., TLR9 agonist, TLR4 (e.g., caTLR4 (constitutively active TLR4)), CD47, or and a STING agonist. In some embodiments, the STING agonist is a cyclic dinucleotide, e.g., cyclic di-GMP (cdGMP) or cyclic di-AMP (cdAMP). In some embodiments, the STING agonist is biotinylated.

In other embodiments, the immune cell engager mediates binding to, or activation of, one or more of a B cell, a macrophage, and/or a dendritic cell. Exemplary B cell, macrophage, and/or dendritic cell engagers can be chosen from one or more of CD40 ligand (CD40L) or a CD70 ligand; an antibody molecule that binds to CD40 or CD70; an antibody molecule to OX40; an OX40 ligand (OX40L); a Toll-like receptor agonist (e.g., a TLR4, e.g., a constitutively active TLR4 (caTLR4) or a TLR9 agonist); a 41BB agonist; a CD2; a CD47; or a STING agonist, or a combination thereof.

In some embodiments, the B cell engager is chosen from one or more of a CD40L, an OX40L, or a CD70 ligand, or an antibody molecule that binds to OX40. CD40 or CD70.

In other embodiments, the macrophage cell engager is chosen from one or more of a CD2 agonist; a CD40L; an OX40L; an antibody molecule that binds to OX40, CD40 or CD70; a Toll-like receptor agonist or a fragment thereof (e.g., a TLR4, e.g., a constitutively active TLR4 (caTLR4)); a CD47 agonist; or a STING agonist.

In other embodiments, the dendritic cell engager is chosen from one or more of a CD2 agonist, an OX40 antibody, an OX40L, 41BB agonist, a Toll-like receptor agonist or a fragment thereof (e.g., a TLR4, e.g., a constitutively active TLR4 (caTLR4)), CD47 agonist, or a STING agonist.

In some embodiments, the OX40L comprises the amino acid sequence: QVSHRYPRIQSIKVQFTEYKKEKGFILTSQKEDEIMKVQNNSVIINCDGFYLISLKGYFSQ EVNISLHYQKDEEPLFQLKKVRSVNSLMVASLTYKDKVYLNVTTDNTSLDDFHVNGGE LILIHQNPGEFCVL (SEQ ID NO: 3303), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3303.

In another embodiment, the CD40L comprises the amino acid sequence: MQKGDQNPQIAAHVISEASSKTTSVLQWAEKGYYTMSNNLVTLENGKQLTVKRQGLY YIYAQVTFCSNREASSQAPFIASLCLKSPGRFERILLRAANTHSSAKPCGQQSIHLGGVFE LQPGASVFVNVTDPSQVSHGTGFTSFGLLKL (SEQ ID NO: 3304), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3304.

In yet other embodiments, the STING agonist comprises a cyclic dinucleotide, e.g., a cyclic di-GMP (cdGMP), a cyclic di-AMP (cdAMP), or a combination thereof, optionally with 2′,5′ or 3′,5′ phosphate linkages.

In some embodiments, the immune cell engager includes 41BB ligand, e.g., comprising the amino acid sequence: ACPWAVSGARASPGSAASPRLREGPELSPDDPAGLLDLRQGMFAQLVAQNVLLIDGPLS WYSDPGLAGVSLTGGLSYKEDTKELVVAKAGVYYVFFQLELRRVVAGEGSGSVSLALH LQPLRSAAGAAALALTVDLPPASSEARNSAFGFQGRLLHLSAGQRLGVHLHTEARARH AWQLTQGATVLGLFRVTPEIPAGLPSPRSE (SEQ ID NO: 3305), a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3305.

Toll-Like Receptors: Toll-Like Receptors (TLRs) are evolutionarily conserved receptors are homologues of the Drosophila Toll protein, and recognize highly conserved structural motifs known as pathogen-associated microbial patterns (PAMPs), which are exclusively expressed by microbial pathogens, or danger-associated molecular patterns (DAMPs) that are endogenous molecules released from necrotic or dying cells. PAMPs include various bacterial cell wall components such as lipopolysaccharide (LPS), peptidoglycan (PGN) and lipopeptides, as well as flagellin, bacterial DNA and viral double-stranded RNA. DAMPs include intracellular proteins such as heat shock proteins as well as protein fragments from the extracellular matrix. Stimulation of TLRs by the corresponding PAMPs or DAMPs initiates signaling cascades leading to the activation of transcription factors, such as AP-1, NF-κB and interferon regulatory factors (IRFs). Signaling by TLRs results in a variety of cellular responses, including the production of interferons (IFNs), pro-inflammatory cytokines and effector cytokines that direct the adaptive immune response. TLRs are implicated in a number of inflammatory and immune disorders and play a role in cancer (Rakoff-Nahoum S. & Medzhitov R., 2009. Toll-like receptors and cancer. Nat Revs Cancer 9:57-63).

TLRs are type I transmembrane proteins characterized by an extracellular domain containing leucine-rich repeats (LRRs) and a cytoplasmic tail that contains a conserved region called the Toll/IL-1 receptor (TIR) domain. Ten human and twelve murine TLRs have been characterized, TLR1 to TLR10 in humans, and TLR1 to TLR9, TLR11, TLR12 and TLR13 in mice, the homolog of TLR10 being a pseudogene. TLR2 is essential for the recognition of a variety of PAMPs from Gram-positive bacteria, including bacterial lipoproteins, lipomannans and lipoteichoic acids. TLR3 is implicated in virus-derived double-stranded RNA. TLR4 is predominantly activated by lipopolysaccharide. TLR5 detects bacterial flagellin and TLR9 is required for response to unmethylated CpG DNA. Finally, TLR7 and TLR8 recognize small synthetic antiviral molecules, and single-stranded RNA was reported to be their natural ligand. TLR11 has been reported to recognize uropathogenic E. coli and a profilin-like protein from Toxoplasma gondii. The repertoire of specificities of the TLRs is apparently extended by the ability of TLRs to heterodimerize with one another. For example, dimers of TLR2 and TLR6 are required for responses to diacylated lipoproteins while TLR2 and TLR1 interact to recognize triacylated lipoproteins. Specificities of the TLRs are also influenced by various adapter and accessory molecules, such as MD-2 and CD14 that form a complex with TLR4 in response to LPS.

TLR signaling consists of at least two distinct pathways: a MyD88-dependent pathway that leads to the production of inflammatory cytokines, and a MyD88-independent pathway associated with the stimulation of IFN-β and the maturation of dendritic cells. The MyD88-dependent pathway is common to all TLRs, except TLR3 (Adachi O. et al., 1998. Targeted disruption of the MyD88 gene results in loss of IL-1- and IL-18-mediated function. Immunity. 9(1):143-50). Upon activation by PAMPs or DAMPs, TLRs hetero- or homodimerize inducing the recruitment of adaptor proteins via the cytoplasmic TIR domain. Individual TLRs induce different signaling responses by usage of the different adaptor molecules. TLR4 and TLR2 signaling requires the adaptor TIRAP/Mal, which is involved in the My D88-dependent pathway TLR3 triggers the production of IFN-β in response to double-stranded RNA, in a MyD88-independent manner, through the adaptor TRIF/TICAM-1. TRAM/TICAM-2 is another adaptor molecule involved in the MyD88-independent pathway which function is restricted to the TLR4 pathway.

TLR3, TLR7, TLR8 and TLR9 recognize viral nucleic acids and induce type I IFNs. The signaling mechanisms leading to the induction of type I IFNs differ depending on the TLR activated. They involve the interferon regulatory factors, IRFs, a family of transcription factors known to play a critical role in antiviral defense, cell growth and immune regulation. Three IRFs (IRF3, IRF5 and IRF7) function as direct transducers of virus-mediated TLR signaling. TLR3 and TLR4 activate IRF3 and IRF7, while TLR7 and TLR8 activate IRF5 and IRF7 (Doyle S. et al., 2002. IRF3 mediates a TLR3/TLR4-specific antiviral gene program. Immunity. 17(3):251-63). Furthermore, type I IFN production stimulated by TLR9 ligand CpG-A has been shown to be mediated by PI(3)K and mTOR (Costa-Mattioli M. & Sonenberg N. 2008. RAPping production of type I interferon in pDCs through mTOR. Nature Immunol. 9: 1097-1099).

TLR-9: TLR9 recognizes unmethylated CpG sequences in DNA molecules. CpG sites are relatively rare (˜1%) on vertebrate genomes in comparison to bacterial genomes or viral DNA. TLR9 is expressed by numerous cells of the immune system such as B lymphocytes, monocytes, natural killer (NK) cells, and plasmacytoid dendritic cells. TLR9 is expressed intracellularly, within the endosomal compartments and functions to alert the immune system of viral and bacterial infections by binding to DNA rich in CpG motifs. TLR9 signals leads to activation of the cells initiating pro-inflammatory reactions that result in the production of cytokines such as type-1 interferon and IL-12.

TLR Agonists: a TLR agonist can agonize one or more TLR, e.g., one or more of human TLR—1, 2, 3, 4, 5, 6, 7, 8, 9, or 10. In some embodiments, an adjunctive agent described herein is a TLR agonist. In some embodiments, the TLR agonist specifically agonizes human TLR-9. In some embodiments, the TLR-9 agonist is a CpG moiety. As used herein, a CpG moiety, is a linear dinucleotide having the sequence: 5′-C-phosphate-G-3′, that is, cytosine and guanine separated by only one phosphate. In some embodiments, the CpG moiety comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more CpG dinucleotides. In some embodiments, the CpG moiety consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 CpG dinucleotides. In some embodiments, the CpG moiety has 1-5, 1-10, 1-20, 1-30, 1-40, 1-50, 5-10, 5-20, 5-30, 10-20, 10-30, 1040, or 10-50 CpG dinucleotides. In some embodiments, the TLR-9 agonist is a synthetic ODN (oligodeoxynucleotides). CpG ODNs are short synthetic single-stranded DNA molecules containing unmethylated CpG dinucleotides in particular sequence contexts (CpG motifs). CpG ODNs possess a partially or completely phosphorothioated (PS) backbone, as opposed to the natural phosphodiester (PO) backbone found in genomic bacterial DNA. There are three major classes of CpG ODNs: classes A, B and C, which differ in their immunostimulatory activities. CpG-A ODNs are characterized by a PO central CpG-containing palindromic motif and a PS-modified 3′ poly-G string. They induce high IFN-α production from pDCs but are weak stimulators of TLR9-dependent NF-κB signaling and pro-inflammatory cytokine (e.g. IL-6) production. CpG-B ODNs contain a full PS backbone with one or more CpG dinucleotides. They strongly activate B cells and TLR9-dependent NF-κB signaling but weakly stimulate IFN-α secretion. CpG-C ODNs combine features of both classes A and B. They contain a complete PS backbone and a CpG-containing palindromic motif. C-Class CpG ODNs induce strong IFN-α production from pDC as well as B cell stimulation.

### Stromal Modifying Moieties

In some embodiments, the multifunctional molecule further includes a stromal modifying moiety. A “stromal modifying moiety,” as used herein refers to an agent, e.g., a protein (e.g., an enzyme), that is capable of altering, e.g., degrading a component of, the stroma. In embodiments, the component of the stroma is chosen from, e.g., an ECM component, e.g., a glycosaminoglycan, e.g., hyaluronan (also known as hyaluronic acid or HA), chondroitin sulfate, chondroitin, dermatan sulfate, heparin sulfate, heparin, entactin, tenascin, aggrecan and keratin sulfate; or an extracellular protein, e.g., collagen, laminin, elastin, fibrinogen, fibronectin, and vitronectin.

Solid tumors have a distinct structure that mimics that of normal tissues and comprises two distinct but interdependent compartments: the parenchyma (neoplastic cells) and the stroma that the neoplastic cells induce and in which they are dispersed. All tumors have stroma and require stroma for nutritional support and for the removal of waste products. In the case of tumors which grow as cell suspensions (e.g., leukemias, ascites tumors), the blood plasma serves as stroma (Connolly J L et al. Tumor Structure and Tumor Stroma Generation. In: Kufe D W et al., editors. Holland-Frei Cancer Medicine. 6th edition. Hamilton: BC Decker; 2003). The stroma includes a variety of cell types, including fibroblasts/myofibroblasts, glial, epithelial, fat, vascular, smooth muscle, and immune cells along with extracellular matrix (ECM) and extracellular molecules (Li Hanchen et al. Tumor Microenvironment: The Role of the Tumor Stroma in Cancer. J of Cellular Biochemistry 101: 805-815 (2007)).

Stromal modifying moieties described herein include moieties (e.g., proteins, e.g., enzymes) capable of degrading a component of the stroma, e.g., an ECM component, e.g., a glycosaminoglycan, e.g., hyaluronan (also known as hyaluronic acid or HA), chondroitin sulfate, chondroitin, dermatan sulfate, heparin sulfate, heparin, entactin, tenascin, aggrecan and keratin sulfate; or an extracellular protein, e.g., collagen, laminin, elastin, fibrinogen, fibronectin, and vitronectin.

### Stromal Modifying Enzymes

In some embodiments, the stromal modifying moiety is an enzyme. For example, the stromal modifying moiety can include, but is not limited to a hyaluronidase, a collagenase, a chondroitinase, a matrix metalloproteinase (e.g., macrophage metalloelastase).

### Hyaluronidases

Hyaluronidases are a group of neutral- and acid-active enzymes found throughout the animal kingdom. Hyaluronidases vary with respect to substrate specificity, and mechanism of action. There are three general classes of hyaluronidases: (1) Mammalian-type hyaluronidases, (EC 3.2.1.35) which are endo-beta-N-acetylhexosaminidases with tetrasaccharides and hexasaccharides as the major end products. They have both hydrolytic and transglycosidase activities, and can degrade hyaluronan and chondroitin sulfates, (2) Bacterial hyaluronidases (EC 4.2.99.1) degrade hyaluronan and, and to various extents, chondroitin sulfate and dermatan sulfate. They are endo-beta-N-acetylhexosaminidases that operate by a beta elimination reaction that yields primarily disaccharide end products: (3) Hyaluronidases (EC 3.2.1.36) from leeches, other parasites, and crustaceans are endo-beta-glucuronidases that generate tetrasaccharide and hexasaccharide end products through hydrolysis of the beta 1-3 linkage.

Mammalian hyaluronidases can be further divided into two groups: (1) neutral active and (2) acid active enzymes. There are six hyaluronidase-like genes in the human genome, HYAL1, HYAL2, HYAL3 HYAL4 HYALP1 and PH20/SPAM1. HYALP1 is a pseudogene, and HYAL3 has not been shown to possess enzyme activity toward any known substrates. HYAL4 is a chondroitinase and lacks activity towards hyaluronan. HYAL1 is the prototypical acid-active enzyme and PH20 is the prototypical neutral-active enzyme. Acid active hyaluronidases, such as HYAL1 and HYAL2 lack catalytic activity at neutral pH. For example, HYAL1 has no catalytic activity in vitro over pH 4.5 (Frost and Stem, “A Microtiter-Based Assay for Hyaluronidase Activity Not Requiring Specialized Reagents”, Analytical Biochemistry, vol. 251, pp. 263-269 (1997). HYAL2 is an acid active enzyme with a very low specific activity in vitro.

In some embodiments the hyaluronidase is a mammalian hyaluronidase. In some embodiments the hyaluronidase is a recombinant human hyaluronidase. In some embodiments, the hyaluronidase is a neutral active hyaluronidase. In some embodiments, the hyaluronidase is a neutral active soluble hyaluronidase. In some embodiments, the hyaluronidase is a recombinant PH20 neutral-active enzyme. In some embodiments, the hyaluronidase is a recombinant PH20 neutral-active soluble enzyme. In some embodiments the hyaluronidase is glycosylated. In some embodiments, the hyaluronidase possesses at least one N-linked glycan. A recombinant hyaluronidase can be produced using conventional methods known to those of skill in the art, e.g., U.S. Pat. No. 7,767,429, the entire contents of which are incorporated by reference herein.

In some embodiments the hyaluronidase is rHuPH20 (also referred to as Hylenex®; presently manufactured by Halozyme; approved by the FDA in 2005 (see e.g., Scodeller P (2014) Hyaluronidase and other Extracellular Matrix Degrading Enzymes for Cancer Therapy: New Uses and Nano-Formulations. J Carcinog. Mutage 5:178; U.S. Pat. Nos. 7,767,429; 8,202,517; 7,431,380; 8,450,470; 8,772,246; 8,580,252, the entire contents of each of which is incorporated by reference herein). rHuPH20 is produced by genetically engineered CHO cells containing a DNA plasmid encoding for a soluble fragment of human hyaluronidase PH20. In some embodiments the hyaluronidase is glycosylated. In some embodiments, the hyaluronidase possesses at least one N-linked glycan. A recombinant hyaluronidase can be produced using conventional methods known to those of skill in the art, e.g., U.S. Pat. No. 7,767,429, the entire contents of which are incorporated by reference herein. In some embodiments, rHuPH20 has a sequence at least 95% (e.g., at least 96%, 97%, 98%, 99%, 100%) identical to the amino acid sequence of

(SEQ ID NO: 3306)
LNFRAPPVIPNVPFLWAWNAPSEFCLGKFDEPLDMSLFSFIGSPRINAT
GQGVTIFYVDRLGYYPYIDSITGVTVNGGIPQKISLQDHLDKAKKDITF
YMPVDNLGMAVIDWEEWRPTWARNWKPKDVYKNRSIELVQQQNVQLSLT
EATEKAKQEFEKAGKDFLVETIKLGKLLRPNHLWGYYLFPDCYNHHYKK
PGYNGSCFNVEIKRNDDLSWLWNESTALYPSIYLNTQQSPVAATLYVRN
RVREAIRVSKIPDAKSPLPVFAYTRIVFTDQVLKFLSQDELVYTFGETV
ALGASGIVIWGTLSIMRSMKSCLLLDNYMETILNPYIINVTLAAKMCSQ
VLCQEQGVCIRKNWNSSDYLHLNPDNFAIQLEKGGKFTVRGKPTLEDLE
QFSEKFYCSCYSTLSCKEKADVKDTDAVDVCIADGVCIDAFLKPPMETE
EPQIFYNASPSTLS.

In any of the methods provided herein, the anti-hyaluronan agent can be an agent that degrades hyaluronan or can be an agent that inhibits the synthesis of hyaluronan. For example, the anti-hyaluronan agent can be a hyaluronan degrading enzyme. In another example, the anti-hyaluronan agent or is an agent that inhibits hyaluronan synthesis. For example, the anti-hyaluronan agent is an agent that inhibits hyaluronan synthesis such as a sense or antisense nucleic acid molecule against an HA synthase or is a small molecule drug. For example, an anti-hyaluronan agent is 4-methylumbelliferone (MU) or a derivative thereof, or leflunomide or a derivative thereof. Such derivatives include, for example, a derivative of 4-methylumbelliferone (MU) that is 6,7-dihydroxy-4-methyl coumarin or 5,7-dihydroxy-4-methyl coumarin.

In further examples of the methods provided herein, the hyaluronan degrading enzyme is a hyaluronidase. In some examples, the hyaluronan-degrading enzyme is a PH20 hyaluronidase or truncated form thereof to lacking a C-terminal gly cosylphosphatidylinositol (GPI) attachment site or a portion of the GPI attachment site. In specific examples, the hyaluronidase is a PH20 selected from a human, monkey, bovine, ovine, rat, mouse or guinea pig PH20. For example, the hyaluronan-degrading enzyme is a human PH20 hyaluronidase that is neutral active and N-glycosylated and is selected from among (a) a hyaluronidase polypeptide that is a full-length PH20 or is a C-terminal truncated form of the PH20, wherein the truncated form includes at least amino acid residues 36-464 of SEQ ID NO: 139, such as 36-481, 36-482, 36-483, where the full-length PH20 has the sequence of amino acids set forth in SEQ ID NO: 139; or (b) a hyaluronidase polypeptide comprising a sequence of amino acids having at least 85%, 86%, 87%, 88%, 89%, 90%, 91%, 92%, 93%, 94%, 95%, 96%, 97%, 98%, 99% or more sequence identity with the polypeptide or truncated form of sequence of amino acids set forth in SEQ ID NO: 139; or (c) a hyaluronidase polypeptide of (a) or (b) comprising amino acid substitutions, whereby the hyaluronidase polypeptide has a sequence of amino acids having at least 85%, 86%, 87%, 88%, 89%, 90%, 91%, 92%, 93%, 94%, 95%, 96%, 97%, 98%, 99% or more sequence identity with the poly peptide set forth in SEQ ID NO: 139 or the with the corresponding truncated forms thereof. In exemplary examples, the hyaluronan-degrading enzyme is a PH20 that comprises a composition designated rHuPH20.

In other examples, the anti-hyaluronan agent is a hyaluronan degrading enzyme that is modified by conjugation to a polymer. The polymer can be a PEG and the anti-hyaluronan agent a PEGylated hyaluronan degrading enzyme. Hence, in some examples of the methods provided herein the hyaluronan-degrading enzyme is modified by conjugation to a polymer. For example, the hyaluronan-degrading enzyme is conjugated to a PEG, thus the hyaluronan degrading enzyme is PEGylated. In an exemplary example, the hyaluronan-degrading enzyme is a PEGylated PH20 enzyme (PEGPH20). In the methods provided herein, the corticosteroid can be a glucocorticoid that is selected from among cortisones, dexamethasones, hydrocortisones, methylprednisolones, prednisolones and prednisones.

### Chondroitinases

Chondroitinases are enzymes found throughout the animal kingdom which degrade glycosaminoglycans, specifically chondroitins and chondroitin sulfates, through an endoglycosidase reaction. In some embodiments the chondroitinase is a mammalian chondroitinase. In some embodiments the chondroitinase is a recombinant human chondroitinase. In some embodiments the chondroitinase is HYAL4. Other exemplary chondroitinases include chondroitinase ABC (derived from Proteus vulgaris: Japanese Patent Application Laid-open No 6-153947, T. Yamagata et al. J. Biol. Chem., 243, 1523 (1968), S. Suzuki et al, J. Biol. Chem., 243, 1543 (1968)), chondroitinase AC (derived from Flavobacterium heparinum; T. Yamagata et al., J. Biol. Chem., 243, 1523 (1968)), chondroitinase AC II (derived from Arthrobacter aurescens; K. Hiyama, and S. Okada, J. Biol. Chem., 250, 1824 (1975), K. Hiyama and S. Okada, J. Biochem. (Tokyo), 80, 1201 (1976)), Hyaluronidase ACIII (derived from Flavobacterium sp. Hp102; Hirofumi Miyazono et al., Seikagaku, 61, 1023 (1989)), chondroitinase B (derived from Flavobacterium heparinum; Y. M. Michelacci and C. P. Dietrich, Biochem. Biophys. Res. Commun., 56, 973 (1974), Y. M. Michelacci and C. P. Dietrich, Biochem. J., 151, 121 (1975), Kenichi Maeyama et al, Seikagaku, 57, 1189 (1985)), chondroitinase C (derived from Flavobacterium sp. Hp102: Hirofumi Miyazono et al, Seikagaku, 61, 1023 (1939)), and the like.

### Matrix Metalloproteinases

Matrix metalloproteases (MMPs) are zinc-dependent endopeptidases that are the major proteases involved in extracellular matrix (ECM) degradation. MMPs are capable of degrading a wide range of extracellular molecules and a number of bioactive molecules. Twenty-four MMP genes have been identified in humans, which can be organized into six groups based on domain organization and substrate preference: Collagenases (MMP-1, -8 and -13), Gelatinases (MMP-2 and MMP-9), Stromelysins (MMP-3, -10 and -11), Matrilysin (MMP-7 and MMP-26), Membrane-type (MT)-MMPs (MMP-14, -15, -16, -17, -24 and -25) and others (MMP-12, -19, -20, -21, -23, -27 and -28). In some embodiments, the stromal modifying moiety is a human recombinant MMP (e.g., MMP-1, -2, -3, -4, -5-6, -7, -8, -9, 10, -11, -12, -13, -14, 15, -15, -17, -18, -19, 20, -21, -22, -23, or -24).

### Collagenases

The three mammalian collagenases (MMP-1, -8, and -13) are the principal secreted endopeptidases capable of cleaving collagenous extracellular matrix. In addition to fibrillar collagens, collagenases can cleave several other matrix and non-matrix proteins including growth factors. Collagenases are synthesized as inactive pro-forms, and once activated, their activity is inhibited by specific tissue inhibitors of metalloproteinases, TIMPs, as well as by non-specific proteinase inhibitors (Ala-aho R et al. Biochimie. Collagenases in cancer. 2005 March-April; 87(3-4):273-86). In some embodiments, the stromal modifying moiety is a collagenase. In some embodiments, the collagenase is a human recombinant collagenase. In some embodiments, the collagenase is MMP-1. In some embodiments, the collagenase is MMP-8. In some embodiments, the collagenase is MMP-13.

### Macrophage Metalloelastase

Macrophage metalloelastase (MME), also known as MMP-12, is a member of the stromelysin subgroup of MMPs and catalyzes the hydrolysis of soluble and insoluble elastin and a broad selection of matrix and nonmatrix substrates including type IV collagen, fibronectin, laminin, vitronectin, entactin, heparan, and chondroitin sulfates (Erja Kerkelä et al. Journal of Investigative Dermatology (2000) 114, 1113-1119; doi:10.1046/j.1523-1747.2000.00993). In some embodiments, the stromal modifying moiety is a MME. In some embodiments, the MME is a human recombinant MME. In some embodiments, the MME is MMP-12.

### Additional Stromal Modifying Moieties

In some embodiments, the stromal modifying moiety causes one or more of: decreases the level or production of a stromal or extracellular matrix (ECM) component; decreases tumor fibrosis; increases interstitial tumor transport; improves tumor perfusion; expands the tumor microvasculature; decreases interstitial fluid pressure (IFP) in a tumor, or decreases or enhances penetration or diffusion of an agent, e.g., a cancer therapeutic or a cellular therapy, into a tumor or tumor vasculature.

In some embodiments, the stromal or ECM component decreased is chosen from a glycosaminoglycan or an extracellular protein, or a combination thereof. In some embodiments, the glycosaminoglycan is chosen from hyaluronan (also known as hyaluronic acid or HA), chondroitin sulfate, chondroitin, dermatan sulfate, heparin, heparin sulfate, entactin, tenascin, aggrecan and keratin sulfate. In some embodiments, the extracellular protein is chosen from collagen, laminin, elastin, fibrinogen, fibronectin, or vitronectin. In some embodiments, the stromal modifying moiety includes an enzyme molecule that degrades a tumor stroma or extracellular matrix (ECM). In some embodiments, the enzyme molecule is chosen from a hyaluronidase molecule, a collagenase molecule, a chondroitinase molecule, a matrix metalloproteinase molecule (e.g., macrophage metalloelastase), or a variant (e.g., a fragment) of any of the aforesaid. The term “enzyme molecule” includes a full length, a fragment or a variant of the enzyme, e.g., an enzyme variant that retains at least one functional property of the naturally-occurring enzyme.

In some embodiments, the stromal modifying moiety decreases the level or production of hyaluronic acid. In other embodiments, the stromal modifying moiety comprises a hyaluronan degrading enzyme, an agent that inhibits hyaluronan synthesis, or an antibody molecule against haluronic acid.

In some embodiments, the hyaluronan degrading enzyme is a hyaluronidase molecule, e.g., a full length or a variant (e.g., fragment thereof) thereof. In some embodiments, the hyaluronan degrading enzyme is active in neutral or acidic pH, e.g., pH of about 4-5. In some embodiments, the hyaluronidase molecule is a mammalian hyaluronidase molecule, e.g., a recombinant human hyaluronidase molecule, e.g., a full length or a variant (e.g., fragment thereof, e.g., a truncated form) thereof. In some embodiments, the hyaluronidase molecule is chosen from HYAL1, HYAL2, or PH-20/SPAM1, or a variant thereof (e.g., a truncated form thereof). In some embodiments, the truncated form lacks a C-terminal glycosylphosphatidylinositol (GPI) attachment site or a portion of the GPI attachment site. In some embodiments, the hyaluronidase molecule is glycosylated, e.g., comprises at least one N-linked glycan.

In some embodiments, the hyaluronidase molecule comprises the amino acid sequence: LNFRAPPVIPNVPFLWAWNAPSEFCLGKFDEPLDMSLFSFIGSPRINATGQGVTIFYVDRL GYYPYIDSITGVTVNGGIPQKISLQDHLDKAKKDITFYMPVDNLGMAVIDWEEWRPTW ARNWKPKDVYKNRSIELVQQQNVQLSLTEATEKAKQEFEKAGKDFLVETIKLGKLLRP NHLWGYYLFPDCYNHHYKKPGYNGSCFNVEIKRNDDLSWLWNESTALYPSIYLNTQQS PVAATLYVRNRVREAIRVSKIPDAKSPLPVFAYTRIVFTDQVLKFLSQDELVYTFGETVA LGASGIVIWGTLSIMRSMKSCLLLDNYMETILNPYIINVTLAAKMCSQVLCQEQGVCIRK NWNSSDYLHLNPDNFAIQLEKGGKFTVRGKPTLEDLEQFSEKFYCSCYSTLSCKEKADV KDTDAVDVCIADGVCIDAFLKPPMETEEPQIFYNASPSTLS (SEQ ID NO:3311), or a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3311.

In some embodiments, the hyaluronidase molecule comprises: (i) the amino acid sequence of 36-464 of SEQ ID NO: 3311; (ii) the amino acid sequence of 36-481, 36-482, or 36-483 of PH20, wherein PH20 has the sequence of amino acids set forth in SEQ ID NO: 3311; or (iii) an amino acid sequence having at least 95% to 100% sequence identity to the polypeptide or truncated form of sequence of amino acids set forth in SEQ ID NO: 3311; or (iv) an amino acid sequence having 30, 20, 10, 5 or fewer amino acid substitutions to the amino acid sequence set forth in SEQ ID NO: 3311. In some embodiments, the hyaluronidase molecule comprises an amino acid sequence at least 95% (e.g., at least 95%, 96%, 97%, 98%, 99%, 100%) identical to the amino acid sequence of SEQ ID NO: 3311. In some embodiments, the hyaluronidase molecule is encoded by a nucleotide sequence at least 95% (e.g., at least 96%, 97%, 98%, 99%, 100%) identical to the nucleotide sequence of SEQ ID NO: 3311.

In some embodiments, the hyaluronidase molecule is PH20, e.g., rHuPH20. In some embodiments, the hyaluronidase molecule is HYAL1 and comprises the amino acid sequence: FRGPLLPNRPFTTVWNANTQWCLERHGVDVDVSVFDVVANPGQTFRGPDMTIFYSSQG TYPYYTPTGEPVFGGLPQNASLIAHLARTFQDILAAIPAPDFSGLAVIDWEAWRPRWAFN WDTKDIYRQRSRALVQAQHPDWPAPQVEAVAQDQFQGAARAWMAGTLQLGRALRPR GLWGFYGFPDCYNYDFLSPNYTGQCPSGIRAQNDQLGWLWGQSRALYPSIYMPAVLEG TGKSQMYVQHRVAEAFRVAVAAGDPNLPVLPYVQIFYDTTNHFLPLDELEHSLGESAA QGAAGVVLWVSWENTRTKESCQAIKEYMDTTLGPFILNVTSGALLCSQALCSGHGRCV RRTSHPKALLLLNPASFSIQLTPGGGPLSLRGALSLEDQAQMAVEFKCRCYPGWQAPWC ERKSMW (SEQ ID NO: 3312), or a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3312.

In some embodiments, the hyaluronan degrading enzyme, e.g., the hyaluronidase molecule, further comprises a polymer, e.g., is conjugated to a polymer, e.g., PEG. In some embodiments, the hyaluronan-degrading enzyme is a PEGylated PH20 enzyme (PEGPH20). In some embodiments, the hyaluronan degrading enzyme, e.g., the hyaluronidase molecule, further comprises an immunoglobulin chain constant region (e.g., Fc region) chosen from, e.g., the heavy chain constant regions of IgG1, IgG2, IgG3, and IgG4, more particularly, the heavy chain constant region of human IgG1, IgG2, IgG3, or IgG4. In some embodiments, the immunoglobulin constant region (e.g., the Fc region) is linked, e.g., covalently linked to, the hyaluronan degrading enzyme. e.g., the hyaluronidase molecule. In some embodiments, the immunoglobulin chain constant region (e.g., Fc region) is altered, e.g., mutated, to increase or decrease one or more of: Fc receptor binding, antibody glycosylation, the number of cysteine residues, effector cell function, or complement function. In some embodiments, the hyaluronan degrading enzyme, e.g., the hyaluronidase molecule forms a dimer.

In some embodiments, the stromal modifying moiety comprises an inhibitor of the synthesis of hyaluronan, e.g., an HA synthase. In some embodiments, the inhibitor comprises a sense or an antisense nucleic acid molecule against an HA synthase or is a small molecule drug.

In some embodiments, the inhibitor is 4-methylumbelliferone (MU) or a derivative thereof (e.g., 6,7-dihydroxy-4-methyl coumarin or 5,7-dihydroxy-4-methyl coumarin), or leflunomide or a derivative thereof.

In some embodiments, the stromal modifying moiety comprises antibody molecule against hyaluronic acid.

In some embodiments, the stromal modifying moiety comprises a collagenase molecule, e.g., a mammalian collagenase molecule, or a variant (e.g., fragment) thereof. In some embodiments, the collagenase molecule is collagenase molecule IV, e.g., comprising the amino acid sequence of: YNFFPRKPKWDKNQITYRIIGYTPDLDPETVDDAFARAFQVWSDVTPLRFSRIHDGEADI MINFGRWEHGDGYPFDGKDGLLAHAFAPGTGVGGDSHFDDDELWTLGEGQVVRVKY GNADGEYCKFPFLFNGKEYNSCTDTGRSDGFLWCSTTYNFEKDGKYGFCPHEALFTMG GNAEGQPCKFPFRFQGTSYDSCTTEGRTDGYRWCGTTEDYDRDKKYGFCPETAMSTVG GNSEGAPCVFPFTFLGNKYESCTSAGRSDGKMWCATTANYDDDRKWGFCPDQGYSLF LVAAHEFGHAMGLEHSQDPGALMAPIYTYTKNFRLSQDDIKGIQELYGASPDIDLGTGP TPTLGPVTPEICKQDIVFDGIAQIRGEIFFFKDRFIWRTVTPRDKPMGPLLVATFWPELPEK IDAVYEAPQEEKAVFFAGNEYWIYSASTLERGYPKPLTSLGLPPDVQRVDAAFNWSKNK KTYIFAGDKFWRYNEVKKKMDPGFPKLIADAWNAIPDNLDAVVDLQGGGHSYFFKGA YYLKLENQSLKSVKFGSIKSDWLGC (SEQ ID NO: 3313), or a fragment thereof, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions) to the amino acid sequence of SEQ ID NO: 3313.

### Tumor Antigen Moiety

In some embodiments, the multifunctional molecule further includes a tumor antigen moiety. In some embodiments, the tumor-targeting moiety is an antigen, e.g., a cancer antigen. In some embodiments, the cancer antigen is a tumor antigen or stromal antigen, or a hematological antigen.

“Cancer” as used herein can encompass all types of oncogenic processes and/or cancerous growths. In embodiments, cancer includes primary tumors as well as metastatic tissues or malignantly transformed cells, tissues, or organs. In embodiments, cancer encompasses all histopathologies and stages, e.g., stages of invasiveness/severity, of a cancer. In embodiments, cancer includes relapsed and/or resistant cancer. The terms “cancer” and “tumor” can be used interchangeably. For example, both terms encompass solid and liquid tumors. As used herein, the term “cancer” or “tumor” includes premalignant, as well as malignant cancers and tumors.

In some embodiments, the tumor-targeting moiety. e.g., cancer antigen, is chosen from: BCMA, FcRH5, CD19, CD20, CD22, CD30, CD33, CD38, CD47, CD99, CD123, FcRH5, CLEC12, CD179A, SLAMF7, or NY-ESO1, PDL1, CD47, gangloside 2 (GD2), prostate stem cell antigen (PSCA), prostate specific membrane antigen (PMSA), prostate-specific antigen (PSA), carcinoembryonic antigen (CEA), Ron Kinase, c-Met, Immature laminin receptor, TAG-72, BING-4, Calcium-activated chloride channel 2, Cyclin-B1, 9D7, Ep-CAM, EphA3, Her2/neu, Telomerase, SAP-1, Survivin, NY-ESO-1/LAGE-1, PRAME, SSX-2, Melan-A/MART-1, Gp100/pmel17, Tyrosinase, TRP-1/-2, MC1R, β-catenin, BRCA1/2, CDK4, CML66, Fibronectin, p53, Ras, TGF-B receptor, AFP, ETA, MAGE, MUC-1, CA-125, BAGE, GAGE, NY-ESO-1, β-catenin, CDK4, CDC27, α actinin-4, TRP1/gp75, TRP2, gp100, Melan-A/MART1, gangliosides, WT1, EphA3, Epidermal growth factor receptor (EGFR), MART-2, MART-1, MUC1, MUC2, MUM1, MUM2, MUM3, NA88-1, NPM, OA1, OGT, RCC, RUI1, RUI2, SAGE, TRG, TRP1, TSTA, Folate receptor alpha. L1-CAM, CAX, gpA33, GD3, GM2, VEGFR, Intergrins (Integrin alphaVbeta3, Integrin alpha5Beta1), Carbohydrates (Le), IGF1R, EPHA3, TRAILR1, TRAILR2, RANKL, (FAP), TGF-beta, hyaluronic acid, collagen, e.g., collagen IV, tenascin C, or tenascin W. In some embodiments, the tumor-targeting moiety, e.g., cancer antigen, is BCMA. In some embodiments, the tumor-targeting moiety, e.g., cancer antigen, is FcRH5.

In some embodiments, the tumor-targeting moiety, e.g., cancer antigen, is chosen from: CD19, CD123, CD22, CD30, CD171, CS-1, C-type lectin-like molecule-1, CD33, epidermal growth factor receptor variant III (EGFRvIII), ganglioside G2 (GD2), ganglioside GD3. TNF receptor family member B cell maturation (BCMA), Tn antigen ((Tn Ag) or (GalNAcα-Ser/Thr)), prostate-specific membrane antigen (PSMA), Receptor tyrosine kinase-like orphan receptor 1 (ROR1), Fms-Like Tyrosine Kinase 3 (FLT3), Tumor-associated glycoprotein 72 (TAG72), CD38, CD44v6, Carcinoembryonic antigen (CEA), Epithelial cell adhesion molecule (EPCAM), B7H3 (CD276), KIT (CD117), Interleukin-13 receptor subunit alpha-2, mesothelin, Interleukin 11 receptor alpha (IL-11Ra), prostate stem cell antigen (PSCA), Protease Serine 21, vascular endothelial growth factor receptor 2 (VEGFR2), Lewis (Y) antigen, CD24, Platelet-derived growth factor receptor beta (PDGFR-beta), Stage-specific embryonic antigen-4 (SSEA-4), CD20, Folate receptor alpha, Receptor tyrosine-protein kinase ERBB2 (Her2/neu), Mucin 1, cell surface associated (MUC1), epidermal growth factor receptor (EGFR), neural cell adhesion molecule (NCAM), Prostase, prostatic acid phosphatase (PAP), elongation factor 2 mutated (ELF2M), Ephrin B2, fibroblast activation protein alpha (FAP), insulin-like growth factor 1 receptor (IGF-1 receptor), carbonic anhydrase IX (CAIX), Proteasome (Prosome, Macropain) Subunit, Beta Type, 9 (LMP2), glycoprotein 100 (gp100), oncogene fusion protein consisting of breakpoint cluster region (BCR) and Abelson murine leukemia viral oncogene homolog 1 (Abl) (bcr-abl), tyrosinase, ephrin type-A receptor 2 (EphA2), Fucosyl GM1, sialyl Lewis adhesion molecule (sLe), ganglioside GM3, transglutaminase 5 (TGS5), high molecular weight-melanoma-associated antigen (HMWMAA), o-acetyl-GD2 ganglioside (OAcGD2), Folate receptor beta, tumor endothelial marker 1 (TEM1/CD248), tumor endothelial marker 7-related (TEM7R), claudin 6 (CLDN6), thyroid stimulating hormone receptor (TSHR), G protein-coupled receptor class C group 5, member D (GPRC5D), chromosome X open reading frame 61 (CXORF61), CD97, CD179a, anaplastic lymphoma kinase (ALK), Polysialic acid, placenta-specific 1 (PLAC1), hexasaccharide portion of globoH glycoceramide (GloboH), mammary gland differentiation antigen (NY-BR-1), uroplakin 2 (UPK2), Hepatitis A virus cellular receptor 1 (HAVCR1), adrenoceptor beta 3 (ADRB3), pannexin3 (PANX3). G protein-coupled receptor 20 (GPR20), lymphocyte antigen 6 complex, locus K 9 (LY6K), Olfactory receptor 51E2 (OR51E2), TCR Gamma Alternate Reading Frame Protein (TARP), Wilms tumor protein (WT1), Cancer/testis antigen 1 (NY-ESO-1), Cancer/testis antigen 2 (LAGE-1a), Melanoma-associated antigen 1 (MAGE-A1), ETS translocation-variant gene 6, located on chromosome 12p (ETV6-AML), sperm protein 17 (SPA17), X Antigen Family, Member 1A (XAGE1), angiopoietin-binding cell surface receptor 2 (Tie 2), melanoma cancer testis antigen-1 (MAD-CT-1), melanoma cancer testis antigen-2 (MAD-CT-2), Fos-related antigen 1, tumor protein p53 (p53), p53 mutant, prostein, surviving, telomerase, prostate carcinoma tumor antigen-1, melanoma antigen recognized by T cells 1, Rat sarcoma (Ras) mutant, human Telomerase reverse transcriptase (hTERT), sarcoma translocation breakpoints, melanoma inhibitor of apoptosis (ML-1AP), ERG (transmembrane protease, serine 2 (TMPRSS2) ETS fusion gene), N-Acetyl glucosaminyl-transferase V (NA17), paired box protein Pax-3 (PAX3), Androgen receptor, Cyclin B1, v-myc avian myelocytomatosis viral oncogene neuroblastoma derived homolog (MYCN), Ras Homolog Family Member C (RhoC), Tyrosinase-related protein 2 (TRP-2), Cytochrome P450 1B1 (CYP1B1), CCCTC-Binding Factor (Zinc Finger Protein)-Like, Squamous Cell Carcinoma Antigen Recognized By T Cells 3 (SART3), Paired box protein Pax-5 (PAX5), proacrosin binding protein sp32 (OY-TES1), lymphocyte-specific protein tyrosine kinase (LCK), A kinase anchor protein 4 (AKAP-4), synovial sarcoma, X breakpoint 2 (SSX2), Receptor for Advanced Glycation Endproducts (RAGE-1), renal ubiquitous 1 (RU1), renal ubiquitous 2 (RU2), legumain, human papilloma virus E6 (HPV E6), human papilloma virus E7 (HPV E7), intestinal carboxyl esterase, heat shock protein 70-2 mutated (mut hsp70-2), CD79a, CD79b, CD72, Leukocyte-associated immunoglobulin-like receptor 1 (LAIR1), Fc fragment of IgA receptor (FCAR or CD89), Leukocyte immunoglobulin-like receptor subfamily A member 2 (LILRA2), CD300 molecule-like family member f (CD300LF), C-type lectin domain family 12 member A (CLEC12A), bone marrow stromal cell antigen 2 (BST2), EGF-like module-containing mucin-like hormone receptor-like 2 (EMR2), lymphocyte antigen 75 (LY75), Glypican-3 (GPC3), Fc receptor-like 5 (FCRL5), or immunoglobulin lambda-like polypeptide 1 (IGLL1).

### FcRH5 Targeting Moieties:

In some embodiments, the multispecific molecules as described herein include a targeting moiety that binds to FcRH5 (e.g., a FcRH5 targeting moiety). The FcRH5 targeting moiety can be chosen from an antibody molecule (e.g., an antigen binding domain as described herein), a receptor or a receptor fragment, or a ligand or a ligand fragment, or a combination thereof. In some embodiments, the FcRH5 targeting moiety associates with, e.g., binds to, a cancer or hematopoietic cell (e.g., a molecule, e.g., antigen, present on the surface of the cancer or hematopoietic cell). In certain embodiments, the FcRH5 targeting moiety targets, e.g., directs the multispecific molecules as described herein to a cancer or hematopoietic cell. In some embodiments, the cancer is a hematological cancer, e.g., multiple myeloma.

In some embodiments, the multispecific molecule, e.g., the FcRH5 targeting moiety, binds to a FcRH5 antigen on the surface of a cell, e.g., a cancer or hematopoietic cell. The FcRH5 antigen can be present on a primary tumor cell, or a metastatic lesion thereof. In some embodiments, the cancer is a hematological cancer, e.g., multiple myeloma. For example, the FcRH5 antigen can be present on a tumor, e.g., a tumor of a class typified by having one or more of: limited tumor perfusion, compressed blood vessels, or fibrotic tumor interstitium.

The multispecific molecules described herein includes a FcRH5 targeting moiety that comprises an anti-FcRH5 antibody or antigen-binding fragment thereof described in U.S. Pat. No. 7,999,077, US20150098900, U.S. Pat. Nos. 8,299,220, 7,105,149, 8,362,213, 8,466,260, 8,617,559, US20160368985, US20150166661, and US20080247944, the entire contents of any of the aforesaid publications are herein incorporated by reference.

In some embodiments, the multispecific molecules described herein includes a FcRH5 targeting moiety that comprises an anti-FcRH5 antibody or antigen-binding fragment thereof described in U.S. Pat. No. 7,999,077, the entire contents of which are herein incorporated by reference.

### BCMA Targeting Moieties:

In certain embodiments, the multispecific molecules as described herein include a targeting moiety that binds to BCMA (e.g., a BCMA targeting moiety). The BCMA targeting moiety can be chosen from an antibody molecule (e.g., an antigen binding domain as described herein), a receptor or a receptor fragment, or a ligand or a ligand fragment, or a combination thereof. In some embodiments, the BCMA targeting moiety associates with, e.g., binds to, a cancer or hematopoietic cell (e.g., a molecule, e.g., antigen, present on the surface of the cancer or hematopoietic cell). In certain embodiments, the BCMA targeting moiety targets, e.g., directs the multispecific molecules as described herein to a cancer or hematopoietic cell. In some embodiments, the cancer is a hematological cancer, e.g., multiple myeloma.

In some embodiments, the multispecific molecule, e.g., the BCMA targeting moiety, binds to a BCMA antigen on the surface of a cell, e.g., a cancer or hematopoietic cell. The BCMA antigen can be present on a primary tumor cell, or a metastatic lesion thereof. In some embodiments, the cancer is a hematological cancer, e.g., multiple myeloma. For example, the BCMA antigen can be present on a tumor, e.g., a tumor of a class typified by having one or more of: limited tumor perfusion, compressed blood vessels, or fibrotic tumor interstitium.

Exemplary BCMA targeting moieties: the multispecific molecules described herein can include a BCMA targeting moiety that comprises an anti-BCMA antibody or antigen-binding fragment thereof described in U.S. Pat. Nos. 8,920,776, 9,243,058, 9,340,621, 8,846,042, 7,083,785, 9,545,086, 7,276,241, 9,034,324, 7,799,902, 9,387,237, 8,821,883, US861745, US20130273055, US20160176973, US20150368351, US20150376287, US20170022284, US20160015749, US20140242077, US20170037128, US20170051068, US20160368988, US20160311915, US20160131654, US20120213768, US20110177093, US20160297885, EP3137500, EP2699259, EP2982694, EP3029068, EP3023437, WO2016090327, WO2017021450, WO2016110584, WO2016118641, WO2016168149, the entire contents of which are incorporated herein by reference.

In some embodiments, the BCMA-targeting moiety includes an antibody molecule (e.g., Fab or scFv) that binds to BCMA. In some embodiments, the antibody molecule to BCMA comprises one, two, or three CDRs from any of the heavy chain variable domain sequences of Table 7, or a closely related CDR, e.g., CDRs which have at least one amino acid alteration, but not more than two, three or four alterations (e.g., substitutions, deletions, or insertions, e.g., conservative substitutions) from any of the CDR sequences of Table 7. In some embodiments, the antibody molecule to BCMA comprises a heavy chain variable domain sequence chosen from any of the amino acid sequences of Table 7, or an amino acid sequence substantially identical thereto (e.g., 95% to 99.9% identical thereto, or having at least one amino acid alteration, but not more than five, ten or fifteen alterations (e.g., substitutions, deletions, or insertions. e.g., conservative substitutions)).

Alternatively, or in combination with the heavy chain to BCMA as described herein, the antibody molecule to BCMA comprises one, two, or three CDRs from any of the light chain variable domain sequences of Table 7, or a closely related CDR, e.g., CDRs which have at least one amino acid alteration, but not more than two, three or four alterations (e.g., substitutions, deletions, or insertions, e.g., conservative substitutions) from any of the CDR sequences of Table 7. In some embodiments, the antibody molecule to BCMA comprises a light chain variable domain sequence chosen from any of the amino acid sequences of Table 7, or an amino acid sequence substantially identical thereto (e.g., 95% to 99.9% identical thereto, or having at least one amino acid alteration, but not more than five, ten or fifteen alterations (e.g., substitutions, deletions, or insertions, e.g., conservative substitutions)).

### Tumor-Targeting Moieties

In some embodiments, the multifunctional or multispecific (e.g., bi-, tri-, tetra-specific) molecules as described herein further include, e.g., are engineered to further contain, one or more tumor specific targeting moieties that direct the molecule to a tumor cell.

In certain embodiments, the multispecific molecules as described herein further include a tumor-targeting moiety. The tumor targeting moiety can be chosen from an antibody molecule (e.g., an antigen binding domain as described herein), a receptor or a receptor fragment, or a ligand or a ligand fragment, or a combination thereof. In some embodiments, the tumor targeting moiety associates with, e.g., binds to, a tumor cell (e.g., a molecule, e.g., antigen, present on the surface of the tumor cell). In certain embodiments, the tumor targeting moiety targets, e.g., directs the multispecific molecules as described herein to a cancer (e.g., a cancer or tumor cells). In some embodiments, the cancer is chosen from a hematological cancer, a solid cancer, a metastatic cancer, or a combination thereof.

In some embodiments, the multispecific molecule, e.g., the tumor-targeting moiety, binds to a solid tumor antigen or a stromal antigen. The solid tumor antigen or stromal antigen can be present on a solid tumor, or a metastatic lesion thereof. In some embodiments, the solid tumor is chosen from one or more of pancreatic (e.g., pancreatic adenocarcinoma), breast, colorectal, lung (e.g., small or non-small cell lung cancer), skin, ovarian, or liver cancer. In some embodiments, the solid tumor is a fibrotic or desmoplastic solid tumor. For example, the solid tumor antigen or stromal antigen can be present on a tumor, e.g., a tumor of a class typified by having one or more of: limited tumor perfusion, compressed blood vessels, or fibrotic tumor interstitium.

In certain embodiments, the solid tumor antigen is chosen from one or more of: PDL1, CD47, gangloside 2 (GD2), prostate stem cell antigen (PSCA), prostate specific membrane antigen (PMSA), prostate-specific antigen (PSA), carcinoembryonic antigen (CEA), Ron Kinase, c-Met, Immature laminin receptor, TAG-72, BING-4, Calcium-activated chloride channel 2, Cyclin-B1, 9D7, Ep-CAM, EphA3, Her2/neu, Telomerase, SAP-1, Survivin, NY-ESO-1/LAGE-1, PRAME, SSX-2, Melan-A/MART-1, Gp100/pmel17, Tyrosinase, TRP-1/-2, MC1R, β-catenin, BRCA1/2, CDK4, CML66, Fibronectin, p53, Ras, TGF-B receptor, AFP, ETA, MAGE, MUC-1, CA-125, BAGE, GAGE, NY-ESO-1, β-catenin, CDK4, CDC27, CD47, α actinin-4, TRP1/gp75, TRP2, gp100, Melan-A/MART1, gangliosides, WT1, EphA3, Epidermal growth factor receptor (EGFR), MART-2, MART-1, MUC1, MUC2, MUM1, MUM2, MUM3, NA88-1, NPM, OA1, OGT, RCC, RUI1, RUI2, SAGE, TRG, TRP1, TSTA, Folate receptor alpha. L1-CAM, CAIX, EGFRvIII, gpA33, GD3, GM2, VEGFR, Intergrins (Integrin alphaVbeta3, Integrin alpha5Beta1), Carbohydrates (Le), IGF1R, EPHA3, TRAILR1, TRAILR2, or RANKL.

In other embodiments, the multispecific molecule, e.g., the tumor-targeting moiety, binds to a molecule, e.g., antigen, present on the surface of a hematological cancer, e.g., a leukemia or a lymphoma. In some embodiments, the hematological cancer is a B-cell or T cell malignancy. In some embodiments, the hematological cancer is chosen from one or more of a Hodgkin's lymphoma, Non-Hodgkin's lymphoma (e.g., B cell lymphoma, diffuse large B cell lymphoma, follicular lymphoma, chronic lymphocytic leukemia, mantle cell lymphoma, marginal zone B-cell lymphoma, Burkitt lymphoma, lymphoplasmacytic lymphoma, hairy cell leukemia), acute myeloid leukemia (AML), chronic myeloid leukemia, myelodysplastic syndrome (MDS), multiple myeloma, or acute lymphocytic leukemia. In embodiments, the cancer is other than acute myeloid leukemia (AML) or myelodysplastic syndrome (MDS). In embodiments, the hematological antigen is chosen from CD47, CD99, CD30, CD38, SLAMF7, or NY-ESO1. In some embodiments, the hematological antigen is chosen from is chosen from one or more of: BCMA, CD19, CD20, CD22, CD33, CD123, FcRH5, CLEC12, or CD179A.

### Antibody Molecules

In some embodiments, the antibody molecule binds to a cancer antigen, e.g., a tumor antigen or a stromal antigen. In some embodiments, the cancer antigen is, e.g., a mammalian, e.g., a human, cancer antigen. In other embodiments, the antibody molecule binds to an immune cell antigen, e.g., a mammalian, e.g., a human, immune cell antigen. For example, the antibody molecule binds specifically to an epitope, e.g., linear or conformational epitope, on the cancer antigen or the immune cell antigen.

In some embodiments, an antibody molecule is a monospecific antibody molecule and binds a single epitope. E.g., a monospecific antibody molecule having a plurality of immunoglobulin variable domain sequences, each of which binds the same epitope.

In some embodiments, an antibody molecule is a multispecific or multifunctional antibody molecule, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In some embodiments, the first and second epitopes are on the same antigen, e.g., the same protein (or subunit of a multimeric protein). In some embodiments, the first and second epitopes overlap. In some embodiments, the first and second epitopes do not overlap. In some embodiments, the first and second epitopes are on different antigens. e.g., the different proteins (or different subunits of a multimeric protein). In some embodiments, a multispecific antibody molecule comprises a third, fourth or fifth immunoglobulin variable domain. In some embodiments, a multispecific antibody molecule is a bispecific antibody molecule, a trispecific antibody molecule, or a tetraspecific antibody molecule.

In some embodiments, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In some embodiments, the first and second epitopes are on the same antigen. e.g., the same protein (or subunit of a multimeric protein). In some embodiments, the first and second epitopes overlap. In some embodiments, the first and second epitopes do not overlap. In some embodiments, the first and second epitopes are on different antigens, e.g., the different proteins (or different subunits of a multimeric protein). In some embodiments, a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In some embodiments, a bispecific antibody molecule comprises a half antibody having binding specificity for a first epitope and a half antibody having binding specificity for a second epitope. In some embodiments, a bispecific antibody molecule comprises a half antibody, or fragment thereof, having binding specificity for a first epitope and a half antibody, or fragment thereof, having binding specificity for a second epitope. In some embodiments, a bispecific antibody molecule comprises a scFv or a Fab, or fragment thereof, have binding specificity for a first epitope and a scFv or a Fab, or fragment thereof, have binding specificity for a second epitope.

In some embodiments, an antibody molecule comprises a diabody, and a single-chain molecule, as well as an antigen-binding fragment of an antibody (e.g., Fab, F(ab′)₂, and Fv). For example, an antibody molecule can include a heavy (H) chain variable domain sequence (abbreviated herein as VH), and a light (L) chain variable domain sequence (abbreviated herein as VL). In some embodiments, an antibody molecule comprises or consists of a heavy chain and a light chain (referred to herein as a half antibody. In another example, an antibody molecule includes two heavy (H) chain variable domain sequences and two light (L) chain variable domain sequence, thereby forming two antigen binding sites, such as Fab, Fab′, F(ab′)₂, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The preparation of antibody molecules can be monoclonal or polyclonal. An antibody molecule can also be a human, humanized, CDR-grafted, or in vitro generated antibody. The antibody can have a heavy chain constant region chosen from, e.g., IgG1. IgG2, IgG3, or IgG4. The antibody can also have a light chain chosen from, e.g., kappa or lambda. The term “immunoglobulin” (Ig) is used interchangeably with the term “antibody” herein.

Examples of antigen-binding fragments of an antibody molecule include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. These antibody fragments are obtained using conventional techniques known to those with skill in the art, and the fragments are screened for utility in the same manner as are intact antibodies.

Antibody molecules include intact molecules as well as functional fragments thereof. Constant regions of the antibody molecules can be altered, e.g., mutated, to modify the properties of the antibody (e.g., to increase or decrease one or more of: Fc receptor binding, antibody glycosylation, the number of cysteine residues, effector cell function, or complement function).

Antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. According to another aspect of the disclosure, a single domain antibody is a naturally occurring single domain antibody known as heavy chain antibody devoid of light chains. Such single domain antibodies are disclosed in WO 9404678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the disclosure.

The VH and VL regions can be subdivided into regions of hypervariability, termed “complementarity determining regions” (CDR), interspersed with regions that are more conserved, termed “framework regions” (FR or FW).

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg).

The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3).

The precise amino acid sequence boundaries of a given CDR can be determined using any of a number of known schemes, including those described by Kabat et al. (1991), “Sequences of Proteins of Immunological Interest,” 5th Ed. Public Health Service, National Institutes of Health, Bethesda. MD (“Kabat” numbering scheme), Al-Lazikani et al., (1997) JMB 273, 927-948 (“Chothia” numbering scheme). As used herein, the CDRs defined according the “Chothia” number scheme are also sometimes referred to as “hypervariable loops.”

For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia, the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3).

Each VH and VL typically includes three CDRs and four FRs, arranged from amino-terminus to carboxy-terminus in the following order: FR1, CDR1, FR2, CDR2, FR3, CDR3, FR4.

The antibody molecule can be a polyclonal or a monoclonal antibody.

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

The antibody can be recombinantly produced, e.g., produced by phage display or by combinatorial methods, or by yeast display.

Phage display and combinatorial methods for generating antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibody Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

The yeast display method for generating or identifying antibodies is known in the art, e.g., as described in Chao et al. (2006) Nature Protocols 1(2):755-68, the entire contents of which is incorporated by reference herein.

In some embodiments, the antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. Preferably, the non-human antibody is a rodent (mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An antibody molecule can be one in which the variable region, or a portion thereof, e.g., the CDRs, are generated in a non-human organism, e.g., a rat or mouse. Chimeric, CDR-grafted, and humanized antibodies are within the disclosure. Antibody molecules generated in a non-human organism, e.g., a rat or mouse, and then modified, e.g., in the variable framework or constant region, to decrease antigenicity in a human are within the disclosure.

An “effectively human” protein is a protein that does substantially not evoke a neutralizing antibody response, e.g., the human anti-murine antibody (HAMA) response. HAMA can be problematic in a number of circumstances, e.g., if the antibody molecule is administered repeatedly. e.g., in treatment of a chronic or recurrent disease condition. A HAMA response can make repeated antibody administration potentially ineffective because of an increased antibody clearance from the serum (see, e.g., Saleh et al, Cancer Immunol. Immunother., 32:180-190 (1990)) and also because of potential allergic reactions (see, e.g., LoBuglio et al., Hybridoma, 5:5117-5123 (1986)).

Chimeric antibodies can be produced by recombinant DNA techniques known in the art (see Robinson et al., International Patent Publication PCT/US86/02269; Akira, et al., European Patent Application 184,187; Taniguchi, M., European Patent Application 171,496; Morrison et al., European Patent Application 173.494; Neuberger et al., International Application WO 86/01533; Cabilly et al. U.S. Pat. No. 4,816,567; Cabilly et al, European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDRs (of heavy and or light immuoglobulin chains) replaced with a donor CDR. The antibody may be replaced with at least a portion of a non-human CDR or only some of the CDRs may be replaced with non-human CDRs. It is only necessary to replace the number of CDRs required for binding to the antigen. Preferably, the donor will be a rodent antibody, e.g., a rat or mouse antibody, and the recipient will be a human framework or a human consensus framework. Typically, the immunoglobulin providing the CDRs is called the “donor” and the immunoglobulin providing the framework is called the “acceptor.” In some embodiments, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft. Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody molecule can be humanized by methods known in the art (see e.g., Morrison, S. L., 1985, Science 229:1202-1207, by Oi et al., 1986, BioTechniques 4:214, and by Queen et al. U.S. Pat. No. 5,585,089. U.S. Pat. Nos. 5,693,761 and 5,693,762, the contents of all of which are hereby incorporated by reference).

Humanized or CDR-grafted antibody molecules can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present disclosure (UK Patent Application GB 2188638A, filed on Mar. 26, 1987; Winter U.S. Pat. No. 5,225,539), the contents of which is expressly incorporated by reference.

Also within the scope of the disclosure are humanized antibody molecules in which specific amino acids have been substituted, deleted or added. Criteria for selecting amino acids from the donor are described in U.S. Pat. No. 5,585,089, e.g., columns 12-16 of U.S. Pat. No. 5,585,089, e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

The antibody molecule can be a single chain antibody. A single-chain antibody (scFV) may be engineered (see, for example, Colcher, D. et al. (1999) Ann NY Acad Sci 880:263-80; and Reiter, Y. (1996) Clin Cancer Res 2:245-52). The single chain antibody can be dimerized or multimerized to generate multivalent antibodies having specificities for different epitopes of the same target protein.

In yet other embodiments, the antibody molecule has a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from. e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In some embodiments the antibody has: effector function; and can fix complement. In other embodiments the antibody does not: recruit effector cells; or fix complement. In another embodiment, the antibody has reduced or no ability to bind an Fc receptor. For example, it is a isotype or subtype, fragment or other mutant, which does not support binding to an Fc receptor, e.g., it has a mutagenized or deleted Fc receptor binding region.

Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. Nos. 5,624,821 and 5,648,260, the contents of all of which are hereby incorporated by reference). Similar type of alterations could be described which if applied to the murine, or other species immunoglobulin would reduce or eliminate these functions.

An antibody molecule can be derivatized or linked to another functional molecule (e.g., another peptide or protein). As used herein, a “derivatized” antibody molecule is one that has been modified. Methods of derivatization include but are not limited to the addition of a fluorescent moiety, a radionucleotide, a toxin, an enzyme or an affinity ligand such as biotin. Accordingly, the antibody molecules of the disclosure are intended to include derivatized and otherwise modified forms of the antibodies described herein, including immunoadhesion molecules. For example, an antibody molecule can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate association of the antibody or antibody portion with another molecule (such as a streptavidin core region or a polyhistidine tag).

One type of derivatized antibody molecule is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

### CDR-Grafted Scaffolds

In some embodiments, the antibody molecule is a CDR-grafted scaffold domain. In some embodiments, the scaffold domain is based on a fibronectin domain, e.g., fibronectin type III domain. The overall fold of the fibronectin type III (Fn3) domain is closely related to that of the smallest functional antibody fragment, the variable domain of the antibody heavy chain. There are three loops at the end of Fn3; the positions of BC, DE and FG loops approximately correspond to those of CDR1, 2 and 3 of the VH domain of an antibody. Fn3 does not have disulfide bonds; and therefore Fn3 is stable under reducing conditions, unlike antibodies and their fragments (see, e.g., WO 98/56915: WO 01/64942; WO 00/34784). An Fn3 domain can be modified (e.g., using CDRs or hypervariable loops described herein) or varied, e.g., to select domains that bind to an antigen/marker/cell described herein.

In some embodiments, a scaffold domain, e.g., a folded domain, is based on an antibody, e.g., a “minibody” scaffold created by deleting three beta strands from a heavy chain variable domain of a monoclonal antibody (see, e.g., Tramontano et al., 1994, J Mol. Recognit. 7:9; and Martin et al., 1994, EMBO J. 13:5303-5309). The “minibody” can be used to present two hypervariable loops. In some embodiments, the scaffold domain is a V-like domain (see, e.g., Coia et al. WO 99/45110) or a domain derived from tendamistatin, which is a 74 residue, six-strand beta sheet sandwich held together by two disulfide bonds (see, e.g., McConnell and Hoess, 1995, J Mol. Biol. 250:460). For example, the loops of tendamistatin can be modified (e.g., using CDRs or hypervariable loops) or varied, e.g., to select domains that bind to a marker/antigen/cell described herein. Another exemplary scaffold domain is a beta-sandwich structure derived from the extracellular domain of CTLA-4 (see, e.g., WO 00/60070).

Other exemplary scaffold domains include but are not limited to T-cell receptors; MHC proteins; extracellular domains (e.g., fibronectin Type III repeats, EGF repeats): protease inhibitors (e.g., Kunitz domains, ecotin, BPTI, and so forth); TPR repeats; trifoil structures; zinc finger domains: DNA-binding proteins; particularly monomeric DNA binding proteins; RNA binding proteins; enzymes, e.g., proteases (particularly inactivated proteases), RNase; chaperones, e.g., thioredoxin, and heat shock proteins; and intracellular signaling domains (such as SH2 and SH3 domains). See, e.g., US 20040009530 and U.S. Pat. No. 7,501,121, incorporated herein by reference.

In some embodiments, a scaffold domain is evaluated and chosen, e.g., by one or more of the following criteria: (1) amino acid sequence, (2) sequences of several homologous domains, (3) 3-dimensional structure, and/or (4) stability data over a range of pH, temperature, salinity, organic solvent, oxidant concentration. In some embodiments, the scaffold domain is a small, stable protein domain, e.g., a protein of less than 100, 70, 50, 40 or 30 amino acids. The domain may include one or more disulfide bonds or may chelate a metal, e.g., zinc.

### Antibody-Based Fusions

A variety of formats can be generated which contain additional binding entities attached to the N or C terminus of antibodies. These fusions with single chain or disulfide stabilized Fvs or Fabs result in the generation of tetravalent molecules with bivalent binding specificity for each antigen. Combinations of scFvs and scFabs with IgGs enable the production of molecules which can recognize three or more different antigens.

### Antibody-Fab Fusion

Antibody-Fab fusions are bispecific antibodies comprising a traditional antibody to a first target and a Fab to a second target fused to the C terminus of the antibody heavy chain. Commonly the antibody and the Fab will have a common light chain. Antibody fusions can be produced by (1) engineering the DNA sequence of the target fusion, and (2) transfecting the target DNA into a suitable host cell to express the fusion protein. It seems like the antibody-scFv fusion may be linked by a (Gly)-Ser linker between the C-terminus of the CH3 domain and the N-terminus of the scFv, as described by Coloma. J. et al. (1997) Nature Biotech 15:159.

### Antibody-scFv Fusion

Antibody-scFv Fusions are bispecific antibodies comprising a traditional antibody and a scFv of unique specificity fused to the C terminus of the antibody heavy chain. The scFv can be fused to the C terminus through the Heavy Chain of the scFv either directly or through a linker peptide. Antibody fusions can be produced by (1) engineering the DNA sequence of the target fusion, and (2) transfecting the target DNA into a suitable host cell to express the fusion protein. It seems like the antibody-scFv fusion may be linked by a (Gly)-Ser linker between the C-terminus of the CH3 domain and the N-terminus of the scFv, as described by Coloma. J. et al. (1997) Nature Biotech 15:159.

### Variable Domain Immunoglobulin DVD

A related format is the dual variable domain immunoglobulin (DVD), which are composed of VH and VL domains of a second specificity place upon the N termini of the V domains by shorter linker sequences.

Other exemplary multispecific antibody formats include, e.g., those described in the following US20160114057A1, US20130243775A1. US20140051833, US20130022601, US20150017187A1, US20120201746A1, US20150133638A1, US20130266568A1, US20160145340A1, WO2015127158A1, US20150203591A1, US20140322221A1, US20130303396A1, US20110293613, US20130017200A1, US20160102135A1, WO2015197598A2, WO2015197582A1, U.S. Pat. No. 9,359,437, US20150018529, WO2016115274A1, WO2016087416A1, US20080069820A1, U.S. Pat. Nos. 9,145,588B, 7,919,257, and US20150232560A. Exemplary multispecific molecules utilizing a full antibody-Fab/scFab format include those described in the following, U.S. Pat. No. 9,382,323B2, US20140072581A1, US20140308285A1, US20130165638A1, US20130267686A1, US20140377269A1, U.S. Pat. No. 7,741,446B2, and WO1995009917A1. Exemplary multispecific molecules utilizing a domain exchange format include those described in the following, US20150315296A1, WO2016087650A1, US20160075785A1, WO2016016299A1, US20160130347A1, US20150166670, U.S. Pat. No. 8,703,132B2, US20100316645, U.S. Pat. No. 8,227,577B2, US20130078249.

### Fc-Containing Multispecific Molecules

In some embodiments, the multispecific molecules as described herein includes an immunoglobulin constant region (e.g., an Fc region). Exemplary Fc regions can be chosen from the heavy chain constant regions of IgG1, IgG2, IgG3 or IgG4; more particularly, the heavy chain constant region of human IgG1, IgG2, IgG3, or IgG4.

In some embodiments, the immunoglobulin chain constant region (e.g., the Fc region) is altered, e.g., mutated, to increase or decrease one or more of: Fc receptor binding, antibody glycosylation, the number of cysteine residues, effector cell function, or complement function.

In other embodiments, an interface of a first and second immunoglobulin chain constant regions (e.g., a first and a second Fc region) is altered, e.g., mutated, to increase or decrease dimerization, e.g., relative to a non-engineered interface, e.g., a naturally-occurring interface. For example, dimerization of the immunoglobulin chain constant region (e.g., the Fc region) can be enhanced by providing an Fc interface of a first and a second Fc region with one or more of: a paired protuberance-cavity (“knob-in-a hole”), an electrostatic interaction, or a strand-exchange, such that a greater ratio of heteromultimer to homomultimer forms, e.g., relative to a non-engineered interface.

In some embodiments, the multispecific molecules include a paired amino acid substitution at a position chosen from one or more of 347, 349, 350, 351, 366, 368, 370, 392, 394, 395, 397, 398, 399, 405, 407, or 409, e.g., of the Fc region of human IgG1 For example, the immunoglobulin chain constant region (e.g., Fc region) can include a paired an amino acid substitution chosen from: T366S, L368A, or Y407V (e.g., corresponding to a cavity or hole), and T366W (e.g., corresponding to a protuberance or knob).

In other embodiments, the multifunctional molecule includes a half-life extender, e.g., a human serum albumin or an antibody molecule to human serum albumin.

In some embodiments, Fc contains exemplary Fc modifications listed in Table 6.

### Heterodimerized Antibody Molecules & Methods of Making

Various methods of producing multispecific antibodies have been disclosed to address the problem of incorrect heavy chain pairing. Exemplary methods are described below. Exemplary multispecific antibody formats and methods of making said multispecific antibodies are also disclosed in e.g., Speiss et al. Molecular Immunology 67 (2015) 95-106; and Klein et al mAbs 4:6, 653-663; November/December 2012; the entire contents of each of which are incorporated by reference herein.

Heterodimerized bispecific antibodies are based on the natural IgG structure, wherein the two binding arms recognize different antigens. IgG derived formats that enable defined monovalent (and simultaneous) antigen binding are generated by forced heavy chain heterodimerization, combined with technologies that minimize light chain mispairing (e.g., common light chain). Forced heavy chain heterodimerization can be obtained using, e.g., knob-in-hole OR strand exchange engineered domains (SEED).

### Knob-in-Hole

Knob-in-Hole as described in U.S. Pat. Nos. 5,731,116, 7,476,724 and Ridgway, J. et al. (1996) Prot. Engineering 9(7): 617-621, broadly involves: (I) mutating the CH3 domain of one or both antibodies to promote heterodimerization; and (2) combining the mutated antibodies under conditions that promote heterodimerization. “Knobs” or “protuberances” are typically created by replacing a small amino acid in a parental antibody with a larger amino acid (e.g., T366Y or T366W); “Holes” or “cavities” are created by replacing a larger residue in a parental antibody with a smaller amino acid (e.g., Y407T, T366S, L368A and/or Y407V).

For bispecific antibodies including an Fc domain, introduction of specific mutations into the constant region of the heavy chains to promote the correct heterodimerization of the Fc portion can be utilized. Several such techniques are reviewed in Klein et al. (mAbs (2012) 4:6, 1-11), the contents of which are incorporated herein by reference in their entirety. These techniques include the “knobs-into-holes” (KiH) approach which involves the introduction of a bulky residue into one of the CH3 domains of one of the antibody heavy chains. This bulky residue fits into a complementary “hole” in the other CH3 domain of the paired heavy chain so as to promote correct pairing of heavy chains (see e.g., U.S. Pat. No. 7,642,228).

Exemplary KiH mutations include S354C, T366W in the “knob” heavy chain and Y349C, T366S, L368A, Y407V in the “hole” heavy chain. Other exemplary KiH mutations are provided in Table 2, with additional optional stabilizing Fc cysteine mutations.

Other Fc mutations are provided by Igawa and Tsunoda who identified 3 negatively charged residues in the CH3 domain of one chain that pair with three positively charged residues in the CH3 domain of the other chain. These specific charged residue pairs are: E356-K439, E357-K370, D399-K409 and vice versa. By introducing at least two of the following three mutations in chain A: E356K, E357K and D399K, as well as K370E, K409D, K439E in chain B, alone or in combination with newly identified disulfide bridges, they were able to favor very efficient heterodimerization while suppressing homodimerization at the same time (Martens T et al. A novel one-armed antic-Met antibody inhibits glioblastoma growth in vivo. Clin Cancer Res 2006; 12:6144-52; PMID:17062691). Xencor defined 41 variant pairs based on combining structural calculations and sequence information that were subsequently screened for maximal heterodimerization, defining the combination of S364H, F405A (HA) on chain A and Y349T, T394F on chain B (TF) (Moore G L et al. A novel bispecific antibody format enables simultaneous bivalent and monovalent co-engagement of distinct target antigens. MAbs 2011; 3:546-57; PMID: 22123055).

Other exemplary Fc mutations to promote heterodimerization of multispecific antibodies include those described in the following references, the contents of each of which is incorporated by reference herein, WO2016071377A1, US20140079689A1, US20160194389A1, US20160257763, WO2016071376A2, WO2015107026A1, WO2015107025A1, WO2015107015A1, US20150353636A1, US20140199294A1, U.S. Pat. No. 7,750,128B2, US20160229915A1, US20150344570A1, U.S. Pat. No. 8,003,774A1, US20150337049A1, US20150175707A1, US20140242075A1, US20130195849A1, US20120149876A1, US20140200331A1, U.S. Pat. No. 9,309,311B2, U.S. Pat. No. 8,586,713, US20140037621A1, US20130178605A1, US20140363426A1, US20140051835A1 and US20110054151A1.

Stabilizing cysteine mutations have also been used in combination with KiH and other Fc heterodimerization promoting variants, see e.g., U.S. Pat. No. 7,183,076. Other exemplary cysteine modifications include, e.g., those disclosed in US20140348839A1, U.S. Pat. No. 7,855,275B2, and U.S. Pat. No. 9,000,130B2.

### Strand Exchange Engineered Domains (SEED)

Heterodimeric Fc platform that support the design of bispecific and asymmetric fusion proteins by devising strand-exchange engineered domain (SEED) C(H)3 heterodimers are known. These derivatives of human IgG and IgA C(H)3 domains create complementary human SEED C(H)3 heterodimers that are composed of alternating segments of human IgA and IgG C(H)3 sequences. The resulting pair of SEED C(H)3 domains preferentially associates to form heterodimers when expressed in mammalian cells. SEEDbody (Sb) fusion proteins consist of [IgG1 hinge]-C(H)2-[SEED C(H)3], that may be genetically linked to one or more fusion partners (see e.g., Davis J H et al. SEEDbodies: fusion proteins based on strand exchange engineered domain (SEED) CH3 heterodimers in an Fc analogue platform for asymmetric binders or immunofusions and bispecific antibodies. Protein Eng Des Sel 2010; 23:195-202; PMID:20299542 and U.S. Pat. No. 8,871,912. The contents of each of which are incorporated by reference herein).

### Fc-Containing Entities (Mini-Antibodies)

Fc-containing entities, also known as mini-antibodies, can be generated by fusing scFv to the C-termini of constant heavy region domain 3 (CH3-scFv) and/or to the hinge region (scFv-hinge-Fc) of an antibody with a different specificity. Trivalent entities can also be made which have disulfide stabilized variable domains (without peptide linker) fused to the C-terminus of CH3 domains of IgGs.

### Duobody

“Duobody” technology to produce bispecific antibodies with correct heavy chain pairing are known. The DuoBody technology involves three basic steps to generate stable bispecific human IgG1 antibodies in a post-production exchange reaction. In a first step, two IgG1s, each containing single matched mutations in the third constant (CH3) domain, are produced separately using standard mammalian recombinant cell lines. Subsequently, these IgG1 antibodies are purified according to standard processes for recovery and purification. After production and purification (post-production), the two antibodies are recombined under tailored laboratory conditions resulting in a bispecific antibody product with a very high yield (typically >95%)(see e.g., Labrijn et al. PNAS 2013; 110(13):5145-5150 and Labrijn et al. Nature Protocols 2014; 9(10):2450-63, the contents of each of which are incorporated by reference herein).

### Electrostatic Interactions

Methods of making multispecific antibodies using CH3 amino acid changes with charged amino acids such that homodimer formation is electrostatically unfavorable are disclosed. EP1870459 and WO 2009089004 describe other strategies for favoring heterodimer formation upon co-expression of different antibody domains in a host cell. In these methods, one or more residues that make up the heavy chain constant domain 3 (CH3), CH3-CH3 interfaces in both CH3 domains are replaced with a charged amino acid such that homodimer formation is electrostatically unfavorable and heterodimerization is electrostatically favorable. Additional methods of making multispecific molecules using electrostatic interactions are described in the following references, the contents of each of which is incorporated by reference herein, include US20100015133, U.S. Pat. No. 8,592,562B2, U.S. Pat. No. 9,200,060B2, US20140154254A1, and US9358286A1.

### Common Light Chain

Light chain mispairing needs to be avoided to generate homogenous preparations of bispecific IgGs. One way to achieve this is through the use of the common light chain principle, i.e. combining two binders that share one light chain but still have separate specificities. An exemplary method of enhancing the formation of a desired bispecific antibody from a mixture of monomers is by providing a common variable light chain to interact with each of the heteromeric variable heavy chain regions of the bispecific antibody. Compositions and methods of producing bispecific antibodies with a common light chain as disclosed in, e.g., U.S. Pat. No. 7,183,076B2, US20110177073A1, EP2847231A1, WO2016079081 A1, and EP3055329A1, the contents of each of which is incorporated by reference herein.

### CrossMab

Another option to reduce light chain mispairing is the CrossMab technology which avoids non-specific L chain mispairing by exchanging CH1 and CL domains in the Fab of one half of the bispecific antibody. Such crossover variants retain binding specificity and affinity, but make the two arms so different that L chain mispairing is prevented. The CrossMab technology (as reviewed in Klein et al. Supra) involves domain swapping between heavy and light chains so as to promote the formation of the correct pairings. Briefly, to construct a bispecific IgG-like CrossMab antibody that could bind to two antigens by using two distinct light chain-heavy chain pairs, a two-step modification process is applied. First, a dimerization interface is engineered into the C-terminus of each heavy chain using a heterodimerization approach, e.g., Knob-into-hole (KiH) technology, to ensure that only a heterodimer of two distinct heavy chains from one antibody (e.g., Antibody A) and a second antibody (e.g., Antibody B) is efficiently formed. Next, the constant heavy 1 (CH1) and constant light (CL) domains of one antibody are exchanged (Antibody A), keeping the variable heavy (VH) and variable light (VL) domains consistent. The exchange of the CH1 and CL domains ensured that the modified antibody (Antibody A) light chain would only efficiently dimerize with the modified antibody (antibody A) heavy chain, while the unmodified antibody (Antibody B) light chain would only efficiently dimerize with the unmodified antibody (Antibody B) heavy chain; and thus only the desired bispecific CrossMab would be efficiently formed (see e.g., Cain, C. SciBX 4(28); doi:10.1038/scibx.2011.783, the contents of which are incorporated by reference herein).

### Common Heavy Chain

An exemplary method of enhancing the formation of a desired bispecific antibody from a mixture of monomers is by providing a common variable heavy chain to interact with each of the heteromeric variable light chain regions of the bispecific antibody. Compositions and methods of producing bispecific antibodies with a common heavy chain are disclosed in, e.g., US20120184716, US20130317200, and US20160264685A1, the contents of each of which is incorporated by reference herein.

### Amino Acid Modifications

Alternative compositions and methods of producing multispecific antibodies with correct light chain pairing include various amino acid modifications. For example, Zymeworks describes heterodimers with one or more amino acid modifications in the CH1 and/or CL domains, one or more amino acid modifications in the VH and/or VL domains, or a combination thereof, which are part of the interface between the light chain and heavy chain and create preferential pairing between each heavy chain and a desired light chain such that when the two heavy chains and two light chains of the heterodimer pair are co-expressed in a cell, the heavy chain of the first heterodimer preferentially pairs with one of the light chains rather than the other (see e.g., WO2015181805). Other exemplary methods are described in WO2016026943 (Argen-X), US20150211001, US20140072581A1, US20160039947A1, and US20150368352.

### Lambda/Kappa Formats

Multispecific molecules (e.g., multispecific antibody molecules) that include the lambda light chain polypeptide and a kappa light chain polypeptides, can be used to allow for heterodimerization. Methods for generating bispecific antibody molecules comprising the lambda light chain polypeptide and a kappa light chain polypeptides are disclosed in PCT/US17/53053 filed on Sep. 22, 2017 and designated publication number WO 2018/057955, incorporated herein by reference in its entirety.

In some embodiments, the multispecific molecule includes a multispecific antibody molecule, e.g., an antibody molecule comprising two binding specificities, e.g., a bispecific antibody molecule. The multispecific antibody molecule includes:
a lambda light chain polypeptide 1 (LLCP1) specific for a first epitope;
a heavy chain polypeptide 1 (HCP1) specific for the first epitope;
a kappa light chain polypeptide 2 (KLCP2) specific for a second epitope; and
a heavy chain poly peptide 2 (HCP2) specific for the second epitope.

“Lambda light chain polypeptide 1 (LLCP1)”, as that term is used herein, refers to a polypeptide comprising sufficient light chain (LC) sequence, such that when combined with a cognate heavy chain variable region, can mediate specific binding to its epitope and complex with an HCP1. In some embodiments, it comprises all or a fragment of a CH1 region. In some embodiments, an LLCP1 comprises LC-CDR1, LC-CDR2, LC-CDR3, FR1, FR2, FR3, FR4, and CH1, or sufficient sequence therefrom to mediate specific binding of its epitope and complex with an HCP1, LLCP1, together with its HCP1, provide specificity for a first epitope (while KLCP2, together with its HCP2, provide specificity for a second epitope). As described elsewhere herein, LLCP1 has a higher affinity for HCP1 than for HCP2.

“Kappa light chain polypeptide 2 (KLCP2)”, as that term is used herein, refers to a polypeptide comprising sufficient light chain (LC) sequence, such that when combined with a cognate heavy chain variable region, can mediate specific binding to its epitope and complex with an HCP2. In some embodiments, it comprises all or a fragment of a CH1 region. In some embodiments, a KLCP2 comprises LC-CDR1, LC-CDR2, LC-CDR3, FR1, FR2, FR3, FR4, and CH1, or sufficient sequence therefrom to mediate specific binding of its epitope and complex with an HCP2, KLCP2, together with its HCP2, provide specificity for a second epitope (while LLCP1, together with its HCP1, provide specificity for a first epitope).

“Heavy chain polypeptide 1 (HCP1)”, as that term is used herein, refers to a polypeptide comprising sufficient heavy chain (HC) sequence, e.g., HC variable region sequence, such that when combined with a cognate LLCP1, can mediate specific binding to its epitope and complex with an HCP1. In some embodiments, it comprises all or a fragment of a CH1 region. In some embodiments, it comprises all or a fragment of a CH2 and/or CH3 region. In some embodiments, an HCP1 comprises HC-CDR1, HC-CDR2, HC-CDR3, FR1, FR2, FR3, FR4, CH1, CH2, and CH3, or sufficient sequence therefrom to: (i) mediate specific binding of its epitope and complex with an LLCP1, (ii) to complex preferentially, as described herein to LLCP1 as opposed to KLCP2; and (iii) to complex preferentially, as described herein, to an HCP2, as opposed to another molecule of HCP1, HCP1, together with its LLCP1, provide specificity for a first epitope (while KLCP2, together with its HCP2, provide specificity for a second epitope).

“Heavy chain polypeptide 2 (HCP2)”, as that term is used herein, refers to a poly peptide comprising sufficient heavy chain (HC) sequence, e.g., HC variable region sequence, such that when combined with a cognate LLCP1, can mediate specific binding to its epitope and complex with an HCP1. In some embodiments, it comprises all or a fragment of a CH1region. In some embodiments, it comprises all or a fragment of a CH2 and/or CH3 region. In some embodiments, an HCP1 comprises HC-CDR1, HC-CDR2, HC-CDR3, FR1, FR2, FR3, FR4, CH1, CH2, and CH3, or sufficient sequence therefrom to: (i) mediate specific binding of its epitope and complex with an KLCP2, (ii) to complex preferentially, as described herein to KLCP2 as opposed to LLCP1; and (iii) to complex preferentially, as described herein, to an HCP1, as opposed to another molecule of HCP2. HCP2, together with its KLCP2, provide specificity for a second epitope (while LLCP1, together with its HCP1, provide specificity for a first epitope).

In some embodiments, in the multifunctional polypeptide molecule as described herein: LLCP1 has a higher affinity for HCP1 than for HCP2; and/or KLCP2 has a higher affinity for HCP2 than for HCP1.

In some embodiments, the affinity of LLCP1 for HCP1 is sufficiently greater than its affinity for HCP2, such that under preselected conditions, e.g., in aqueous buffer. e.g., at pH 7, in saline, e.g., at pH 7, or under physiological conditions, at least 75, 80, 90, 95, 98, 99, 99.5, or 99.9% of the multispecific antibody molecule molecules have a LLCP1 complexed, or interfaced with, a HCP1.

In some embodiments, in the multifunctional polypeptide molecule as described herein: the HCP1 has a greater affinity for HCP2, than for a second molecule of HCP1; and/or the HCP2 has a greater affinity for HCP1, than for a second molecule of HCP2.

In some embodiments, the affinity of HCP1 for HCP2 is sufficiently greater than its affinity for a second molecule of HCP1, such that under preselected conditions, e.g., in aqueous buffer, e.g., at pH 7, in saline, e.g., at pH 7, or under physiological conditions, at least 75%, 80, 90, 95, 98, 99 99.5 or 99.9% of the multispecific antibody molecule molecules have a HCP1 complexed, or interfaced with, a HCP2.

In another aspect, described herein is a method for making, or producing, a multispecific antibody molecule. The method includes:
(i) providing a first heavy chain polypeptide (e.g., a heavy chain polypeptide comprising one, two, three or all of a first heavy chain variable region (first VH), a first CH1, a first heavy chain constant region (e.g., a first CH2, a first CH3, or both));
(ii) providing a second heavy chain polypeptide (e.g., a heavy chain polypeptide comprising one, two, three or all of a second heavy chain variable region (second VH), a second CH1, a second heavy chain constant region (e.g., a second CH2, a second CH3, or both));
(iii) providing a lambda chain polypeptide (e.g., a lambda light variable region (VLλ), a lambda light constant chain (VLλ), or both) that preferentially associates with the first heavy chain polypeptide (e.g., the first VH); and
(iv) providing a kappa chain polypeptide (e.g., a lambda light variable region (VLλ), a lambda light constant chain (VLλ), or both) that preferentially associates with the second heavy chain polypeptide (e.g., the second VH), under conditions where (i)-(iv) associate.

In some embodiments, the first and second heavy chain polypeptides form an Fc interface that enhances heterodimerization.

In some embodiments, (i)-(iv) (e.g., nucleic acid encoding (i)-(iv)) are introduced in a single cell, e.g., a single mammalian cell, e.g., a CHO cell. In some embodiments, (i)-(iv) are expressed in the cell. In some embodiments, (i)-(iv) (e.g., nucleic acid encoding (i)-(iv)) are introduced in different cells, e.g., different mammalian cells, e.g., two or more CHO cell. In some embodiments, (i)-(iv) are expressed in the cells.

In some embodiments, the method further comprises purifying a cell-expressed antibody molecule, e.g., using a lambda- and/or-kappa-specific purification, e.g., affinity chromatography.

In some embodiments, the method further comprises evaluating the cell-expressed multispecific antibody molecule. For example, the purified cell-expressed multispecific antibody molecule can be analyzed by techniques known in the art, include mass spectrometry. In some embodiments, the purified cell-expressed antibody molecule is cleaved. e.g., digested with papain to yield the Fab moieties and evaluated using mass spectrometry.

In some embodiments, the method produces correctly paired kappa/lambda multispecific, e.g., bispecific, antibody molecules in a high yield, e.g., at least 75%, 80, 90, 95, 98, 99 99.5 or 99.9%.

In other embodiments, the multispecific, e.g., a bispecific, antibody molecule that includes:
(i) a first heavy chain polypeptide (HCP1)(e.g., a heavy chain polypeptide comprising one, two, three or all of a first heavy chain variable region (first VH), a first CH1, a first heavy chain constant region (e.g., a first CH2, a first CH3, or both)), e.g., wherein the HCP1 binds to a first epitope;
(ii) a second heavy chain polypeptide (HCP2)(e.g., a heavy chain polypeptide comprising one, two, three or all of a second heavy chain variable region (second VH), a second CH1, a second heavy chain constant region (e.g., a second CH2, a second CH3, or both)), e.g., wherein the HCP2 binds to a second epitope;
(iii) a lambda light chain polypeptide (LLCP1) (e.g., a lambda light variable region (VLλ), a lambda light constant chain (VLλ), or both) that preferentially associates with the first heavy chain polypeptide (e.g., the first VH), e.g., wherein the LLCP1 binds to a first epitope; and (iv) a kappa light chain polypeptide (KLCP2)(e.g., a kappa light variable region (VLκ), a kappa light constant chain (VLκ), or both) that preferentially associates with the second heavy chain polypeptide (e.g., the second VH), e.g., wherein the KLCP2 binds to a second epitope.

In some embodiments, the first and second heavy chain polypeptides form an Fc interface that enhances heterodimerization. In some embodiments, the multispecific antibody molecule has a first binding specificity that includes a hybrid Vλ-CLλ heterodimerized to a first heavy chain variable region connected to the Fc constant, CH2-CH3 domain (having a knob modification) and a second binding specificity that includes a hybrid VLκ-CLκ heterodimerized to a second heavy chain variable region connected to the Fc constant, CH2-CH3 domain (having a hole modification).

### Multispecific or Multifunctional Antibody Molecules

Exemplary structures of multispecific and multifunctional molecules defined herein are described throughout. Exemplary structures are further described in: Weidle U et al. (2013) The Intriguing Options of Multispecific Antibody Formats for Treatment of Cancer. Cancer Genomics & Proteomics 10: 1-18 (2013); and Spiess C et al. (2015) Alternative molecular formats and therapeutic applications for bispecific antibodies. Molecular Immunology 67: 95-106; the full contents of each of which is incorporated by reference herein).

In some embodiments, multispecific antibody molecules can comprise more than one antigen-binding site, where different sites are specific for different antigens. In some embodiments, multispecific antibody molecules can bind more than one (e.g., two or more) epitopes on the same antigen. In some embodiments, multispecific antibody molecules comprise an antigen-binding site specific for a target cell (e.g., cancer cell) and a different antigen-binding site specific for an immune effector cell. In some embodiments, the multispecific antibody molecule is a bispecific antibody molecule. Bispecific antibody molecules can be classified into five different structural groups: (i) bispecific immunoglobulin G (BsIgG); (ii) IgG appended with an additional antigen-binding moiety; (iii) bispecific antibody fragments; (iv) bispecific fusion proteins; and (v) bispecific antibody conjugates.

BsIgG is a format that is monovalent for each antigen. Exemplary BsIgG formats include but are not limited to crossMab, DAF (two-in-one), DAF (four-in-one), DutaMab, DT-IgG, knobs-in-holes common LC, knobs-in-holes assembly, charge pair, Fab-arm exchange, SEEDbody, triomab, LUZ-Y, Fcab, κλ-body, orthogonal Fab. See Spiess et al. Mol. Immunol. 67(2015):95-106. Exemplary BsIgGs include catumaxomab (Fresenius Biotech, Trion Pharma, Neopharm), which contains an anti-CD3 arm and an anti-EpCAM arm; and ertumaxomab (Neovii Biotech, Fresenius Biotech), which targets CD3 and HER2. In some embodiments, BsIgG comprises heavy chains that are engineered for heterodimerization. For example, heavy chains can be engineered for heterodimerization using a “knobs-into-holes” strategy, a SEED platform, a common heavy chain (e.g., in κλ-bodies), and use of heterodimeric Fc regions. See Spiess et al. Mol. Immunol. 67(2015):95-106. Strategies that have been used to avoid heavy chain pairing of homodimers in BsIgG include knobs-in-holes, duobody, azymetric, charge pair, HA-TF, SEEDbody, and differential protein A affinity. See Id. BsIgG can be produced by separate expression of the component antibodies in different host cells and subsequent purification/assembly into a BsIgG. BsIgG can also be produced by expression of the component antibodies in a single host cell. BsIgG can be purified using affinity chromatography, e.g., using protein A and sequential pH elution.

IgG appended with an additional antigen-binding moiety is another format of bispecific antibody molecules. For example, monospecific IgG can be engineered to have bispecificity by appending an additional antigen-binding unit onto the monospecific IgG, e.g., at the N- or C-terminus of either the heavy or light chain. Exemplary additional antigen-binding units include single domain antibodies (e.g., variable heavy chain or variable light chain), engineered protein scaffolds, and paired antibody variable domains (e.g., single chain variable fragments or variable fragments). See Id. Examples of appended IgG formats include dual variable domain IgG (DVD-Ig), IgG(H)-scFv, scFv-(H)IgG, IgG(L)-scFv, scFv-(L)IgG, IgG(L,H)-Fv, IgG(H)-V, V(H)—IgG, IgG(L)-V, V(L)-IgG, KIH IgG-scFab, 2scFv-IgG, IgG-2scFv, scFv4-Ig, zybody, and DVI-IgG (four-in-one). See Spiess et al. Mol. Immunol. 67(2015):95-106. An example of an IgG-scFv is MM-141 (Merrimack Pharmaceuticals), which binds IGF-1R and HER3. Examples of DVD-Ig include ABT-981 (AbbVie), which binds IL-1α and IL-1β; and ABT-122 (AbbVie), which binds TNF and IL-17A.

Bispecific antibody fragments (BsAb) are a format of bispecific antibody molecules that lack some or all of the antibody constant domains. For example, some BsAb lack an Fc region. In some embodiments, bispecific antibody fragments include heavy and light chain regions that are connected by a peptide linker that permits efficient expression of the BsAb in a single host cell. Exemplary bispecific antibody fragments include but are not limited to nanobody, nanobody-HAS, BiTE, Diabody, DART, TandAb, scDiabody, scDiabody-CH3, Diabody-CH3, triple body, miniantibody, minibody, TriBi minibody, scFv-CH3 KIH, Fab-scFv, scFv-CH-CL-scFv, F(ab′)2, F(ab′)2-scFv2, scFv-KIH, Fab-scFv-Fc, tetravalent HCAb, scDiabody-Fc, Diabody-Fc, tandem scFv-Fc, and intrabody. See Id. For example, the BiTE format comprises tandem scFvs, where the component scFvs bind to CD3 on T cells and a surface antigen on cancer cells.

Bispecific fusion proteins include antibody fragments linked to other proteins, e.g., to add additional specificity and/or functionality. An example of a bispecific fusion protein is an immTAC, which comprises an anti-CD3 scFv linked to an affinity-matured T-cell receptor that recognizes HLA-presented peptides. In some embodiments, the dock-and-lock (DNL) method can be used to generate bispecific antibody molecules with higher valency. Also, fusions to albumin binding proteins or human serum albumin can be extend the serum half-life of antibody fragments. See Id.

In some embodiments, chemical conjugation, e.g., chemical conjugation of antibodies and/or antibody fragments, can be used to create BsAb molecules. See Id. An exemplary bispecific antibody conjugate includes the CovX-body format, in which a low molecular weight drug is conjugated site-specifically to a single reactive lysine in each Fab arm or an antibody or fragment thereof. In some embodiments, the conjugation improves the serum half-life of the low molecular weight drug. An exemplary CovX-body is CVX-241 (NCT01004822), which comprises an antibody conjugated to two short peptides inhibiting either VEGF or Ang2. See Id.

The antibody molecules can be produced by recombinant expression, e.g., of at least one or more component, in a host system. Exemplary host systems include eukaryotic cells (e.g., mammalian cells, e.g., CHO cells, or insect cells, e.g., SF9 or S2 cells) and prokaryotic cells (e.g., E. coli). Bispecific antibody molecules can be produced by separate expression of the components in different host cells and subsequent purification/assembly. Alternatively, the antibody molecules can be produced by expression of the components in a single host cell. Purification of bispecific antibody molecules can be performed by various methods such as affinity chromatography, e.g., using protein A and sequential pH elution. In other embodiments, affinity tags can be used for purification. e.g., histidine-containing tag, myc tag, or streptavidin tag.

### Linkers

The multispecific or multifunctional molecule as described herein can further include a linker, e.g., a linker between one or more of, the antigen binding domain and the cytokine molecule, the antigen binding domain and the immune cell engager, the antigen binding domain and the stromal modifying moiety, the cytokine molecule and the immune cell engager, the cytokine molecule and the stromal modifying moiety, the immune cell engager and the stromal modifying moiety, the antigen binding domain and the immunoglobulin chain constant region, the cytokine molecule and the immunoglobulin chain constant region, the immune cell engager and the immunoglobulin chain constant region, or the stromal modifying moiety and the immunoglobulin chain constant region. In some embodiments, the linker is chosen from: a cleavable linker, a non-cleavable linker, a peptide linker, a flexible linker, a rigid linker, a helical linker, or a non-helical linker, or a combination thereof.

In some embodiments, the multispecific molecule can include one, two, three or four linkers, e.g., a peptide linker. In some embodiments, the peptide linker includes Gly and Ser. In some embodiments, the peptide linker is selected from GGGGS (SEQ ID NO: 3307); GGGGSGGGGS (SEQ ID NO: 3308); GGGGSGGGGSGGGGS (SEQ ID NO: 3309); DVPSGPGGGGGSGGGGS (SEQ ID NO: 3310); and GGGGSGGGGSGGGGGS(SEQ ID NO: 3643). In some embodiments, the peptide linker is a A(EAAAK)nA (SEQ ID NO: 3437) family of linkers (e.g., as described in Protein Eng. (2001) 14 (8): 529-532). These are stiff helical linkers with n ranging from 2-5. In some embodiments, the peptide linker is selected from AEAAAKEAAAKAAA (SEQ ID NO: 3314); AEAAAKEAAAKEAAAKAAA (SEQ ID NO: 3315); AEAAAKEAAAKEAAAKEAAAKAAA (SEQ ID NO: 3316); and AEAAAKEAAAKEAAAKEAAAKEAAAKAAA(SEQ ID NO: 3317).

### CARs and Cells Comprising the CAR

Disclosed herein, in some embodiments, is a recombinant T cell receptor or a CAR that may comprise an extracellular domain (e.g., an antigen binding domain) that binds TCRαV, a transmembrane domain, and an intracellular signaling domain, wherein the intracellular signaling domain may comprise a costimulatory signaling region.

### The Extracellular Domain

The extracellular domain that binds TCRαV may be any anti-TCRαV antibodies or any fragments thereof disclosed herein. In certain embodiments, the extracellular domain/antigen binding domain may comprise a heavy chain variable region that may comprise three heavy chain complementarity determining regions (HCDRs), and a light chain variable region that may comprise three light chain complementarity determining regions (LCDRs). In some embodiment, the extracellular domain that binds TCRαV may be a Fab or a scFv.

In some instances, it is beneficial that the antigen binding domain is derived from the same species in which the CAR will ultimately be used. For example, for use in humans, it may be beneficial that the antigen binding domain of the CAR may comprise a human antigen receptor that binds a human antigen or a fragment thereof. In one exemplary embodiment, the CAR may bind a TCRαV in a mammal (e.g., a human).

Transmembrane Domain and/or Hinge Domain

With respect to the transmembrane domain, in various embodiments, the CAR may comprise a transmembrane domain that is fused to the extracellular domain of the CAR. In one embodiment, the CAR may comprise a transmembrane domain that naturally is associated with one of the domains in the CAR. In some embodiments, the transmembrane domain is selected or modified by amino acid substitution to avoid binding to the transmembrane domains of the same or different surface membrane proteins in order to minimize interactions with other members of the receptor complex.

The transmembrane domain may be derived either from a natural or from a synthetic source. When the source is natural, the domain may be derived from any membrane-bound or transmembrane protein. In one embodiment, the transmembrane domain may be synthetic, in which case it may comprise predominantly hydrophobic residues such as leucine and valine. In one aspect, a triplet of phenylalanine, tryptophan and valine may be found at each end of a synthetic transmembrane domain. Optionally, a short oligo- or poly peptide linker, between 2 and 10 amino acids in length may form the linkage between the transmembrane domain and the cytoplasmic signaling domain of the CAR. A glycine-serine (GS) double provides a particularly suitable linker.

In some embodiments, a variety of human hinges can be employed as well, including, but not limited to, the human Ig (immunoglobulin) hinge domain and the CD8 alpha hinge domain. Examples of the hinge and/or transmembrane domain include, but are not limited to, a hinge and/or transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD 134, CD137, CD154, KIR, 0X40, CD2, CD27, LFA-1 (CD 11 a, CD18), ICOS (CD278), 4-1BB (CD 137), GITR, CD40, BAFFR, HVEM (LIGHTR), SLAMF7, NKp80 (KLRF1), CD160, CD 19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGA1, CD11a, LFA-1, IT GAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD 160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C.

### Intracellular Signaling Domain

The CAR of the present disclosure may comprise an intracellular signaling domain, wherein the intracellular signaling domain may comprise a costimulatory signaling region. The intracellular signaling domain of the CAR is responsible for activation of at least one of the effector functions of the cell in which the CAR is expressed. The intracellular domain transduces the effector function signal and directs the cell to perform its specialized function. Examples of an intracellular signaling domain include, but are not limited to, the cytoplasmic portion of a surface receptor, a co-stimulatory molecule, and any molecule that acts in concert to initiate signal transduction in the T cell, as well as any derivative or variant of these elements and any synthetic sequence that has the same functional capability.

JAs used herein, a “costimulatory molecule,” refers to a molecule on an antigen presenting cell (e.g., an APC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. Exemplary costimulatory molecules including but are not limited to CD27, CD28, 4-1BB (CD137), 0X40, CD30, CD40, PD-1, ICOS, lymphocyte function-associated antigen-1 (LFA-1), CD2, CD7, LIGHT, NKG2C, B7-H3, or a ligand that specifically binds with CD83.

Examples of the intracellular signaling domain include, without limitation, the (chain of the T cell receptor complex or any of its homologs, e.g., η chain, FcsRFγ and β chains, MB 1 (Iga) chain, B29 (Ig) chain, etc., human CD3 zeta chain, CD3 polypeptides (Δ, δ and ε), syk family tyrosine kinases (Syk, ZAP 70, etc.), src family tyrosine kinases (Lck, Fyn, Lyn, etc.), and other molecules involved in T cell transduction, such as CD2, CD5 and CD28. In one embodiment, the intracellular signaling domain may be human CD3 zeta chain, FcγRIII, FcsRI, cytoplasmic tails of Fc receptors, an immunoreceptor tyrosine-based activation motif (ITAM) bearing cytoplasmic receptors, and combinations thereof.

In certain embodiments, the intracellular signaling domain of the CAR includes any portion of one or more co-stimulatory molecules, such as at least one signaling domain from CD2, CD3, CD8, CD27, CD28, ICOS (CD278), 4-1BB, PD-1, any derivative or variant thereof, any synthetic sequence thereof that has the same functional capability, and any combination thereof. In certain embodiments, the intracellular domain may comprise a costimulatory domain of a protein selected from the group consisting of proteins in the TNFR superfamily, CD28, 4-IBB (CD 137), 0X40 (CD 134), PD-1, CD7, LIGHT, CD83L, DAPIO, DAP 12, CD27, CD2, CD5, ICAM-1, LFA-1, Lck, TNFR-I, TNFR-II, Fas, CD30, CD40, ICOS, NKG2C, and B7-H3 (CD276), or a variant thereof, or an intracellular domain derived from a killer immunoglobulin-like receptor (KIR).

In one embodiment, the CAR of the disclosure may comprise a CD137 (4-1BB) signaling domain. For example, inclusion of the CD137 (4-1BB) signaling domain significantly increased CAR mediated activity and in vivo persistence of CAR T cells compared to an otherwise identical CAR T cell not engineered to express CD137 (4-1BB). However, the disclosure is not limited to a specific CAR. Rather, any CAR that targets TCRαV, can be used in the present disclosure. Compositions and methods of making and using CARs have been described in PCT/US11/64191, which is incorporated by reference in its entirety herein. In certain embodiments, the intracellular signaling domain may comprise CD3zeta. In certain embodiments, the intracellular signaling domain may comprise CD28 and CD3zeta. In certain embodiments, the intracellular signaling domain may comprise 4-1BB and CD3zeta.

In certain embodiments, the intracellular signaling domain may comprise CD3zeta. In certain embodiments, the intracellular signaling domain may comprise CD28 and CD3zeta. In certain embodiments, the intracellular signaling domain may comprise 4-1BB and CD3zeta.

In another aspect, provided herein is a T cell genetically modified to express a recombinant T cell receptor, wherein the recombinant T cell receptor comprises a domain that binds a TCRαV. In some embodiments, the domain that binds a Vα region of a T cell receptor may be an α/β heterodimer of the recombinant T cell receptor.

In some embodiments, provide herein are T cells genetically modified to express any of the TCRαV-specific CAR disclosed herein. In some embodiments, the cell may have high affinity for cells expressing TCRαV.

In some embodiments, the genetically modified cell may be a T cell, such as a helper T cell, a cytotoxic T cell, a memory T ceil, regulatory T cell, gamma delta T cell, a natural killer cell, cytokine induced killer cell, a cell line thereof, a T memory stem cell, or other T effector cell. It may be also useful for the T cell to have limited toxicity toward healthy cells and to have specificity to cells expressing the TCRαV. In some embodiments, the genetically modified T cell may be specific for the TCRαV from a specific T cell clone. Such specificity may prevent or reduce off-target toxicity that is prevalent in current therapies that are not specific. In one embodiment, the T cell may have limited toxicity toward healthy cells. In one embodiment the T cell may be an autologous cell. In another embodiment, the T cell may be an allogeneic cell.

In some embodiments, the disclosure includes genetically modified immune cells derived from pluripotent stem cells that were differentiated in vitro. In other embodiments, the disclosure includes T cells, such as primary cells, expanded T cells derived from primary T cells, T cells derived from stem cells differentiated in vitro, T cell lines such as Jurkat cells, other sources of T cells, combinations thereof, and other effector cells.

### Nucleic Acids

Described herein, in certain embodiments, is an isolated nucleic acid molecule comprising a nucleotide sequence having at least 75%, 80%, 85%, 90%, 95%, 99%, 99.5%, 99.9%, or 100% sequence identity to the nucleotide sequence encoding the multifunctional polypeptide molecule as described herein.

Nucleic acids encoding the aforementioned antibody molecules, e.g., anti-TCRαV antibody molecules, multispecific or multifunctional molecules are also disclosed.

Nucleic acids encoding the aforementioned recombinant T cell receptor or CAR are also disclosed.

In certain embodiments, the disclosure features nucleic acids comprising nucleotide sequences that encode heavy and light chain variable regions and CDRs or hypervariable loops of the antibody molecules, as described herein. For example, the disclosure features a first and second nucleic acid encoding heavy and light chain variable regions, respectively, of an antibody molecule chosen from one or more of the antibody molecules as described herein. The nucleic acid can comprise a nucleotide sequence as set forth in the tables herein, or a sequence substantially identical thereto (e.g., a sequence at least about 85%, 90%, 95%, 99% or more identical thereto, or which differs by no more than 3, 6, 15, 30, or 45 nucleotides from the sequences shown in the tables herein.

In certain embodiments, the nucleic acid can comprise a nucleotide sequence encoding at least one, two, or three CDRs or hypervariable loops from a heavy chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In other embodiments, the nucleic acid can comprise a nucleotide sequence encoding at least one, two, or three CDRs or hypervariable loops from a light chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In yet another embodiment, the nucleic acid can comprise a nucleotide sequence encoding at least one, two, three, four, five, or six CDRs or hypervariable loops from heavy and light chain variable regions having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions).

In certain embodiments, the nucleic acid can comprise a nucleotide sequence encoding at least one, two, or three CDRs or hypervariable loops from a heavy chain variable region having the nucleotide sequence as set forth in the tables herein, a sequence substantially homologous thereto (e.g., a sequence at least about 85%, 90%, 95%, 99% or more identical thereto, and/or capable of hybridizing under the stringency conditions described herein). In another embodiment, the nucleic acid can comprise a nucleotide sequence encoding at least one, two, or three CDRs or hypervariable loops from a light chain variable region having the nucleotide sequence as set forth in the tables herein, or a sequence substantially homologous thereto (e.g., a sequence at least about 85%, 90%, 95%, 99% or more identical thereto, and/or capable of hybridizing under the stringency conditions described herein). In yet another embodiment, the nucleic acid can comprise a nucleotide sequence encoding at least one, two, three, four, five, or six CDRs or hypervariable loops from heavy and light chain variable regions having the nucleotide sequence as set forth in the tables herein, or a sequence substantially homologous thereto (e.g., a sequence at least about 85%, 90%, 95%, 99% or more identical thereto, and/or capable of hybridizing under the stringency conditions described herein).

In certain embodiments, the nucleic acid can comprise a nucleotide sequence encoding a cytokine molecule, an immune cell engager, or a stromal modifying moiety as described herein.

In another aspect, the application features host cells and vectors containing the nucleic acids described herein. The nucleic acids may be present in a single vector or separate vectors present in the same host cell or separate host cell, as described in more detail hereinbelow.

### Vectors

Described herein, in certain embodiments, is a vector comprising one or more of the nucleic acid molecules as described herein.

Further provided herein are vectors comprising the nucleotide sequences encoding antibody molecules, e.g., anti-TCRαV antibody molecules, a multispecific or multifunctional molecule, or a CAR described herein. In some embodiments, the vectors comprise nucleic acid sequences encoding antibody molecules, e.g., anti-TCRαV antibody molecules, multispecific or multifunctional molecule, or a CAR described herein. In some embodiments, the vectors comprise the nucleotide sequences described herein. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes may be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors may be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. In the case of protoplast fusion, the cells are grown in media and screened for the appropriate activity.

Methods and conditions for culturing the resulting transfected cells and for recovering the antibody molecule produced are known to those skilled in the art, and may be varied or optimized depending upon the specific expression vector and mammalian host cell employed, based upon the present description.

### Cells

Described herein, in certain embodiments, is a cell comprising the nucleic acid as described herein or the vector as described herein.

In another aspect, described herein are host cells and vectors containing the nucleic acids. The nucleic acids may be present in a single vector or separate vectors present in the same host cell or separate host cell. The host cell can be a eukaryotic cell, e.g., a mammalian cell, an insect cell, a yeast cell, or a prokaryotic cell, e.g., E. coli. For example, the mammalian cell can be a cultured cell or a cell line. Exemplary mammalian cells include lymphocytic cell lines (e.g., NSO), Chinese hamster ovary cells (CHO), COS cells, oocyte cells, and cells from a transgenic animal, e.g., mammary epithelial cell.

In some embodiments, described herein are host cells comprising a nucleic acid encoding an antibody molecule as described herein.

In some embodiments, described herein are the host cells genetically engineered to comprise nucleic acids encoding the antibody molecule.

In some embodiments, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

In some embodiments, described herein are host cells comprising the vectors described herein. The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

Method of Expanding Cells with Anti-TCRVA Antibodies

Any of the compositions and methods described herein can be used to expand an immune cell population. An immune cell provided herein includes an immune cell derived from a hematopoietic stem cell or an immune cell derived from a non-hematopoietic stem cell, e.g., by differentiation or de-differentiation.

An immune cell includes a hematopoietic stem cell, progeny thereof and/or cells that have differentiated from said HSC, e.g., lymphoid cells or myeloid cells. An immune cell can be an adaptive immune cell or an innate immune cell. Examples of immune cells include T cells, B cells, Natural Killer cells, Natural Killer T cells, neutrophils, dendritic cells, monocytes, macrophages, and granulocytes.

In some embodiments, an immune cell is a T cell. In some embodiments, a T cell includes a CD4+ T cell, a CD8+ T cell, a TCR alpha-beta T cell, a TCR gamma-delta T cell. In some embodiments, a T cell comprises a memory T cell (e.g., a central memory T cell, or an effector memory T cell (e.g., a TEMRA) or an effector T cell. In some embodiments, a T cell comprises a tumor infiltrating lymphocyte (TIL).

In some embodiments, an immune cell is an NK cell.

In some embodiments, an immune cell is a TIL. TILs are immune cells (e.g., T cells, B cells or NK cells) that can be found in a tumor or around a tumor (e.g., in the stroma or tumor microenvironment of a tumor), e.g., a solid tumor, e.g., as described herein. TILs can be obtained from a sample from a subject having cancer, e.g., a biopsy or a surgical sample. In some embodiments, TILs can be expanded using a method as described herein. In some embodiments, a population of expanded TILs, e.g., expanded using a method as described herein, can be administered to a subject to treat a disease, e.g., a cancer.

In some embodiments, immune cells, e.g., T cells (e.g., TILs), can be obtained from a unit of blood collected from a subject using any number of techniques known to the skilled artisan, such as Ficoll™ separation. In one aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and, optionally, to place the cells in an appropriate buffer or media for subsequent processing steps. In some embodiments, the cells are washed with phosphate buffered saline (PBS). In an alternative embodiment, the wash solution lacks calcium and may lack magnesium or may lack many if not all divalent cations. The methods described herein can include more than one selection step, e.g., more than one depletion step.

In some embodiments, the methods of the application can utilize culture media conditions comprising DMEM. DMEM F12, RPMI 1640, and/or AIM V media. The media can be supplemented with glutamine, HEPES buffer (e.g., 10 mM), serum (e.g., heat-inactivated serum, e.g., 10%), and/or beta mercaptoethanol (e.g., 55 uM). IN some embodiments, the culture conditions as described herein comprise one or more supplements, cytokines, growth factors, or hormones. In some embodiments, the culture condition comprises one or more of IL-2, IL-15, or IL-7, or a combination thereof.

Immune effector cells such as T cells may be activated and expanded generally using methods as described, for example, in U.S. Pat. No. 6,352,694; 6,534,055; or 6,905,680. Generally, a population of immune cells, may be expanded by contact with an agent that stimulates a CD3/TCR complex associated signal and a ligand that stimulates a costimulatory molecule on the surface of the T cells; and/or by contact with a cytokine, e.g., IL-2, IL-15 or IL-7. T cell expansion protocols can also include stimulation, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody can be used. Examples of an anti-CD28 antibody include 9.3, B-T3, XR-CD28 (Diaclone, Besançon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

A TIL population can also be expanded by methods known in the art. For example, a population of TILs can be expanded as described in Hall et al., Journal for ImmunoTherapy of Cancer (2016) 4:61, the entire contents of which are hereby incorporated by reference. Briefly, TILs can be isolated from a sample by mechanical and/or physical digestion. The resultant TIL population can be stimulated with an anti-CD3 antibody in the presence of non-dividing feeder cells. In some embodiments, the TIL population can be cultured, e.g., expanded, in the presence of IL-2, e.g., human IL-2. In some embodiments, the TIL cells can be cultured, e.g., expanded for a period of at least 1-21 day s, e.g., at least 1, 2, 3, 4, 5, 6, 7, 8, 9, 10, 11, 12, 13, 14, 15, 16, 17, 18, 19, 20 or 21 days.

As described herein, in some embodiments, an immune cell population (e.g., a T cell (e.g., a T_{EMRA} cell or a TIL population) can be expanded by contacting the immune cell population with an anti-TCRVA antibody, e.g., as described herein.

In some embodiments, the expansion occurs in vivo, e.g., in a subject. In some embodiments, a subject is administered the multispecific or multifunctional molecules comprising TCRαV-binding moieties as described herein resulting in expansion of immune cells in vivo.

In some embodiments, the expansion occurs ex vivo, e.g., in vitro. In some embodiments, cells from a subject, e.g., T cells, e.g., TIL cells, are expanded in vitro with the multispecific or multifunctional molecules as described herein. In some embodiments, the expanded TILs are administered to the subject to treat a disease or a symptom of a disease.

In some embodiments, a method of expansion as described herein results in an expansion of at least 1.1-10 fold, 10-20 fold, or 20-50 fold expansion. In some embodiments, the expansion is at least 1.1, 1.2, 1.3, 1.4, 1.5, 2, 3, 4, 5, 6, 7, 8, 9, 10, 15, 20, 25, 30, 35, 40, 45 or 50 fold expansion.

In some embodiments, a method of expansion as described herein comprises culturing, e.g., expanding, the cells for at least about 4 hours, 6 hours, 10 hours, 12 hours, 15 hours, 18 hours, 20 hours, or 22 hours. In some embodiments, a method of expansion as described herein comprises culturing, e.g., expanding, the cells for at least 1, 2, 3, 4, 5, 6, 7, 8, 9, 10, 11, 12, 13, 14, 15, 16, 17, 18, 19, 20 or 21 days. In some embodiments, a method of expansion as described herein comprises culturing, e.g., expanding, the cells for at least about 1 week, 2 weeks, 3 weeks, 4 weeks, 5 weeks, 6 weeks, 7 weeks or 8 weeks.

In some embodiments, a method of expansion as described herein is performed on immune cells obtained from a healthy subject.

In some embodiments, a method of expansion as described herein is performed on immune cells (e.g., TILs) obtained from a subject having a disease, e.g., a cancer, e.g., a solid tumor as described herein.

In some embodiments, a method of expansion as described herein further comprises contacting the population of cells with an agent, that promotes, e.g., increases, immune cell expansion. In some embodiments, the agent comprises an immune checkpoint inhibitor, e.g., a PD-1 inhibitor, a LAG-3 inhibitor, a CTLA4 inhibitor, or a TIM-3 inhibitor. In some embodiments, the agent comprises a 4-1BB agonist, e.g., an anti-4-1BB antibody.

Without wishing to be bound by theory, in some embodiments, the multispecific or multifunctional molecules as described herein can expand, e.g., selectively or preferentially expand. T cells expressing a T cell receptor (TCR) comprising a TCR alpha and/or TCR beta molecule, e.g., TCR alpha-beta T cells (αβ T cells). In some embodiments, the multispecific or multifunctional molecules as described herein do not expand, or induce proliferation of T cells expressing a TCR comprising a TCR gamma and/or TCR delta molecule, e.g., TCR gamma-delta T cells (γδ T cells). In some embodiments, the multispecific or multifunctional molecules as described herein selectively or preferentially expand αβ T cells over γδ T cells.

Without wishing to be bound by theory, it is believed that, in some embodiments, γβ T cells are associated with cytokine release syndrome (CRS) and/or neurotoxicity (NT). In some embodiments, the multispecific or multifunctional molecules as described herein result in selective expansion of non-γδ T cells, e.g., expansion of αβ T cells, thus reducing CRS and/or NT.

In some embodiments, any of the compositions or methods as described herein result in an immune cell population having a reduction of, e.g., depletion of, γδ T cells. In some embodiments, the immune cell population is contacted with an agent that reduces, e.g., inhibits or depletes, γδ T cells, e.g., an anti-IL-17 antibody or an agent that binds to a TCR gamma and/or TCR delta molecule.

### CRS Grading

In some embodiments, CRS (Cytokine Release Syndrome) can be graded in severity from 1-5 as follows. Grades 1-3 are less than severe CRS. Grades 4-5 are severe CRS. For Grade 1 CRS, only symptomatic treatment is needed (e.g., nausea, fever, fatigue, myalgias, malaise, headache) and symptoms are not life threatening. For Grade 2 CRS, the symptoms require moderate intervention and generally respond to moderate intervention. Subjects having Grade 2 CRS develop hypotension that is responsive to either fluids or one low-dose vasopressor; or they develop grade 2 organ toxicity or mild respiratory symptoms that are responsive to low flow oxygen (<40% oxygen). In Grade 3 CRS subjects, hypotension generally cannot be reversed by fluid therapy or one low-dose vasopressor. These subjects generally require more than low flow oxygen and have grade 3 organ toxicity (e.g., renal or cardiac dysfunction or coagulopathy) and/or grade 4 transaminitis. Grade 3 CRS subjects require more aggressive intervention, e.g., oxygen of 40% or higher, high dose vasopressor(s), and/or multiple vasopressors. Grade 4 CRS subjects suffer from immediately life-threatening symptoms, including grade 4 organ toxicity or a need for mechanical ventilation. Grade 4 CRS subjects generally do not have transaminitis. In Grade 5 CRS subjects, the toxicity causes death. Sets of criteria for grading CRS are provided herein as Table 3, Table 4, and Table 5. Unless otherwise specified, CRS as used herein refers to CRS according to the criteria of Table 4.

In some embodiments, CRS is graded according to Table 3.

The term “cytokine profile” as used herein, refers to the level and/or activity of on one or more cytokines or chemokines, e.g., as described herein. In some embodiments, a cytokine profile comprises the level and/or activity of a naturally occurring cytokine, a fragment or a variant thereof. In some embodiments, a cytokine profile comprises the level and/or activity of one or more cytokines and/or one or more chemokines (e.g., as described herein). In some embodiments, a cytokine profile comprises the level and/or activity of a naturally occurring cytokine, a fragment or a variant thereof. In some embodiments, a cytokine profile comprises the level and/or activity of a naturally occurring chemokine, a fragment or a variant thereof. In some embodiments, a cytokine profile comprises the level and/or activity of one or more of: IL-2 (e.g., full length, a variant, or a fragment thereof); IL-1 beta (e.g., full length, a variant, or a fragment thereof); IL-6 (e.g., full length, a variant, or a fragment thereof); TNFα (e.g., full length, a variant, or a fragment thereof); IFNgamma (e.g., full length, a variant, or a fragment thereof) IL-10 (e.g., full length, a variant, or a fragment thereof); IL-4 (e.g., full length, a variant, or a fragment thereof); TNF alpha (e.g., full length, a variant, or a fragment thereof); IL-12p70 (e.g., full length, a variant, or a fragment thereof); IL-13 (e.g., full length, a variant, or a fragment thereof); IL-8 (e.g., full length, a variant, or a fragment thereof); Eotaxin (e.g., full length, a variant, or a fragment thereof); Eotaxin-3 (e.g., full length, a variant, or a fragment thereof); IL-8 (HA) (e.g., full length, a variant, or a fragment thereof); IP-10 (e.g., full length, a variant, or a fragment thereof); MCP-1 (e.g., full length, a variant, or a fragment thereof); MCP-4 (e.g., full length, a variant, or a fragment thereof); MDC (e.g., full length, a variant, or a fragment thereof); MIP-1a (e.g., full length, a variant, or a fragment thereof); MIP-1b (e.g., full length, a variant, or a fragment thereof); TARC (e.g., full length, a variant, or a fragment thereof); GM-CSF (e.g., full length, a variant, or a fragment thereof); IL-12 23p40 (e.g., full length, a variant, or a fragment thereof); IL-15 (e.g., full length, a variant, or a fragment thereof); IL-16 (e.g., full length, a variant, or a fragment thereof); IL-17a (e.g., full length, a variant, or a fragment thereof); IL-1a (e.g., full length, a variant, or a fragment thereof); IL-5 (e.g., full length, a variant, or a fragment thereof); IL-7 (e.g., full length, a variant, or a fragment thereof); TNF-beta (e.g., full length, a variant, or a fragment thereof); or VEGF (e.g., full length, a variant, or a fragment thereof). In some embodiments, a cytokine profile includes secretion of one or more cytokines or chemokines. In some embodiments, a cytokine in a cytokine profile can be modulated, e.g., increased or decreased, by an anti-TCRAV antibody molecule described herein. In some embodiments, the cytokine profile includes cytokines associated with a cytokine storm or cytokine release syndrome (CRS), e.g., IL-6, IL-1beta, TNFalpha and IL-10.

### Pharmaceutical Compositions

Described herein, in certain embodiments, is a pharmaceutical composition comprising the multifunctional polypeptide molecule as described herein, the nucleic acid molecules as described herein, the vector as described herein, or the cell as described herein, and a pharmaceutically acceptable carrier, excipient, or diluent.

Pharmaceutical compositions or formulations comprising the agent, e.g., the multifunctional or multispecific molecules, of the described compositions and for use in any of the described methods can be prepared according to conventional techniques well known in the pharmaceutical industry and described in the published literature. In some embodiments, a pharmaceutical composition or formulation for treating a subject comprises an effective amount of any the multifunctional or multispecific molecules or the compositions as described herein, or a pharmaceutically acceptable salt, solvate, hydrate or ester thereof. The pharmaceutical formulation comprising the multifunctional or multispecific molecules as described herein may further comprise a pharmaceutically acceptable excipient, diluent or carrier.

Pharmaceutically acceptable salts are suitable for use in contact with the tissues of humans and lower animals without undue toxicity, irritation, allergic response, etc., and are commensurate with a reasonable benefit/risk ratio. (See, e.g., S. M. Berge, et al., J. Pharmaceutical Sciences, 66: 1-19 (1977), incorporated herein by reference for this purpose. The salts can be prepared in situ during the final isolation and purification of the compounds, or separately by reacting the free base form with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other documented methodologies such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

In some embodiments, the compositions are formulated into any of many possible dosage forms such as, but not limited to, tablets, capsules, gel capsules, liquid syrups, soft gels, suppositories, and enemas. In some embodiments, the compositions are formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers. In some embodiments, a pharmaceutical formulation or composition as described herein includes, but is not limited to, a solution, emulsion, microemulsion, foam or liposome-containing formulation (e.g., cationic or noncationic liposomes).

The pharmaceutical composition or formulation described herein may comprise one or more penetration enhancers, carriers, excipients or other active or inactive ingredients as appropriate and well known to those of skill in the art or described in the published literature. In some embodiments, liposomes also include sterically stabilized liposomes. e.g., liposomes comprising one or more specialized lipids. These specialized lipids result in liposomes with enhanced circulation lifetimes. In some embodiments, a sterically stabilized liposome comprises one or more glycolipids or is derivatized with one or more hydrophilic polymers, such as a polyethylene glycol (PEG) moiety. In some embodiments, a surfactant is included in the pharmaceutical formulation or compositions. The use of surfactants in drug products, formulations and emulsions is well known in the art. In some embodiments, the present disclosure employs a penetration enhancer to effect the efficient delivery of the multifunctional or multispecific molecules or the compositions as described herein, e.g., to aid diffusion across cell membranes and/or enhance the permeability of a lipophilic drug. In some embodiments, the penetration enhancers are a surfactant, fatty acid, bile salt, chelating agent, or non-chelating nonsurfactant.

In some embodiments, the pharmaceutical formulation comprises multiple multifunctional or multispecific molecules as described herein. In some embodiments, the multifunctional or multispecific molecules or the compositions as described herein is administered in combination with another drug or therapeutic agent.

### Treatment of Subjects

Any of the compositions provided herein may be administered to an individual. “Individual” may be used interchangeably with “subject” or “patient.” An individual may be a mammal, for example a human or animal such as a non-human primate, a rodent, a rabbit, a rat, a mouse, a horse, a donkey, a goat, a cat, a dog, a cow, a pig, or a sheep. In some embodiments, the individual is a human. In some embodiments, the individual is a fetus, an embryo, or a child. In other embodiments, the individual may be another eukaryotic organism, such as a plant. In some embodiments, the compositions provided herein are administered to a cell ex vivo.

In some embodiments, the compositions provided herein are administered to an individual as a method of treating a disease or disorder. In some embodiments, the individual has a genetic disease, such as any of the diseases described herein. In some embodiments, the individual is at risk of having a disease, such as any of the diseases described herein. In some embodiments, the individual is at increased risk of having a disease or disorder caused by insufficient amount of a protein or insufficient activity of a protein. If an individual is “at an increased risk” of having a disease or disorder caused insufficient amount of a protein or insufficient activity of a protein, the method involves preventative or prophylactic treatment. For example, an individual may be at an increased risk of having such a disease or disorder because of family history of the disease. Typically, individuals at an increased risk of having such a disease or disorder benefit from prophylactic treatment (e.g., by preventing or delaying the onset or progression of the disease or disorder). In some embodiments, a fetus is treated in utero, e.g., by administering the multifunctional or multispecific molecules or the compositions as described herein to the fetus directly or indirectly (e.g., via the mother).

Suitable routes for administration of the multifunctional or multispecific molecules or the compositions as described herein may vary depending on cell type to which delivery of the multifunctional or multispecific molecules or the compositions is desired. The multifunctional or multispecific molecules or the compositions as described herein may be administered to patients parenterally, for example, by intrathecal injection, intracerebroventricular injection, intraperitoneal injection, intramuscular injection, subcutaneous injection, or intravenous injection.

In some embodiments, the multifunctional or multispecific molecules or the compositions as described herein are administered with one or more agents capable of promoting penetration of the subject the multifunctional or multispecific molecules or the compositions as described herein across the blood-brain barrier by any method known in the art. For example, delivery of agents by administration of an adenovirus vector to motor neurons in muscle tissue is described in U.S. Pat. No. 6,632,427, “Adenoviral-vector-mediated gene transfer into medullary motor neurons,” incorporated herein by reference. Delivery of vectors directly to the brain, e.g., the striatum, the thalamus, the hippocampus, or the substantia nigra, is described, e.g., in U.S. Pat. No. 6,756,523, “Adenovirus vectors for the transfer of foreign genes into cells of the central nervous system particularly in brain,” incorporated herein by reference.

In some embodiments, the multifunctional or multispecific molecules or the compositions as described herein are linked or conjugated with agents that provide desirable pharmaceutical or pharmacodynamic properties. In some embodiments, the multifunctional or multispecific molecules or the compositions as described herein are coupled to a substance, known in the art to promote penetration or transport across the blood-brain barrier, e.g., an antibody to the transferrin receptor. In some embodiments, the multifunctional or multispecific molecules or the compositions as described herein are linked with a viral vector.

In some embodiments, subjects treated using the methods and compositions are evaluated for improvement in condition using any methods known and described in the art.

The terms “treat,” “treating”, and “treatment,” and the like are used herein to generally mean obtaining a desired pharmacological and/or physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly, a human, and includes: (a) preventing the disease from occurring in a subject which may be predisposed to the disease but has not yet been diagnosed as having it; (b) inhibiting the disease, i.e., arresting its development; or (c) relieving the disease, i.e., mitigating or ameliorating the disease and/or its symptoms or conditions. The term “prophylaxis” is used herein to refer to a measure or measures taken for the prevention or partial prevention of a disease or condition. In some embodiments, the terms “condition,” “disease,” or “disorder,” as used herein, are interchangeable.

By “treating or preventing a disease or a disorder” is meant ameliorating any of the conditions or signs or symptoms associated with the disorder before or after it has occurred. As compared with an equivalent untreated control, such reduction or degree of prevention is at least 3%, 5%, 10%, 20%, 40%, 50%, 60%, 80%, 90%, 95%, or 100% as measured by any standard technique. A patient who is being treated for a disease or a disorder, is one who a medical practitioner has diagnosed as having such a condition. Diagnosis may be by any suitable means. Diagnosis and monitoring may involve, for example, detecting the presence of pathological cells in a biological sample (e.g., tissue biopsy, blood test, or urine test), detecting the level of a surrogate marker of the disorder in a biological sample, or detecting symptoms associated with the disorder. A patient in whom the development of a disorder is being prevented may or may not have received such a diagnosis. One in the art will understand that these patients may have been subjected to the same standard tests as described above or may have been identified, without examination, as one at high risk due to the presence of one or more risk factors (e.g., family history or genetic predisposition).

### Methods of Treatment

Described herein, in certain embodiments, is a method of treating a condition or disease in a subject in need therefor comprising administering to the subject a therapeutically effective amount of the multifunctional polypeptide molecule as described herein, the nucleic acid molecules as described herein, the vector as described herein, the cell as described herein, the pharmaceutical composition as described herein, or a combination thereof, wherein the administering is effective to treat the condition or disease in the subject. Any condition or disease that is related to TCRαV may be subject of the methods of treatment disclosed herein. For example, a condition or disease that expresses or overexpresses a TCRαV can be treated with a CAR-T cell containing a CAR with an anti-TCRαV binding domain that binds to the TCRαV expressed or overexpressed by diseased cells. Examples of TCRαV-related diseases include, but are not limited to, those listed in Table 8.

TABLE 8
TCRαV-related diseases or conditions

| TCRαV Subfamily | Related conditions or diseases |
|---|---|
| TCRαV1 (including | S. paratyphi infection; Bacteroidetes |
| TCRαV1-1 and | infection; Proteobacteria infection; |
| TCRα1-2) | multiple sclerosis; M. tuberculosis |
| | infection; breast cancer; ER+ |
| | breast cancer |
| TCRαV2 | Crohn's disease |
| TCRαV3 | ER+ Breast cancer |
| TCRαV4 | |
| TCRαV5 | |
| TCRαV6 | Diffuse large B-cell lymphoma; |
| | respiratory virus infection |
| TCRαV7 | SARS-CoV-2 infection |
| TCRαV8 (including | Diffuse large B-cell lymphoma |
| TCRαV8-1, TCRαV8-2, | |
| TCRαV8-3, TCRαV8-4, | |
| and TCRαV8-6) | |
| TCRαV9 (including | Breast cancer; SARS-CoV-2 |
| TCRαV9-1 and | infection |
| TCRαV9-2) | |
| TCRαV10 | |
| TCRαV11 | SARS-CoV-2 infection |
| TCRαV12 (including | Diffuse large B-cell |
| TCRαV12-1, | lymphoma; S. pyogenes |
| TCRαV12-2, and | infection; SARS-CoV-2 |
| TCRαV12-3) | infection; melanoma; |
| | yellow fever |
| TCRαV13 (including | Sjogren's syndrome; influenza |
| TCRαV13-1, | |
| TCRαV13-2) | |
| TCRαV14 (including | |
| TCRαV14/DV4) | |
| TCRαV15 | |
| TCRαV16 | Respiratory virus |
| TCRαV17 | HIV infection; |
| | Cytomegalovirus infection |
| TCRαV18 | SARS-CoV-2 infection |
| TCRαV19 | Multiple myeloma |
| TCRαV20 | Celiac disease |
| TCRαV21 | Diffuse large B-cell lymphoma |
| TCRαV22 | Diffuse large B-cell lymphoma; |
| | Crohn's disease |
| TCRαV23 (including | |
| TCRαV23/DV6) | |
| TCRαV24 | HIV infection |
| TCRαV25 | Diffuse large B-cell lymphoma |
| TCRαV26 (including | Celiac disease |
| TCRαV26-1, and | |
| TCRαV26-2) | |
| TCRαV27 | Influenza |
| TCRαV29 (including | Breast cancer; leukemia; melanoma |
| TCRαV29/DV5) | |
| TCRαV30 | Endometrial cancer |
| TCRαV34 | |
| TCRαV35 | |
| TCRαV36 | |
| TCRαV38 (including | Leukemia; Epstein-Barr |
| TCRαV38-1, and | Virus infection |
| TCRαV38-2/DV8) | |
| TCRαV39 | Esophageal squamous cell carcinoma |
| TCRαV40 | Crohn's disease |
| TCRαV41 | Joint implant failure |
| TCRαV236/DV7 | |

### REFERENCES USED IN TABLE 8

1. He et al., (2012) Neoplasma. 59(6):693-9
2. Howson et al. (2018) Nat. Commun. 9, 253
3. Tastan et al. (2018) Mucosal Immunol. 11, 1591-1605
4. Camero et al., (2019) Front. Immunol. 10, 2690
5. Huang et al. (2019) Proc. Natl. Acad. Sci. U.S.A. 116, 8995-9001
6. Hong et al., (2022) Front Cell Infect Microbiol. 10; 12:932373
7. Tan et al., (2010) Hematology, 15:2, 81-87
8. Gedda et al., (2022) J Transl Med. 20(1):587
9. Meermeier et al. (2016) Nat. Commun. 7, 12506
10. Yang et al., J Biol Chem. 2017 Nov. 10; 292(45):18618-18627.
11. Surman et al., J Immunol. 2011 Jul. 15:187(2):835-41.
12. Motozono et al., 2014 Apr. 1; 192(7):3428-34.
13. Brennan et al., (2007) J Virol. (13):7269-73.
14. Huda et al., (2021) Leuk Lymphoma. 62(7):1711-1720.
15. Petersen et al., 2016 Oct. 4:24(10):1643-1657.
16. Benati et al., J Clin Invest. 2016 Jun. 1; 126(6):2093-108.
17. Frick et al., (2020) Eur J Immunol. 50(1):142-145.
18. Valkenburg et al., (2016) Proc Natl Acad Sci USA. 113(16):4440-5.
19. Lepore et al., (2017) Elife 6, e24476
20. Zhou et al., (2020) Cancer Cell Int. 20:240.
21. Rowntree et al., (2020) J immunol; 205(6), 1524-1534
22. Crowther et al., (2020) Nat. Immunol. 21, 178-185
23. Xu et al., (2022) J Oncol. 3152114
24. Chen et al., (2021) Int J Mol Sci. 22(5):2428
25. Bovay et al., (2018) Eur J Immunol. 48(2):258-272.
26. Godfrey et al., (2015) Nat Immunol. 16(11):1114-23.
27. Cole et al., (2009) J Biol Chem. 284(40):27281-9.
28. Szeto et al., (2021) Cells. 2021 Oct. 3:10(10):2646.

In some embodiments, the condition or disease is cancer. In some embodiments, the cancer is a solid tumor, a hematological cancer, a metastatic cancer, a soft tissue tumor, or a combination thereof. In some embodiments, the cancer is the solid tumor, and wherein the solid tumor is selected from the group consisting of melanoma, pancreatic cancer, breast cancer, colorectal cancer, lung cancer, skin cancer, ovarian cancer, liver cancer, and a combination thereof. In some embodiments, the cancer is the hematological cancer, and wherein the hematological cancer is selected from the group consisting of Hodgkin's lymphoma, Non-Hodgkin's lymphoma, acute myeloid leukemia (AML), chronic myeloid leukemia, myelodysplastic syndrome, multiple myeloma, T-cell lymphoma, acute lymphocytic leukemia, and a combination thereof. In some embodiments, the Non-Hodgkin's lymphoma is selected from the group consisting of B cell lymphoma, diffuse large B cell lymphoma (DLBCL), follicular lymphoma, chronic lymphocytic leukemia (B-CLL), mantle cell lymphoma, marginal zone B-cell lymphoma, Burkitt lymphoma, lymphoplasmacytic lymphoma, hairy cell leukemia, and a combination thereof. In some embodiments, the T-cell lymphoma is peripheral T-cell lymphoma.

In some embodiments, the cancer is characterized by a cancer antigen present on the cancer. In some embodiments, the cancer antigen is a tumor antigen, a stromal antigen, or a hematological antigen. In some embodiments, the cancer antigen is selected from the group consisting of BCMA, CD19, CD20, CD22, FcRH5, PDL1, CD47, gangloside 2 (GD2), prostate stem cell antigen (PSCA), prostate specific membrane antigen (PMSA), prostate-specific antigen (PSA), carcinoembryonic antigen (CEA), Ron Kinase, c-Met, Immature laminin receptor, TAG-72, BING-4, Calcium-activated chloride channel 2, Cyclin-B1, 9D7, Ep-CAM, EphA3, Her2/neu, Telomerase, SAP-1, Survivin, NY-ESO-1/LAGE-1, PRAME, SSX-2, Melan-A/MART-1, Gp100/pmel17, Tyrosinase, TRP-1/-2, MC1R, β-catenin, BRCA1/2, CDK4, CML66, Fibronectin, p53, Ras. TGF-B receptor, AFP, ETA, MAGE, MUC-1, CA-125, BAGE, GAGE, NY-ESO-1, β-catenin, CDK4, CDC27, α actinin-4, TRP1/gp75, TRP2, gp100, Melan-A/MART1, gangliosides, WT1, EphA3, Epidermal growth factor receptor (EGFR), MART-2, MART-1, MUC1, MUC2, MUM1, MUM2, MUM3, NA88-1, NPM, OA1, OGT, RCC, RUI1, RUI2, SAGE, TRG, TRP1, TSTA, Folate receptor alpha, L1-CAM. CAIX, gpA33, GD3, GM2, VEGFR, Intergrins, carbohydrates, IGF1R, EPHA3, TRAILR1, TRAILR2, RANKL, FAP, TGF-beta, hyaluronic acid, collagen, tenascin C, and tenascin W.

Methods described herein include treating a cancer in a subject by using multispecific or multifunctional molecules as described herein, e.g., using a pharmaceutical composition described herein. Also provided are methods for reducing or ameliorating a symptom of a cancer in a subject, as well as methods for inhibiting the growth of a cancer and/or killing one or more cancer cells. In some embodiments, the methods described herein decrease the size of a tumor and/or decrease the number of cancer cells in a subject administered with a described herein or a pharmaceutical composition described herein.

Described herein are methods of treating a subject having a cancer comprising acquiring a status of one or more TCRAV molecules in a subject. In some embodiments, a higher, e.g., increased, level or activity of one or more TCRαV molecules in a subject, e.g., in a sample from a subject, is indicative of a bias, e.g., a preferential expansion, e.g., clonal expansion, of T cells expressing said one or more TCRαV molecules in the subject.

Without wishing to be bound by theory, it is believed that a biased T cell population, e.g., a T cell population expressing a TCRαV molecule, is antigen-specific for a disease antigen, e.g., a cancer antigen (Wang C Y, et al., Int J Oncol. (2016) 48(6):2247-56). In some embodiments, the cancer antigen comprises a cancer associated antigen or a neoantigen. In some embodiments, a subject having a cancer, e.g., as described herein, has a higher, e.g., increased, level or activity of one or more TCRαV molecules associated with the cancer. In some embodiments, the TCRαV molecule is associated with, e.g., recognizes, a cancer antigen, e.g., a cancer associated antigen or a neoantigen.

Accordingly, as described herein are methods of expanding an immune effector cell population obtained from a subject, comprising acquiring a status of one or more TCRαV molecules in a sample from the subject, comprising contacting said immune effector cell population with an anti-TCRαV antibody molecule as described herein, e.g., an anti-TCRαV antibody molecule that binds to the same TCRαV molecule that is higher, e.g., increased in the immune effector cell population in the sample from the subject. In some embodiments, contacting the population of immune effector cells (e.g., comprising T cells that express one or more TCRαV molecules) with an anti-TCRαV molecule results in expansion of the population of immune effector cells expressing one or more TCRαV molecules. In some embodiments, the expanded population, or a portion thereof, is administered to the subject (e.g., same subject from whom the immune effector cell population was obtained), to treat the cancer. In some embodiments, the expanded population, or a portion thereof, is administered to a different subject (e.g., not the same subject from whom the immune effector cell population was obtained), to treat the cancer.

Also described herein are methods of treating a subject having a cancer, comprising, acquiring a status of one or more TCRαV molecules in a sample from the subject, and determining whether the one or more TCRαV molecules is higher, e.g., increased, in a sample from the subject compared to a reference value, wherein responsive to said determination, administering to the subject an effective amount of an anti-TCRαV antibody molecule, e.g., an agonistic anti-TCRαV antibody molecule, e.g., as described herein.

In some embodiments, the subject has B-CLL. In some embodiments, a subject having B-CLL has a higher, e.g., increased, level or activity of one or more TCRαV molecules.

In some embodiments, the subject is administered the multifunctional polypeptide molecule as described herein comprising an anti-TCRαV molecule (e.g., an agonistic anti-TCRαV molecule as described herein) that binds to one or more members of the TCRα V12 subfamily.

In some embodiments, acquiring a value for the status, e.g., presence, level and/or activity, of one or more TCRαV molecules comprises acquiring a measure of the T cell receptor (TCR) repertoire of a sample. In some embodiments, the value comprises a measure of the clonotype of a population of T cells in the sample.

In some embodiments, a value for the status of one or more TCRαV molecules is obtained, e.g., measured, using an assay described in Wang C Y, et al., Int J Oncol. (2016) 48(6):2247-56, the entire contents of which are hereby incorporated by reference.

In some embodiments, a value for the status of one or more TCRαV molecules is obtained, e.g., measured, using flow cytometry.

### Combination Therapies

In some embodiments, the method as described herein further comprises administering a second therapeutic agent or therapy to the subject.

In some embodiments, the second therapeutic agent or therapy comprises a chemotherapeutic agent, a biologic agent, a hormonal therapy, radiation, or surgery.

In some embodiments, the second therapeutic agent or therapy is administered in combination with the multifunctional polypeptide molecule as described herein, the nucleic acid molecules as described herein, the vector as described herein, the cell as described herein, the pharmaceutical composition as described herein, sequentially, simultaneously, or concurrently.

The multispecific or multifunctional molecules as described herein can be used in combination with a second therapeutic agent or procedure.

In some embodiments, the multispecific or multifunctional molecules as described herein and the second therapeutic agent or procedure are administered/performed after a subject has been diagnosed with a cancer, e.g., before the cancer has been eliminated from the subject. In some embodiments, the multispecific or multifunctional molecules as described herein and the second therapeutic agent or procedure are administered/performed simultaneously or concurrently. For example, the delivery of one treatment is still occurring when the delivery of the second commences, e.g., there is an overlap in administration of the treatments. In other embodiments, the multispecific or multifunctional molecules as described herein and the second therapeutic agent or procedure are administered/performed sequentially. For example, the delivery of one treatment ceases before the delivery of the other treatment begins.

In some embodiments, combination therapy can lead to more effective treatment than monotherapy with either agent alone. In some embodiments, the combination of the first and second treatment is more effective (e.g., leads to a greater reduction in symptoms and/or cancer cells) than the first or second treatment alone. In some embodiments, the combination therapy permits use of a lower dose of the first or the second treatment compared to the dose of the first or second treatment normally required to achieve similar effects when administered as a monotherapy. In some embodiments, the combination therapy has a partially additive effect, wholly additive effect, or greater than additive effect.

In some embodiments, the anti-TCRAV antibody, multispecific or multifunctional molecule is administered in combination with a therapy, e.g., a cancer therapy (e.g., one or more of anti-cancer agents, immunotherapy, photodynamic therapy (PDT), surgery and/or radiation). The terms “chemotherapeutic,” “chemotherapeutic agent,” and “anti-cancer agent” are used interchangeably herein. The administration of the multispecific or multifunctional molecule and the therapy, e.g., the cancer therapy, can be sequential (with or without overlap) or simultaneous. Administration of the anti-TCRAV antibody, multispecific or multifunctional molecule can be continuous or intermittent during the course of therapy (e.g., cancer therapy). Certain therapies described herein can be used to treat cancers and non-cancerous diseases. For example, PDT efficacy can be enhanced in cancerous and non-cancerous conditions (e.g., tuberculosis) using the methods and compositions described herein (reviewed in, e.g., Agostinis, P. et al. (2011) CA Cancer J. Clin. 61:250-281).

Methods described herein include treating a cancer in a subject by using the multispecific or multifunctional molecules as described herein, e.g., using a pharmaceutical composition as described herein. Also provided are methods for reducing or ameliorating a symptom of a cancer in a subject, as well as methods for inhibiting the growth of a cancer and/or killing one or more cancer cells. In some embodiments, the methods described herein decrease the size of a tumor and/or decrease the number of cancer cells in a subject administered with a described herein or a pharmaceutical composition described herein.

In some embodiments, the cancer is a hematological cancer. In some embodiments, the hematological cancer is a leukemia or a lymphoma. As used herein, a “hematologic cancer” refers to a tumor of the hematopoietic or lymphoid tissues, e.g. a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma plasma cell neoplasm, myelodysplastic syndrome, or myelodysplastic/myeloproliferative neoplasm.

In some embodiments, the cancer is a myeloproliferative neoplasm, e.g., primary or idiopathic myelofibrosis (MF), essential thrombocytosis (ET), polycythemia vera (PV), or chronic myelogenous leukemia (CML). In some embodiments, the cancer is myelofibrosis. In some embodiments, the subject has myelofibrosis. In some embodiments, the subject has a calreticulin mutation, e.g., a calreticulin mutation as described herein. In some embodiments, the subject does not have the JAK2-V617F mutation. In some embodiments, the subject has the JAK2-V617F mutation. In some embodiments, the subject has a MPL mutation. In some embodiments, the subject does not have a MPL mutation.

In some embodiments, the cancer is a solid cancer. Exemplary solid cancers include, but are not limited to, ovarian cancer, rectal cancer, stomach cancer, testicular cancer, cancer of the anal region, uterine cancer, colon cancer, rectal cancer, renal-cell carcinoma, liver cancer, non-small cell carcinoma of the lung, cancer of the small intestine, cancer of the esophagus, melanoma, Kaposi's sarcoma, cancer of the endocrine system, cancer of the thyroid gland, cancer of the parathyroid gland, cancer of the adrenal gland, bone cancer, pancreatic cancer, skin cancer, cancer of the head or neck, cutaneous or intraocular malignant melanoma, uterine cancer, brain stem glioma, pituitary adenoma, epidermoid cancer, carcinoma of the cervix squamous cell cancer, carcinoma of the fallopian tubes, carcinoma of the endometrium, carcinoma of the vagina, sarcoma of soft tissue, cancer of the urethra, carcinoma of the vulva, cancer of the penis, cancer of the bladder, cancer of the kidney or ureter, carcinoma of the renal pelvis, spinal axis tumor, neoplasm of the central nervous system (CNS), primary CNS lymphoma, tumor angiogenesis, metastatic lesions of said cancers, or combinations thereof.

In some embodiments, the cancer is acute lymphoblastic leukemia, acute lymphocytic leukemia, acute myelogenous leukemia, aplastic anemia, chronic myelogenous leukemia, desmoplastic small round cell tumor, Ewing's sarcoma, Hodgkin's disease, multiple myeloma, myelodysplasia, Non-Hodgkin's lymphoma, paroxysmal nocturnal hemoglobinuria, radiation poisoning, chronic lymphocytic leukemia, AL amyloidosis, essential thrombocytosis, polycythemia vera, severe aplastic anemia, neuroblastoma, breast tumors, ovarian tumors, renal cell carcinoma, autoimmune disorders, such as systemic sclerosis, osteopetrosis, inherited metabolic disorders, juvenile chronic arthritis, adrenoleukodystrophy, amegakaryocytic thrombocytopenia, sickle cell disease, severe congenital immunodeficiency, Griscelli syndrome type II, Hurler syndrome, Kostmann syndrome, Krabbe disease, metachromatic leukodystrophy, thalassemia, hemophagocytic lymphohistiocytosis, and Wiskott-Aldrich syndrome, leukemias, lymphomas, melanomas, neuroendocrine tumors, carcinomas and sarcomas. Exemplary cancers that may be treated with a compound, pharmaceutical composition, or method provided herein include lymphoma, sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma. Paget's Disease of the Nipple. Phyllodes Tumors, Lobular Carcinoma, Ductal Carcinoma, cancer of the pancreatic stellate cells, cancer of the hepatic stellate cells, or prostate cancer. In some embodiments, the cancer is a solid tumor. In some embodiments, the cancer is hematological.

In some embodiments, the multispecific or multifunctional molecules as described herein (or pharmaceutical composition as described herein) are administered in a manner appropriate to the disease to be treated or prevented. The quantity and frequency of administration will be determined by such factors as the condition of the patient, and the type and severity of the patient's disease. Appropriate dosages may be determined by clinical trials. For example, when “an effective amount” or “a therapeutic amount” is indicated, the precise amount of the pharmaceutical composition (or multispecific or multifunctional molecules) to be administered can be determined by a physician with consideration of individual differences in tumor size, extent of infection or metastasis, age, weight, and condition of the subject. In some embodiments, the pharmaceutical composition described herein can be administered at a dosage of 10⁴ to 10⁹ cells/kg body weight, e.g., 10⁵ to 10⁶ cells/kg body weight, including all integer values within those ranges. In some embodiments, the pharmaceutical composition described herein can be administered multiple times at these dosages. In some embodiments, the pharmaceutical composition described herein can be administered using infusion techniques described in immunotherapy (see, e.g., Rosenberg et al., New Eng. J. of Med. 319:1676, 1988).

In some embodiments, the multispecific or multifunctional molecules as described herein or the pharmaceutical composition as described herein is administered to the subject parentally. In some embodiments, the cells are administered to the subject intravenously, subcutaneously, intratumorally, intranodally, intramuscularly, intradermally, or intraperitoneally. In some embodiments, the cells are administered, e.g., injected, directly into a tumor or lymph node. In some embodiments, the cells are administered as an infusion (e.g., as described in Rosenberg et al., New Eng. J. of Med. 319:1676, 1988) or an intravenous push. In some embodiments, the cells are administered as an injectable depot formulation.

In some embodiments, the subject is a mammal. In some embodiments, the subject is a human, monkey, pig, dog, cat, cow, sheep, goat, rabbit, rat, or mouse. In some embodiments, the subject is a human. In some embodiments, the subject is a pediatric subject, e.g., less than 18 years of age, e.g., less than 17, 16, 15, 14, 13, 12, 11, 10, 9, 8, 7, 6, 5, 4, 3, 2, 1 or less years of age. In some embodiments, the subject is an adult, e.g., at least 18 years of age, e.g., at least 19, 20, 21, 22, 23, 24, 25, 25-30, 30-35, 35-40, 40-50, 50-60, 60-70, 70-80, or 80-90 years of age.

### Anti-Cancer Therapies

In other embodiments, the multispecific or multifunctional molecules as described herein is administered in combination with a low or small molecular weight chemotherapeutic agent. Exemplary low or small molecular weight chemotherapeutic agents include, but not limited to, 13-cis-retinoic acid (isotretinoin, ACCUTANE®), 2-CdA (2-chlorodeoxyadenosine, cladribine, LEUSTATIN™), 5-azacitidine (azacitidine, VIDAZA®), 5-fluorouracil (5-FU, fluorouracil, ADRUCIL®), 6-mercaptopurine (6-MP, mercaptopurine, PURINETHOL®), 6-TG (6-thioguanine, thioguanine, THIOGUANINE TABLOID®), abraxane (paclitaxel protein-bound), actinomycin-D (dactinomycin, COSMEGEN®), alitretinoin (PANRETIN®), all-transretinoic acid (ATRA, tretinoin, VESANOID®), altretamine (hexamethylmelamine, HMM, HEXALEN®), amethopterin (methotrexate, methotrexate sodium, MTX, TREXALL™, RHEUMATREX®), amifostine (ETHYOL®), arabinosylcytosine (Ara-C, cytarabine, CYTOSAR-U®), arsenic trioxide (TRISENOX®), asparaginase (Erwinia L-asparaginase, L-asparaginase. ELSPAR®, KIDROLASE®), BCNU (carmustine, BiCNU®), bendamustine (TREANDA®), bexarotene (TARGRETIN®), bleomycin (BLENOXANE®), busulfan (BUSULFEX®, MYLERAN®), calcium leucovorin (Citrovorum Factor, folinic acid, leucovorin), camptothecin-11 (CPT-11, irinotecan, CAMPTOSAR®), capecitabine (XELODA®,), carboplatin (PARAPLATIN®), carmustine wafer (prolifeprospan 20 with carmustine implant, GLIADEL® wafer), CCI-779 (temsirolimus, TORISEL®), CCNU (lomustine, CeeNU), CDDP (cisplatin, PLATINOL®, PLATINOL-AQ9®), chlorambucil (leukeran), cyclophosphamide (CYTOXAN®, NEOSAR®), dacarbazine (DIC, DTIC, imidazole carboxamide, DTIC-DOME®), daunomycin (daunorubicin, daunorubicin hydrochloride, rubidomycin hydrochloride. CERUBIDINE®), decitabine (DACOGEN®), dexrazoxane (ZINECARD®), DHAD (mitoxantrone, NOVANTRONE®), docetaxel (TAXOTERE®), doxorubicin (ADRIAMYCIN®, RUBEX®), epirubicin (ELLENCE™), estramustine (EMCYT®), etoposide (VP-16, etoposide phosphate, TOPOSAR®, VEPESID®, ETOPOPHOS®), floxuridine (FUDR®), fludarabine (FLUDARA®), fluorouracil (cream) (CARAC™, EFUDEX®, FLUOROPLEX®), gemcitabine (GEMZAR®), hydroxyurea (HYDREA®, DROXIA™, MYLOCEL™), idarubicin (IDAMYCIN®), ifosfamide (IFEX®), ixabepilone (IXEMPRA®), LCR (leurocristine, vincristine, VCR, ONCOVIN®, VINCASAR PFS®), L-PAM (L-sarcolysin, melphalan, phenylalanine mustard, ALKERAN®), mechlorethamine (mechlorethamine hydrochloride, mustine, nitrogen mustard, MUSTARGEN®), mesna (MESNEX™), mitomycin (mitomycin-C, MTC, MUTAMYCIN®), nelarabine (ARRANON®), oxaliplatin (ELOXATIN™), paclitaxel (TAXOL®, ONXAL™), pegaspargase (PEG-L-asparaginase, ONCOSPAR®), PEMETREXED (ALIMTA®), pentostatin (NIPENT®), procarbazine (MATULANE®), streptozocin (ZANOSAR®), temozolomide (TEMODAR®), teniposide (VM-26, VUMON®), TESPA (thiophosphoamide, thiotepa, TSPA, THIOPLEX®), topotecan (HYCAMTIN®), vinblastine (vinblastine sulfate, vincaleukoblastine, VLB, ALKABAN-AQ®, VELBAN®), vinorelbine (vinorelbine tartrate, NAVELBINE®), and vorinostat (ZOLINZA®).

In another embodiment, the multispecific or multifunctional molecules as described herein is administered in conjunction with a biologic. Biologics useful in the treatment of cancers are known in the art and a binding molecule as described herein may be administered, for example, in conjunction with such known biologics. For example, the FDA has approved the following biologics for the treatment of breast cancer: HERCEPTIN® (trastuzumab. Genentech Inc., South San Francisco, Calif.; a humanized monoclonal antibody that has anti-tumor activity in HER2-positive breast cancer); FASLODEX® (fulvestrant, AstraZeneca Pharmaceuticals, LP, Wilmington, Del.: an estrogen-receptor antagonist used to treat breast cancer); ARIMIDEX® (anastrozole, AstraZeneca Pharmaceuticals, LP; a nonsteroidal aromatase inhibitor which blocks aromatase, an enzyme needed to make estrogen); Aromasin® (exemestane, Pfizer Inc., New York, N.Y.; an irreversible, steroidal aromatase inactivator used in the treatment of breast cancer); FEMARA® (letrozole, Novartis Pharmaceuticals, East Hanover, N.J.; a nonsteroidal aromatase inhibitor approved by the FDA to treat breast cancer); and NOLVADEX® (tamoxifen, AstraZeneca Pharmaceuticals, LP; a nonsteroidal antiestrogen approved by the FDA to treat breast cancer). Other biologics with which the binding molecules as described herein may be combined include: AVASTIN® (bevacizumab, Genentech Inc.; the first FDA-approved therapy designed to inhibit angiogenesis); and ZEVALIN® (ibritumomab tiuxetan, Biogen Idec, Cambridge, Mass.; a radiolabeled monoclonal antibody currently approved for the treatment of B-cell lymphomas).

In addition, the FDA has approved the following biologics for the treatment of colorectal cancer: AVASTIN®; ERBITUX® (cetuximab, ImClone Systems Inc., New York, N.Y., and Bristol-Myers Squibb, New York, N.Y.; is a monoclonal antibody directed against the epidermal growth factor receptor (EGFR)); GLEEVEC®, (imatinib mesylate; a protein kinase inhibitor); and ERGAMISOL® (levamisole hydrochloride, Janssen Pharmaceutica Products. LP, Titusville, N.J.; an immunomodulator approved by the FDA in 1990 as an adjuvant treatment in combination with 5-fluorouracil after surgical resection in patients with Dukes' Stage C colon cancer).

For the treatment of lung cancer, exemplary biologics include TARCEVA® (erlotinib HCL, OSI Pharmaceuticals Inc., Melville, N.Y.; a small molecule designed to target the human epidermal growth factor receptor 1 (HER1) pathway).

For the treatment of multiple myeloma, exemplary biologics include VELCADE® (bortezomib, Millennium Pharmaceuticals, Cambridge Mass.; a proteasome inhibitor). Additional biologics include THALIDOMID® (thalidomide, Clegene Corporation, Warren, N.J.; an immunomodulatory agent and appears to have multiple actions, including the ability to inhibit the growth and survival of myeloma cells and anti-angiogenesis).

Additional exemplary cancer therapeutic antibodies include, but are not limited to, 3F8, abagovomab, adecatumumab, afutuzumab, alacizumab pegol, alemtuzumab (CAMPATH®, MABCAMPATH®), altumomab pentetate (HYBRI-CEAKER®), anatumomab mafenatox, anrukinzumab (IMA-638), apolizumab, arcitumomab (CEA-SCAN®), bavituximab, bectumomab (LYMPHOSCAN®), belimumab (BENLYSTA®, LYMPHOSTAT-B®), besilesomab (SCINTIMUN®), bevacizumab (AVASTIN®), bivatuzumab mertansine, blinatumomab, brentuximab vedotin, cantuzumab mertansine, capromab pendetide (PROSTASCINT®), catumaxomab (REMOVAB®), CC49, cetuximab (C225, ERBITUX®), citatuzumab bogatox, cixutumumab, clivatuzumab tetraxetan, conatumumab, dacetuzumab, denosumab (PROLIA®), detumomab, ecromeximab, edrecolomab (PANOREX®), elotuzumab, epitumomab cituxetan, epratuzumab, ertumaxomab (REXOMUN®), etaracizumab, farletuzumab, figitumumab, fresolimumab, galiximab, gemtuzumab ozogamicin (MYLOTARG®)), girentuximab, glembatumumab vedotin, ibritumomab (ibritumomab tiuxetan, ZEVALIN®), igovomab (INDIMACIS-125®), intetumumab, inotuzumab ozogamicin, ipilimumab, iratumumab, labetuzumab (CEA-CIDE®), lexatumumab, lintuzumab, lucatumumab, lumiliximab, mapatumumab, matuzumab, milatuzumab, minretumomab, mitumomab, nacolomab tafenatox, naptumomab estafenatox, necitumumab, nimotuzumab (THERACIM®, THERALOC®), nofetumomab merpentan (VERLUMA®), ofatumumab (ARZERRA®), olaratumab, oportuzumab monatox, oregovomab (OVAREX®), panitumumab (VECTIBIX®), pemtumomab (THERAGYN®), pertuzumab (OMNITARG®), pintumomab, pritumumab, ramucirumab, ranibizumab (LUCENTIS®), rilotumumab, rituximab (MABTHERA®, RITUXAN®), robatumumab, satumomab pendetide, sibrotuzumab, siltuximab, sontuzumab, tacatuzumab tetraxetan (AFP-CIDE®), taplitumomab paptox, tenatumomab, TGN1412, ticilimumab (tremelimumab), tigatuzumab, TNX-650, tositumomab (BEXXAR®), trastuzumab (HERCEPTIN®), tremelimumab, tucotuzumab celmoleukin, veltuzumab, volociximab, votumumab (HUMASPECT®), zalutumumab (HUMAX-EGFR®), and zanolimumab (HUMAX-CD4®).

In some embodiments, the multispecific or multifunctional molecules as described herein are administered in combination with a viral cancer therapeutic agent. Exemplary viral cancer therapeutic agents include, but not limited to, vaccinia virus (vvDD-CDSR), carcinoembryonic antigen-expressing measles virus, recombinant vaccinia virus (TK-deletion plus GM-CSF), Seneca Valley virus-001, Newcastle virus, coxsackie virus A21, GL-ONC1, EBNA1 C-terminal/LMP2 chimeric protein-expressing recombinant modified vaccinia Ankara vaccine, carcinoembryonic antigen-expressing measles virus, G207 oncolytic virus, modified vaccinia virus Ankara vaccine expressing p53, OncoVEX GM-CSF modified herpes-simplex 1 virus, fowlpox virus vaccine vector, recombinant vaccinia prostate-specific antigen vaccine, human papillomavirus 16/18 L1 virus-like particle/AS04 vaccine, MVA-EBNA1/LMP2 Inj, vaccine, quadrivalent HPV vaccine, quadrivalent human papillomavirus (types 6, 11, 16, 18) recombinant vaccine (GARDASIL®), recombinant fowlpox-CEA(6D)/TRICOM vaccine; recombinant vaccinia-CEA(6D)-TRICOM vaccine, recombinant modified vaccinia Ankara-5T4 vaccine, recombinant fowlpox-TRICOM vaccine, oncolytic herpes virus NV1020, HPV L1 VLP vaccine V504, human papillomavirus bivalent (types 16 and 18) vaccine (CERVARIX®), herpes simplex virus HF10, Ad5CMV-p53 gene, recombinant vaccinia DF3/MUC1 vaccine, recombinant vaccinia-MUC-1 vaccine, recombinant vaccinia-TRICOM vaccine, ALVAC MART-1 vaccine, replication-defective herpes simplex virus type 1 (HSV-1) vector expressing human Preproenkephalin (NP2), wild-type reovirus, reovirus type 3 Dearing (REOLYSIN®), oncolytic virus HSV1716, recombinant modified vaccinia Ankara (MVA)-based vaccine encoding Epstein-Barr virus target antigens, recombinant fowlpox-prostate specific antigen vaccine, recombinant vaccinia prostate-specific antigen vaccine, recombinant vaccinia-B7.1 vaccine, rAd-p53 gene, Ad5-delta24RGD, HPV vaccine 580299, JX-594 (thymidine kinase-deleted vaccinia virus plus GM-CSF), HPV-16/18 L1/AS04, fowlpox virus vaccine vector, vaccinia-tyrosinase vaccine, MEDI-517 HPV-16/18 VLP AS04 vaccine, adenoviral vector containing the thymidine kinase of herpes simplex virus TK99UN, HspE7, FP253/Fludarabine, ALVAC(2) melanoma multi-antigen therapeutic vaccine, ALVAC-hB7.1, canarypox-hIL-12 melanoma vaccine, Ad-REIC/Dkk-3, rAd-IFN SCH 721015, TIL-Ad-INFg, Ad-ISF35, and coxsackievirus A21 (CVA21, CAVATAK®).

In some embodiments, the multispecific or multifunctional molecules as described herein are administered in combination with a nanopharmaceutical. Exemplary cancer nanopharmaceuticals include, but not limited to, ABRAXANE® (paclitaxel bound albumin nanoparticles), CRLX101 (CPT conjugated to a linear cyclodextrin-based polymer), CRLX288 (conjugating docetaxel to the biodegradable polymer poly (lactic-co-glycolic acid)), cytarabine liposomal (liposomal Ara-C, DEPOCYT™), daunorubicin liposomal (DAUNOXOME®), doxorubicin liposomal (DOXIL®, CAELYX®), encapsulated-daunorubicin citrate liposome (DAUNOXOME®), and PEG anti-VEGF aptamer (MACUGEN®).

In some embodiments, the multispecific or multifunctional molecules as described herein are administered in combination with paclitaxel or a paclitaxel formulation, e.g., TAXOL®, protein-bound paclitaxel (e.g., ABRAXANE®). Exemplary paclitaxel formulations include, but are not limited to, nanoparticle albumin-bound paditaxel (ABRAXANE®, marketed by Abraxis Bioscience), docosahexaenoic acid bound-paclitaxel (DHA-paclitaxel, Taxoprexin, marketed by Protarga), polyglutamate bound-paclitaxel (PG-paclitaxel, paclitaxel poliglumex, CT-2103, XYOTAX, marketed by Cell Therapeutic), the tumor-activated prodrug (TAP), ANG105 (Angiopep-2 bound to three molecules of paclitaxel, marketed by ImmunoGen), paclitaxel-EC-1 (paclitaxel bound to the erbB2-recognizing peptide EC-1; see Li et al., Biopolymers (2007) 87:225-230), and glucose-conjugated paclitaxel (e.g., 2′-paclitaxel methyl 2-glucopyranosyl succinate, see Liu et al., Bioorganic & Medicinal Chemistry Letters (2007) 17:617-620).

Exemplary RNAi and antisense RNA agents for treating cancer include, but not limited to, CALAA-01, siG12D LODER (Local Drug EluteR), and ALN-VSP02.

Other cancer therapeutic agents include, but not limited to, cytokines (e.g., aldesleukin (IL-2, Interleukin-2, PROLEUKIN®), alpha Interferon (IFN-alpha. Interferon alfa, INTRON® A (Interferon alfa-2b), ROFERON-A® (Interferon alfa-2a)), Epoetin alfa (PROCRIT®), filgrastim (G-CSF, Granulocyte—Colony Stimulating Factor, NEUPOGEN®), GM-CSF (Granulocyte Macrophage Colony Stimulating Factor, sargramostim, LEUKINE™), IL-11 (Interleukin-11, oprelvekin, NEUMEGA®), Interferon alfa-2b (PEG conjugate) (PEG interferon, PEG-INTRON™), and pegfilgrastim (NEULASTA™)), hormone therapy agents (e.g., aminoglutethimide (CYTADREN®), anastrozole (ARIMIDEX®), bicalutamide (CASODEX®), exemestane (AROMASIN®), fluoxymesterone (HALOTESTIN®), flutamide (EULEXIN®), fulvestrant (FASLODEX®), goserelin (ZOLADEX®), letrozole (FEMARA®), leuprolide (ELIGARD™, LUPRON®, LUPRON DEPOT®, VIADUR™), megestrol (megestrol acetate, MEGACE®), nilutamide (ANANDRON®, NILANDRON®), octreotide (octreotide acetate. SANDOSTATIN®, SANDOSTATIN LAR®), raloxifene (EVISTA®), romiplostim (NPLATE®), tamoxifen (NOVALDEX®), and toremifene (FARESTON®)), phospholipase A2 inhibitors (e.g., anagrelide (AGRYLIN®)), biologic response modifiers (e.g., BCG (THERACYS®, TICE®), and Darbepoetin alfa (ARANESP®)), target therapy agents (e.g., bortezomib (VELCADE®), dasatinib (SPRYCEL™), denileukin diftitox (ONTAK®), erlotinib (TARCEVA®), everolimus (AFINITOR®), gefitinib (IRESSA®), imatinib mesylate (STI-571, GLEEVEC™), lapatinib (TYKERB®), sorafenib (NEXAVAR®), and SU11248 (sunitinib, SUTENT®)), immunomodulatory and antiangiogenic agents (e.g., CC-5013 (lenalidomide, REVLIMID®), and thalidomide (THALOMID®)), glucocorticosteroids (e.g., cortisone (hydrocortisone, hydrocortisone sodium phosphate, hydrocortisone sodium succinate, ALA-CORT®, HYDROCORT ACETATE®, hydrocortone phosphate LANACORT®, SOLU-CORTEF®), decadron (dexamethasone, dexamethasone acetate, dexamethasone sodium phosphate, DEXASONE®, DIODEX®, HEXADROL®, MAXIDEX®), methylprednisolone (6-methylprednisolone, methylprednisolone acetate, methylprednisolone sodium succinate, DURALONE®, MEDRALONE®, MEDROL®, M-PREDNISOL®, SOLU-MEDROL®), prednisolone (DELTA-CORTEF®, ORAPRED®, PEDIAPRED®, PRELONE®), and prednisone (DELTASONE®, LIQUID PRED®, METICORTEN®, ORASONE®)), and bisphosphonates (e.g., pamidronate (AREDIA®), and zoledronic acid (ZOMETA®)).

In some embodiments, the multispecific or multifunctional molecules as described herein are used in combination with a tyrosine kinase inhibitor (e.g., a receptor tyrosine kinase (RTK) inhibitor). Exemplary tyrosine kinase inhibitor include, but are not limited to, an epidermal growth factor (EGF) pathway inhibitor (e.g., an epidermal growth factor receptor (EGFR) inhibitor), a vascular endothelial growth factor (VEGF) pathway inhibitor (e.g., an antibody against VEGF, a VEGF trap, a vascular endothelial growth factor receptor (VEGFR) inhibitor (e.g., a VEGFR-1 inhibitor, a VEGFR-2 inhibitor, a VEGFR-3 inhibitor)), a platelet derived growth factor (PDGF) pathway inhibitor (e.g., a platelet derived growth factor receptor (PDGFR) inhibitor (e.g., a PDGFR-β inhibitor)), a RAF-1 inhibitor, a KIT inhibitor and a RET inhibitor. In some embodiments, the anti-cancer agent used in combination with the AHCM agent is selected from the group consisting of, axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab(AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), alemtuzumab (CAMPATH®), gemtuzumab ozogamicin (MYLOTARG®), ENMD-2076, PCI-32765, AC220, dovitinib lactate (TK1258, CHIR-258), BIBW 2992 (TOVOK™), SGX523, PCI-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, XL228, AEE788, AG-490, AST-6, BMS-599626, CUDC-101, PD153035, pelitinib (EKB-569), vandetanib (zactima), WZ3146, WZ4002, WZ8040, ABT-869 (linifanib), AEE788, AP24534 (ponatinib), AV-951 (tivozanib), axitinib, BAY 73-4506 (regorafenib), brivanib alaninate (BMS-582664), brivanib (BMS-540215), cediranib (AZD2171), CHIR-258 (dovitinib), CP 673451, CYC116, E7080, Ki8751, masitinib (AB1010), MGCD-265, motesanib diphosphate (AMG-706), MP-470, OSI-930, Pazopanib Hydrochloride. PD173074, Sorafenib Tosylate (Bay 43-9006), SU 5402, TSU-68 (SU6668), vatalanib, XL880 (GSK1363089, EXEL-2880). Selected tyrosine kinase inhibitors are chosen from sunitinib, erlotinib, gefitinib, or sorafenib. In some embodiments, the tyrosine kinase inhibitor is sunitinib.

In some embodiments, the multispecific or multifunctional molecules as described herein are administered in combination with one of more of: an anti-angiogenic agent, or a vascular targeting agent or a vascular disrupting agent. Exemplary anti-angiogenic agents include, but are not limited to, VEGF inhibitors (e.g., anti-VEGF antibodies (e.g., bevacizumab); VEGF receptor inhibitors (e.g., itraconazole); inhibitors of cell proliferatin and/or migration of endothelial cells (e.g., carboxy amidotriazole, TNP-470); inhibitors of angiogenesis stimulators (e.g., suramin), among others. A vascular-targeting agent (VTA) or vascular disrupting agent (VDA) is designed to damage the vasculature (blood vessels) of cancer tumors causing central necrosis (reviewed in, e.g., Thorpe, P. E. (2004) Clin. Cancer Res. Vol. 10:415-427). VTAs can be small-molecule. Exemplary small-molecule VTAs include, but are not limited to, microtubule destabilizing drugs (e.g., combretastatin A-4 disodium phosphate (CA4P), ZD6126, AVE8062, Oxi 4503); and vadimezan (ASA404).

### Immune Checkpoint Inhibitors

In other embodiments, methods described herein comprise use of an immune checkpoint inhibitor in combination with the multispecific or multifunctional molecules as described herein. The methods can be used in a therapeutic protocol in vivo.

In some embodiments, an immune checkpoint inhibitor inhibits a checkpoint molecule. Exemplary checkpoint molecules include but are not limited to CTLA4, PD1, PD-L1, PD-L2, TIM3, LAG3, CD160, 2B4, CD80, CD86, B7-H3 (CD276), B7-H4 (VTCN1), HVEM (TNFRSF14 or CD270), BTLA, KIR, MHC class I, MHC class II, GAL9, VISTA, BTLA, TIGIT, LAIR1, and A2aR. See, e.g., Pardoll. Nat. Rev. Cancer 12.4(2012):252-64, incorporated herein by reference.

In some embodiments, the immune checkpoint inhibitor is a PD-1 inhibitor, e.g., an anti-PD-1 antibody such as Nivolumab, Pembrolizumab or Pidilizumab. Nivolumab (also called MDX-1106, MDX-1106-04, ONO-4538, or BMS-936558) is a fully human IgG4 monoclonal antibody that specifically inhibits PD1. See, e.g., U.S. Pat. No. 8,008,449 and WO2006/121168. Pembrolizumab (also called Lambrolizumab, MK-3475, MK03475, SCH-900475 or KEYTRUDA®; Merck) is a humanized IgG4 monoclonal antibody that binds to PD-1. See, e.g., Hamid, O. et al. (2013) New England Journal of Medicine 369 (2): 134-44, U.S. Pat. No. 8,354,509 and WO2009/114335. Pidilizumab (also called CT-011 or Cure Tech) is a humanized IgG1k monoclonal antibody that binds to PD1. See, e.g., WO2009/101611. In some embodiments, the inhibitor of PD-1 is an antibody molecule having a sequence substantially identical or similar thereto, e.g., a sequence at least 85%, 90%, 95% identical or higher to the sequence of Nivolumab, Pembrolizumab or Pidilizumab. Additional anti-PD1 antibodies, e.g., AMP 514 (Amplimmune), are described, e.g., in U.S. Pat. No. 8,609,089, US 2010028330, and/or US 20120114649.

In some embodiments, the PD-1 inhibitor is an immunoadhesin, e.g., an immunoadhesin comprising an extracellular/PD-1 binding portion of a PD-1 ligand (e.g., PD-L1 or PD-L2) that is fused to a constant region (e.g., an Fc region of an immunoglobulin). In some embodiments, the PD-1 inhibitor is AMP-224 (B7-DCIg, e.g., described in WO2011/066342 and WO2010/027827), a PD-L2 Fc fusion soluble receptor that blocks the interaction between B7-H1 and PD-1.

In some embodiments, the immune checkpoint inhibitor is a PD-L1 inhibitor, e.g., an antibody molecule. In some embodiments, the PD-L1 inhibitor is YW243.55.S70, MPDL3280A, MEDI-4736, MSB-0010718C, or MDX-1105. In some embodiments, the anti-PD-L1 antibody is MSB0010718C (also called A09-246-2; Merck Serono), which is a monoclonal antibody that binds to PD-LL. Exemplary humanized anti-PD-L1 antibodies are described, e.g., in WO2013/079174. In some embodiments, the PD-L1 inhibitor is an anti-PD-L1 antibody, e.g., YW243.55.570. The YW243.55.S70 antibody is described, e.g., in WO 2010/077634. In some embodiments, the PD-L1 inhibitor is MDX-1105 (also called BMS-936559), which is described, e.g., in WO2007/005874. In some embodiments, the PD-L1 inhibitor is MDPL3280A (Genentech/Roche), which is a human Fc-optimized IgG1 monoclonal antibody against PD-L1. See, e.g., U.S. Pat. No. 7,943,743 and U.S. Publication No.: 20120039906. In some embodiments, the inhibitor of PD-L1 is an antibody molecule having a sequence substantially identical or similar thereto. e.g., a sequence at least 85%, 90%, 95% identical or higher to the sequence of YW243.55.S70, MPDL3280A, MEDI-4736, MSB-0010718C, or MDX-1105.

In some embodiments, the immune check-point inhibitor is a PD-L2 inhibitor, e.g., AMP-224 (which is a PD-L2 Fc fusion soluble receptor that blocks the interaction between PD1 and B7-H1. See, e.g., WO2010/027827 and WO2011/066342.

In some embodiments, the immune checkpoint inhibitor is a LAG-3 inhibitor, e.g., an anti LAG-3 antibody molecule. In some embodiments, the anti-LAG-3 antibody is BMS-986016 (also called BMS986016; Bristol-Myers Squibb). BMS-986016 and other humanized anti-LAG-3 antibodies are described, e.g., in US 2011/0150892, WO2010/019570, and WO2014/008218.

In some embodiments, the immune checkpoint inhibitor is a TIM-3 inhibitor, e.g., anti-TIM3 antibody molecule, e.g., described in U.S. Pat. No. 8,552,156, WO 2011/155607. EP 2581113 and U.S. Publication No.: 2014/044728.

In some embodiments, the immune checkpoint inhibitor is a CTLA-4 inhibitor, e.g., anti-CTLA-4 antibody molecule. Exemplary anti-CTLA4 antibodies include Tremelimumab (IgG2 monoclonal antibody from Pfizer, formerly known as ticilimumab, CP-675,206); and Ipilimumab (also called MDX-010, CAS No. 477202-00-9). Other exemplary anti-CTLA-4 antibodies are described, e.g., in U.S. Pat. No. 5,811,097.

### Method of Expanding Cells

Any of the compositions and methods described herein can be used to expand an immune cell population. An immune cell provided herein includes an immune cell derived from a hematopoietic stem cell or an immune cell derived from a non-hematopoietic stem cell, e.g., by differentiation or de-differentiation.

An immune cell includes a hematopoietic stem cell, progeny thereof and/or cells that have differentiated from said HSC, e.g., lymphoid cells or myeloid cells. An immune cell can be an adaptive immune cell or an innate immune cell. Examples of immune cells include T cells, B cells, Natural Killer cells, Natural Killer T cells, neutrophils, dendritic cells, monocytes, macrophages, and granulocytes.

In some embodiments, an immune cell is a T cell. In some embodiments, a T cell includes a CD4+ T cell, a CD8+ T cell, a TCR alpha-beta T cell, a TCR gamma-delta T cell. In some embodiments, a T cell comprises a memory T cell (e.g., a central memory T cell, or an effector memory T cell (e.g., a TEMRA) or an effector T cell. In some embodiments, a T cell comprises a tumor infiltrating lymphocyte (TIL).

In some embodiments, an immune cell is an NK cell.

In some embodiments, an immune cell is a TIL. TILs are immune cells (e.g., T cells, B cells or NK cells) that can be found in a tumor or around a tumor (e.g., in the stroma or tumor microenvironment of a tumor), e.g., a solid tumor, e.g., as described herein. TILs can be obtained from a sample from a subject having cancer, e.g., a biopsy or a surgical sample. In some embodiments, TILs can be expanded using a method as described herein. In some embodiments, a population of expanded TILs, e.g., expanded using a method as described herein, can be administered to a subject to treat a disease, e.g., a cancer.

In some embodiments, immune cells, e.g., T cells (e.g., TILs), can be obtained from a unit of blood collected from a subject using any number of techniques known to the skilled artisan, such as Ficoll™ separation. In one aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and, optionally, to place the cells in an appropriate buffer or media for subsequent processing steps. In one embodiment, the cells are washed with phosphate buffered saline (PBS). In an alternative embodiment, the wash solution lacks calcium and may lack magnesium or may lack many if not all divalent cations. The methods described herein can include more than one selection step, e.g., more than one depletion step.

In one embodiment, the methods as described herein can utilize culture media conditions comprising DMEM, DMEM F12, RPMI 1640, and/or AIM V media. The media can be supplemented with glutamine, HEPES buffer (e.g., 10 mM), serum (e.g., heat-inactivated serum, e.g., 10%), and/or beta mercaptoethanol (e.g., 55 uM). In some embodiments, the culture conditions as described herein comprise one or more supplements, cytokines, growth factors, or hormones. In some embodiments, the culture condition comprises one or more of IL-2, IL-15, or IL-7, or a combination thereof.

Immune effector cells such as T cells may be activated and expanded generally using methods as described, for example, in U.S. Pat. Nos. 6,352,694; 6,534,055; or 6,905,680. Generally, a population of immune cells, may be expanded by contact with an agent that stimulates a CD3/TCR complex associated signal and a ligand that stimulates a costimulatory molecule on the surface of the T cells; and/or by contact with a cytokine, e.g., IL-2, IL-15 or IL-7. T cell expansion protocols can also include stimulation, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody can be used. Examples of an anti-CD28 antibody include 9.3, B-T3, XR-CD28 (Diaclone, Besangon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In some embodiments, a TIL population can also be expanded by methods known in the art. For example, a population of TILs can be expanded as described in Hall et al., Journal for ImmunoTherapy of Cancer (2016) 4:61, the entire contents of which are hereby incorporated by reference. Briefly, TILs can be isolated from a sample by mechanical and/or physical digestion. The resultant TIL population can be stimulated with an anti-CD3 antibody in the presence of non-dividing feeder cells. In some embodiments, the TIL population can be cultured, e.g., expanded, in the presence of IL-2, e.g., human IL-2. In some embodiments, the TIL cells can be cultured, e.g., expanded for a period of at least 1-21 days, e.g., at least 1, 2, 3, 4, 5, 6, 7, 8, 9, 10, 11, 12, 13, 14, 15, 16, 17, 18, 19, 20 or 21 days.

As described herein, in some embodiments, an immune cell population (e.g., a T cell (e.g., a TEMRA cell or a TIL population) can be expanded by contacting the immune cell population with the multifunctional polypeptide molecule as described herein.

In some embodiments, the expansion occurs in vivo, e.g., in a subject. In some embodiments, a subject is administered the multifunctional polypeptide molecule as described herein resulting in expansion of immune cells in vivo.

In some embodiments, the expansion occurs ex vivo, e.g., in vitro. In some embodiments, cells from a subject, e.g., T cells, e.g., TIL cells, are expanded in vitro with the multifunctional polypeptide molecule as described herein. In some embodiments, the expanded TILs are administered to the subject to treat a disease or a symptom of a disease.

In some embodiments, a method of expansion as described herein results in an expansion of at least 1.1-10 fold, 10-20 fold, or 20-50 fold expansion. In some embodiments, the expansion is at least 1.1, 1.2, 1.3, 1.4, 1.5, 2, 3, 4, 5, 6, 7, 8, 9, 10, 15, 20, 25, 30, 35, 40, 45, 50, 55, 60, 65, 70, 75, 80, 85, 90, 100, 200, 300, 400, 500, 600, 700, 800, 900, 1000, 1500, 2000, 2500, 3000, 3500, 400, 4500, 5000, 5500, 6000, 6500, 7000, 7500, 8000, 8500, 9000, 9500, or 10000 fold expansion.

In some embodiments, a method of expansion as described herein comprises culturing, e.g., expanding, the cells for at least about 4 hours, 6 hours, 10 hours, 12 hours, 15 hours, 18 hours, 20 hours, or 22 hours. In some embodiments, a method of expansion as described herein comprises culturing, e.g., expanding, the cells for at least 1, 2, 3, 4, 5, 6, 7, 8, 9, 10, 11, 12, 13, 14, 15, 1, 6 17, 18, 19, 20 or 21 days. In some embodiments, a method of expansion as described herein comprises culturing, e.g., expanding, the cells for at least about 1 week, 2 weeks, 3 weeks, 4 weeks, 5 weeks, 6 weeks, 7 weeks or 8 weeks.

In some embodiments, a method of expansion as described herein is performed on immune cells obtained from a healthy subject.

In some embodiments, a method of expansion as described herein is performed on immune cells (e.g., TILs) obtained from a subject having a disease, e.g., a cancer, e.g., a solid tumor as described herein.

In some embodiments, a method of expansion as described herein further comprises contacting the population of cells with an agent, that promotes, e.g., increases, immune cell expansion. In some embodiments, the agent comprises an immune checkpoint inhibitor, e.g., a PD-1 inhibitor, a LAG-3 inhibitor, a CTLA4 inhibitor, or a TIM-3 inhibitor. In some embodiments, the agent comprises a 4-1BB agonist, e.g., an anti-4-1BB antibody.

Without wishing to be bound by theory, in some embodiments, the multifunctional polypeptide molecule as described herein can expand, e.g. selectively or preferentially expand, T cells expressing a T cell receptor (TCR) comprising a TCR alpha and/or TCR beta molecule, e.g., TCR alpha-beta T cells (αβ T cells). In some embodiments, the multifunctional polypeptide molecule as described herein does not expand, or induce proliferation of T cells expressing a TCR comprising a TCR gamma and/or TCR delta molecule, e.g., TCR gamma-delta T cells (γδ T cells). In some embodiments, the multifunctional polypeptide molecule as described herein selectively or preferentially expands αβ T cells over γδ T cells.

Without wishing to be bound by theory, in some embodiments, γδ T cells are associated with cytokine release syndrome (CRS) and/or neurotoxicity (NT). In some embodiments, the multispecific or multifunctional molecules as described herein result in selective expansion of non-γδ T cells, e.g., expansion of αβ T cells, thus reducing CRS and/or NT.

In some embodiments, any of the compositions or methods as described herein result in an immune cell population having a reduction of, e.g., depletion of, γδ T cells. In some embodiments, the immune cell population is contacted with an agent that reduces. e.g., inhibits or depletes, γδ T cells, e.g., an anti-IL-17 antibody or an agent that binds to a TCR gamma and/or TCR delta molecule.

In some embodiments, the multifunctional polypeptide molecule as described herein results in expansion of an immune cell, e.g., a T cell, a tumor infiltrating lymphocyte (TIL), an NK cell, or other immune cells (e.g., as described herein).

In some embodiments, binding of the multifunctional polypeptide molecule as described herein to a TCRαV region results in one, two, three or all of: (i) reduced T cell proliferation kinetics; (ii) cell killing, e.g., target cell killing, e.g. cancer cell killing, e.g., as measured by an assay of Example 4; (iii) increased Natural Killer (NK) cell proliferation, e.g., expansion; or (iv) expansion, e.g., at least about 1.1-10 expansion (e.g., at least about 1.1, 1.2, 1.3, 1.4, 1.5, 2, 3, 4, 5, 6, 7, 8, 9, or 10 fold expansion), of a population of T cells having a memory-like phenotype, e.g., as described herein, e.g., wherein (i)-(iv) are relative to the non-TCRαV-binding T cell engager.

In some embodiments, the method further comprises contacting the population of cells with an agent that promotes, e.g., increases, immune cell expansion. In some embodiments, the agent includes an immune checkpoint inhibitor, e.g., as described herein. In some embodiments, the agent includes a 4-1BB (CD127) agonist, e.g., an anti-4-1BB antibody.

In some embodiments, the method further comprises comprising contacting the population of cells with a non-dividing population of cells, e.g., feeder cells, e.g., irradiated allogenic human PBMCs.

In some embodiments, expansion of the population of immune cells, is compared to expansion of a similar population of cells with an antibody that binds to: a CD3 molecule, e.g., CD3 epsilon (CD3e) molecule; or a TCR alpha (TCRα) molecule.

In some embodiments, expansion of the population of immune cells, is compared to expansion of a similar population of cells not contacted with the anti-TCRαV antibody molecule or the multispecific or multifunctional molecules as described herein.

In some embodiments, expansion of the population of memory effector T cells, e.g., TEM cells, e.g., TEMRA cells, is compared to expansion of a similar population of cells with an antibody that binds to: a CD3 molecule, e.g., CD3 epsilon (CD3e) molecule; or a TCR alpha (TCRα) molecule.

In some embodiments, the method results in expansion of, e.g., selective or preferential expansion of, T cells expressing a T cell receptor (TCR) comprising a TCR alpha and/or TCR beta molecule, e.g., TCR alpha-beta T cells (αβ T cells).

In some embodiments, the method results in expansion of αβT cells over expansion of T cells expressing a TCR comprising a TCR gamma and/or TCR delta molecule, e.g., TCR gamma-delta T cells (γδ T cells). In some embodiments, expansion of αβT cells over γδ T cells results in reduced production of cytokines associated with CRS. In some embodiments, expansion of αβT cells over γδ T cells results in immune cells that have reduced capacity to, e.g., are less prone to, induce CRS upon administration into a subject.

In some embodiments, an immune cell population (e.g., T cells (e.g., TEMRA cells or TILs) or NK cells) cultured in the presence of, e.g., expanded with, the multifunctional polypeptide molecule as described herein does not induce CRS and/or NT when administered into a subject, e.g., a subject having a disease or condition as described herein.

In some embodiments, provided herein is a multifunctional polypeptide molecule as described herein comprising a non-murine, e.g., a human-like antibody molecule (e.g., a human or humanized antibody molecule), which binds, e.g., specifically binds, to a T cell receptor alpha variable (TCRαV) region. In some embodiments, binding of the multifunctional polypeptide molecule as described herein results in expansion, e.g., at least about 1.1-50 fold expansion (e.g., at least about 1.5-40 fold, 2-35 fold, 3-30 fold, 5-25 fold, 8-20 fold, or 10-15 fold expansion), of a population of T cells, e.g., a population of T cells having a memory-like phenotype, e.g., CD45RA+ CCR7− T cells. In some embodiments, the population of T cells having a memory-like phenotype comprises CD4+ and/or CD8+ T cells. In some embodiments, the population of T cells having a memory-like phenotype comprises a population of memory T cells, e.g., T effector memory (TEM) cells, e.g., TEM cells expressing CD45RA (TEMRA) cells, e.g., CD4+ or CD8+ TEMRA cells. In some embodiments, the population of T cells having a memory-like phenotype does not express a senescent marker, e.g., CD57. In some embodiments, the population of T cells having a memory-like phenotype does not express an inhibitory receptor, e.g., OX40, 4-1BB, and/or ICOS.

In some embodiments, the population of T cells having a memory-like phenotype is a population of T cells with CD45RA+ CCR7− CD57−. In some embodiments, the population of T cells having a memory-like phenotype does not express an inhibitory receptor, e.g., OX40, 4-1BB, and/or ICOS.

In some embodiments, the population of T cells having a memory-like phenotype, e.g., as described herein, has increased proliferative capacity. e.g., as compared to a reference cell population, e.g., an otherwise similar population of cells that has not been contacted with an anti-TCRαV antibody or the multispecific or multifunctional molecules as described herein.

In some embodiments, the expansion is at least about 1.1-10 fold expansion (e.g., at least about 1.1, 1.2, 1.3, 1.4, 1.5, 2, 3, 4, 5, 6, 7, 8, 9, or 10 fold expansion).

In some embodiments, expansion of the population of T cells having a memory-like phenotype. e.g., memory effector T cells, e.g., TEM cells, e.g., TEMRA cells, e.g., CD4+ or CD8+ TEMRA cells, is compared to expansion of a similar population of cells with an antibody that binds to: a CD3 molecule, e.g., CD3 epsilon (CD3e) molecule; or a TCR alpha (TCRα) molecule.

In some embodiments, the population of expanded T cells having a memory-like phenotype, e.g., T effector memory cells, comprises cells T cells, e.g., CD3+, CD8+ or CD4+ T cells. In some embodiments, the population of expanded T cells having a memory-like phenotype, T effector memory cells, comprises CD3+ and CD8+ T cells. In some embodiments, the population of expanded T cells having a memory-like phenotype, e.g., T effector memory cells comprises CD3+ and CD4+ T cells.

In some embodiments, the population of expanded T cells having a memory-like phenotype. T effector memory (TEM) cells, comprises cells T cells, e.g., CD3+, CD8+ or CD4+ T cells, which express or re-express, CD45RA, e.g., CD45RA+. In some embodiments, the population comprises TEM cells expressing CD45RA, e.g., TEMRA cells. In some embodiments, expression of CD45RA on TEMRA cells, e.g., CD4+ or CD8+ TEMRA cells, can be detected by a method as described herein, e.g., flow cytometry.

In some embodiments, the population of T cells having a memory-like phenotype, e.g., TEMRA cells have low or no expression of CCR7, e.g., CCR7− or CCR7 low. In some embodiments, expression of CCR7 on TEMRA cells cannot be detected by a method as described herein, e.g., flow cytometry.

In some embodiments, the population of T cells having a memory-like phenotype, e.g., TEMRA cells express CD95, e.g., CD95+. In some embodiments, expression of CD95 on TEMRA cells can be detected by a method as described herein, e.g., flow cytometry.

In some embodiments, the population of T cells having a memory-like phenotype, e.g., TEMRA cells express CD45RA, e.g., CD45RA+, have low or no expression of CCR7, e.g., CCR7− or CCR7 low, and express CD95, e.g., CD95+. In some embodiments, the population of T cells having a memory-like phenotype, e.g., TEMRA cells can be identified as CD45RA+. CCR7− and CD95+ cells. In some embodiments, the population of T cells having a memory-like phenotype, e.g., TEMRA cells comprise CD3+, CD4+ or CD8+ T cells (e.g., CD3+ T cells, CD3+ CD8+ T cells, or CD3+ CD4+ T cells).

In some embodiments, the population of T cells having a memory-like phenotype does not express a senescent marker, e.g., CD57.

In some embodiments, the population of T cells having a memory-like phenotype does not express an inhibitory receptor, e.g., OX40, 4-1BB, and/or ICOS.

In some embodiments, binding of the multifunctional polypeptide molecule as described herein results in expansion, e.g., at least about 1.1-50 fold expansion (e.g., at least about 1.5-40 fold, 2-35 fold, 3-30 fold, 5-25 fold, 8-20 fold, or 10-15 fold expansion), of a subpopulation of T cells. In some embodiments, the multifunctional polypeptide molecule as described herein-activated (e.g., expanded) subpopulation of T cells resemble TEMRA cells in high expression of CD45RA and/or low expression of CCR7. In some embodiments, the multifunctional polypeptide molecule as described herein-activated (e.g., expanded) subpopulation of T cells do not display upregulation of the senescence markers CD57 and/or KLRG1. In some embodiments, the multifunctional polypeptide molecule as described herein-activated (e.g., expanded) subpopulation of T cells do not display upregulation of co-stimulatory molecules CD27 and/or CD28. In some embodiments, the multifunctional polypeptide molecule as described herein-activated (e.g., expanded) subpopulation of T cells are highly proliferative. In some embodiments, the multifunctional polypeptide molecule as described herein-activated (e.g., expanded) subpopulation of T cells secrete IL-2. In some embodiments, expression of surface markers on T cells can be detected by a method as described herein, e.g., flow cytometry. In some embodiments, the proliferative capability of T cells can be detected by a method as described herein, e.g., a method described in Example 4. In some embodiments, cytokine expression of T cells can be detected by a method as described herein, e.g., a method described in Examples 10 and 35. In some embodiments, the expansion is at least about 1.1-10 fold expansion (e.g., at least about 1.1, 1.2, 1.3, 1.4, 1.5, 2, 3, 4, 5, 6, 7, 8, 9, or 10 fold expansion). In some embodiments, the expansion is compared to expansion of a similar population of cells with an antibody that binds to a CD3 molecule, e.g., CD3 epsilon (CD3e) molecule; or a TCR alpha (TCRα) molecule.

In some embodiments, binding of the multifunctional polypeptide molecule as described herein results in proliferation, e.g., expansion, e.g., at least about 1.1-50 fold expansion (e.g., at least about 1.540 fold, 2-35 fold, 3-30 fold, 5-25 fold, 8-20 fold, or 10-15 fold expansion), of a population of Natural Killer (NK) cells. In some embodiments, the expansion of NK cells is at least about 1.1-30 fold expansion (e.g., at least about 1.1, 1.2, 1.3, 1.4, 1.5, 2, 3, 4, 5, 6, 7, 8, 9, 10, 15, 20, 25, or at least about 1.1-5, 5-10, 10-15, 15-20, 20-25, or 25-30 fold expansion). In some embodiments, the expansion of NK cells is measure by an assay of Example 4. In some embodiments, the expansion of NK cells by, e.g., binding of, the multifunctional polypeptide molecule as described herein is compared to expansion of an otherwise similar population not contacted with the multifunctional polypeptide molecule as described herein.

In some embodiments, binding of the multifunctional polypeptide molecule as described herein results in cell killing, e.g., target cell killing. e.g. cancer cell killing. In some embodiments, the cancer cell is a hematological cancer cell or a solid tumor cell. In some embodiments, the cancer cell is a multiple myeloma cell. In some embodiments, binding of the multifunctional polypeptide molecule as described herein results in cell killing in vitro or in vivo. In some embodiments, cell killing is measured by an assay of Example 4.

In some embodiments, binding of the multifunctional poly peptide molecule as described herein to a TCRαV region results in an increase or decrease of at least 2, 5, 10, 20, 50, 100, 200, 300, 400, 500, 600, 700, 800, 900, 1000, or 2000 fold, or at least 2-2000 fold (e.g., 5-1000, 10-900, 20-800, 50-700, 100-600, 200-500, or 300-400 fold) of any of the activities described herein compared the activity of 16G8 or TM23 murine antibody, or a humanized version thereof as described in U.S. Pat. No. 5,861,155.

In some embodiments, the method comprises expanding, e.g., increasing the number of, an immune cell population in the subject. In some embodiments, provided herein is a method of expanding. e.g., increasing the number of, an immune cell population comprising, contacting the immune cell population with an effective amount of the multifunctional polypeptide molecule as described herein. In some embodiments, the expansion occurs in vivo or ex vivo (e.g., in vitro).

In some embodiments, provided herein is a method of expanding, e.g., increasing the number of, an immune cell population comprising, contacting the immune cell population with a multifunctional poly peptide molecule as described herein comprising an antibody molecule, e.g., humanized antibody molecule, which binds, e.g., specifically binds, to a T cell receptor alpha variable chain (TCRαV) region (e.g., anti-TCRαV antibody molecule), thereby expanding the immune cell population. In some embodiments, the expansion occurs in vivo or ex vivo (e.g., in vitro).

In some embodiments, provided herein is a method of expanding a population of immune effector cells from a subject having a cancer, the method comprising: (i) isolating a biological sample comprising a population of immune effector cells from the subject; e.g., a peripheral blood sample, biopsy sample, or bone marrow sample; (ii) acquiring a value of the status of one or more TCRαV molecules for the subject, e.g., in the biological sample from the subject, wherein said value comprises a measure of the presence of, e.g., level or activity of, a TCRαV molecule in a sample from the subject compared to a reference value, e.g., a sample from a health subject, wherein a value that is higher, e.g., increased, in the subject relative to the reference, e.g., healthy subject, is indicative of the presence of cancer in the subject, and (iii) contacting the biological sample comprising a population of immune effector cells with the multifunctional polypeptide molecule as described herein.

In some embodiments, the method further comprises administering the population of immune effector cells contacted with the multifunctional polypeptide molecule as described herein to the subject.

In some embodiments, a higher, e.g., increased, level or activity of one or more TCRαV molecules in a subject, e.g., in a sample from a subject, is indicative of a bias, e.g., a preferential expansion, e.g., clonal expansion, of T cells expressing said one or more TCRαV molecules in the subject.

Accordingly, provided herein are, inter alia, multispecific or multifunctional molecules comprising TCRαV-binding moieties as described herein (e.g., multispecific or multifunctional antibody molecules that comprise anti-TCRαV antibody molecules), CARs as described herein, nucleic acids encoding the same, vectors as described herein, cells as described herein (e.g., the T cell genetically modified to express the CAR), methods of producing the aforesaid molecules, pharmaceutical compositions comprising aforesaid molecules, and methods of treating a disease or disorder, e.g., cancer, using the aforesaid molecules. The antibody molecules and pharmaceutical compositions as described herein can be used (alone or in combination with other agents or therapeutic modalities) to treat, prevent and/or diagnose disorders and conditions, e.g., cancer. e.g., as described herein.

TABLE 1
Constant region amino acid sequences of human IgG heavy chains and human kappa
light chain

| | | |
|---|---|---|
| Human kappa | LC | RTVAAPSVFI FPPSDEQLKS GTASVVCLLN NFYPREAKVQ |
| constant region | | WKVDNALQSG NSQESVTEQD SKDSTYSLSS TLTLSKADYE |
| SEQ ID NO: 39 | | KHKVYACEVT HQGLSSPVTK SFNRGEC |
| Human | LC | RTVAAPSVFIFPPSDEQLKSGTASVVCLLNNFYPREAKVQWKVD |
| Immunoglobulin | | NALQSGNSQESVTEQDSKDSTYSLSSTLTLSKADYEKHKVYACE |
| kappa constant | | VTHQGLSSPVTKSFNRGEC |
| C region | | |
| SEQ ID NO: 3644 | | |
| IgG4 (S228P) | HC | ASTKGPSVFPLAPCSRSTSESTAALGCLVKDYFPEPVTVSWNSGA |
| mutant | | LTSGVHTFPAVLQSSGLYSLSSVVTVPSSSLGTKTYTCNVDHKPS |
| constant region | | NTKVDKRVESKYGPPCPPCPAPEFLGGPSVFLFPPKPKDTLMISRT |
| (EU Numbering) | | PEVTCVVVDVSQEDPEVQFNWYVDGVEVHNAKTKPREEQFNST |
| SEQ ID NO: 40 | | YRVVSVLTVLHQDWLNGKEYKCKVSNKGLPSSIEKTISKAKGQP |
| | | REPQVYTLPPSQEEMTKNQVSLTCLVKGFYPSDIAVEWESNGQP |
| | | ENNYKTTPPVLDSDGSFFLYSRLTVDKSRWQEGNVFSCSVMHEA |
| | | LHNHYTQKSLSLSLG |
| IgG1 wild type | HC | ASTKGPSVFPLAPSSKSTSGGTAALGCLVKDYFPEPVTVSWNSG |
| SEQ ID NO: 41 | | ALTSGVHTFPAVLQSSGLYSLSSVVTVPSSSLGTQTYICNVNHKP |
| | | SNTKVDKRVEPKSCDKTHTCPPCPAPELLGGPSVFLFPPKPKDTL |
| | | MISRTPEVTCVVVDVSHEDPEVKFNWYVDGVEVHNAKTKPREE |
| | | QYNSTYRVVSVLTVLHQDWLNGKEYKCKVSNKALPAPIEKTISK |
| | | AKGQPREPQVYTLPPSREEMTKNQVSLTCLVKGFYPSDIAVEWE |
| | | SNGQPENNYKTTPPVLDSDGSFFLYSKLTVDKSRWQQGNVFSCS |
| | | VMHEALHNHYTQKSLSLSPGK |
| IgG1 (N297A) | HC | ASTKGPSVFPLAPSSKSTSGGTAALGCLVKDYFPEPVTVSWNSG |
| mutant | | ALTSGVHTFPAVLQSSGLYSLSSVVTVPSSSLGTQTYICNVNHKP |
| constant region | | SNTKVDKRVEPKSCDKTHTCPPCPAPELLGGPSVFLFPPKPKDTL |
| (EU Numbering) | | MISRTPEVTCVVVDVSHEDPEVKFNWYVDGVEVHNAKTKPREE |
| SEQ ID NO: 42 | | QYASTYRVVSVLTVLHQDWLNGKEYKCKVSNKALPAPIEKTISK |
| | | AKGQPREPQVYTLPPSREEMTKNQVSLTCLVKGFYPSDIAVEWE |
| | | SNGQPENNYKTTPPVLDSDGSFFLYSKLTVDKSRWQQGNVFSCS |
| | | VMHEALHNHYTQKSLSLSPGK |
| IgM constant | HC | GSASAPTLFPLVSCENSPSDTSSVAVGCLAQDFLPDSITFSWKYK |
| delta CDC | | NNSDISSTRGFPSVLRGGKYAATSQVLLPSKDVMQGTDEHVVCK |
| (P311A, P313S) | | VQHPNGNKEKNVPLPVIAELPPKVSVFVPPRDGFFGNPRKSKLIC |
| SEQ ID NO: 73 | | QATGFSPRQIQVSWLREGKQVGSGVTTDQVQAEAKESGPTTYK |
| | | VTSTLTIKESDWLGQSMFTCRVDHRGLTFQQNASSMCVPDQDT |
| | | AIRVFAIPPSFASIFLTKSTKLTCLVTDLTTYDSVTISWTRQNGEA |
| | | VKTHTNISESHPNATFSAVGEASICEDDWNSGERFTCTVTHTDLA |
| | | SSLKQTISRPKGVALHRPDVYLLPPAREQLNLRESATITCLVTGFS |
| | | PADVFVQWMQRGQPLSPEKYVTSAPMPEPQAPGRYFAHSILTVS |
| | | EEEWNTGETYTCVVAHEALPNRVTERTVDKSTGKPTLYNVSLV |
| | | MSDTAGTCY |
| IgGA1 | HC | ASPTSPKVFPLSLCSTQPDGNVVIACLVQGFFPQEPLSVTWSESG |
| SEQ ID NO: 74 | | QGVTARNFPPSQDASGDLYTTSSQLTLPATQCLAGKSVTCHVKH |
| | | YTNPSQDVTVPCPVPSTPPTPSPSTPPTPSPSCCHPRLSLHRPALED |
| | | LLLGSEANLTCTLTGLRDASGVTFTWTPSSGKSAVQGPPERDLC |
| | | GCYSVSSVLPGCAEPWNHGKTFTCTAAYPESKTPLTATLSKSGN |
| | | TFRPEVHLLPPPSEELALNELVTLTCLARGFSPKDVLVRWLQGSQ |
| | | ELPREKYLTWASRQEPSQGTTTFAVTSILRVAAEDWKKGDTFSC |
| | | MVGHEALPLAFTQKTIDRLAGKPTHVNVSVVMAEVDGTCY |
| IgGA2 | HC | ASPTSPKVFPLSLDSTPQDGNVVVACLVQGFFPQEPLSVTWSESG |
| SEQ ID NO: 75 | | QNVTARNFPPSQDASGDLYTTSSQLTLPATQCPDGKSVTCHVKH |
| | | YTNSSQDVTVPCRVPPPPPCCHPRLSLHRPALEDLLLGSEANLTC |
| | | TLTGLRDASGATFTWTPSSGKSAVQGPPERDLCGCYSVSSVLPG |
| | | CAQPWNHGETFTCTAAHPELKTPLTANITKSGNTFRPEVHLLPPP |
| | | SEELALNELVTLTCLARGFSPKDVLVRWLQGSQELPREKYLTWA |
| | | SRQEPSQGTTTYAVTSILRVAAEDWKKGETFSCMVGHEALPLAF |
| | | TQKTIDRMAGKPTHINVSVVMAEADGTCY |
| Human Ig_J | HC | MKNHLLFWGVLAVFIKAVHVKAQEDERIVLVDNKCKCARITSRI |
| chain | | IRSSEDPNEDIVERNIRIIVPLNNRENISDPTSPLRTRFVYHLSDLCK |
| SEQ ID NO: 76 | | KCDPTEVELDNQIVTATQSNICDEDSATETCYTYDRNKCYTAVV |
| | | PLVYGGETKMVETALTPDACYPD |
| Human | HC | ASTKGPSVFPLAPSSKSTSGGTAALGCLVKDYFPEPVTVSWNSG |
| Immunoglobulin | | ALTSGVHTFPAVLQSSGLYSLSSVVTVPSSSLGTQTYICNVNHKP |
| heavy chain | | SNTKVDKKVEPKSCDKTHTCPPCPAPELLGGPSVFLFPPKPKDTL |
| SEQ ID NO: 3645 | | MISRTPEVTCVVVDVSHEDPEVKFNWYVDGVEVHNAKTKPREE |
| | | QYASTYRVVSVLTVLHQDWLNGKEYKCKVSNKALPAPIEKTISK |
| | | AKGQPREPQVYTLPPSREEMTKNQVSLTCLVKGFYPSDIAVEWE |
| | | SNGQPENNYKTTPPVLDSDGSFFLYSKLTVDKSRWQQGNVFSCS |
| | | VMHEALHNHYTQKSLSLSPGK |
| Human | HC | ASTKGPSVFPLAPSSKSTSGGTAALGCLVKDYFPEPVTVSWNSG |
| Immunoglobulin | | ALTSGVHTFPAVLQSSGLYSLSSVVTVPSSSLGTQTYICNVNHKP |
| heavy chain | | SNTKVDKKVEPKSCDKTHTCPPCPAPELLGGPSVFLFPPKPKDTL |
| SEQ ID NO: 3646 | | MISRTPEVTCVVVDVSHEDPEVKFNWYVDGVEVHNAKTKPREE |
| | | QYASTYRVVSVLTVLHQDWLNGKEYKCKVSNKALPAPIEKTISK |
| | | AKGQPREPQVYTLPPSREEMTKNQVSLTCLVKGFYPSDIAVEWE |
| | | SNGQPENNYKTTPPVLDSDGSFFLYSKLTVDKSRWQQGNVFSCS |
| | | VMHEALHNHYTQKSLSLSPGKEPEA |
| Human | HC | ASTKGPSVFPLAPSSKSTSGGTAALGCLVKDYFPEPVTVSWNSG |
| Immunoglobulin | | ALTSGVHTFPAVLQSSGLYSLSSVVTVPSSSLGTQTYICNVNHKP |
| heavy chain | | SNTKVDKRVEPKSCDKTHTCPPCPAPELLGGPSVFLFPPKPKDTL |
| SEQ ID NO: 3647 | | MISRTPEVTCVVVDVSHEDPEVKFNWYVDGVEVHNAKTKPREE |
| | | QYNSTYRVVSVLTVLHQDWLNGKEYKCKVSNKALPAPIEKTISK |
| | | AKGQPREPQVYTLPPSREEMTKNQVSLTCLVKGFYPSDIAVEWE |
| | | SNGQPENNYKTTPPVLDSDGSFFLYSKLTVDKSRWQQGNVFSCS |
| | | VMHEALHNHYTQKSLSLSPGK |
| Human Fc | HC | DKTHTCPPCPAPELLGGPSVFLFPPKPKDTLMISRTPEVTCVVVD |
| Knob cys | | VSHEDPEVKFNWYVDGVEVHNAKTKPREEQYASTYRVVSVLTV |
| N297A | | LHQDWLNGKEYKCKVSNKALPAPIEKTISKAKGQPREPQVYTLP |
| SEQ ID NO: 3648 | | PCREEMTKNQVSLWCLVKGFYPSDIAVEWESNGQPENNYKTTPP |
| | | VLDSDGSFFLYSKLTVDKSRWQQGNVFSCSVMHEALHNHYTQK |
| | | SLSLSPGK |
| Human IgG1 | HC | ASTKGPSVFPLAPSSKSTSGGTAALGCLVKDYFPEPVTVSWNSG |
| hole cys | | ALTSGVHTFPAVLQSSGLYSLSSVVTVPSSSLGTQTYICNVNHKP |
| N297A | | SNTKVDKRVEPKSCDKTHTCPPCPAPELLGGPSVFLFPPKPKDTL |
| SEQ ID NO: 3649 | | MISRTPEVTCVVVDVSHEDPEVKFNWYVDGVEVHNAKTKPREE |
| | | QYASTYRVVSVLTVLHQDWLNGKEYKCKVSNKALPAPIEKTISK |
| | | AKGQPREPQVCTLPPSREEMTKNQVSLSCAVKGFYPSDIAVEWE |
| | | SNGQPENNYKTTPPVLDSDGSFFLVSKLTVDKSRWQQGNVFSCS |
| | | VMHEALHNHYTQKSLSLSPGK |

TABLE 2
Exemplary Fc KiH mutations and optional Cysteine mutations

| Position | Knob Mutation | Hole Mutation |
|---|---|---|
| T366 | T366W | T366S |
| L368 | — | L368A |
| Y407 | — | Y407V |
| Additional Cysteine Mutations to | | |
| form a stabilizing disulfide bridge | | |
| Position | Knob CH3 | Hole CH3 |
| S354 | S354C | — |
| Y349 | — | Y349C |

TABLE 3
CRS grading

| | |
|---|---|
| Gr1 | Supportive care only |
| Gr2 | IV therapies +/−hospitalization. |
| Gr3 | Hypotension requiring IV fluids or low-dose vasoactives |
| | or hypoxemia requiring oxygen, CPAP, or BIPAP. |
| Gr4 | Hypotension requiring high-dose vasoactives or |
| | hypoxemia requiring mechanical ventilation. |
| Gr5 | Death |

TABLE 4
CTCAE v 4.0 CRS grading scale

| CRS | |
|---|---|
| grade | Characteristics |
| Grade 1 | Mild; No infusion interruption; No intervention |
| Grade 2 | Infusion interruption indicated but responds promptly |
| | to symptomatic treatment (e.g., antihistamines, NSAIDS, |
| | narcotics, IV fluids); prophylactic medications |
| | indicated for <=24 hrs |
| Grade 3 | Prolonged (e.g., not rapidly responsive to symptomatic |
| | medications and/or brief interruption of infusion); |
| | recurrence of symptoms following initial improvement; |
| | hospitalization indicated for clinical sequelae (e.g., |
| | renal impairment, pulmonary infiltrates) |
| Grade 4 | Life threatening consequences; pressor or ventilator support |

TABLE 5
NCI CRS grading scale

| CRS | |
|---|---|
| grade | Characteristics |
| Grade 1 | Symptoms are not life threatening and require |
| | symptomatic treatment only; e.g., fever, nausea, |
| | fatigue, headache, myalgias, malaise |
| Grade 2 | Symptoms require and respond to moderate |
| | intervention; Oxygen requirement <40% or |
| | hypotension responsive to fluids or low |
| | dose pressors or Grade 2 organ toxicity |
| Grade 3 | Symptoms require and respond to aggressive |
| | intervention; Oxygen requirement >=40% or |
| | Hypotension requiring high dose or multiple |
| | pressors or grade 3 organ toxicity or grade 4 |
| | transaminitis |
| Grade 4 | Life threatening symptoms Requirement for |
| | ventilator support or Grade 4; organ toxicity |
| | (excluding transaminitis) |

TABLE 6
Exemplary Fc modifications

| | Altered |
|---|---|
| Modification or mutation | effector function |
| Leu235Glu | ADCC; |
| Leu234Ala/Leu235Ala (LALA) | ADCC; ADCP; CDC |
| Ser228Pro/Leu235Glu | |
| Leu234Ala/Leu235Ala/Pro329Gly | ADCP |
| Pro331Ser/Leu234Glu/Leu235Phe | CDC |
| Asp265Ala | ADCC, ADCP |
| Gly237Ala | ADCP |
| Glu318Ala | ADCP |
| Glu233Pro | |
| Gly236Arg/Leu328Arg | ADCC |
| His268Gln/Val309Leu/Ala330Ser/Pro331Ser | ADCC; ADCP; CDC |
| Val234Ala/Gly237Ala/Pro238Ser/ | ADCC; ADCP; CDC |
| His268Ala/Val309Leu/Ala330Ser/Pro331Ser | |
| Leu234Ala/L235Ala/Gly237Ala/P238Ser/ | ADCC; CDC |
| His268Ala/Ala330Ser/Pro331Ser | |
| Ala330Leu | CDC |
| Asp270Ala | CDC |
| Lys322Ala | CDC |
| Pro329Ala | CDC |
| Pro331Ala | CDC |
| Val264Ala | CDC |
| High mannose glycosylation | CDC |
| Phe241Ala | CDC |
| Asn297Ala or Gly or Gln | ADCC; ADCP; CDC |
| S228P/Phe234Ala/Leu235Ala | ADCC; CDC |

TABLE 7
Amino acid sequences of exemplary variable regions of anti-BCMA antibodies.

| SEQ ID | | |
|---|---|---|
| NO | Description | Sequence |
| 3439 | 83A10 | EVQLLESGGGLVQPGGSLRLSCAASGFTFSSYAMSWVRQAPG |
| | VH | KGLEWVSAISGSGGSTYYADSVKGRFTISRDNSKNTLYLQMN |
| | | SLRAEDTAVYYCAKVLGWFDYWGQGTLVTVSS |
| 3440 | 83A10 | EIVLTQSPGTLSLSPGERATLSCRASQSVSSSYLAWYQQKPGQ |
| | VL | APRLLIYGASSRATGIPDRFSGSGSGTDFTLTISRLEPEDFAVY |
| | | YCQQYGYPPDFTFGQGTKVEIK |
| 3441 | 17A5 VH | EVQLLESGGGLVQPGGSLRLSCAASGFTFSSYAMSWVRQAPG |
| | | KGLEWVSAISGSGGSTYYADSVKGRFTISRDNSKNTLYLQMN |
| | | SLRAEDTAVYYCAKVAPYFAPFDYWGQGTLVTVSS |
| 3442 | 17A5 VL | EIVLTQSPGTLSLSPGERATLSCRASQSVSSSYLAWYQQKPGQ |
| | | APRLLIYGASSRATGIPDRFSGSGSGTDFTLTISRLEPEDFAVY |
| | | YCQQYGNPPLYTFGQGTKVEIK |
| 3443 | 13A4 VH | EVQLVQSGAEVKKPGESLKISCKGSGYSFTSYWIGWVRQMPG |
| | | KGLEWMGIIYPGDSDTRYSPSFQGQVTISADKSISTAYLQWSS |
| | | LKASDTAMYYCARNGYLGDYWGQGTLVTVSS |
| 3444 | 13A4 VL | DIVMTQSPLSLPVTPGEPASISCRSSQSLLHSNGYNYLDWYLQ |
| | | KPGQSPQLLIYLGSNRASGVPDRFSGSGSGTDFTLKISRVEAED |
| | | VGVYYCMQAMQIPTFGQGTKVEIK |
| 3445 | J22.9-xi | QVQLQQSGGGLVQPGGSLKLSCAASGIDFSRYWMSWVRRAP |
| | VH | GKGLEWIGEINPDSSTINYAPSLKDKFIISRDNAKNTLYLQMSK |
| | | VRSEDTALYYCASLYYDYGDAMDYWGQGTSVTVSS |
| 3446 | J22.9-xi | DIVMTQSQRFMTTSVGDRVSVTCKASQSVDSNVAWYQQKPR |
| | VL | QSPKALIFSASLRFSGVPARFTGSGSGTDFTLTISNLQSEDLAE |
| | | YFCQQYNNYPLTFGAGTKLELKR |
| 3447 | 2A1 VH | EVQLVESGGGLVKPGGSLRLSCAASGFTFGDYALSWFRQAPG |
| | | KGLEWVGVSRSKAYGGTTDYAASVKGRFTISRDDSKSTAYL |
| | | QMNSLKTEDTAVYYCASSGYSSGWTPFDYWGQGTLVTVSS |
| 3448 | 2A1 VL | QSVLTQPPSASGTPGQRVTISCSGSSSNIGSNTVNWYQQLPGT |
| | | APKLLIFNYHQRPSGVPDRFSGSKSGSSASLAISGLQSEDEADY |
| | | YCAAWDDSLNGWVFGGGTKLTVLG |

### EXAMPLES

The present disclosure will be more specifically illustrated by the following Examples. However, it should be understood that the present disclosure is not limited by these examples in any manner.

### Example 1: Relative Abundance of TRAV Genes

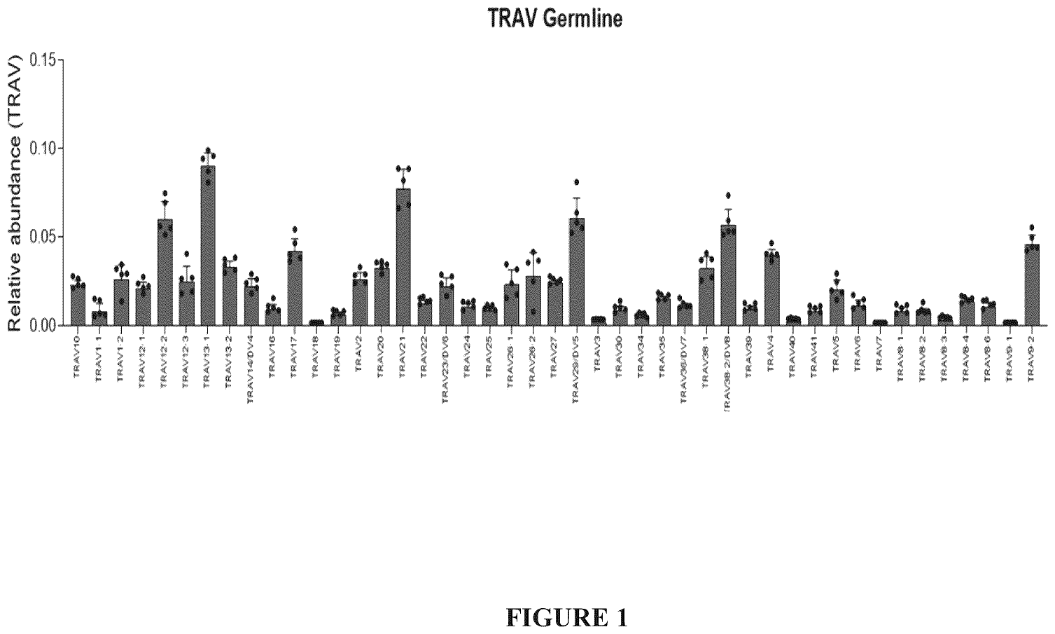

0.5×10⁵ T cells from healthy donors were collected using standard T cell isolation kits and stored at −80° C. until further use. TCR repertoire sequencing was performed. Total RNA was isolated using RNA Kits (Qiagen) and TCR transcripts were amplified using specific primers (SMARTer human TCR α/β profiling kit; TaKRa) that contained Illumina-specific adapter sequences. The indexed transcripts were further sequenced using Illumina platforms and the sequence reads were further analyzed using the MiXCR pipeline tool. The MiXCR tool specifically aligned the sequence reads to TCR germline segments of TRA, TRB, TRD and TRG genes in the datasets to identify germline assignments, CDR3 sequences and clonotype abundances. TRAV genes were counted and grouped together in their specific germlines and plotted as a bar chart representing frequency/abundance (FIG. 1). Each TRAV gene is plotted against relative abundance (where 1 equals 100% of total TRAV gene), equivalent to total T-cell abundance. The average relative abundance for each TRAV is calculated from five donors with each individual donor shown as (⋅).

### Example 2: Expansion of T-Cells Using an Anti-TRAV Antibody

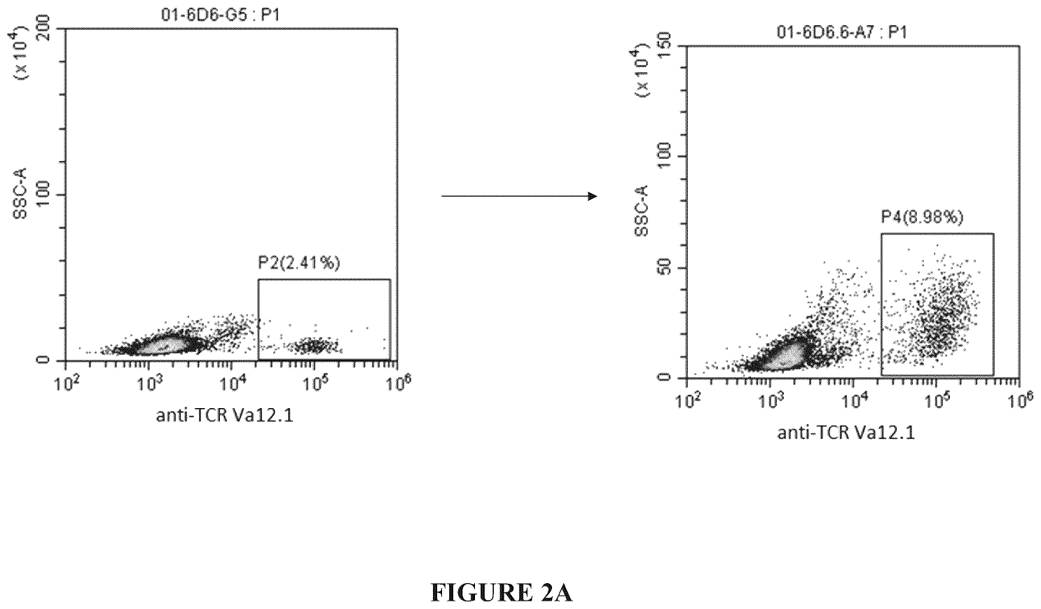

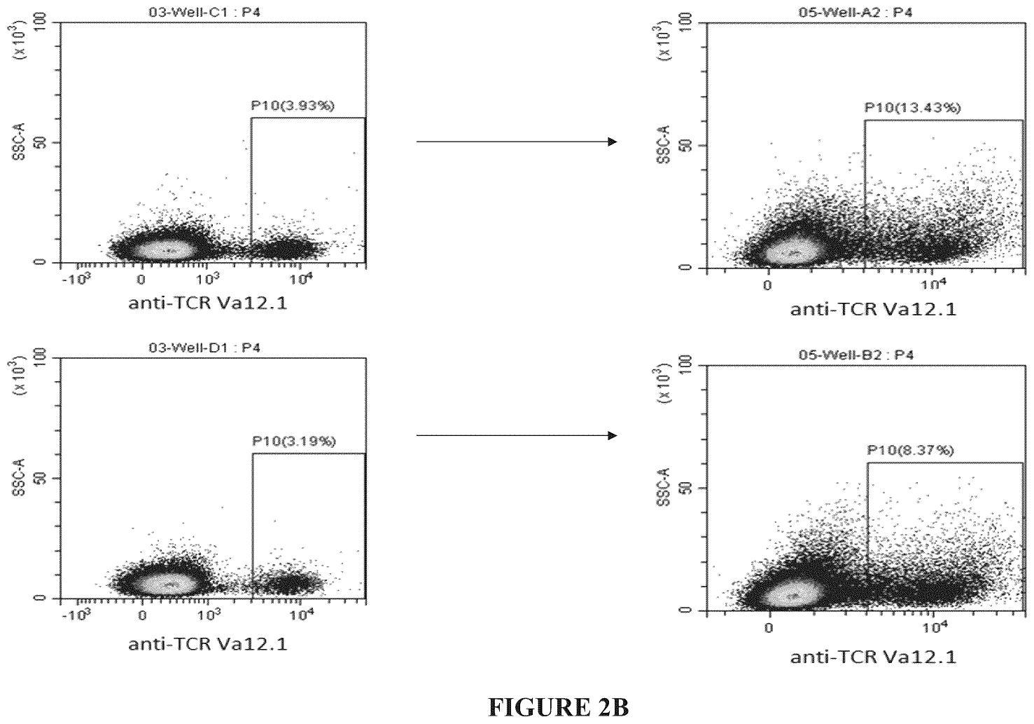

Untouched purified naïve human T-cells from healthy donors were directly procured. A 96-well plate was coated with an anti-TCR V alpha 12.1 (Novus) for 24 hours at 4° C. 2×10⁵ T cells were seeded into each well and for incubated at 37° C. for 5 days. After the 5-day culture, cell pellets were washed with FACS buffer (0.1% BSA, PBS) and stained with Live/Dead Zombie-UV, PerCPcy5.5/anti-CD3. BV421/anti-CD4, BV605/anti-CD8 (Biolegend) and anti-TCR V alpha 12.1 labeled using the Alexa Fluor™ 647 NHS Ester (ThermoFisher) for 20 mins. After FACS-antibody staining, cells were washed with FACS buffer and ran on flow cytometry analyzer, Cytoflex (Beckman Coulter) for data collection. FACS plots represents T-cells gated on CD3+ T-cells for TCR V alpha 12.1 expansion (FIG. 2A). Unstimulated T-cells serves as baseline TCR V alpha 12.1 levels.

### Example 3: Expansion of T-Cells Using an Anti-TRAV Antibody and IL-2

Untouched purified naïve human T-cells from healthy donors were directly procured. A 96-well plate was coated with 100 nM of anti-TCR V alpha 12.1 (Novus) for 24 hours at 4° C. 2×10⁵ T cells were seeded into each well and for incubated at 37° C. for 10 days; followed by change to fresh media containing 100 U/mL of recombinant human IL-2 and continued incubation for additional 2 days to allow further expansion of activated TCR V alpha 12.1 T-cells. After the 2-day culture, cell pellets were washed with FACS buffer (0.1% BSA, PBS) and stained with Live/Dead Zombie-UV, PerCPcy5.5/anti-CD3, BV421/anti-CD4, BV605/anti-CD8 (Biolegend) and anti-TCR V alpha 12.1 labeled using the Alexa Fluor™ 647 NHS Ester (ThermoFisher) for 20 mins. After FACS-antibody staining, cells were washed with FACS buffer and ran on flow cytometry analyzer, Cytoflex (Beckman Coulter) for data collection. FACS plots represents T-cells gated on CD3+ T-cells for TCR V alpha 12.1 expansion (FIG. 2B). Unstimulated T-cells serves as baseline TCR V alpha 12.1 levels.

### Example 4: Expansion of Human T-Cells Using an Anti-TCRαV-19/IL-2 Bispecific Antibody

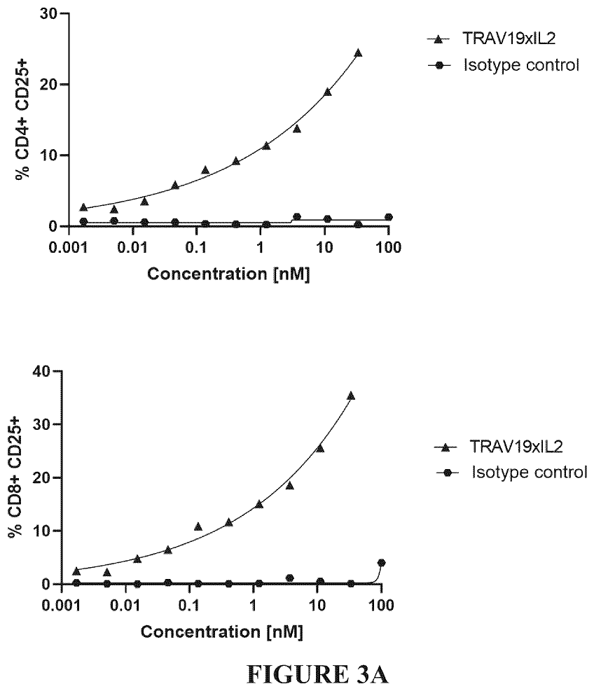

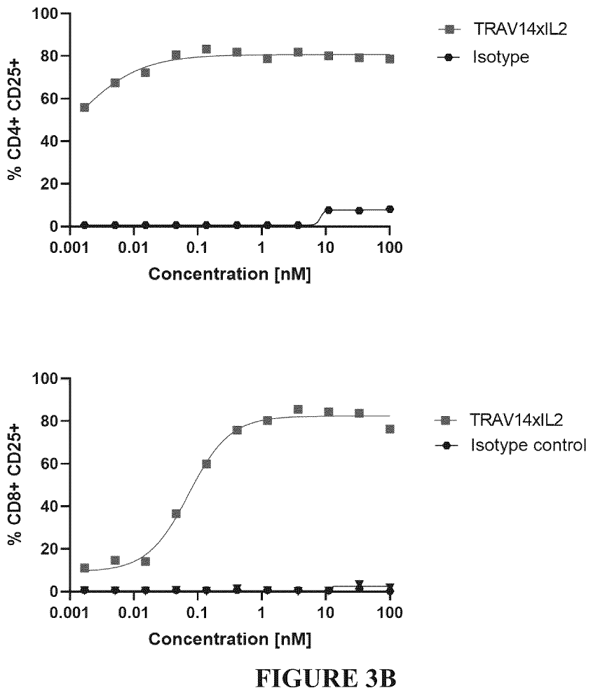

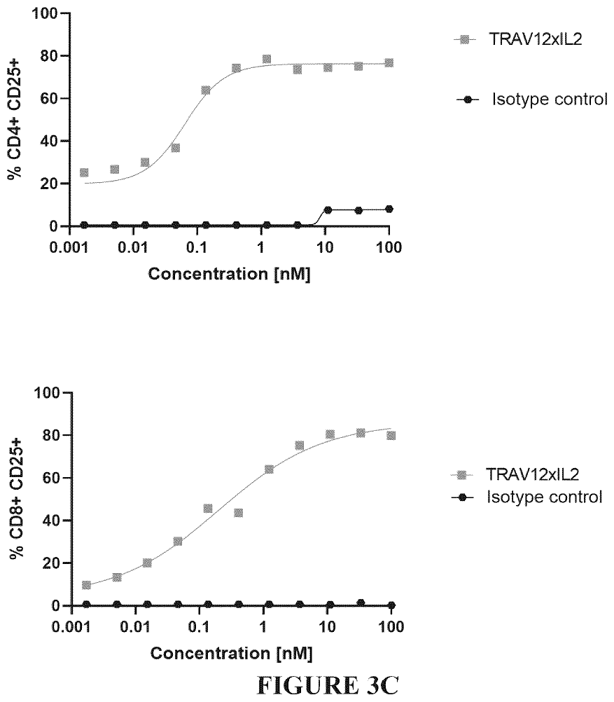

Human peripheral blood mononuclear cells (PBMCs) were thawed and plated at 3×10⁵ cells/well in a 96-well flat-bottom plate with 0.001, 0.01, 0.1, 1, 10, or 100 nM of bispecific antibody molecule with specificity for human TCRαV-19 and IL-2 (anti-TCRαV-19/IL-2) for 5 days at 37° C. After the 5-day culture, cell pellets were washed with FACS buffer (0.1% BSA, PBS) and stained with anti-CD4, anti-CD25 (IL2RA), and anti-CD8 antibodies for 20 mins. After FACS-antibody staining, cells were washed with FACS buffer and ran on flow cytometry analyzer, Cytoflex (Beckman Coulter) for data collection. Dot plots represent expansion and activation of CD4+ CD25+ and CD8+ CD25+ T-cells in anti-TCRαV-19/IL-2 treated cells compared to isotype controls (FIG. 3A).

### Example 5: Expansion of Murine T-Cells Using an Anti-TCRαV-14/IL-2 Bispecific Antibody

Spleens from two naïve BALB/c or C57BL/6 albino mice were mechanically dissociated with the backend of syringe through a 70 μM cell strainer in RPMI to obtain single-cell suspensions. Cells were centrifuged at 300G for 10 mins and the supernatant was removed. Red blood cells were then lysed by addition of 1 mL ACK lysis buffer to the pellet. Well-resuspended cells were incubated for 3 mins on ice. 10 mL RPMI was added, and cells were centrifuged at 300G for 10 mins. Cell pellet was resuspended in splenocyte media (RPMI+10% FBS+L-Glut+Pen/Strep+1.5 mM HEPES+0.5 mM BME), then filtered with a 70 μM cell strainer. T-cells were isolated using T Cell Isolation Kit II, mouse (Miltenyi Biotec cat #130-095-130). Isolated murine T-cells were plated at 0.001, 0.01, 0.1, 1, 10, or 100 nM of bispecific antibody molecule with specificity for murine TCRαV-14 (corresponding to human TCRαV-23/DV6) and IL-2 (anti-TCRαV-14/IL-2) for 4 days at 37° C. After the 4-day culture, cell pellets were washed with FACS buffer (0.1% BSA, PBS) and stained with anti-CD4, anti-CD25 (IL2RA), and anti-CD8 antibodies for 20 mins. After FACS-antibody staining, cells were washed with FACS buffer and ran on flow cytometry analyzer, Cytoflex (Beckman Coulter) for data collection. Dot plots represent expansion and activation of CD4+ CD25+ and CD8+ CD25+ T-cells in anti-TCRαV-14/IL-2 treated cells compared to isotype controls (FIG. 3B).

### Example 6: Expansion of Murine T Cells Using an Anti-TCRαV-12/IL-2 Bispecific Antibody

Murine T-cells were isolated as described in Example 5. Isolated murine T-cells were plated at 0.001, 0.01, 0.1, 1, 10, or 100 nM of bispecific antibody molecule with specificity for murine TCRαV-12 (corresponding to human TCRαV-18) and IL-2 (anti-TCRαV-12/IL-2) for 4 days at 37° C. After the 4-day culture, cell pellets were washed with FACS buffer (0.1% BSA, PBS) and stained with anti-CD4, anti-CD25 (IL2RA), and anti-CD8 antibodies for 20 mins. After FACS-antibody staining, cells were washed with FACS buffer and ran on flow cytometry analyzer, Cytoflex (Beckman Coulter) for data collection. Dot plots represent expansion and activation of CD4+ CD25+ and CD8+ CD25+ T-cells in anti-TCRαV-12/IL-2 treated cells compared to isotype controls (FIG. 3C).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby. Those skilled in the art will recognize, or be able to ascertain using no more than routine experimentation, many equivalents to the specific embodiments of the invention described herein. Such equivalents are intended to be encompassed by the following claims.

What is claimed is:

1.-160. (canceled)

161. A method of activating a T cell comprising:
(1) contacting the T cell with a multispecific molecule comprising a T cell receptor alpha variable region (TCRαV)-binding moiety linked to a targeting moiety, wherein the TCRαV-binding moiety binds to a TCRαV on the T cell and activates the T cell,
(2) thereby generating an activated T cell.

162. The method of claim 161, wherein the targeting moiety is configured to bind to and stimulate a costimulatory molecule on a surface of the T cell.

163. The method of claim 162, wherein the costimulatory molecule comprises CD27, CD28, 4-1BB (CD137), 0X40, CD30, CD40, PD-1, ICOS, lymphocyte function-associated antigen-1 (LFA-1), CD2, CD7, LIGHT, NKG2C, B7-H3, or a ligand that specifically binds with CD83.

164. The method of claim 161, wherein the targeting moiety comprises a cytokine molecule.

165. The method of claim 164, wherein the cytokine molecule is selected from the group consisting of interleukin-2 (IL-2) or a functional fragment or variant thereof, interleukin-7 (IL-7) or a functional fragment or variant thereof, interleukin-12 (IL-12) or a functional fragment or variant thereof, interleukin-15 (IL-15) or a functional fragment or variant thereof, interleukin-18 (IL-18) or a functional fragment or variant thereof, interleukin-21 (IL-21) or a functional fragment or variant thereof, and interferon gamma (IFNγ) or a functional fragment or variant thereof.

166. The method of claim 161, wherein the TCRαV-binding moiety is a Fab, a F(ab′)2, a Fv, a single domain antibody, a single chain Fv (scFv) a diabody (dAb), a bivalent antibody, a bispecific antibody or fragment thereof, or a camelid antibody.

167. The method of claim 161, wherein the multispecific molecule further comprises a tumor-targeting moiety, and wherein the tumor-targeting moiety binds to a cancer antigen on a surface of a target cell.

168. The method of claim 167, wherein the cancer antigen comprises a hematological cancer antigen, a solid tumor antigen, a metastatic cancer antigen, a soft tissue tumor antigen, a cancer antigen of a metastatic lesion or a stromal antigen.

169. The method of claim 167, further comprising redirecting the activated T cell to the target cell to promote cell lysis of the target cell.

170. The method of claim 161, wherein the contacting occurs in vivo.

171. The method of claim 161, wherein the TCRαV-binding moiety binds to one or more of a TCRαV subfamily selected from the group consisting of: a TCRα V1 subfamily, a TCRα V2 subfamily, a TCRα V3 subfamily, a TCRα V4, a TCRα V5 subfamily, a TCRα V6 subfamily, a TCRα V7 subfamily, a TCRα V8 subfamily, a TCRα V9 subfamily, a TCRα V10 subfamily, a TCRα V12 subfamily, a TCRα V13 subfamily, a TCRα V14 subfamily, a TCRα V16 subfamily, a TCRα V17 subfamily, a TCRα V18 subfamily, a TCRα V19 subfamily, a TCRα V20 subfamily, a TCRα V21 subfamily, a TCRα V22 subfamily, a TCRα V23 subfamily, a TCRα V24 subfamily, TCRα V25 subfamily, a TCRα V26 subfamily, a TCRα V27 subfamily, a TCRα V29 subfamily, a TCRα V30 subfamily, a TCRα V34 subfamily, a TCRα V35 subfamily, a TCRα V36 subfamily, a TCRα V38 subfamily, a TCRα V39 subfamily, a TCRα V40 subfamily, and a TCRα V41 subfamily.

172. The method of claim 161, wherein the multispecific molecule comprises at least two non-contiguous polypeptide chains,
wherein a first polypeptide chain of the at least two non-contiguous polypeptide chains comprises a first member of a dimerization module and a second polypeptide chain of the at least two non-contiguous polypeptide chains comprises a second member of the dimerization module, wherein the first polypeptide chain and the second polypeptide chain form a complex via the first member of the dimerization module and the second member of the dimerization module.

173. The method of claim 172, wherein the first member of the dimerization module is a first Fc region, and the second member of the dimerization module is a second Fc region.

174. The method of claim 172, wherein the multispecific molecule further comprises a linker between the TCRαV-binding moiety and the first member of the dimerization module, a linker between the targeting moiety and the second member of the dimerization module, a linker between a first portion of the TCRαV-binding moiety and the first member of the dimerization module, a linker between a first portion of the targeting moiety and the second member of the dimerization module, a linker between the first member of the dimerization module and the targeting moiety, a linker between the first member of the dimerization module and the first portion of the targeting moiety, or any combination thereof.

175. The method of claim 174, wherein the linker is selected from the group consisting of a cleavable linker, a non-cleavable linker, a peptide linker, a flexible linker, a rigid linker, a helical linker, and a non-helical linker.

176. A pharmaceutical composition comprising a multispecific molecule and a pharmaceutically acceptable diluent, carrier, excipient, or stabilizer, wherein the multispecific molecule comprises a T cell receptor alpha variable region (TCRαV)-binding moiety linked to a targeting moiety, and wherein the TCRαV-binding moiety binds to a TCRαV on a T cell and activates the T cell.

177. A method of treating a disease or condition in a subject in need thereof comprising: administering a therapeutically effective amount of the pharmaceutical composition of claim 176 to the subject in need thereof.

178. The method of claim 177, wherein the disease or condition is a cancer, an autoimmune disease, or an infection.

179. The method of claim 178, wherein:
(i) the cancer is breast cancer, and the TCRαV-binding moiety binds to a TCRα V1 subfamily;
(ii) the cancer is ER+ breast cancer, and the TCRαV-binding moiety binds to a TCRα V1 subfamily;
(iii) the cancer is ER+ breast cancer, and the TCRαV-binding moiety binds to a TCRα V3 subfamily;
(iv) the cancer is diffuse large B-cell lymphoma, and the TCRαV-binding moiety binds to a TCRα V6 subfamily;
(v) the cancer is diffuse large B-cell lymphoma, and the TCRαV-binding moiety binds to a TCRα V8 subfamily;
(vi) the cancer is breast cancer, and the TCRαV-binding moiety binds to a TCRα V9 subfamily;
(vii) the cancer is diffuse large B-cell lymphoma, and the TCRαV-binding moiety binds to a TCRα V12 subfamily;
(viii) the cancer is melanoma, and the TCRαV-binding moiety binds to a TCRα V12 subfamily;
(ix) the cancer is multiple myeloma, and the TCRαV-binding moiety binds to a TCRα V19 subfamily;
(x) the cancer is diffuse large B-cell lymphoma, and the TCRαV-binding moiety binds to a TCRα V21 subfamily;
(xi) the cancer is diffuse large B-cell lymphoma, and the TCRαV-binding moiety binds to a TCRα V22 subfamily;
(xii) the cancer is diffuse large B-cell lymphoma, and the TCRαV-binding moiety binds to a TCRα V25 subfamily;
(xiii) the cancer is leukemia, and the TCRαV-binding moiety binds to a TCRα V29 subfamily;
(xiv) the cancer is melanoma, and the TCRαV-binding moiety binds to a TCRα V29 subfamily;
(xv) the cancer is breast cancer, and the TCRαV-binding moiety binds to a TCRα V29 subfamily;
(xvi) the cancer is endometrial cancer, and the TCRαV-binding moiety binds to a TCRα V30 subfamily;
(xvii) the cancer is leukemia, and the TCRαV-binding moiety binds to a TCRα V38 subfamily;
(xviii) the cancer is esophageal squamous cell carcinoma, and the TCRαV-binding moiety binds to a TCRα V39 subfamily;
(xix) the autoimmune disease is multiple sclerosis, and the TCRαV-binding moiety binds to a TCRα V1 subfamily;
(xx) the autoimmune disease is Crohn's disease, and the TCRαV-binding moiety binds to a TCRα V2 subfamily;
(xxi) the autoimmune disease is Sjögren's syndrome, and the TCRαV-binding moiety binds to a TCRα V13 subfamily;
(xxii) the autoimmune disease is celiac disease, and the TCRαV-binding moiety binds to a TCRα V20 subfamily;
(xxiii) the autoimmune disease Crohn's disease, and the TCRαV-binding moiety binds to a TCRα V22 subfamily;
(xxiv) the autoimmune disease celiac disease, and the TCRαV-binding moiety binds to a TCRα V26 subfamily; or
(xxv) the autoimmune disease Crohn's disease, and the TCRαV-binding moiety binds to a TCRα V40 subfamily.

180. The method of claim 178, wherein:
(i) the infection is a S. parathyphi infection, and the TCRαV-binding moiety binds to a TCRα V1 subfamily;
(ii) the infection is a Bacteroidetes infection, and the TCRαV-binding moiety binds to a TCRα V1 subfamily;
(iii) the infection is a Proteobacteria infection, and the TCRαV-binding moiety binds to a TCRα V1 subfamily;
(iv) the infection is a M. tuberculosis infection, and the TCRαV-binding moiety binds to a TCRα V1 subfamily;
(v) the infection is a respiratory virus infection, and the TCRαV-binding moiety binds to a TCRα V6 subfamily;
(vi) the infection is a SARS-CoV-2 infection, and the TCRαV-binding moiety binds to a TCRα V7 subfamily;
(vii) the infection is a SARS-CoV-2 infection, and the TCRαV-binding moiety binds to a TCRα V9 subfamily;
(viii) the infection is a SARS-CoV-2 infection, and the TCRαV-binding moiety binds to a TCRα V11 subfamily;
(ix) the infection is a S. pyogenes infection, and the TCRαV-binding moiety binds to a TCRα V12 subfamily;
(x) the infection is a SARS-CoV-2 infection, and the TCRαV-binding moiety binds to a TCRα V12 subfamily;
(xi) the infection is yellow fever, and the TCRαV-binding moiety binds to a TCRα V12 subfamily;
(xii) the infection is influenza, and the TCRαV-binding moiety binds to a TCRα V13 subfamily;
(xiii) the infection is a respiratory virus infection, and the TCRαV-binding moiety binds to a TCRα V16 subfamily;
(xiv) the infection is a HIV infection, and the TCRαV-binding moiety binds to a TCRα V17 subfamily;
(xv) the infection is a Cytomegalovirus infection, and the TCRαV-binding moiety binds to a TCRα V17 subfamily;
(xvi) the infection is a SARS-CoV-2 infection, and the TCRαV-binding moiety binds to a TCRα V18 subfamily;
(xvii) the infection is a HIV infection, and the TCRαV-binding moiety binds to a TCRα V24 subfamily;
(xviii) the infection influenza, and the TCRαV-binding moiety binds to a TCRα V27 subfamily; and/or
(xix) the infection is an Epstein-Barr Virus infection, and the TCRαV-binding moiety binds to a TCRα V38 subfamily.
